SEQUENCE LISTING (17 sequences)

SEQ ID NO: 1 - protein, 300 aa, synthetic construct
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 2 - protein, 300 aa, synthetic construct
FACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 3 - RNA, 1356 nt, synthetic construct
  source 1..1356: mol_type = other RNA
aggaggactggtcgaacctgcatagtgatcataaggtcagcatagccaccatggagaccgacacactgctgctgtgggtgctgctgctgtgggtgcccggctccaccggcttcgcttgcaagacagccagcggaaccgccatccctatcggcgcagcaagcgccaacgtctacgtgaatctggctcccgcagtgaacgtgggacagaatctggtggtggatctgagcacccagatcttctgccacaatgactaccccgagaccatcacagactacgtgacactgcagagaggaagcgcctacggcggcgtgctgagcagcttcagcggaaccgtgaaatacagcggctccagctaccccttccccacaaccagcgagacccctagagtggtctataactctagaacagacaagccttggcccgtggctctgtatctgacccccgtgtccagcgctggaggagtggccatcaaggccggcagcctcatcgccgtcctcattctgaggcagaccaacaactacaacagcgacgacttccagttcgtgtggaacatctacgccaacaacgacgtggtggtccccaccggcggatgtgacgtgtccgccagagacgtgaccgtgacactgcccgattaccccggaagcgtccctatccctctgacagtgtactgcgccaagagccaaaatctgggctactatctgtccggaaccacagccgacgccggaaactccatcttcaccaacaccgccagcttttcccccgcccaaggagtgggagtccagctgacaagaagcggcaccatcatccccgccagcaacacagtgtctctgggcgctgtgggcacatccgctgtgtctctgggactgacagctaattatgccagaaccggaggccaagtgaccgctggaaatgtgcagagcattattggggtgaccttcgtgtaccagggcggaagtagcggaggcggtgccgacgtgaccatcaccgtgaacggcaaggtggtggccaagagttctggtggcggtggttcaagtggtagtggcagttcaagtgggacaacacgactgttgagcgggcatacgtgttttacgctgacaggtcttctgggcacgctggttactatgggcttgcttacgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - RNA, 47 nt, synthetic construct
  source 1..47: mol_type = other RNA
aggtgtcagagtttaacttgaagactatttctagggataatatccct

SEQ ID NO: 5 - RNA, 47 nt, synthetic construct
  source 1..47: mol_type = other RNA
agatgtcagagtttaacttgaagactatttctagggataatatccct

SEQ ID NO: 6 - RNA, 51 nt, synthetic construct
  source 1..51: mol_type = other RNA
aggaaataagagaggataagacgactaaggagacatacagaataagaggca

SEQ ID NO: 7 - RNA, 44 nt, synthetic construct
  source 1..44: mol_type = other RNA
aggaggactggtcgaacctgcatagtgatcataaggtcagcata

SEQ ID NO: 8 - RNA, 55 nt, synthetic construct
  source 1..55: mol_type = other RNA
aggaaataagagaggataagacgactaaggagacatacagaataagaaacaggca

SEQ ID NO: 9 - RNA, 55 nt, synthetic construct
  source 1..55: mol_type = other RNA
aggaaataagagaaagaggataagacgactaaggagacatacagaataagaggca

SEQ ID NO: 10 - RNA, 53 nt, synthetic construct
  source 1..53: mol_type = other RNA
aggacatttgcttctgacacaactgtgttcactagcaacctcaaacagccacc

SEQ ID NO: 11 - RNA, 54 nt, synthetic construct
  source 1..54: mol_type = other RNA
aggaataaactagtattcttctggtccccacagactcagagagaacccgccacc

SEQ ID NO: 12 - RNA, 134 nt, synthetic construct
  source 1..134: mol_type = other RNA
gctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 13 - RNA, 134 nt, synthetic construct; identical to SEQ ID NO: 12
  source 1..134: mol_type = other RNA

SEQ ID NO: 14 - RNA, 134 nt, synthetic construct; identical to SEQ ID NO: 12
  source 1..134: mol_type = other RNA

SEQ ID NO: 15 - RNA, 134 nt, synthetic construct; identical to SEQ ID NO: 12
  source 1..134: mol_type = other RNA

SEQ ID NO: 16 - RNA, 134 nt, synthetic construct; identical to SEQ ID NO: 12
  source 1..134: mol_type = other RNA

SEQ ID NO: 17 - RNA, 295 nt, synthetic construct
  source 1..295: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

(19) United States
(12) Patent Application Publication
(10) Pub. No.: US 2026/0166135 A1
(43) Pub. Date: Jun. 18, 2026

(54) LIPIDATED TLR7/8 MODULATORS AS ADJUVANTS AND USES THEREOF

(71) Applicant: Pfizer Inc., New York, NY (US)

(72) Inventors: Derek William Bartlett, Chapel Hill, NC (US); Bo Chen, Ridgewood, NJ (US); Matthew Scott Dowling, Old Lyme, CT (US); David Christopher Ebner, Newton, MA (US); KariAnn Sweeney Efferen, Lagrangeville, NY (US); Mark Edward Flanagan, Gales Ferry, CT (US); David Hepworth, Concord, MA (US); Ksenia Krylova, New Windsor, NY (US); Seyedehmahsa Moghimi, Paterson, NJ (US); Shibu Simon Philip, New Milford, NJ (US); Shuai Shi, Whippany, NJ (US); Yujie Su, Nanuet, NY (US); Joseph Scott Warmus, Ledyard, CT (US)

(21) Appl. No.: 19/366,679

(22) Filed: Oct. 23, 2025

Related U.S. Application Data
(60) Provisional application No. 63/711,459, filed on Oct. 24, 2024.
(60) Provisional application No. 63/886,205, filed on Sep. 23, 2025.

(51) Int. Cl. A61K 39/108; A61K 39/00; A61K 39/39; A61P 37/04; C07D 471/04

(52) U.S. Cl. CPC A61K 39/0258; A61K 39/39; A61P 37/04; C07D 471/04; A61K 2039/53; A61K 2039/55555; A61K 2039/55577

(57) ABSTRACT

The disclosure relates to compounds of Formula (I)

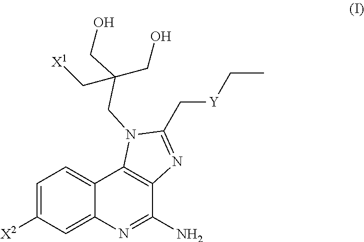

and pharmaceutically acceptable salts thereof, wherein X¹, X², and Y are as defined in the description; to their use in medicine; to compositions containing them; to processes for their preparation; and to intermediates used in such processes. The compounds of Formula (I) may modulate the activity of antigens of interest and may be useful in inducing or enhancing an immune response against diseases, disorders and conditions mediated by antigens of interest. In a particular embodiment, the compounds of Formula (I) may be useful as a component of a liposomal adjuvant formulation.

### RELATED APPLICATIONS

This application claims priority to U.S. Provisional Application No. 63/711,459 filed Oct. 24, 2024 and U.S. Provisional Application No. 63/886,205 filed Sep. 23, 2025. The entire content of each of the foregoing applications is herein incorporated by reference in its entirety.

### REFERENCE TO SEQUENCE LISTING

This application is being filed electronically via EFS-Web and includes an electronically submitted sequence listing in .xml format. The .xml file contains a sequence listing entitled “PC073184A Sequence Listing.xml” created on Sep. 24, 2025 and having a size of 19,033 bytes. The sequence listing contained in this xml file is part of the specification and is incorporated herein by reference in its entirety.

### BACKGROUND

The present disclosure relates to novel lipidated Toll-like receptor 7 (TLR7), Toll-like receptor 8 (TLR8), and Toll-like receptor 7/8 (TLR7/8) modulating adjuvant compounds. The disclosure also relates to the preparation of the compounds and intermediates used in the preparation, compositions containing the compounds, and uses of the compounds, including as adjuvants for antigens of interest.

Studies and research regarding adjuvant use as a vaccine component have significantly increased nowadays. Adjuvants are compounds that enhance immune system activation and recognition of a vaccine's active component, concerning subunit-based vaccines, as well as RNA-based vaccines.

Adjuvants act by enhancing the innate immune system's response magnitude, breadth, and durability. The potency of adjuvants is closely related to their ability to be recognized as pathogens/foreign bodies through pattern recognition receptors (PRRs). It is expected that a vaccine's active component will become recognized, thereby triggering a specific and long-lasting immune response to be mounted through the adaptive immune system (Excler et al., Nat. Med., 27 (2021), pp. 591-600).

Toll-like receptors (TLRs) are a family of transmembrane proteins that recognize structurally conserved molecules that are derived from and unique to pathogens, referred to as pathogen-associated molecular patterns (PAMPs), a sub-class of PRR. As such, TLRs function in the mammalian immune system as front-line sensors of pathogen-associated molecular patterns, detecting the presence of invading pathogens (Takeuchi and Akira 2010 Cell 140:805-820). TLR engagement in sentinel immune cells causes biosynthesis of selected cytokines (e.g., type I interferons), induction of costimulatory molecules, and increased antigen presentation capacity. These are important molecular mechanisms that activate innate and adaptive immune responses. Accordingly, agonists and antagonists of TLRs find use in modulating immune responses. TLR agonists are typically employed to stimulate immune responses, whereas TLR antagonists are typically employed to inhibit immune responses (Gosu et al 2012. Molecules 17:13503-13529).

The human genome contains 10 known functional TLRs, of these TLR3, TLR7, TLR8, and TLR9 sense nucleic acids and their degradation products. The distribution of TLR7, TLR8, and TLR9 is restricted to the endosomal compartments of cells and they are preferentially expressed in cells of the immune system. In the activated, dimeric receptor configuration TLR7 and TLR8 recognize single strand RNA at one ligand binding site and the ribonucleoside degradation products guanosine and uridine, respectively, (as well as small molecule ligands with related structural motifs) at a second ligand binding site (Zhang et al 2016 Immunity 45(4); 737-748: Tanji et al 2015 Nat Struct Mol Biol 22:109-115). Engagement of TLR7 in plasmacytoid dendritic cells leads to the induction of Type I interferon, which plays essential functions in the control of the adaptive immune response (Bao and Liu 2013 Protein Cell 4:40-5). Engagement of TLR8 in myeloid dendritic cells, monocytes and monocyte-derived dendritic cells induces a prominent pro-inflammatory cytokine profile, characterized by increased production of tumor necrosis factor alpha, interleukin-12, and IL-18 (Eigenbrod et al J Immunol, 2015, 195, 1092-1099). Thus, virtually all major types of monocytic and dendritic cells can be activated by agonists of TLR7 and TLR8 to become highly effective antigen-presenting cells, thereby promoting an effective innate and adaptive immune response. Most antigen presenting cell types express only one of these two receptors, accordingly small molecules with potent agonist activity against both TLR7 and TLR8 receptors are potentially more effective immune adjuvants than agonists specific for only one of these TLRs. Thus, a TLR7/TLR8 (TLR7/8) small molecule agonist with dual bioactivity would cause innate immune responses in a wider range of antigen presenting cells and other key immune cell types, including plasmacytoid and myeloid dendritic cells, monocytes, and B cells (van Haren et al 2016 J Immunol 197:4413-4424; Ganapathi et al 2015 Plos One 10(8).e0134640).

Albumin has been extensively studied as a natural vector for lymph node targeted drug delivery (Adv. Drug Delivery Rev. 2018, 130, 73-89). First, albumin (about 7 nm in size) is the most abundant protein in the blood, reaching a concentration of 40 mg/mL, but maintaining a relatively lower concentration, about 14 mg/mL, in the interstitial fluid. This concentration difference tends to drive albumin to the lymphatics instead of blood capillaries. Second, albumin has an extraordinarily long half-life and is continuously synthesized in the liver for circulation. Third, natural ligands, such as long aliphatic fatty acids and hydrophobic molecules, have been discovered to bind albumin with their complexed crystal structures also being resolved. The albumin hitchhiking lymph node targeting was first demonstrated with TLR9 ligands conjugated to lipids and natural hydrophobic molecules such as cholesterol (Nature 507, 519-522, 2014).

What is needed in the field is a TLR7/8 agonist targeted to accumulate in the lymph node to (a) activate the immune cells in the lymph node to adjuvant the vaccine antigen, while (b) minimizing systemic exposure to the TLR7/8 agonist in order to circumvent systemic inflammation.

Accordingly, there remains a need for improved adjuvants targeted to accumulate in the lymph node. The current disclosure relates to the use of potent dual TLR7/8 agonists conjugated to a lipid.

### SUMMARY OF THE DISCLOSURE

The present disclosure provides, in part, compounds of Formula (I) and pharmaceutically acceptable salts thereof. Such compounds may agonize or modulate the activity of TLR7 and/or TLR8 and may be useful as vaccine adjuvants. Also provided are pharmaceutical compositions comprising the compounds or salts of the disclosure, alone or in combination with additional therapeutic agents. The present disclosure also provides, in part, methods for preparing such compounds, pharmaceutically acceptable salts and compositions of the disclosure, and methods of using the foregoing. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

According to an embodiment of the disclosure there is provided a compound of Formula (I)

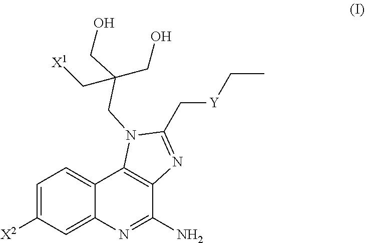

or a pharmaceutically acceptable salt thereof, wherein:
Y is —O— or —CH₂—; and
one of X¹ and X² is H, and the other of X¹ and X² has a formula selected from the group consisting of formula (a), formula (b), and formula (c):

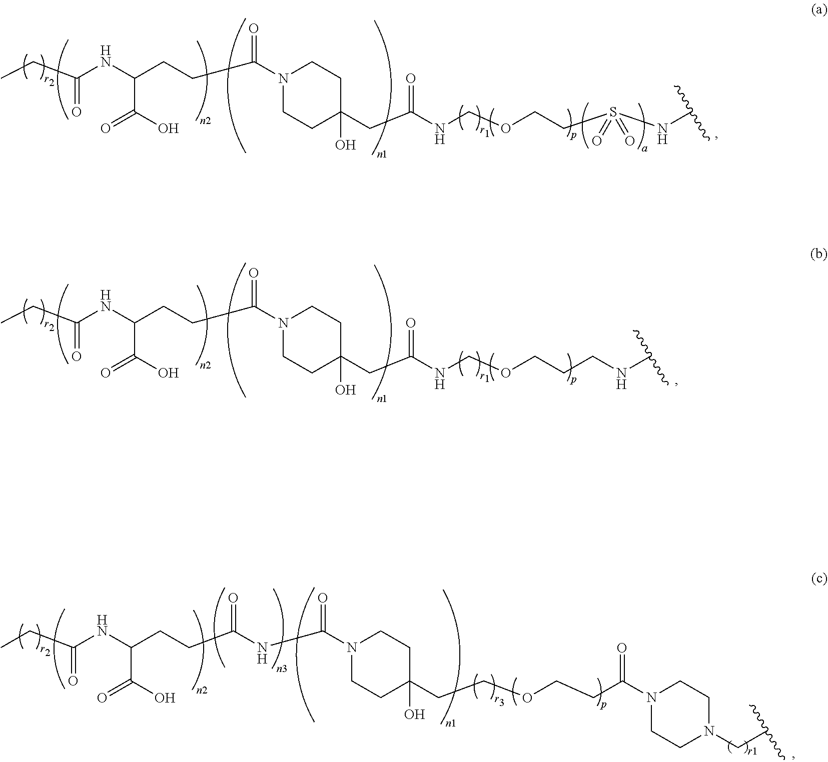

wherein:
a is 0 or 1;
r1 is an integer from 2 to 6;
r2 is an integer from 10 to 20;
r3 is an integer from 0 to 6;
n1 is 0 or 1 and n2 is 0 or 1, wherein at least one of n1 and n2 is 1;
n3 is 0 or 1; and
p is an integer from 0 to 6.

Described below are embodiments of the disclosure, where for convenience Embodiment 1 (E1) is identical to the embodiment of Formula (I) provided above.

It is to be understood that both the foregoing general description and the following detailed description are exemplary and explanatory only and are not restrictive of the disclosure, as claimed.

SEQUENCE IDENTIFIERS
SEQ ID NO: 1 sets forth an amino acid sequence
for wild type E. coli full-length FimH, including
the donor strand FimG peptide connected through
a linker (FimH-DSG_WT)
FACKTASGTAIPIGGGSANVYVNLAPVVNVGQNLVVDLSTQIFCHNDYP
ETITDYVTLQRGSAYGGVLSSFSGTVKYSGSSYPFPTTSETPRVVYNSR
TDKPWPVALYLTPVSSAGGVAIKAGSLIAVLILRQTNNYNSDDFQFVWN
IYANNDVVVPTGGCDVSARDVTVTLPDYPGSVPIPLTVYCAKSQNLGYY
LSGTTADAGNSIFTNTASFSPAQGVGVQLTRQGTIIPANNTVSLGAVGT
SAVSLGLTANYARTGGQVTAGNVQSIIGVTFVYQGGSSGGGADVTITVN
GKVVAK
SEQ ID NO: 2 sets forth an amino acid sequence
for the mutant E. coli FimHDSG_G15A_G16A_V27A
FACKTASGTAIPIGAASANVYVNLAPAVNVGQNLVVDLSTQIFCHNDYP
ETITDYVTLQRGSAYGGVLSSFSGTVKYSGSSYPFPTTSETPRVVYNSR
TDKPWPVALYLTPVSSAGGVAIKAGSLIAVLILRQTNNYNSDDFQFVWN
IYANNDVVVPTGGCDVSARDVTVTLPDYPGSVPIPLTVYCAKSQNLGYY
LSGTTADAGNSIFTNTASFSPAQGVGVQLTRQGTIIPANNTVSLGAVGT
SAVSLGLTANYARTGGQVTAGNVQSIIGVTFVYQGGSSGGGADVTITVN
GKVVAK
SEQ ID NO: 3 sets forth the nucleic acid sequence
for BMD576/FimHDSG-SerGlyGPI/hHBB_80pA
AGGAGGACUGGUCGAACCUGCAUAGUGAUCAUAAGGUCAGCAUAGCCAC
CAUGGAGACCGACACACUGCUGCUGUGGGUGCUGCUGCUGUGGGUGCCC
GGCUCCACCGGCUUCGCUUGCAAGACAGCCAGCGGAACCGCCAUCCCUA
UCGGCGCAGCAAGCGCCAACGUCUACGUGAAUCUGGCUCCCGCAGUGAA
CGUGGGACAGAAUCUGGUGGUGGAUCUGAGCACCCAGAUCUUCUGCCAC
AAUGACUACCCCGAGACCAUCACAGACUACGUGACACUGCAGAGAGGAA
GCGCCUACGGCGGCGUGCUGAGCAGCUUCAGCGGAACCGUGAAAUACAG
CGGCUCCAGCUACCCCUUCCCCACAACCAGCGAGACCCCUAGAGUGGUC
UAUAACUCUAGAACAGACAAGCCUUGGCCCGUGGCUCUGUAUCUGACCC
CCGUGUCCAGCGCUGGAGGAGUGGCCAUCAAGGCCGGCAGCCUCAUCGC
CGUCCUCAUUCUGAGGCAGACCAACAACUACAACAGCGACGACUUCCAG
UUCGUGUGGAACAUCUACGCCAACAACGACGUGGUGGUCCCCACCGGCG
GAUGUGACGUGUCCGCCAGAGACGUGACCGUGACACUGCCCGAUUACCC
CGGAAGCGUCCCUAUCCCUCUGACAGUGUACUGCGCCAAGAGCCAAAAU
CUGGGCUACUAUCUGUCCGGAACCACAGCCGACGCCGGAAACUCCAUCU
UCACCAACACCGCCAGCUUUUCCCCCGCCCAAGGAGUGGGAGUCCAGCU
GACAAGAAGCGGCACCAUCAUCCCCGCCAGCAACACAGUGUCUCUGGGC
GCUGUGGGCACAUCCGCUGUGUCUCUGGGACUGACAGCUAAUUAUGCCA
GAACCGGAGGCCAAGUGACCGCUGGAAAUGUGCAGAGCAUUAUUGGGGU
GACCUUCGUGUACCAGGGCGGAAGUAGCGGAGGCGGUGCCGACGUGACC
AUCACCGUGAACGGCAAGGUGGUGGCCAAGAGUUCUGGUGGCGGUGGUU
CAAGUGGUAGUGGCAGUUCAAGUGGGACAACACGACUGUUGAGCGGGCA
UACGUGUUUUACGCUGACAGGUCUUCUGGGCACGCUGGUUACUAUGGGC
UUGCUUACGUGAUGAGCUCGCUUUCUUGCUGUCCAAUUUCUAUUAAAGG
UUCCUUUGUUCCCUAAGUCCAACUACUAAACUGGGGGAUAUUAUGAAGG
GCCUUGAGCAUCUGGAUUCUGCCUAAUAAAAAACAUUUAUUUUCAUUGC
AAAAAAAAAAAAAAAAAAAAAAAAAAAAAAAAAAAAAAAAAAAAAAAAA
AAAAAAAAAAAAAAAAAAAAAAAAAAAAAAAAA

Sequence annotations are as follows: AUG, first methionine of the gene of interest (bold); UGAUGA stop codons after gene of interest (bold and italics); 5′UTR is underlined; polyA, 80nt tract (italics).

SEQ ID NO: 4 sets forth the nucleic acid sequence
for 5′UTR_BMD582
AGGTGTCAGAGTTTAACTTGAAGACTATTTCTAGGGATAATATCCCT
SEQ ID NO: 5 sets forth the nucleic acid sequence
for 5′UTR_BMD582-AGA
AGATGTCAGAGTTTAACTTGAAGACTATTTCTAGGGATAATATCCCT
SEQ ID NO: 6 sets forth the nucleic acid sequence
for 5′UTR_BMD2
AGGAAAUAAGAGAGGAUAAGACGACUAAGGAGACAUACAGAAUAAGAGG
CA
SEQ ID NO: 7 sets forth the nucleic acid sequence
for 5′UTR_BMD576
AGGAGGACUGGUCGAACCUGCAUAGUGAUCAUAAGGUCAGCAUA
SEQ ID NO: 8 sets forth the nucleic acid sequence
for 5′UTR_BMD563
AGGAAAUAAGAGAGGAUAAGACGACUAAGGAGACAUACAGAAUAAGAAA
CAGGCA
SEQ ID NO: 9 sets forth the nucleic acid sequence
for 5′UTR_BMD562
AGGAAAUAAGAGAAAGAGGAUAAGACGACUAAGGAGACAUACAGAAUAA
GAGGCA
SEQ ID NO: 10 sets forth the nucleic acid sequence
for 5′UTR_hHBB
AGGACAUUUGCUUCUGACACAACUGUGUUCACUAGCAACCUCAAACAGC
CACC
SEQ ID NO: 11 sets forth the nucleic acid sequence
for 5′UTR_WHO
AGGAAUAAACUAGUAUUCUUCUGGUCCCCACAGACUCAGAGAGAACCCG
CCACC
SEQ ID NO: 12 sets forth the nucleic acid sequence
for 3′UTR_hHBB
GCTCGCTTTCTTGCTGTCCAATTTCTATTAAAGGTTCCTTTGTTCCCTA
AGTCCAACTACTAAACTGGGGGATATTATGAAGGGCCTTGAGCATCTGG
ATTCTGCCTAATAAAAAACATTTATTTTCATTGCAA
SEQ ID NO: 13 sets forth the nucleic acid sequence
for 3′UTR_BMD2
GCUCGCUUUCUUGCUGUCCAAUUUCUAUUAAAGGUUCCUUUGUUCCCUA
AGUCCAACUACUAAACUGGGGGAUAUUAUGAAGGGCCUUGAGCAUCUGG
AUUCUGCCUAAUAAAAAACAUUUAUUUUCAUUGCAA
SEQ ID NO: 14 sets forth the nucleic acid sequence
for 3′UTR_BMD576
GCUCGCUUUCUUGCUGUCCAAUUUCUAUUAAAGGUUCCUUUGUUCCCUA
AGUCCAACUACUAAACUGGGGGAUAUUAUGAAGGGCCUUGAGCAUCUGG
AUUCUGCCUAAUAAAAAACAUUUAUUUUCAUUGCAA
SEQ ID NO: 15 sets forth the nucleic acid sequence
for 3′UTR_BMD563
GCUCGCUUUCUUGCUGUCCAAUUUCUAUUAAAGGUUCCUUUGUUCCCUA
AGUCCAACUACUAAACUGGGGGAUAUUAUGAAGGGCCUUGAGCAUCUGG
AUUCUGCCUAAUAAAAAACAUUUAUUUUCAUUGCAA
SEQ ID NO: 16 sets forth the nucleic acid sequence
for 3′UTR_BMD562
GCUCGCUUUCUUGCUGUCCAAUUUCUAUUAAAGGUUCCUUUGUUCCCUA
AGUCCAACUACUAAACUGGGGGAUAUUAUGAAGGGCCUUGAGCAUCUGG
AUUCUGCCUAAUAAAAAACAUUUAUUUUCAUUGCAA
SEQ ID NO: 17 sets forth the nucleic acid sequence
for 3′UTR_WHO
CUCGAGCUGGUACUGCAUGCACGCAAUGCUAGCUGCCCCUUUCCCGUCC
UGGGUACCCCGAGUCUCCCCCGACCUCGGGUCCCAGGUAUGCUCCCACC
UCCACCUGCCCCACUCACCACCUCUGCUAGUUCCAGACACCUCCCAAGC
ACGCAGCAAUGCAGCUCAAAACGCUUAGCCUAGCCACACCCCCACGGGA
AACAGCAGUGAUUAACCUUUAGCAAUAAACGAAAGUUUAACUAAGCUAU
ACUAACCCCAGGGUUGGUCAAUUUCGUGCCAGCCACACCCUGGAGCUAG
C

### DETAILED DESCRIPTION OF THE DISCLOSURE

The lipidated compounds, combinations, and methods of the present disclosure are believed to have one or more advantages, such as delivering the TLR7/8 modulating adjuvant to the lymph node via albumin trafficking. Without being bound to a particular mechanism or theory, the addition of a lipid moiety to the TLR7/8 modulating molecule may enhance binding of said molecule to interstitial albumin at the injection site. Binding to interstitial albumin may in turn, enhance trafficking of the TLR7/8 modulating molecule to the draining lymph node through afferent lymphatic vessels. Retention of the adjuvant in the lymph node via albumin trafficking may provide sustained exposure and accompanied stimulation of the immune system to allow for optimal immune response to the vaccine antigen.

Furthermore, the lipidated compounds of the present disclosure provide advantages versus non-lipidated compounds in facilitating the integration of said compounds into a liposomal adjuvant formulation. For example, when the compounds of the disclosure are combined with lipid components (i.e., phospholipids, cholesterols, etc.), the hydrophobic moieties within the TLR 7/8 modulating compounds of the present disclosure may facilitate the formation of a liposome comprising said compounds. Without being bound to a particular mechanism or theory, via integration into a liposome, the lipidated compounds of the present disclosure may have an enhanced ability to move throughout the lymphatic system which results in increased adjuvant activity.

The present disclosure may be understood more readily by reference to the following detailed description of the embodiments of the disclosure and the Examples included herein. It is to be understood that this disclosure is not limited to specific synthetic methods of making that may of course vary. It is to be also understood that the terminology used herein is for the purpose of describing specific embodiments only and is not intended to be limiting.

Exemplary embodiments (E) of the disclosure provided herein include:
E1 A compound of Formula (I) or a pharmaceutically acceptable salt thereof, as defined above.
E2 The compound of embodiment E1, wherein X¹ has formula a.
E3 The compound of embodiment E2, wherein a is 1, n1 is 0, and n2 is 1.
E4 The compound of any one of embodiments E1-E3, wherein r1 is 4.
E5 The compound of any one of embodiments E1-E4, wherein p is 0.
E6 The compound of embodiment E2, wherein a is 1, n1 is 0, and n2 is 1.
E7 The compound of embodiment E6, wherein r1 is 2 and p is 3.
E8 The compound of embodiment E1, wherein X¹ has formula b.
E9 The compound of embodiment E8, wherein n1 is 0 and n2 is 1.
E10 The compound of embodiment E9, wherein r1 is 2 and p is 3.
E11 The compound of embodiment E8, wherein n1 is 1 and n2 is 1.
E12 The compound of embodiment E11, wherein r1 is 3 and p is 0.
E13 The compound of embodiment E1, wherein X² has formula c.
E14 The compound of embodiment E13, wherein n1 is 1, n2 is 0, and n3 is 0.
E15 The compound of embodiment E14, wherein r1 is 3, r3 is 0, and p is 0.
E16 The compound of embodiment E13, wherein n1 is 0, n2 is 1, and n3 is 1.
E17 The compound of embodiment E16, wherein r1 is 3, r3 is 2, and p is 3.
E18 The compound of any one of embodiments E1-E17, or a pharmaceutically acceptable salt thereof, wherein r2 is 13, 14, or 15.
E19 The compound of any one of embodiments E1-E18, or a pharmaceutically acceptable salt thereof, wherein r2 is 14.
E20 A compound, which is N⁵-(4-(N-(3-(4-Amino-2-butyl-1H-imidazo[4,5-c]quinolin-1-yl)-2,2-bis(hydroxymethyl)propyl)sulfamoyl)butyl)-N²-palmitoyl-L-glutamine, or a pharmaceutically acceptable salt thereof.
E21 A compound, which is (S)-1-(N-(3-(4-Amino-2-butyl-1H-imidazo[4,5-c]quinolin-1-yl)-2,2-bis(hydroxymethyl)propyl)sulfamoyl)-13-oxo-16-palmitamido-3,6,9-trioxa-12-azaheptadecan-17-oic acid, or a pharmaceutically acceptable salt thereof.
E22 A compound, which is (S)-1-(4-Amino-2-butyl-1H-imidazo[4,5-c]quinolin-1-yl)-2,2-bis(hydroxymethyl)-18-oxo-21-palmitamido-8,11,14-trioxa-4,17-diazadocosan-22-oic acid, or a pharmaceutically acceptable salt thereof.
E23 A compound, which is (S)-5-(4-(2-((4-((3-(4-Amino-2-butyl-1H-imidazo[4,5-c]quinolin-1-yl)-2,2-bis(hydroxymethyl)propyl)amino)butyl)amino)-2-oxoethyl)-4-hydroxypiperidin-1-yl)-5-oxo-2-palmitamidopentanoic acid, or a pharmaceutically acceptable salt thereof.
E24 A compound, which is 1-(4-(2-(4-(3-(4-Amino-2-butyl-1-(3-hydroxy-2-(hydroxymethyl)-2-methylpropyl)-1H-imidazo[4,5-c]quinolin-7-yl)propyl) piperazin-1-yl)-2-oxoethyl)-4-hydroxypiperidin-1-yl)hexadecan-1-one, or a pharmaceutically acceptable salt thereof.
E25 A compound, which is (S)-1-(4-(3-(4-Amino-2-(ethoxymethyl)-1-(3-hydroxy-2-(hydroxymethyl)-2-methylpropyl)-1H-imidazo[4,5-c]quinolin-7-yl)propyl) piperazin-1-yl)-1,14-dioxo-17-palmitamido-4,7,10-trioxa-13-azaoctadecan-18-oic acid, or a pharmaceutically acceptable salt thereof.
E26 A pharmaceutical composition comprising the compound according to any one of embodiments E1-E25, or a pharmaceutically acceptable salt thereof, and at least one pharmaceutically acceptable excipient.
E27 A crystalline form of the compound according to any one of embodiments E1-E25 or a pharmaceutically acceptable salt thereof.
E28 A method of inducing an immune response to an antigen of interest in a subject, comprising administering to the subject the pharmaceutical composition of embodiment E26, wherein the composition further comprises the antigen of interest.
E29 A method for immunizing a subject against a disease or disorder caused by or associated with an antigen of interest, comprising administering to the subject the pharmaceutical composition of embodiment E26, wherein the composition further comprises the antigen of interest.
E30 A method for preventing a disease or disorder caused by or associated with an antigen of interest in a subject, comprising administering to the subject the pharmaceutical composition of embodiment E26, wherein the composition further comprises the antigen of interest.
E31 A method for treating a disease or disorder caused by or associated with an antigen of interest in a subject, comprising administering to the subject the pharmaceutical composition of embodiment E26, wherein the composition further comprises the antigen of interest.
E32 A method for increasing an immune response to an antigen of interest in a subject, comprising administering to the subject the pharmaceutical composition of embodiment E26, wherein the composition further comprises the antigen of interest.
E33 The method of any one of embodiments E28 to E32, wherein the antigen of interest is an infectious disease antigen.
E34 The method of any one of embodiments E28 to E33, wherein the antigen of interest is a viral antigen, a bacterial antigen, a fungal antigen, or a parasitic antigen.
E35 The method of any one of embodiments E28 to E32, wherein the antigen of interest is a cancer antigen.
E36 The method of any one of embodiments E28-E35, wherein the method induces an immune response in the subject to the antigen of interest and the immune response is at least 10%, at least 20%, at least 30%, at least 40%, at least 50%, at least 60%, at least 70%, at least 80%, at least 90%, or at least 100%, higher than the immune response of the subject induced by a composition comprising the antigen of interest without the compound according to any one of embodiments E1 to E25.
E37 The method of embodiment E36, wherein the immune response is measured by opsonophagocytic activity (OPA) geometric mean antibody titers.
E38 The method of embodiment E36, wherein the immune response is measured by neutralization geometric mean antibody titers.
E39 The method of any one of embodiments E28 to E38, wherein the subject is human.
E40 A compound according to any one of embodiments E1 to E25 for use as a medicament.
E41 A compound according to any one of embodiments E1 to E25 for use in inducing an immune response to an antigen of interest in a subject.
E42 A compound according to any one of embodiments E1 to E25 for use in immunizing a subject against a disease or disorder caused by or associated with an antigen of interest in a subject.
E43 A compound according to any one of embodiments E1 to E25 for use in preventing a disease or disorder caused by or associated with an antigen of interest in a subject.
E44 A compound according to any one of embodiments E1 to E25 for use in treating a disease or disorder caused by or associated with an antigen of interest in a subject.
E45 A compound according to any one of embodiments E1 to E25 for use in increasing an immune response to an antigen of interest in a subject.
E46 The compound according to any one of embodiments E40-E45, wherein the subject is a human.
E47 Use of a compound according to any one of embodiments E1 to E25 for the manufacture of a medicament for inducing an immune response to an antigen of interest in a subject.
E48 Use of a compound according to any one of embodiments E1 to E25 for the manufacture of a medicament for immunizing a subject against a disease or disorder caused by or associated with an antigen of interest.
E49 Use of a compound according to any one of embodiments E1 to E25 for the manufacture of a medicament for use in preventing a disease or disorder caused by or associated with an antigen of interest in the subject.
E50 Use of a compound according to any one of embodiments E1 to E25 for the manufacture of a medicament for treating a disease or disorder caused by or associated with an antigen of interest in the subject.
E51 Use of a compound according to any one of embodiments E1 to E25 for the manufacture of a medicament for increasing an immune response to an antigen of interest in a subject.
E52 The use according to any one of embodiments E47 to E51, wherein the subject is a human.
E53 A lipid nanoparticle (LNP) formulation, comprising a plurality of LNPs, wherein the LNPs comprise a compound of Formula (I):

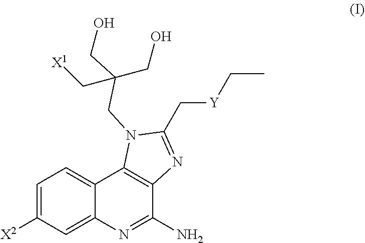

or a pharmaceutically acceptable salt thereof, wherein:
Y is —O— or —CH₂—; and
one of X¹ and X² is H, and the other of X¹ and X² has a formula selected from the group consisting of formula (a), formula (b), and formula (c):

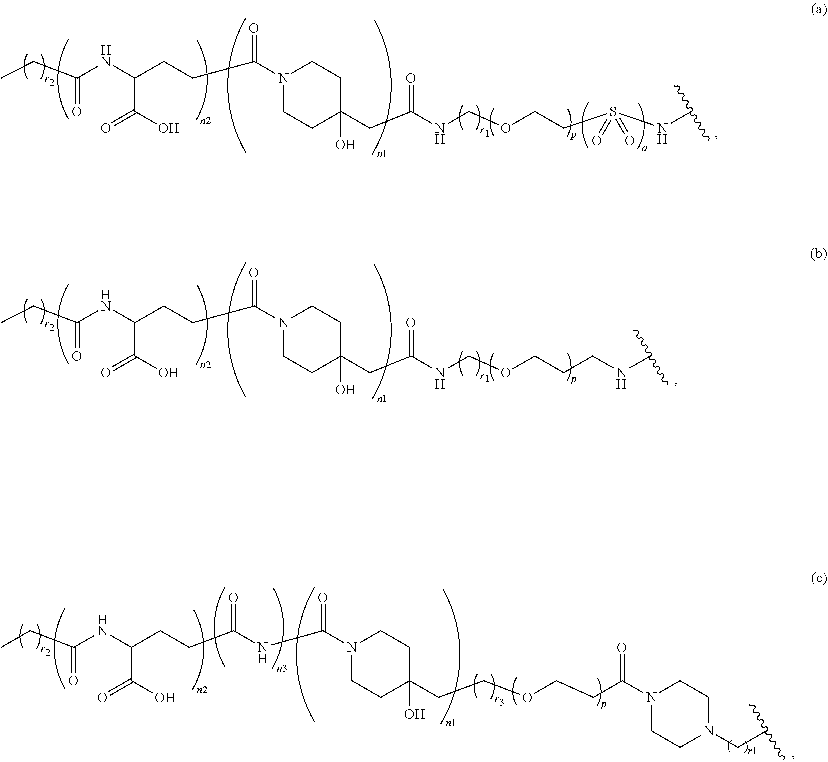

wherein:
a is 0 or 1;
r1 is an integer from 2 to 6;
r2 is an integer from 10 to 20;
r3 is an integer from 0 to 6;
n1 is 0 or 1 and n2 is 0 or 1, wherein at least one of n1 and n2 is 1;
n3 is 0 or 1; and
p is an integer from 0 to 6.
E54 The LNP formulation of E53, wherein X¹ has formula a.
E55 The LNP formulation of E54, wherein a is 1, n1 is 0, and n2 is 1.
E56 The LNP formulation of any one of E53-E55, wherein r1 is 4.
E57 The LNP formulation of any one of E53-E56, wherein p is 0.
E58 The LNP formulation of E54, wherein a is 1, n1 is 0, and n2 is 1.
E59 The LNP formulation of E58, wherein r1 is 2 and p is 3.
E60 The LNP formulation of E53, wherein X¹ has formula b.
E61 The LNP formulation of E60, wherein n1 is 0 and n2 is 1.
E62 The LNP formulation of E61, wherein r1 is 2 and p is 3.
E63 The LNP formulation of E60, wherein n1 is 1 and n2 is 1.
E64 The LNP formulation of E63, wherein r1 is 3 and p is 0.
E65 The LNP formulation of E53, wherein X² has formula c.
E66 The LNP formulation of E65, wherein n1 is 1, n2 is 0, and n3 is 0.
E67 The LNP formulation of E66, wherein r1 is 3, r3 is 0, and p is 0.
E68 The LNP formulation of E65, wherein n1 is 0, n2 is 1, and n3 is 1.
E69 The LNP formulation of E68, wherein r1 is 3, r3 is 2, and p is 3.
E70 The LNP formulation of any one of E53-E69, wherein r2 is 13, 14, or 15.
E71 The LNP formulation of any one of E53-E70, wherein r2 is 14.
E72 A lipid nanoparticle (LNP) formulation, comprising a plurality of LNPs, wherein the LNPs comprise a compound, which is N⁵-(4-(N-(3-(4-Amino-2-butyl-1H-imidazo[4,5-c]quinolin-1-yl)-2,2-bis(hydroxymethyl)propyl)sulfamoyl)butyl)-N²-palmitoyl-L-glutamine, or a pharmaceutically acceptable salt thereof.
E73 A lipid nanoparticle (LNP) formulation, comprising a plurality of LNPs, wherein the LNPs comprise a compound, which is (S)-1-(N-(3-(4-Amino-2-butyl-1H-imidazo[4,5-c]quinolin-1-yl)-2,2-bis(hydroxymethyl)propyl)sulfamoyl)-13-oxo-16-palmitamido-3,6,9-trioxa-12-azaheptadecan-17-oic acid, or a pharmaceutically acceptable salt thereof.
E74 A lipid nanoparticle (LNP) formulation, comprising a plurality of LNPs, wherein the LNPs comprise a compound, which is (S)-1-(4-Amino-2-butyl-1H-imidazo[4,5-c]quinolin-1-yl)-2,2-bis(hydroxymethyl)-18-oxo-21-palmitamido-8,11,14-trioxa-4,17-diazadocosan-22-oic acid, or a pharmaceutically acceptable salt thereof.
E75 A lipid nanoparticle (LNP) formulation, comprising a plurality of LNPs, wherein the LNPs comprise a compound, which is (S)-5-(4-(2-((4-((3-(4-Amino-2-butyl-1H-imidazo[4,5-c]quinolin-1-yl)-2,2-bis(hydroxymethyl)propyl)amino)butyl)amino)-2-oxoethyl)-4-hydroxypiperidin-1-yl)-5-oxo-2-palmitamidopentanoic acid, or a pharmaceutically acceptable salt thereof.
E76 A lipid nanoparticle (LNP) formulation, comprising a plurality of LNPs, wherein the LNPs comprise a compound, which is 1-(4-(2-(4-(3-(4-Amino-2-butyl-1-(3-hydroxy-2-(hydroxymethyl)-2-methylpropyl)-1H-imidazo[4,5-c]quinolin-7-yl)propyl) piperazin-1-yl)-2-oxoethyl)-4-hydroxypiperidin-1-yl)hexadecan-1-one, or a pharmaceutically acceptable salt thereof.
E77 A lipid nanoparticle (LNP) formulation, comprising a plurality of LNPs, wherein the LNPs comprise a compound, which is (S)-1-(4-(3-(4-Amino-2-(ethoxymethyl)-1-(3-hydroxy-2-(hydroxymethyl)-2-methylpropyl)-1H-imidazo[4,5-c]quinolin-7-yl)propyl) piperazin-1-yl)-1, 14-dioxo-17-palmitamido-4,7,10-trioxa-13-azaoctadecan-18-oic acid, or a pharmaceutically acceptable salt thereof.
E78 The LNP formulation of any one of E53-E77, wherein the LNPs further comprise:
   a) an ionizable cationic lipid;
   b) cholesterol, a cholesterol analog, or cholesterol and a cholesterol analog;
   c) a neutral lipid; and
   d) a polymer-conjugated lipid.
E79 The LNP formulation of E78, wherein the LNPs further comprise RNA.
E80 The LNP formulation of E79, wherein the RNA is modified RNA (modRNA) or self-amplifying RNA (saRNA).
E81 The LNP formulation of E79 or E80, wherein the RNA comprises a 5′ cap, a 5′ UTR, a 3′ UTR, and a poly-A tail.
E82 The LNP formulation of E81, wherein the 5′ cap is a 5′ cap analog.
E83 The LNP formulation of any one of E79-E82, wherein the RNA comprises a modified nucleotide selected from the group consisting of pseudouridine, 1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, 2′-O-methyl uridine, and N1-Methylpseudourodine-5′-triphosphate (m1ΨTP).
E84 The LNP formulation of any one of E79-E83, wherein the RNA comprises N1-Methylpseudourodine-5′-triphosphate (m1ΨTP).
E85 The LNP formulation of any one of E79-E84, wherein the RNA comprises a codon-optimized open reading frame.
E86 The LNP formulation of any one of E79-E85, wherein the molar ratio of the nitrogen atoms in the ionizable cationic lipid to the phosphate groups in the RNA (N:P ratio) is between about 2:1 and about 20:1.
E87 The LNP formulation of E86, wherein the N:P ratio is about 6:1.
E88 The LNP formulation of any one of E78-E87, wherein the LNPs further comprise a saponin.
E89 The LNP formulation of E88, wherein the saponin is QS-7, QS-18, QS-21, or a mixture thereof.
E90 The LNP formulation of E89, wherein the saponin is QS-21.
E91 The LNP formulation of any one of E78-E90, wherein the ionizable cationic lipid is N,N-dimethyl-2,3-dioleyloxy)propylamine (DODMA), N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-KC2-DMA), 2,2-dilinoleyl-4-dimethylaminomethyl-[1,3]-dioxolane (DLin-K-DMA), (4-hydroxybutyl)azanediyl)bis(hexane-6,1-diyl)bis(2-hexyldecanoate) (ALC-0315), 2-hexyldecyl 6-[(2-{[4-(heptylcarbonylamino)butyl]-N-methylamino}ethyl) [5-(2-hexyldecyloxycarbonyl)pentyl]amino]hexanoate (ALC-0515), 2-(7-(4-hydroxybutyl)-2,7-diazaspiro[3.5]nonan-2-yl)propane-1,3-diyl bis(2-heptylnonanoate) (PF-9032), heptadecane-9-yl 8-((2-hydroxyethyl) (6-oxo-6-(undecyloxy) hexyl)amino) octanoate (SM-102), or a mixture thereof.
E92 The LNP formulation of any one of E78-E91, wherein the ionizable cationic lipid is ALC-0315 (4-hydroxybutyl)azanediyl)bis(hexane-6,1-diyl)bis(2-hexyldecanoate) (ALC-0315) having the structure:

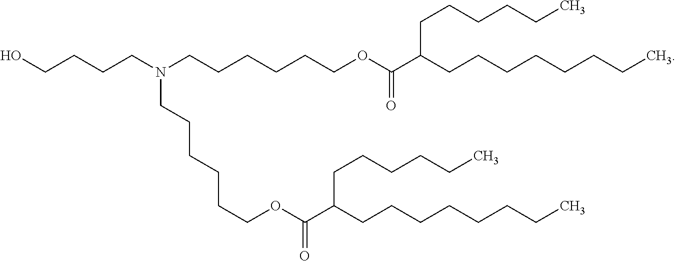 [SMILES: CCCCCCCCC(CCCCCC)C(=O)OCCCCCCN(CCCCO)CCCCCCOC(=O)C(CCCCCC)CCCCCCCC]

E93 The LNP formulation of any one of E78-E91, wherein the ionizable cationic lipid is 2-hexyldecyl 6-[(2-{[4-(heptylcarbonylamino)butyl]-N-methylamino}ethyl) [5-(2-hexyldecyloxycarbonyl)pentyl]amino]hexanoate (ALC-0515) having the structure:

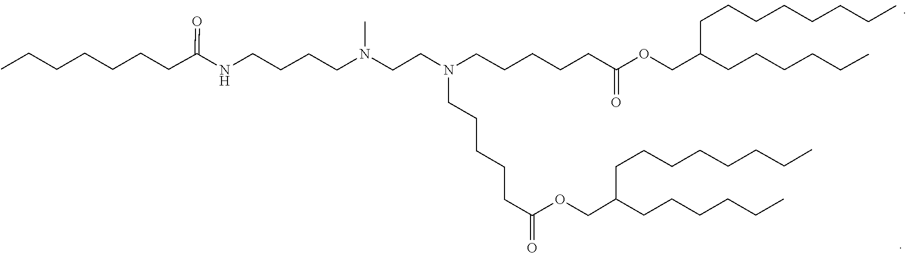 [SMILES: CCCCCCCCC(CCCCCC)COC(=O)CCCCCN(CCCCCC(=O)OCC(CCCCCC)CCCCCCCC)CCN(C)CCCCNC(=O)CCCCCCC]

E94 The LNP formulation of any one of E78-E91, wherein the ionizable cationic lipid is 2-(7-(4-hydroxybutyl)-2,7-diazaspiro[3.5]nonan-2-yl)propane-1,3-diyl bis(2-heptylnonanoate) (PF-9032) having the structure:

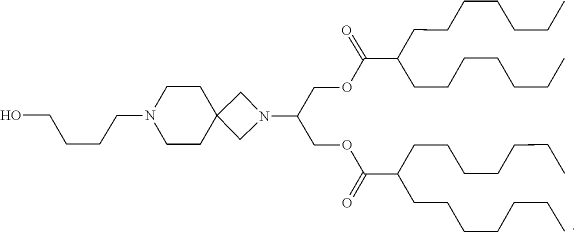 [SMILES: CCCCCCCC(CCCCCCC)C(=O)OCC(COC(=O)C(CCCCCCC)CCCCCCC)N1CC2(CCN(CCCCO)CC2)C1]

E95 The LNP formulation of any one of E78-E94, wherein the LNPs comprise a cholesterol analog selected from the group consisting of sitosterol, stigmasterol, campesterol, sitostanol, campestanol, brassicasterol, fucosterol, β-sitosterol, stigmastanol, β-sitostanol, ergosterol, fecosterol, lupeol, cycloartenol, Δ5-avenasterol, Δ7-avenasterol, Δ7-stigmasterol, tomatidine, ursolic acid, and alpha-tocopherol, including analogs, salts or esters thereof.
E96 The LNP formulation of any one of E78-E95, wherein the LNPs comprise cholesterol and a cholesterol analog, and wherein the cholesterol analog is β-sitosterol.
E97 The LNP formulation of E96, wherein the molar ratio of β-sitosterol:cholesterol is between about 8:2 and about 1:9.
E98 The LNP formulation of E96 or E97, wherein the molar ratio of β-sitosterol:cholesterol is about 6:4 or about 4:6.
E99 The LNP formulation of any one of E78-E98, wherein the neutral lipid is a phospholipid.
E100 The LNP formulation of any one of E78-E99, wherein the neutral lipid is distearoylphosphatidylcholine, 1,2-distearoyl-sn-glycero-3-phosphocholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyl-oleoyl-phosphatidylethanolamine (POPE), dioleoyl-phosphatidylethanolamine 4-(N-maleimidomethyl)-cyclohexane-1-carboxylate (DOPE-mal), dipalmitoylphosphatidylethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearoyl-2-oleoylphosphatidyethanolamine (SOPE), 1,2-dielaidoyl-sn-glycero-3-phosphoethanolamine (transDOPE), hydrogenated soy phosphatidylcholine (HSPC), egg phosphatidylcholine (EPC), dioleoylphosphatidyl serine (DOPS), sphingomyelin (SM), dimyristoyl phosphatidylcholine (DMPC), dimyristoyl phosphatidylglycerol (DMPG), distearoylphosphatidylglycerol (DSPG), diemcoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or a mixture thereof.
E101 The LNP formulation of any one of E78-E100, wherein the neutral lipid is 1,2-distearoyl-sn-glycero-3-phosphocholine (DSPC).
E102 The LNP formulation of any one of E78-E101, wherein the polymer-conjugated lipid is a pegylated lipid.
E103 The LNP formulation of E102, wherein the pegylated lipid is selected from the group consisting of 2-[(polyethylene glycol)-2000]-N,N-ditetradecylacetamide (ALC-0159), PEG dialkyoxypropylcarba PEGylated diacylglycerol (PEG-DAG), 1-(monomethoxy-polyethyleneglycol)-2,3-dimyristoylglycerol (PEG-DMG), PEGylated phosphatidylethanolamine (PEG-PE), PEG succinate diacylglycerol (PEG-S-DAG), 4-O-(2′,3′-di(tetradecanoyloxy)propyl-1-O—((O-methoxy (polyethoxy)ethyl) butanedioate (PEG-S-DMG), PEG-ceramide, and α-(3′-[(1,2-di[myristyloxy]propanoxy)carbonylamino]propyl)-ω-methoxy, polyoxyethylene (PEG-C-DMG).
E104 The LNP formulation of E102 or E103, wherein the pegylated lipid is ALC-0159 (2-[(polyethylene glycol)-2000]-N,N-ditetradecylacetamide) having the structure:

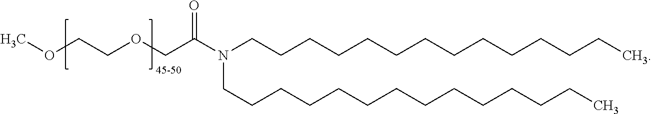

E105 The LNP formulation of any one of E78-E101, wherein the polymer-conjugated lipid is a terminally activated polyoxazoline (POZ)-conjugated lipid.
E106 The LNP formulation of E105, wherein the POZ-conjugated lipid is polyethyloxazoline-dimyristoylamide (PEOZ-dma) having the structure:

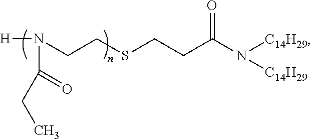

wherein n is an integer from 18-22.
E107 The LNP formulation of any one of E78-E106, wherein the LNPs comprise lipids from any one of the groups from a) to l):
a) ALC-0315, beta-sitosterol, cholesterol, DSPC, and ALC-0159;
b) ALC-0315, beta-sitosterol, cholesterol, ESM, and ALC-0159;
c) ALC-0315, beta-sitosterol, cholesterol, DSPC, and PEOZ;
d) ALC-0315, beta-sitosterol, cholesterol, ESM, and PEOZ;
e) ALC-0515, beta-sitosterol, cholesterol, DSPC, and ALC-0159;
f) ALC-0515, beta-sitosterol, cholesterol, ESM, and ALC-0159;
g) ALC-0515, beta-sitosterol, cholesterol, DSPC, and PEOZ;
h) ALC-0515, beta-sitosterol, cholesterol, ESM, and PEOZ;
i) PF-9032, beta-sitosterol, cholesterol, DSPC, and ALC-0159;
j) PF-9032, beta-sitosterol, cholesterol, ESM, and ALC-0159;
k) PF-9032, beta-sitosterol, cholesterol, DSPC, and PEOZ; and
l) PF-9032, beta-sitosterol, cholesterol, ESM, and PEOZ.
E108 The LNP formulation of any one of E78-E106, wherein the LNPs comprise lipids from any one of the groups from a) to l):
a) ALC-0315, cholesterol, DSPC, and ALC-0159;
b) ALC-0315, cholesterol, ESM, and ALC-0159;
c) ALC-0315, cholesterol, DSPC, and PEOZ;
d) ALC-0315, cholesterol, ESM, and PEOZ;
e) ALC-0515, cholesterol, DSPC, and ALC-0159;
f) ALC-0515, cholesterol, ESM, and ALC-0159;
g) ALC-0515, cholesterol, DSPC, and PEOZ;
h) ALC-0515, cholesterol, ESM, and PEOZ;
i) PF-9032, cholesterol, DSPC, and ALC-0159;
j) PF-9032, cholesterol, ESM, and ALC-0159;
k) PF-9032, cholesterol, DSPC, and PEOZ; and
l) PF-9032, cholesterol, ESM, and PEOZ.
E109 The LNP formulation of any one of E78-E92, E95-E104, or E107, wherein the LNPs comprise the following lipids: ALC-0315, beta-sitosterol, cholesterol, DSPC, and ALC-0159.
E110 The LNP formulation of any one of E53-E109, wherein the LNPs have a mean diameter size between about 1 nm and about 500 nm.
E111 The LNP formulation of any one of E53-E110, wherein the LNPs have a mean diameter size that is less than about 150 nm.
E112 The LNP formulation of any one of E53-E111, wherein the LNPs have a mean diameter size between about 60 nm and about 140 nm.
E113 The LNP formulation of any one of E53-E112, wherein the LNPs have a mean diameter size selected from the group consisting of about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, and about 140 nm.
E114 The LNP formulation of any one of E53-E113, wherein the LNPs have a polydispersity index (PDI) that is less than about 0.15, about 0.2, about 0.25, or about 0.3.
E115 The LNP formulation of any one of E53-E114, wherein the LNPs have a PDI between about 0.05 and about 0.2.
E116 The LNP formulation of any one of E78-E115, wherein the ionizable cationic lipid comprises between about 30 mol % and about 60 mol % of the total lipid.
E117 The LNP formulation of any one of E78-E116, wherein the ionizable cationic lipid comprises between about 40 mol % and about 50 mol % of the total lipid.
E118 The LNP formulation of any one of E78-E117, wherein the ionizable cationic lipid comprises about 47.5 mol % of the total lipid.
E119 The LNP formulation of any one of E78-E118, wherein the cholesterol, the cholesterol analog, or the cholesterol and the cholesterol analog comprise between about 30 mol % and about 50 mol % of the total lipid.
E120 The LNP formulation of any one of E78-E119, wherein the cholesterol, the cholesterol analog, or the cholesterol and the cholesterol analog comprise between about 40 mol % and about 41 mol % of the total lipid.
E121 The LNP formulation of any one of E78-E120, wherein the cholesterol, the cholesterol analog, or the cholesterol and the cholesterol analog comprise about 40.7 mol % of the total lipid.
E122 The LNP formulation of any one of E78-E121, wherein the neutral lipid comprises between about 1 mol % and about 30 mol % of the total lipid.
E123 The LNP formulation of any one of E78-E122, wherein the neutral lipid comprises between about 5 mol % and about 15 mol % of the total lipid.
E124 The LNP formulation of any one of E78-E123, wherein the neutral lipid comprises about 10 mol % of the total lipid.
E125 The LNP formulation of any one of E78-E124, wherein the polymer-conjugated lipid comprises between about 0.5 mol % and about 10 mol % of the total lipid.
E126 The LNP formulation of any one of E78-E125, wherein the polymer-conjugated lipid comprises between about 1 mol % and about 2 mol % of the total lipid.
E127 The LNP formulation of any one of E78-E126, wherein the polymer-conjugated lipid comprises about 1.8 mol % of the total lipid.
E128 An immunogenic composition comprising the LNP formulation of any one of E53-E127, wherein the LNPs comprise RNA, and wherein the RNA comprises at least one open reading frame (ORF) encoding an immunogen of interest.
E129 The immunogenic composition of E128, wherein the immunogen is derived from E. coli.
E130 The immunogenic composition of E129, wherein the immunogen is a fimbrial adhesin (FimH) polypeptide, or a functional fragment thereof.
E131 The immunogenic composition of E130, wherein the FimH polypeptide, or functional fragment thereof, comprises each of the mutations of G15A, G16A, and V27A, and wherein the amino acid positions are numbered according to SEQ ID NO: 1.
E132 The immunogenic composition of any one of E128-E131, wherein the RNA comprises or consists of the sequence of SEQ ID NO: 3.
E133 The immunogenic composition of any one of E128-E132, wherein the composition further comprises a fatty acid, a fatty acid derivative, or a salt thereof.
E134 The immunogenic composition of E133, wherein the fatty acid is selected from the group consisting of oleic acid, arachidonic acid, eruric acid, linolenic acid, ricinoleic acid, palmitoleic acid, and linoleic acid.
E135 The immunogenic composition of E133 or E134, wherein the salt is selected from the group consisting of sodium, potassium, magnesium, and calcium.
E136 The immunogenic composition of any one of E128-E135, wherein the composition comprises oleic acid or sodium oleate.
E137 The immunogenic composition of E136, wherein the immunogenic composition has a oleic acid to RNA mass ratio (g/g) selected from the group consisting of about 1:1, about 2:1, about 3:1, about 4:1, about 5:1, about 6:1, about 7:1, about 8:1, about 9:1, about 10:1, about 11:1, about 12:1, and about 13:1.
E138 The immunogenic composition of E137, wherein the immunogenic composition has a oleic acid to RNA mass ratio (g/g) of about 8:1.
E139 The immunogenic composition of any one of E128-E138, wherein the composition comprises a buffer and a cryoprotectant.
E140 The immunogenic composition of E139, wherein the buffer is Tris and the cryoprotectant is sucrose.
E141 The immunogenic composition of E139 or E140, wherein the composition comprises 10 mM Tris and 300 mM sucrose.
E142 The immunogenic composition of any one of E128-E141, wherein the percentage of intact RNA in the composition is at least about 80%, about 85%, or about 90%.
E143 The immunogenic composition of any one of E128-E142, wherein at least about 60%, about 65%, about 70%, about 75%, about 80%, about 85%, about 90%, or about 95% of the total RNA in the composition is encapsulated in the plurality of LNPs.
E144 The immunogenic composition of any one of E128-E143, wherein at least about 40%, about 45%, about 50%, about 55%, about 60%, about 65%, about 70%, about 75%, about 80%, about 85%, about 90%, or about 95% of the compound is encapsulated in the plurality of LNPs.
E145 The immunogenic composition of any one of E128-E144, wherein the concentration of the compound is between about 1 μg/mL and about 1000 μg/mL.
E146 The immunogenic composition of any one of E128-E145, wherein the concentration of the compound is between about 10 μg/mL and about 100 μg/mL.
E147 The immunogenic composition of any one of E128-E146, wherein the concentration of the compound is about 17 μg/mL or about 86 μg/mL.
E148 The immunogenic composition of any one of E128-E147, wherein the LNPs comprise QS-21, and wherein the concentration of QS-21 in the composition is between about 0.001 mg/mL and about 0.2 mg/mL.
E149 The immunogenic composition of any one of E128-E148, wherein the LNPs comprise QS-21, and wherein the concentration of QS-21 in the composition is about 0.009 mg/mL, about 0.023 mg/mL, about 0.045 mg/mL, or about 0.1 mg/mL.
E150 The immunogenic composition of any one of E128-E149, wherein the concentration of the RNA in the composition is less than about 1 mg/mL.
E151 The immunogenic composition of any one of E128-E150, wherein the concentration of the RNA in the composition is about 0.1 mg/mL.
E152 The immunogenic composition of any one of E128-E151, wherein the in-vitro expression (IVE) of the immunogen in a cell line treated with the immunogenic composition is at least about 40%, about 50%, about 60%, about 70%, about 80%, or about 90%.
E153 A method of inducing an immune response in a subject against the immunogen of interest, comprising administering to the subject the immunogenic composition of any one of E128-E152.
E154 A method for immunizing a subject against a disease or disorder caused by or associated with the immunogen of interest, comprising administering to the subject the immunogenic composition of any one of E128-E152.
E155 A method for preventing a disease or disorder caused by or associated with the immunogen of interest in a subject, comprising administering to the subject the immunogenic composition of any one of E128-E152.
E156 A method for treating a disease or disorder caused by or associated with the immunogen of interest in a subject, comprising administering to the subject the immunogenic composition of any one of E128-E152.
E157 A method for increasing an immune response to the immunogen of interest in a subject, comprising administering to the subject the immunogenic composition of any one of E128-E152.
E158 The method of any one of E153-E157, wherein the method induces an immune response in the subject to the immunogen of interest and the immune response is at least about 10%, at least about 20%, at least about 30%, at least about 40%, at least about 50%, at least about 60%, at least about 70%, at least about 80%, at least about 90%, or at least about 100% higher than the immune response of the subject induced by a composition comprising the immunogen of interest without the LNP formulation of any one of E53-E127.
E159 The method of any one of E153-E157, wherein the method induces an immune response in the subject to the immunogen of interest and the immune response is at least about 150%, at least about 200%, at least about 250%, at least about 300%, at least about 350%, at least about 400%, at least about 450%, or at least about 500% higher than the immune response of the subject induced by a composition comprising the immunogen of interest without the LNP formulation of any one of E53-E127.
E160 The method of any one of E153-E157, wherein the method induces an immune response in the subject to the immunogen of interest and the immune response is at least about 10%, at least about 20%, at least about 30%, at least about 40%, at least about 50%, at least about 60%, at least about 70%, at least about 80%, at least about 90%, or at least about 100% higher than the immune response of the subject induced by a composition comprising the immunogen of interest and an LNP formulation wherein the LNPs in the LNP formulation do not comprise the compound of any one of E1-E25.
E161 The method of any one of E153-E157, wherein the method induces an immune response in the subject to the immunogen of interest and the immune response is at least about 150%, at least about 200%, at least about 250%, at least about 300%, at least about 350%, at least about 400%, at least about 450%, or at least about 500% higher than the immune response of the subject induced by a composition comprising the immunogen of interest and an LNP formulation wherein the LNPs in the LNP formulation do not comprise the compound of any one of E1-E25.
E162 The method of any one of E153 or E157-E161, wherein the immune response is measured by opsonophagocytic activity (OPA) geometric mean antibody titers.
E163 The method of any one of E153 or E157-E161, wherein the immune response is measured by neutralization geometric mean antibody titers.
E164 The method of any one of E153-E163, wherein the subject is human.
E165 The immunogenic composition of any one of E128-E152, for use as a medicament.
E166 The immunogenic composition of any one of E128-E152, for use in inducing an immune response to the immunogen of interest in a subject.
E167 The immunogenic composition of any one of E128-E152, for use in immunizing a subject against a disease or disorder caused by or associated with the immunogen of interest.
E168 The immunogenic composition of any one of E128-E152, for use in preventing a disease or disorder caused by or associated with the immunogen of interest in a subject.
E169 The immunogenic composition of any one of E128-E152, for use in treating a disease or disorder caused by or associated with the immunogen of interest in a subject.
E170 The immunogenic composition of any one of E128-E152, for use in increasing an immune response to the immunogen of interest in a subject.
E171 Use of the immunogenic composition of any one of E128-E152, for the manufacture of a medicament for inducing an immune response to the immunogen of interest in a subject.
E172 Use of the immunogenic composition of any one of E128-E152, for the manufacture of a medicament for immunizing a subject against a disease or disorder caused by or associated with the immunogen of interest.
E173 Use of the immunogenic composition of any one of E128-E152, for the manufacture of a medicament for use in preventing a disease or disorder caused by or associated with the immunogen of interest in a subject.
E174 Use of the immunogenic composition of any one of E128-E152, for the manufacture of a medicament for treating a disease or disorder caused by or associated with the immunogen of interest in a subject.
E175 Use of the immunogenic composition of any one of E128-E152, for the manufacture of a medicament for increasing an immune response to the immunogen of interest in a subject.
E176 The method or use of any one of E153-E175, wherein the expression of TNF-α, IL-6, IL-8, and/or IFN-β in the subject is increased.
E177 A method of making the LNP formulation of any one of E79-E127 or the immunogenic composition of any one of E128-E152, comprising the steps of:
   (i) dissolving the compound, ionizable cationic lipid, cholesterol and/or cholesterol analog, neutral lipid, and polymer-conjugated lipid in an organic solvent to form an organic phase;
   (ii) dissolving the RNA in water or buffer to form an aqueous phase; and
   (iii) mixing the organic phase and the aqueous phase to form the LNP formulation.
E178 The method of E177, wherein in step (i) the organic solvent is ethanol or isopropyl alcohol.
E179 The method of E177 or E178, wherein in step (ii) the aqueous phase comprises citrate buffer at pH 4.
E180 The method of any one of E177-E179, wherein in step (iii) the organic phase and the aqueous phase are mixed in a microfluidic mixer.
E181 The method of E180, wherein the microfluidic mixer is a T-mixer.
E182 The method of any one of E176-E181, wherein in step (iii) the ratio of the aqueous phase to the organic phase by volume is 3:1.
E183 The method of any one of E177-E182, further comprising step (iv) dialyzing the LNP formulation against a buffer.
E184 The method of E183, wherein the buffer of step (iv) is 10 mM Tris (pH 7.4).
E185 The method of any one of E177-E184, further comprising step (v) adding a cryoprotectant to the LNP formulation.
E186 The method of E185, wherein the cryoprotectant is sucrose.
E187 The method of E185 or E186, wherein step (v) further comprises adding sodium oleate to the LNP formulation.

Furthermore, each of the embodiments described herein envisions within its scope pharmaceutically acceptable salts, tautomers, or stereoisomers thereof of the compounds described herein.

### Definitions

Unless otherwise defined herein, scientific and technical terms used in connection with the present disclosure have the meanings that are commonly understood by those of ordinary skill in the art.

The disclosure described herein suitably may be practiced in the absence of any element(s) not specifically disclosed herein.

“Compounds of the disclosure” include compounds of Formula (I) and the novel intermediates used in the preparation thereof. One of ordinary skill in the art will appreciate that compounds of the disclosure include conformational isomers (e.g., cis and trans isomers) and all optical isomers (e.g., enantiomers and diastereomers), racemic, diastereomeric and other mixtures of such isomers, tautomers thereof, where they may exist. One of ordinary skill in the art will also appreciate that compounds of the disclosure include solvates, hydrates, isomorphs, polymorphs, esters, salt forms, prodrugs, derivatives and isotopically labeled versions thereof, where they may be formed.

As used herein, the singular form “a”, “an”, and “the” include plural references unless indicated otherwise. For example, “a” substituent includes one or more substituents.

As used herein, the term “about” when used to modify a numerically defined parameter (e.g., the dose of 5 mg) means that the parameter may vary by as much as 10% below or above the stated numerical value for that parameter. For example, a dose of about 5 mg means 5 mg±10%, i.e., it may vary between 4.5 mg and 5.5 mg.

If substituents are described as being “independently selected” from a group, each substituent is selected independent of the other. Each substituent therefore may be identical to or different from the other substituent(s).

“Optional” or “optionally” means that the subsequently described event or circumstance may, but need not occur, and the description includes instances where the event or circumstance occurs and instances in which it does not.

The terms “optionally substituted” and “substituted or unsubstituted” are used interchangeably to indicate that the particular group being described may have no non-hydrogen substituents (i.e., unsubstituted), or the group may have one or more non-hydrogen substituents (i.e., substituted). If not otherwise specified, the total number of substituents that may be present is equal to the number of H atoms present on the unsubstituted form of the group being described. Where an optional substituent is attached via a double bond, such as an oxo (═O) substituent, the group occupies two available valences, so the total number of other substituents that are included is reduced by two. In the case where optional substituents are selected independently from a list of alternatives, the selected groups may be the same or different. Throughout the disclosure, it will be understood that the number and nature of optional substituent groups will be limited to the extent that such substitutions make chemical sense to one of ordinary skill in the art.

“Halogen” or “halo” refers to fluoro, chloro, bromo and iodo (F, Cl, Br, I).

“Cyano” refers to a substituent having a carbon atom joined to a nitrogen atom by a triple bond, i.e., —C≡N.

“Hydroxy” refers to an —OH group.

“Oxo” refers to a double bonded oxygen (═O).

“Alkyl” refers to a saturated, monovalent aliphatic hydrocarbon radical that has a specified number of carbon atoms, including straight chain or branched chain groups. Alkyl groups may contain, but are not limited to, 1 to 12 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄ alkyl”), 1 to 3 carbon atoms (“C₁-C₃ alkyl”), or 1 to 2 carbon atoms (“C₁-C₂ alkyl”). Examples include, but are not limited to, methyl, ethyl, n-propyl, isopropyl, n-butyl, sec-butyl, isobutyl, tert-butyl, n-pentyl, isopentyl, neopentyl, n-hexyl, n-heptyl, n-octyl, and the like. Alkyl groups may be optionally substituted, unsubstituted or substituted, as further defined herein. In some instances, substituted alkyl groups are specifically named by reference to the substituent group. For example, “haloalkyl” refers to an alkyl group having the specified number of carbon atoms that is substituted by one or more halo substituents, up to the available valence number.

“Alkoxy” refers to an alkyl group, as defined herein, that is single bonded to an oxygen atom. The attachment point of an alkoxy radical to a molecule is through the oxygen atom. An alkoxy radical may be depicted as alkyl-O—. Alkoxy groups may contain, but are not limited to, 1 to 8 carbon atoms (“C₁-C₈ alkoxy”), 1 to 6 carbon atoms (“C₁-C₆ alkoxy”), 1 to 4 carbon atoms (“C₁-C₄ alkoxy”), or 1 to 3 carbon atoms (“C₁-C₃ alkoxy”). Alkoxy groups include, but are not limited to, methoxy, ethoxy, n-propoxy, isobutoxy, and the like.

“Alkenyl” refers to an alkyl group, as defined herein, consisting of at least two carbon atoms and at least one carbon-carbon double bond. For example, as used herein, the term “C₂-C₆ alkenyl” means straight or branched chain unsaturated radicals of 2 to 6 carbon atoms, including, but not limited to, ethenyl, 1-propenyl, 2-propenyl, 1-, 2-, or 3-butenyl, and the like.

“Amino” refers to a group —NH₂, which is unsubstituted. Where the amino is described as substituted or optionally substituted, the term includes groups of the form —NRxRy, where each of Rx and Ry is defined as further described herein. For example, “alkylamino” refers to a group —NRxRy, wherein one of Rx and Ry is an alkyl moiety and the other is H, and “dialkylamino” refers to —NRxRy wherein both of Rx and Ry are alkyl moieties, where the alkyl moieties have the specified number of carbon atoms (e.g., —NH(C₁-C₄ alkyl) or —N(C₁-C₄ alkyl)₂).

“Aryl” or “aromatic” refers to monocyclic, bicyclic (e.g., biaryl, fused) or polycyclic ring systems that contain the specified number of ring atoms, in which all carbon atoms in the ring are of sp² hybridization and in which the pi electrons are in conjugation. Aryl groups may contain, but are not limited to, 6 to 20 carbon atoms (“C₆-C₂₀ aryl”), 6 to 14 carbon atoms (“C₆-C₁₄ aryl”), 6 to 12 carbon atoms (“C₆-C₁₂ aryl”), or 6 to 10 carbon atoms (“C₆-C₁₀ aryl”). Fused aryl groups may include an aryl ring (e.g., a phenyl ring) fused to another aryl ring. Examples include, but are not limited to, phenyl, biphenyl, naphthyl, anthracenyl, phenanthrenyl, indanyl, and indenyl. Aryl groups may be optionally substituted, unsubstituted or substituted, as further defined herein.

The term “pharmaceutically acceptable” means the substance (e.g., the compounds described herein) and any salt thereof, or composition containing the substance or salt of the disclosure is suitable for administration to a subject or patient.

The compounds of the disclosure have asymmetric carbon atoms. The carbon-carbon bonds of the compounds of the disclosure may be depicted herein using a solid line (-), a solid wedge ( [symbol] ) or a dotted wedge ( [symbol] ) The use of a solid line to depict bonds to asymmetric carbon atoms is meant to indicate that all possible stereoisomers (e.g., specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of Formula (I) may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of Formula (I) can exist as enantiomers and diastereomers or as racemates and mixtures thereof. The use of a solid line to depict bonds to one or more asymmetric carbon atoms in a compound of Formula (I) and the use of a solid or dotted wedge to depict bonds to other asymmetric carbon atoms in the same compound is meant to indicate that a mixture of diastereomers is present.

The terms “inhibiting,” “decreasing,” or “reducing” or any variation of these terms includes any measurable decrease (e.g., a 5%, 10%, 15%, 20%, 25%, 30%, 35%, 40%, 45%, 50%, 55%, 60%, 65%, 70%, 75%, 80%, 85%, 90%, 95%, or 99% decrease) or complete inhibition to achieve a desired result. The terms “improve,” “promote,” or “increase” or any variation of these terms includes any measurable increase (e.g., a 5%, 10%, 15%, 20%, 25%, 30%, 35%, 40%, 45%, 50%, 55%, 60%, 65%, 70%, 75%, 80%, 85%, 90%, 95%, or 99% increase) to achieve a desired result or production of a protein or molecule.

As used herein, the terms “reference,” “standard,” or “control” describe a value relative to which a comparison is performed. For example, an agent, subject, population, sample, or value of interest is compared with a reference, standard, or control agent, subject, population, sample, or value of interest. A reference, standard, or control may be tested and/or determined substantially simultaneously and/or with the testing or determination of interest for an agent, subject, population, sample, or value of interest and/or may be determined or characterized under comparable conditions or circumstances to the agent, subject, population, sample, or value of interest under assessment.

The term “isolated” may refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that may be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state and/or that is altered or removed from the natural state through human intervention. For example, a DNA naturally present in a living animal is not “isolated,” but a synthetic DNA, or a DNA partially or completely separated from the coexisting materials of its natural state is “isolated.” An isolated nucleic acid may exist in substantially purified form, or may exist in a non-native environment such as, for example, a cell into which the nucleic acid has been delivered.

A “nucleic acid,” as used herein, is a molecule comprising nucleic acid components and refers to DNA or RNA molecules. It may be used interchangeably with the term “polynucleotide.” A nucleic acid molecule is a polymer comprising or consisting of nucleotide monomers, which are covalently linked to each other by phosphodiester-bonds of a sugar/phosphate-backbone. Nucleic acids may also encompass modified nucleic acid molecules, such as base-modified, sugar-modified or backbone-modified etc. DNA or RNA molecules. Nucleic acids may exist in a variety of forms such as: isolated segments and recombinant vectors of incorporated sequences or recombinant polynucleotides encoding polypeptides, such as antigens or one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, mRNA, modRNA and complementary sequences of the foregoing described herein. Nucleic acids may encode an epitope to which antibodies may bind.

The term “epitope” refers to a moiety that is specifically recognized by an immunoglobulin (e.g., antibody or receptor) binding component. In some aspects, an epitope is comprised of a plurality of chemical atoms or groups on a target molecule. In some aspects, such chemical atoms or groups are surface-exposed when the target molecule adopts a relevant three-dimensional conformation. In some aspects, such chemical atoms or groups are physically near to each other in space when the target molecule adopts such a conformation. In some aspects, at least some such chemical atoms are groups are physically separated from one another when the target molecule adopts an alternative conformation (e.g., is linearized).

Nucleic acids may be single-stranded or double-stranded and may comprise RNA and/or DNA nucleotides and artificial variants thereof (e.g., peptide nucleic acids). In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. A tag or other heterologous polypeptide may be added to the modified polypeptide-encoding sequence, wherein “heterologous” refers to a polypeptide that is not the same as the modified polypeptide.

The term “polynucleotide” refers to a nucleic acid molecule that may be recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA, or synthetic), analogs thereof, or a combination thereof. Additional coding or non-coding sequences may, but need not, be present within a polynucleotide.

In certain aspects, there are polynucleotide variants having substantial identity to the sequences disclosed herein; those comprising equal to any one of, at least any one of, at most any one of, or between any two of 70%, 75%, 80%, 85%, 90%, 95%, 96%, 97%, 98%, or 99% or higher sequence identity, compared to a polynucleotide sequence provided herein using the methods described herein (e.g., BLAST analysis using standard parameters). In certain aspects, the isolated polynucleotide will comprise a nucleotide sequence encoding a polypeptide that has at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) identity to a wild-type polynucleotide encoding a polypeptide over the entire length of the sequence; or a nucleotide sequence complementary to said isolated polynucleotide. In some aspects, the isolated polynucleotide will comprise a nucleotide sequence encoding a polypeptide that has at least 90% identity to an amino acid sequence described herein, over the entire length of the sequence; or a nucleotide sequence complementary to said isolated polynucleotide. In some aspects, the isolated polynucleotide will comprise a nucleotide sequence encoding a polypeptide that has at least 95% identity to an amino acid sequence described herein, over the entire length of the sequence; or a nucleotide sequence complementary to said isolated polynucleotide.

In certain aspects, the isolated polypeptide will comprise an amino acid sequence encoding a polypeptide that has at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) identity to a wild-type amino acid sequence encoding a polypeptide over the entire length of the sequence. In some aspects, the isolated polypeptide will comprise an amino acid sequence encoding a polypeptide that has at least 90% identity to an amino acid sequence described herein, over the entire length of the sequence. In some aspects, the isolated polypeptide will comprise an amino acid sequence encoding a polypeptide that has at least 95% identity to an amino acid sequence described herein, over the entire length of the sequence.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids may be any length. They may be, for example, equal to any one of, at least any one of, at most any one of, or between any two of 5, 10, 15, 20, 25, 30, 35, 40, 45, 50, 75, 100, 125, 175, 200, 250, 300, 350, 400, 450, 500, 750, 1000, 1500, 3000, 5000, 6000, 7000, 8000, 9000, 10000, 11000, 12000, 13000, 14000, 15000 or more nucleotides in length, and/or may comprise one or more additional sequences, for example, regulatory sequences, and/or be a part of a larger nucleic acid, for example, a vector. It is therefore contemplated that a nucleic acid fragment of almost any length may be employed, with the total length being limited by the ease of preparation and use in the intended recombinant nucleic acid protocol.

In this respect, the term “gene” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar polypeptide.

As used herein, the term “expression” of a nucleic acid sequence refers to the generation of any gene product from the nucleic acid sequence. In some aspects, a gene product may be a transcript. In some aspects, a gene product may be a polypeptide. In some aspects, expression of a nucleic acid sequence involves one or more of the following: (1) production of an RNA template from a DNA sequence (e.g., by transcription); (2) processing of an RNA transcript (e.g., by splicing, editing, etc.); (3) translation of an RNA into a polypeptide or protein; and/or (4) post-translational modification of a polypeptide or protein.

In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature.

The term “DNA,” as used herein, means a nucleic acid molecule comprising nucleotides such as deoxy-adenosine-monophosphate, deoxy-thymidine-monophosphate, deoxy-guanosine-monophosphate and deoxy-cytidine-monophosphate monomers which are composed of a sugar moiety (deoxyribose), a base moiety and a phosphate moiety, and polymerize by a characteristic backbone structure. The backbone structure is, typically, formed by phosphodiester bonds between the sugar moiety of the nucleotide, e.g., deoxyribose, of a first and a phosphate moiety of a second, adjacent monomer. The specific order of the monomers, e.g., the order of the bases linked to the sugar/phosphate-backbone, is called the DNA sequence. DNA may be single stranded or double stranded. In the double stranded form, the nucleotides of the first strand typically hybridize with the nucleotides of the second strand, e.g. by A/T-base-pairing and G/C-base-pairing. DNA may contain all, or a majority of, deoxyribonucleotide residues. As used herein, the term “deoxyribonucleotide” means a nucleotide lacking a hydroxyl group at the 2′ position of a β-D-ribofuranosyl group. Without any limitation, DNA may encompass double stranded DNA, antisense DNA, single stranded DNA, isolated DNA, synthetic DNA, DNA that is recombinantly produced, and modified DNA.

The term “RNA,” as used herein, means a nucleic acid molecule comprising nucleotides such as adenosine-monophosphate, uridine-monophosphate, guanosine-monophosphate and cytidine-monophosphate monomers which are connected to each other along a so-called backbone. The backbone is formed by phosphodiester bonds between the sugar, e.g., ribose, of a first and a phosphate moiety of a second, adjacent monomer. RNA may be obtainable by transcription of a DNA-sequence, e.g., inside a cell. In eukaryotic cells, transcription is typically performed inside the nucleus or the mitochondria. In vivo, transcription of DNA may result in premature RNA which is processed into messenger-RNA (mRNA). Processing of the premature RNA, e.g. in eukaryotic organisms, comprises various posttranscriptional modifications such as splicing, 5′ capping, polyadenylation, export from the nucleus or the mitochondria. Mature messenger RNA is processed and provides the nucleotide sequence that may be translated into an amino acid sequence of a peptide or protein. A mature mRNA may comprise a 5′ cap, a 5′ UTR, an open reading frame, a 3′ UTR and a poly-A tail sequence. RNA may contain all, or a majority of, ribonucleotide residues. As used herein, the term “ribonucleotide” means a nucleotide with a hydroxyl group at the 2′ position of a β-D-ribofuranosyl group. In one aspect, RNA may be messenger RNA (mRNA) that relates to a RNA transcript which encodes a peptide or protein. As known to those of skill in the art, mRNA generally contains a 5′ untranslated region (5′ UTR), a polypeptide coding region, and a 3′ untranslated region (3′ UTR). Without any limitation, RNA may encompass double stranded RNA, antisense RNA, single stranded RNA, isolated RNA, synthetic RNA, RNA that is recombinantly produced, and modified RNA (modRNA).

An “isolated RNA” is defined as an RNA molecule that may be recombinant or has been isolated from total genomic nucleic acid. An isolated RNA molecule or protein may exist in substantially purified form, or may exist in a non-native environment such as, for example, a host cell.

A “modified RNA” or “modRNA” refers to an RNA molecule having at least one addition, deletion, substitution, and/or alteration of one or more nucleotides as compared to naturally occurring RNA. Such alterations may refer to the addition of non-nucleotide material to internal RNA nucleotides, or to the 5′ and/or 3′ end(s) of RNA. In one aspect, such modRNA contains at least one modified nucleotide, such as an alteration to the base of the nucleotide. For example, a modified nucleotide may replace one or more uridine and/or cytidine nucleotides. For example, these replacements may occur for every instance of uridine and/or cytidine in the RNA sequence, or may occur for only select uridine and/or cytidine nucleotides. Such alterations to the standard nucleotides in RNA may include non-standard nucleotides, such as chemically synthesized nucleotides or deoxynucleotides. For example, at least one uridine nucleotide may be replaced with N1-methylpseudouridine (denoted by the symbol m¹Ψ) in an RNA sequence. Other such altered nucleotides are known to those of skill in the art. Such altered RNA molecules are considered analogs of naturally-occurring RNA. In some aspects, the RNA is produced by in vitro transcription using a DNA template, where DNA refers to a nucleic acid that contains deoxyribonucleotides. In some aspects, the RNA may be replicon RNA (replicon), in particular self-replicating RNA, or self-amplifying RNA (saRNA).

In some embodiments, the RNA molecule is an saRNA. “saRNA,” “self-amplifying RNA,” and “replicon” refer to RNA with the ability to replicate itself. Self-amplifying RNA molecules may be produced by using replication elements derived from a virus or viruses, e.g., alphaviruses, and substituting the structural viral polypeptides with a nucleotide sequence encoding a polypeptide of interest. A self-amplifying RNA molecule is typically a positive-strand molecule that may be directly translated after delivery to a cell, and this translation provides an RNA-dependent RNA polymerase which then produces both antisense and sense transcripts from the delivered RNA. The delivered RNA leads to the production of multiple daughter RNAs. These daughter RNAs, as well as collinear subgenomic transcripts, may be translated themselves to provide in situ expression of an encoded gene of interest, e.g., a viral antigen, or may be transcribed to provide further transcripts with the same sense as the delivered RNA which are translated to provide in situ expression of the protein of interest, e.g., an antigen. The overall result of this sequence of transcriptions is an amplification in the number of the introduced saRNAs and so the encoded gene of interest, e.g., a viral antigen, can become a major polypeptide product of the cells.

As contemplated herein, without any limitations, RNA may be used as a therapeutic modality to treat and/or prevent a number of conditions in mammals, including humans. Methods described herein comprise administration of the RNA described herein to a mammal, such as a human. In one aspect, the RNA administered is in vitro transcribed RNA.

“Prevent” or “prevention,” as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder, or condition has been delayed for a predefined period of time.

As will be understood from context, “risk” of a disease, disorder, and/or condition refers to a likelihood that a particular individual will develop the disease, disorder, and/or condition. In some aspects, risk is expressed as a percentage. In some aspects, risk is, is at least, or is at most from 0, 1, 2, 3, 4, 5, 6, 7, 8, 9, 10, 20, 30, 40, 50, 60, 70, 80, 90 up to 100%. In some aspects risk is expressed as a risk relative to a risk associated with a reference sample or group of reference samples. In some aspects, a reference sample or group of reference samples have a known risk of a disease, disorder, condition and/or event. In some aspects a reference sample or group of reference samples are from individuals comparable to a particular individual. In some aspects, risk may reflect one or more genetic attributes, e.g., which may predispose an individual toward development (or not) of a particular disease, disorder and/or condition. In some aspects, risk may reflect one or more epigenetic events or attributes and/or one or more lifestyle or environmental events or attributes. Susceptible to: An individual who is “susceptible to” a disease, disorder, and/or condition is one who has a higher risk of developing the disease, disorder, and/or condition than does a member of the general public. In some aspects, an individual who is susceptible to a disease, disorder and/or condition may not have been diagnosed with the disease, disorder, and/or condition. In some aspects, an individual who is susceptible to a disease, disorder, and/or condition may exhibit symptoms of the disease, disorder, and/or condition. In some aspects, an individual who is susceptible to a disease, disorder, and/or condition may not exhibit symptoms of the disease, disorder, and/or condition. In some aspects, an individual who is susceptible to a disease, disorder, and/or condition will develop the disease, disorder, and/or condition. In some aspects, an individual who is susceptible to a disease, disorder, and/or condition will not develop the disease, disorder, and/or condition.

The terms “protein,” “polypeptide,” or “peptide” are used herein as synonyms and refer to a polymer of amino acid monomers, e.g., a molecule comprising at least two amino acid residues. Polypeptides may include gene products, naturally occurring polypeptides, synthetic polypeptides, homologs, orthologs, paralogs, fragments and other equivalents, variants, and analogs of the foregoing. Polypeptides may be a single molecule or may be a multi-molecular complex such as a dimer, trimer or tetramer. A protein comprises one or more peptides or polypeptides, and may be folded into a 3-dimensional form, which may be required for the protein to exert its biological function.

As used herein, the term “wild type” or “WT” or “native” refers to the endogenous version of a molecule that occurs naturally in an organism. In some aspects, wild type versions of a protein or polypeptide are employed, however, in other aspects of the disclosure, a modified protein or polypeptide is employed to generate an immune response. The terms described above may be used interchangeably.

A “modified protein” or “modified polypeptide” refers to a protein or polypeptide whose chemical structure, particularly its amino acid sequence, is altered with respect to the wild type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild type activity or function in other respects, such as immunogenicity. Where a protein is specifically mentioned herein, it is in general a reference to a native (wild type) or recombinant (modified) protein. The protein may be isolated directly from the organism of which it is native, produced by recombinant DNA/exogenous expression methods, produced by solid-phase peptide synthesis (SPPS), or other in vitro methods. In particular aspects, there are isolated nucleic acid segments and recombinant vectors incorporating nucleic acid sequences that encode a polypeptide (e.g., an antigen or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

The term “fragment,” with reference to an amino acid sequence (peptide or protein), relates to a part of an amino acid sequence, e.g., a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90%, or at least 99% of the amino acid residues from an amino acid sequence. In the present disclosure, a fragment of a polypeptide, DNA nucleic acid or RNA nucleic acid sequence refers to a sequence having sequence identity of at least, at most, exactly, or between any two of 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99% with a polypeptide, DNA nucleic acid or RNA nucleic acid sequence, from which it is derived.

In one aspect, a fragment of a polypeptide, DNA nucleic acid or RNA nucleic acid sequence refers to a sequence having sequence identity of at least 70% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 80% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 85% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 90% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 95% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 97% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 99% with a polypeptide, DNA nucleic acid or RNA nucleic acid sequence, from which it is derived.

As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some aspects, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. In some aspects, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some aspects, a variant polypeptide or nucleic acid shows an overall sequence identity with a reference polypeptide or nucleic acid that is at least, at most, exactly, or between any two of 85%, 86%, 87%, 88%, 89%, 90%, 91%, 92%, 93%, 94%, 95%, 96%, 97%, or 99%. In some aspects, a variant polypeptide or nucleic acid does not share at least one characteristic sequence element with a reference polypeptide or nucleic acid. In some aspects, a reference polypeptide or nucleic acid has one or more biological activities. In some aspects, a variant polypeptide or nucleic acid shares one or more of the biological activities of the reference polypeptide or nucleic acid. In some aspects, a variant polypeptide or nucleic acid lacks one or more of the biological activities of the reference polypeptide or nucleic acid. In some aspects, a variant polypeptide or nucleic acid shows a reduced level of one or more biological activities as compared to the reference polypeptide or nucleic acid. In some aspects, a polypeptide or nucleic acid of interest is considered to be a “variant” of a reference polypeptide or nucleic acid if it has an amino acid or nucleotide sequence that is identical to that of the reference but for a small number of sequence alterations at particular positions. Preferably, the variant polypeptide or nucleic acid sequence has at least one modification compared to the reference polypeptide or nucleic acid sequence, e.g., from 1 to about 20 modifications. In one aspect, the variant polypeptide or nucleic acid sequence has from 1 to about 10 modifications compared to the reference polypeptide or nucleic acid sequence. In one aspect, the variant polypeptide or nucleic acid sequence has from 1 to about 5 modifications compared to the reference polypeptide or nucleic acid sequence. In one aspect, the variant polypeptide or nucleic acid sequence has from 1 to about 4 modifications compared to the reference polypeptide or nucleic acid sequence. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. Often, a variant polypeptide or nucleic acid comprises a very small number (e.g., fewer than about 5, about 4, about 3, about 2, or about 1) number of substituted, inserted, or deleted, functional residues (e.g., residues that participate in a particular biological activity) relative to the reference. In some aspects, a variant polypeptide or nucleic acid comprises about 10, about 9, about 8, about 7, about 6, about 5, about 4, about 3, about 2, or about 1 substituted residues as compared to a reference. In some aspects, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some aspects, a variant polypeptide or nucleic acid comprises not more than about 5, about 4, about 3, about 2, or about 1 addition or deletion, and, in some aspects, comprises no additions or deletions, as compared to the reference.

In some aspects, a reference polypeptide or nucleic acid is a wild type (WT) or native sequence found in nature, including allelic variations. For the purposes of the present disclosure, “variants” of an amino acid sequence (peptide, protein, or polypeptide) can comprise amino acid insertion variants, amino acid addition variants, amino acid deletion variants and/or amino acid substitution variants. “Variants” of a nucleotide sequence can comprise nucleotide insertion variants, nucleotide addition variants, nucleotide deletion variants and/or nucleotide substitution variants. The term “variant” includes all mutants, splice variants, post-translationally modified variants, conformations, isoforms, allelic variants, species variants, and species homologs, in particular those which are naturally occurring. The term “variant” also includes, in particular, fragments of an amino acid or nucleic acid sequence.

Changes may be introduced by mutation into a nucleic acid, thereby leading to changes in the amino acid sequence of a polypeptide (e.g., an antigen) that it encodes. Mutations may be introduced using any technique known in the art. In one aspect, one or more particular amino acid residues are changed using, for example, a site-directed mutagenesis protocol. In another aspect, one or more randomly selected residues are changed using, for example, a random mutagenesis protocol. In some aspects, however it is made, a mutant polypeptide may be expressed and screened for a desired property.

Mutations may be introduced into a nucleic acid without significantly altering the biological activity of a polypeptide that it encodes. For example, one may make nucleotide substitutions leading to amino acid substitutions at non-essential amino acid residues. Alternatively, one or more mutations may be introduced into a nucleic acid that selectively changes the biological activity of a polypeptide that it encodes. For example, the mutation may quantitatively or qualitatively change the biological activity. Examples of quantitative changes include increasing, reducing or eliminating the activity. Examples of qualitative changes include altering the antigen specificity of an antibody.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical,” “% identity,” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison,” in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N, and TFASTA in Wisconsin Genetics Software Package, Genetics Computer Group). In some aspects, percent identity of two sequences is determined using the BLASTN or BLASTP algorithm, as available on the United States National Center for Biotechnology Information (NCBI) website.

Percentage identity is obtained by determining the number of identical positions at which the sequences to be compared correspond, dividing this number by the number of positions compared (e.g., the number of positions in the reference sequence) and multiplying this result by 100.

In some aspects, the degree of similarity or identity is given for a region that is at least, at most, exactly, or between any two of about 50%, about 60%, about 70%, about 80%, about 90%, or about 100% of the entire length of the reference sequence. For example, if the reference nucleic acid sequence consists of 200 nucleotides, the degree of identity is given for at least, at most, exactly, or between any two of about 100, about 120, about 140, about 160, about 180, or about 200 nucleotides, in some aspects, continuous nucleotides. In some aspects, the degree of similarity or identity is given for the entire length of the reference sequence.

Homologous amino acid sequences may exhibit at least, at most, exactly, or between any two of 40%, 50%, 60%, 70%, 80%, 90%, 95%, 98%, or 99% identity of the amino acid residues. In one aspect, homologous amino acid sequences exhibit at least 95% identity of the amino acid residues. In one aspect, homologous amino acid sequences exhibit at least 98% identity of the amino acid residues. In one aspect, homologous amino acid sequences exhibit at least 99% identity of the amino acid residues.

A fragment or variant of an amino acid sequence (peptide or protein) may be a “functional fragment” or “functional variant.” The term “functional fragment” or “functional variant” of an amino acid sequence relates to any fragment or variant exhibiting one or more functional properties identical or similar to those of the amino acid sequence from which it is derived, e.g., it is functionally equivalent. With respect to antigens or antigenic sequences, one particular function is one or more immunogenic activities displayed by the amino acid sequence from which the fragment or variant is derived. The term “functional fragment” or “functional variant,” as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In one aspect, the modifications in the amino acid sequence of the parent molecule or sequence do not significantly affect or alter the characteristics of the molecule or sequence. The term “mutant” of a wild-type protein, “mutant” of a protein, “protein mutant,” or “modified protein” refer to a polypeptide that displays introduced mutations relative to a wild-type protein.

An amino acid sequence (peptide, protein, or polypeptide) “derived from” a designated amino acid sequence (peptide, protein, or polypeptide) refers to the origin of the first amino acid sequence. Preferably, the amino acid sequence which is derived from a particular amino acid sequence has an amino acid sequence that is identical, essentially identical, or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

In the present disclosure, a vector refers to a nucleic acid molecule, such as an artificial nucleic acid molecule. A vector may be used to incorporate a nucleic acid sequence, such as a nucleic acid sequence comprising an open reading frame. Vectors include, but are not limited to, storage vectors, expression vectors, cloning vectors, transfer vectors. A vector may be an RNA vector or a DNA vector. In some aspects the vector is a DNA molecule. In some aspects, the vector is a plasmid vector. In some aspects, the vector is a viral vector. Typically, an expression vector will contain a desired coding sequence and appropriate other sequences necessary for the expression of the operably linked coding sequence in a particular host organism (e.g., bacteria, yeast, plant, insect, or mammal) or in in vitro expression systems. Cloning vectors are generally used to engineer and amplify a certain desired fragment (typically a DNA fragment), and may lack functional sequences needed for expression of the desired fragment(s).

As used herein, the term “immunogen” refers to a substance, which is capable of being recognized by the immune system, e.g. by the adaptive immune system, and which is capable of eliciting an antigen-specific immune response, e.g. by formation of antibodies and/or antigen-specific T cells as part of an adaptive immune response. An immunogen may be or may comprise a peptide or protein, which may be presented by the MHC to T-cells. An immunogen may be the product of translation of a provided nucleic acid molecule, e.g. an RNA molecule comprising at least one coding sequence as described herein. In addition, fragments, variants and derivatives of an immunogen, such as a peptide or a protein, comprising at least one epitope are understood as immunogens.

As used herein, the term “vaccine” refers to a pharmaceutical composition comprising an immunogenic agent, immunogen, or antigen, wherein the pharmaceutical composition is intended to generate an immune response, for example to a disease-associated (e.g., disease-causing) agent (e.g., a bacteria). In some aspects, a vaccine generates an immune response to an infectious agent. In some aspects, a vaccine generates an immune response to a tumor; in some such aspects, the vaccine is “personalized” in that it is partly or wholly directed to epitope(s) (e.g., which may be or include one or more neoepitopes) determined to be present in a particular individual's tumors.

An immune response refers to a humoral response, a cellular response, or both a humoral and cellular response in an organism. An immune response may be measured by assays that include, but are not limited to, assays measuring the presence or amount of antibodies that specifically recognize a protein or cell surface protein, assays measuring T-cell activation or proliferation, and/or assays that measure modulation in terms of activity or expression of one or more cytokines.

Those skilled in the art will appreciate that the term “dosing regimen” may be used to refer to a set of unit doses (typically more than one) that are administered individually to a subject, typically separated by periods of time. In some aspects, a given therapeutic agent has a recommended dosing regimen, which may involve one or more doses. In some aspects, a dosing regimen comprises a plurality of doses each of which is separated in time from other doses. In some aspects, individual doses are separated from one another by a time period of the same length; in some aspects, a dosing regimen comprises a plurality of doses and at least two different time periods separating individual doses. In some aspects, all doses within a dosing regimen are of the same unit dose amount. In some aspects, different doses within a dosing regimen are of different amounts. In some aspects, a dosing regimen comprises a first dose in a first dose amount, followed by one or more additional doses in a second dose amount different from the first dose amount. In some aspects, a dosing regimen comprises a first dose in a first dose amount, followed by one or more additional doses in a second dose amount same as the first dose amount. In some aspects, a dosing regimen is correlated with a desired or beneficial outcome when administered across a relevant population (e.g., is a therapeutic dosing regimen).

### Salts

Salts encompassed within the term “pharmaceutically acceptable salts” refer to the compounds of this disclosure which are generally prepared by reacting the free base or free acid with a suitable organic or inorganic acid, or a suitable organic or inorganic base, respectively, to provide a salt of the compound of the disclosure that is suitable for administration to a subject or patient.

In addition, the compounds of Formula I may also include other salts of such compounds which are not necessarily pharmaceutically acceptable salts, which may be useful as intermediates for one or more of the following: 1) preparing compounds of Formula I; 2) purifying compounds of Formula I; 3) separating enantiomers of compounds of Formula I; or 4) separating diastereomers of compounds of Formula I.

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinafoate salts.

Suitable base salts are formed from bases which form non-toxic salts. Examples include, but are not limited to aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see Paulekun, G. S. et al., Trends in Active Pharmaceutical Ingredient Salt Selection Based on Analysis of the Orange Book Database, J. Med. Chem. 2007; 50(26), 6665-6672.

Pharmaceutically acceptable salts of compounds of the disclosure may be prepared by methods well known to one skilled in the art, including but not limited to the following procedures
(i) by reacting a compound of the disclosure with the desired acid or base;
(ii) by removing an acid- or base-labile protecting group from a suitable precursor of a compound of the disclosure or by ring-opening a suitable cyclic precursor, for example, a lactone or lactam, using the desired acid or base; or
(iii) by converting one salt of a compound of the disclosure to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

### Solvates

The compounds of the disclosure, and pharmaceutically acceptable salts thereof, may exist in unsolvated and solvated forms. The term ‘solvate’ is used herein to describe a molecular complex comprising the compound of the disclosure, or a pharmaceutically acceptable salt thereof, and one or more pharmaceutically acceptable solvent molecules, for example, ethanol. The term ‘hydrate’ is employed when said solvent is water.

In addition, the compounds of Formula I may also include other solvates of such compounds which are not necessarily pharmaceutically acceptable solvates, which may be useful as intermediates for one or more of the following: 1) preparing compounds of Formula I; 2) purifying compounds of Formula I; 3) separating enantiomers of compounds of Formula I; or 4) separating diastereomers of compounds of Formula I.

A currently accepted classification system for organic hydrates is one that defines isolated site, channel, or metal-ion coordinated hydrates-see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Complexes

Also included within the scope of the disclosure are multi-component complexes (other than salts and solvates) wherein the drug and at least one other component are present in stoichiometric or non-stoichiometric amounts. In one embodiment, the compounds of the disclosure are in a complex with aluminum. In another embodiment, the compounds of the disclosure are in a complex with aluminum hydroxide. In another embodiment, the compounds of the disclosure are in a complex with aluminum phosphate. Complexes of this type include clathrates (drug-host inclusion complexes) and co-crystals. The latter are typically defined as crystalline complexes of neutral molecular constituents which are bound together through non-covalent interactions, for example, hydrogen bonded complex (cocrystal) may be formed with either a neutral molecule or with a salt. Co-crystals may be prepared by melt crystallization, by recrystallization from solvents, or by physically grinding the components together-see Chem Commun, 17; 1889-1896, by O. Almarsson and M. J. Zaworotko (2004). For a general review of multi-component complexes, see J Pharm Sci, 64(8), 1269-1288, by Haleblian (August 1975).

### Solid form

The compounds of the disclosure may exist in a continuum of solid states ranging from amorphous to crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the disclosure may also exist in a mesomorphic state (mesophase or liquid crystal) when subjected to suitable conditions. The mesomorphic state is intermediate between the true crystalline state and the true liquid state (either melt or solution) and consists of two dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

### Stereoisomers

Compounds of the disclosure may exist as two or more stereoisomers. Stereoisomers of the compounds may include cis and trans isomers (geometric isomers), optical isomers such as R and S enantiomers, diastereomers, rotational isomers, atropisomers, and conformational isomers. For example, compounds of the disclosure containing one or more asymmetric carbon atoms may exist as two or more stereoisomers. Where a compound of the disclosure contains an alkenyl or alkenylene group, geometric cis/trans (or Z/E) isomers are possible. Cis/trans isomers may also exist for saturated rings.

The pharmaceutically acceptable salts of compounds of the disclosure may also contain a counterion which is optically active (e.g., d-lactate or l-lysine) or racemic (e.g., dl-tartrate or dl-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where a compound of the disclosure contains an acidic or basic moiety, a base or acid such as 1-phenylethylamine or tartaric acid. The resulting diastereomeric mixture may be separated by chromatography, fractional crystallization, or by using both of said techniques, and one or both of the diastereoisomers converted to the corresponding pure enantiomer(s) by means well known to a skilled person. Chiral compounds of the disclosure (and chiral precursors thereof) may be obtained in enantiomerically-enriched form using chromatography, typically HPLC Concentration of the eluate affords the enriched mixture. Chiral chromatography using sub- and supercritical fluids may be employed. Methods for chiral chromatography useful in some embodiments of the present disclosure are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art-see, for example, Stereochemistry of Organic Compounds by E. L. Eliel and S. H. Wilen (Wiley, 1994).

### Tautomerism

Where structural isomers are interconvertible via a low energy barrier, tautomeric isomerism (‘tautomerism’) may occur. This may take the form of proton tautomerism in compounds of the disclosure containing, for example, an imino/amino, keto/enol, or oxime/nitroso group, lactam/lactim or so-called valence tautomerism in compounds which contain an aromatic moiety. It follows that a single compound may exhibit more than one type of isomerism.

It must be emphasized that while, for conciseness, the compounds of the disclosure have been drawn herein in a single tautomeric form, all possible tautomeric forms are included within the scope of the disclosure.

### Isotopes

The present disclosure includes all pharmaceutically acceptable isotopically-labeled compounds of the disclosure wherein one or more atoms are replaced by atoms having the same atomic number, but an atomic mass or mass number different from the atomic mass or mass number which predominates in nature.

Examples of isotopes suitable for inclusion in the compounds of the disclosure may include isotopes of hydrogen, such as ²H (D, deuterium) and ³H (T, tritium), carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled compounds of the disclosure, for example those incorporating a radioactive isotope, are useful in one or both of drug or substrate tissue distribution studies. The radioactive isotopes, such as, tritium and ¹⁴C are particularly useful for this purpose in view of their ease of incorporation and ready means of detection. Substitution with positron emitting isotopes, such as, ¹¹C, ¹⁸F, ¹⁵O and ¹³N, may be useful in Positron Emission Topography (PET) studies for examining substrate receptor occupancy. Substitution with deuterium may afford certain therapeutic advantages resulting from greater metabolic stability, for example increased in vivo half-life, reduced dosage requirements, reduced CYP450 inhibition (competitive or time dependent), or an improvement in therapeutic index or tolerability.

In some embodiments, the disclosure provides deuterium-labeled (or deuterated) compounds and salts, where the formula and variables of such compounds and salts are each and independently as described herein. “Deuterated” means that at least one of the atoms in the compound is deuterium in an abundance that is greater than the natural abundance of deuterium (typically approximately 0.015%). A skilled artisan recognized that in chemical compounds with a hydrogen atom, the hydrogen atom actually represents a mixture of H and D, with about 0.015% being D. The concentration of the deuterium incorporated into the deuterium-labeled compounds and salt of the disclosure may be defined by the deuterium enrichment factor. It is understood that one or more deuterium may exchange with hydrogen under physiological conditions.

In some embodiments, one or more hydrogen atoms on certain metabolic sites on the compounds of the disclosure are deuterated.

Isotopically-labeled compounds of the disclosure may generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the disclosure include those wherein the solvent of crystallization may be isotopically substituted, e.g., D₂O, d₆-acetone, d₆-DMSO.

### Metabolites

Also included within the scope of the disclosure are active metabolites of compounds of the disclosure, that is, compounds formed in vivo upon administration of the drug, often by oxidation or dealkylation. Some examples of metabolites in accordance with the disclosure include, but are not limited to,
(i) where the compound of the disclosure contains an alkyl group, a hydroxyalkyl derivative thereof (—CH→—COH):
(ii) where the compound of the disclosure contains an alkoxy group, a hydroxy derivative thereof (—OR→—OH);
(iii) where the compound of the disclosure contains a tertiary amino group, a secondary amino derivative thereof (—NRR′→—NHR or —NHR′);
(iv) where the compound of the disclosure contains a secondary amino group, a primary derivative thereof (—NHR→—NH₂);
(v) where the compound of the disclosure contains a phenyl moiety, a phenol derivative thereof (-Ph→-PhOH);
(vi) where the compound of the disclosure contains an amide group, a carboxylic acid derivative thereof (—CONH₂→COOH); and
(vii) where the compound contains a hydroxy or carboxylic acid group, the compound may be metabolized by conjugation, for example with glucuronic acid to form a glucuronide. Other routes of conjugative metabolism exist. These pathways are frequently known as Phase 2 metabolism and include, for example, sulfation or acetylation. Other functional groups, such as NH groups, may also be subject to conjugation.

### Adjuvants

The compounds of the present disclosure can be used as adjuvants, for example within an immunogenic composition (i.e., vaccine). An adjuvant is a substance that enhances the immune response when administered together with an immunogen or antigen. Adjuvants may act primarily as a delivery system, primarily as an immune modulator or have strong features of both. Suitable adjuvants include those suitable for use in mammals, including humans. For example, adjuvants augment the intrinsic immune response to an immunogen without causing conformational changes in the immunogen that may affect the qualitative form of the immune response. In some embodiments, the adjuvant is a TLR 7/8 modulating molecule described herein.

An effective amount of an adjuvant, such as those described herein, refers to the amount necessary or sufficient to realize a desired biologic effect. For example, an effective amount of an adjuvant administered with an antigen for inducing an antigen-specific immune response is that amount necessary to induce an immune response in response to an antigen upon exposure to the antigen. Combined with the teachings provided herein, by choosing among the various adjuvants and weighing factors such as potency, relative bioavailability, subject body weight, severity of adverse side-effects and preferred mode of administration, an effective prophylactic or therapeutic treatment regimen can be planned which does not cause substantial toxicity and yet is effective to treat the particular subject. The effective amount for any particular application can vary depending on such factors as the disease or condition being treated, the particular adjuvant being administered, the size of the subject, or the severity of the disease or condition.

In some embodiments, a compound of the present disclosure can be used as an adjuvant in combination with one or more additional adjuvants. For example, in some embodiments, a compound of the present disclosure can be used as an adjuvant in combination with 1, 2, 3, 4, or more additional adjuvants. In some embodiments, a compound of the present disclosure can be used as an adjuvant in combination with an additional TLR modulating compound. For example, a compound of the present disclosure can be used as an adjuvant in combination with an additional TLR 7/8 modulating compound. In other embodiments, a compound of the present disclosure can be used as an adjuvant in combination with a TLR 4 modulating compound. In other embodiments, a compound of the present disclosure can be used as an adjuvant in combination with a TLR 5 modulating compound. In other embodiments, a compound of the present disclosure can be used as an adjuvant in combination with a TLR 9 modulating compound. In still other embodiments, a compound of the present disclosure can be used as an adjuvant in combination with a cytokine. In some embodiments, a compound of the present disclosure can be used as an adjuvant in combination with a saponin. In some embodiments, a compound of the present disclosure can be used as an adjuvant in combination with a compound that modulates the stimulator of interferon genes (STING) pathway. In other embodiments, a compound of the present disclosure can be used as an adjuvant in combination with a retinoic acid-inducible gene I (RIG-1) modulating compound. In yet other embodiments, a compound of the present disclosure can be used as an adjuvant in combination with aluminum. In one embodiment, a compound of the present disclosure can be used as an adjuvant in combination with aluminum phosphate. In another embodiment, a compound of the present disclosure can be used as an adjuvant in combination with aluminum hydroxide.

In some embodiments, a TLR 7/8 modulating molecule described herein is combined with lipid nanoparticles (LNPs) to form a liposomal adjuvant formulation comprising LNPs, herein referred to as a “LNP adjuvant formulation”. In some embodiments, a TLR 7/8 modulating molecule described herein is combined with a saponin to form a LNP adjuvant formulation. In one embodiment, a TLR 7/8 modulating molecule described herein is combined with QS-21 to form a LNP adjuvant formulation. In some embodiments, a TLR 7/8 modulating molecule described herein is combined with a phospholipid to form a LNP adjuvant formulation.

In some embodiments, a TLR 7/8 modulating molecule described herein is combined with distearyl phosphatidylcholine (DSPC) to form a LNP adjuvant formulation. In some embodiments, a TLR 7/8 modulating molecule described herein is combined with a sterol to form a LNP adjuvant formulation. In some embodiments, a TLR 7/8 modulating molecule described herein is combined with cholesterol and/or a cholesterol analog to form a LNP adjuvant formulation. In some embodiments, a TLR 7/8 modulating molecule described herein is combined with a cationic lipid to form a LNP adjuvant formulation. In some embodiments, a TLR 7/8 modulating molecule described herein is combined with (4-hydroxybutyl)azanediyl)bis(hexane-6,1-diyl)bis(2-hexyldecanoate) (ALC-0315) to form a LNP adjuvant formulation. In some embodiments, a TLR 7/8 modulating molecule described herein is combined with a polymer conjugated lipid to form a LNP adjuvant formulation. In some embodiments, a TLR 7/8 modulating molecule described herein is combined with α-[2-(ditetradecylamino)-2-oxoethyl]-ω-methoxy-poly(oxy-1,2-ethanediyl) (ALC-0159) to form a LNP adjuvant formulation. In some embodiments, a TLR 7/8 modulating molecule described herein is combined with ALC-0315 and ALC-0159 to form a LNP adjuvant formulation. In some embodiments, a TLR 7/8 modulating molecule described herein is combined with a saponin, a phospholipid, a cationic lipid, a polymer conjugated lipid, and a sterol to form a LNP adjuvant formulation. In some embodiments, a TLR 7/8 modulating molecule described herein is combined with QS-21, a phospholipid, a cationic lipid, a polymer conjugated lipid, as well as cholesterol and/or a cholesterol analog to form a LNP adjuvant formulation. In some embodiments, a TLR 7/8 modulating molecule described herein is combined with QS-21, DSPC, a cationic lipid, a polymer conjugated lipid, and cholesterol to form a LNP adjuvant formulation. In a particular embodiment, a TLR 7/8 modulating molecule described herein is combined with QS-21, DSPC, ALC-0315, ALC-0159, and cholesterol to form a LNP adjuvant formulation.

In some embodiments, an LNP formulation described herein, when administered to a subject in combination with an immunogen of interest (or comprising an immunogen of interest), induces an immune response in the subject to the immunogen of interest and the immune response is at least 10%, at least 15%, at least 20%, least 25%, at least 30%, at least 35%, at least 40%, at least 45%, at least 50%, at least 55%, at least 60%, at least 65%, at least 70%, at least 75%, at least 80%, at least 85%, at least 90%, least 95%, at least 100%, at least 125%, at least 150%, at least 175%, at least 200%, at least 225%, at least 250%, at least 275%, at least 300%, at least 350%, at least 400%, at least 450%, or at least 500% higher than the immune response of the subject induced by the administration of the immunogen of interest to the subject without the LNP formulation. In a preferred embodiment, an LNP formulation described herein, when administered to a subject in combination with an immunogen of interest (or comprising an immunogen of interest), induces an immune response in the subject to the immunogen of interest and the immune response is at least 50% higher than the immune response of the subject induced by the administration of the immunogen of interest to the subject without the LNP formulation. In another preferred embodiment, an LNP formulation described herein, when administered to a subject in combination with an immunogen of interest (or comprising an immunogen of interest), induces an immune response in the subject to the immunogen of interest and the immune response is at least 100% higher than the immune response of the subject induced by the administration of the immunogen of interest to the subject without the LNP formulation. In another preferred embodiment, an LNP formulation described herein, when administered to a subject in combination with an immunogen of interest (or comprising an immunogen of interest), induces an immune response in the subject to the immunogen of interest and the immune response is at least 200% higher than the immune response of the subject induced by the administration of the immunogen of interest to the subject without the LNP formulation. In some embodiments, the immune response is measured by opsonophagocytic activity (OPA) geometric mean antibody titers. In some embodiments, the immune response is measured by neutralization geometric mean antibody titers.

In some embodiments, an LNP formulation comprising a TLR 7/8 modulating molecule described herein, when administered to a subject in combination with an immunogen of interest (or comprising an immunogen of interest), induces an immune response in the subject to the immunogen of interest and the immune response is at least 10%, at least 15%, at least 20%, least 25%, at least 30%, at least 35%, at least 40%, at least 45%, at least 50%, at least 55%, at least 60%, at least 65%, at least 70%, at least 75%, at least 80%, at least 85%, at least 90%, least 95%, at least 100%, at least 125%, at least 150%, at least 175%, at least 200%, at least 225%, at least 250%, at least 275%, at least 300%, at least 350%, at least 400%, at least 450%, or at least 500% higher than the immune response of the subject induced by the administration to the subject of the immunogen of interest and an LNP formulation that does not comprise a TLR 7/8 modulating molecule. In some embodiments, an LNP formulation comprising a TLR 7/8 modulating molecule described herein, when administered to a subject in combination with an immunogen of interest (or comprising an immunogen of interest), induces an immune response in the subject to the immunogen of interest and the immune response is at least 50% higher than the immune response of the subject induced by the administration to the subject of the immunogen of interest and an LNP formulation that does not comprise a TLR 7/8 modulating molecule. In some embodiments, an LNP formulation comprising a TLR 7/8 modulating molecule described herein, when administered to a subject in combination with an immunogen of interest (or comprising an immunogen of interest), induces an immune response in the subject to the immunogen of interest and the immune response is at least 100% higher than the immune response of the subject induced by the administration to the subject of the immunogen of interest and an LNP formulation that does not comprise a TLR 7/8 modulating molecule. In some embodiments, an LNP formulation comprising a TLR 7/8 modulating molecule described herein, when administered to a subject in combination with an immunogen of interest (or comprising an immunogen of interest), induces an immune response in the subject to the immunogen of interest and the immune response is at least 200% higher than the immune response of the subject induced by the administration to the subject of the immunogen of interest and an LNP formulation that does not comprise a TLR 7/8 modulating molecule. In some embodiments, the immune response is measured by opsonophagocytic activity (OPA) geometric mean antibody titers. In some embodiments, the immune response is measured by neutralization geometric mean antibody titers.

### Linkers

In some embodiments, the TLR 7/8 modulating molecules disclosed herein comprise a linker that connects the TLR 7/8 modulating portion of the molecule to a terminal hydrophobic moiety. In some embodiments, the TLR 7/8 modulating molecules disclosed herein comprise a linker that connects the TLR 7/8 modulating portion of the molecule to a terminal hydrocarbon chain. In some embodiments, the TLR 7/8 modulating molecules disclosed herein comprise a linker that connects the TLR 7/8 modulating portion of the molecule to a terminal hydrophobic moiety, wherein the linker comprises polyethylene glycol (PEG). In some embodiments, the linker comprises one or more polyethylene glycol (PEG) repeat units. In some embodiments, the linker comprises PEG repeat units, wherein the number of PEG repeat units is an integer between about 1 and about 10. In some embodiments, the linker comprises PEG repeat units, wherein the number of PEG repeat units is an integer between about 1 and about 5. For example, in some embodiments, the linker comprises PEG repeat units, wherein the number of PEG repeat units is 1, 2, 3, 4, 5, 6, 7, 8, 9, or 10. In some embodiments, the linker comprises PEG repeat units, wherein the number of PEG repeat units is 1. In some embodiments, the linker comprises PEG repeat units, wherein the number of PEG repeat units is 2. In particular embodiments, the linker comprises PEG repeat units, wherein the number of PEG repeat units is 3. In other embodiments, the linker comprises PEG repeat units, wherein the number of PEG repeat units is 4. In other embodiments, the linker comprises PEG repeat units, wherein the number of PEG repeat units is 5.

In some embodiments, the TLR 7/8 modulating molecules disclosed herein comprise a linker that connects the TLR 7/8 modulating portion of the molecule to a terminal hydrophobic moiety, wherein the linker does not comprise PEG.

In various embodiments, the TLR 7/8 modulating molecules disclosed herein comprise a linker that connects the TLR 7/8 modulating portion of the molecule to a terminal hydrophobic moiety, wherein the linker comprises an alkyl group. For example, in some embodiments the linker comprises an alkyl group that contains 1, 2, 3, 4, 5, 6, 7, 8, 9, or 10 carbons. In a particular embodiment, the linker comprises an alkyl group that contains 2 carbons. In another particular embodiment, the linker comprises an alkyl group that contains 3 carbons. In another particular embodiment, the linker comprises an alkyl group that contains 4 carbons. In some embodiments, the TLR 7/8 modulating molecules disclosed herein comprise a linker that connects the TLR 7/8 modulating portion of the molecule to a terminal hydrophobic moiety, wherein the linker comprises a cyclic amine. In some embodiments, the TLR 7/8 modulating molecules disclosed herein comprise a linker that connects the TLR 7/8 modulating portion of the molecule to a terminal hydrophobic moiety, wherein the linker comprises a derivative of an amino acid.

### SAPONINS

In some embodiments, the LNPs disclosed herein comprise a saponin. For the present embodiments, a suitable saponin is Quil A, its derivatives thereof, or any purified component thereof (for example, QS-7, QS-18, QS-21, or a mixture thereof). Quil A is a saponin preparation isolated from the South American tree Quillaja Saponaria Molina and was first found to have adjuvant activity. (See Dalsgaard et al., 1974, Archiv. für die gesanite Virusforschung, 44:243-254). Purified fragments of Quil A have been isolated by HPLC (See EP U.S. Pat. No. 362,279), including, for example, QS-7 and QS-21 (also known as QA7 and QA21, respectively). QS-21 is the 21st fraction purified from the sap of Quillaja Saponaria tree (See Qi et al. A Two-Step Orthogonal Chromatographic Process for Purifying the Molecular Adjuvant QS-21 with High Purity and Yield. J Chromatogr A. 2021 Jan. 4; 1635:461705). QS-21 has been shown to induce CD8+ cytotoxic T cells (CTLs), Th1 cells, and a predominant IgG2a antibody response (See Wong et al.; TCR Vaccines Against T Cell Lymphoma: QS-21 and IL-12 Adjuvants Induce a Protective CD8+ T Cell Response1. J Immunol 15 Feb. 1999; 162 (4): 2251-2258).

In some embodiments, a composition comprising LNPs is provided, wherein the LNPs comprise a saponin, and wherein the concentration of the saponin in the composition (total weight per ml of the composition) is less than about 1 mg/ml. In some embodiments, a composition comprising LNPs is provided, wherein the LNPs comprise a saponin, and wherein the concentration of the saponin in the composition is about 0.9 mg/ml, about 0.8 mg/ml, about 0.7 mg/ml, about 0.6 mg/ml, about 0.5 mg/ml, about 0.4 mg/ml, about 0.3 mg/ml, about 0.2 mg/ml, or about 0.1 mg/ml. In some embodiments, a composition comprising LNPs is provided, wherein the LNPs comprise a saponin, and wherein the concentration of the saponin in the composition is between about 0.001 mg/mL and about 0.1 mg/mL. In some embodiments, a composition comprising LNPs is provided, wherein the LNPs comprise a saponin, and wherein the concentration of the saponin in the composition is about 0.09 mg/ml, about 0.08 mg/ml, about 0.07 mg/ml, about 0.06 mg/ml, about 0.05 mg/ml, about 0.04 mg/ml, about 0.03 mg/ml, about 0.02 mg/ml, or about 0.01 mg/ml.

In a preferred embodiment, a composition comprising LNPs is provided, wherein the LNPs comprise a saponin, and wherein the concentration of the saponin in the composition is about 0.009 mg/mL. In another preferred embodiment, a composition comprising LNPs is provided, wherein the LNPs comprise a saponin, and wherein the concentration of the saponin in the composition is about 0.023 mg/mL. In another preferred embodiment, a composition comprising LNPs is provided, wherein the LNPs comprise a saponin, and wherein the concentration of the saponin in the composition is about 0.045 mg/mL. In yet another preferred embodiment, a composition comprising LNPs is provided, wherein the LNPs comprise a saponin, and wherein the concentration of the saponin in the composition is about 0.1 mg/mL.

In some embodiments, a composition comprising LNPs is provided, wherein the LNPs comprise QS-21, and wherein the concentration of the QS-21 in the composition (total weight per ml of the composition) is less than about 1 mg/ml. In some embodiments, a composition comprising LNPs is provided, wherein the LNPs comprise QS-21, and wherein the concentration of the QS-21 in the composition is about 0.9 mg/ml, about 0.8 mg/ml, about 0.7 mg/ml, about 0.6 mg/ml, about 0.5 mg/ml, about 0.4 mg/ml, about 0.3 mg/ml, about 0.2 mg/ml, or about 0.1 mg/ml. In some embodiments, a composition comprising LNPs is provided, wherein the LNPs comprise QS-21, and wherein the concentration of the QS-21 in the composition is between about 0.001 mg/mL and about 0.1 mg/mL. In some embodiments, a composition comprising LNPs is provided, wherein the LNPs comprise QS-21, and wherein the concentration of the QS-21 in the composition is about 0.09 mg/ml, about 0.08 mg/ml, about 0.07 mg/ml, about 0.06 mg/ml, about 0.05 mg/ml, about 0.04 mg/ml, about 0.03 mg/ml, about 0.02 mg/ml, or about 0.01 mg/ml.

In a preferred embodiment, a composition comprising LNPs is provided, wherein the LNPs comprise QS-21, and wherein the concentration of the QS-21 in the composition is about 0.009 mg/mL. In another preferred embodiment, a composition comprising LNPs is provided, wherein the LNPs comprise QS-21, and wherein the concentration of the QS-21 in the composition is about 0.023 mg/mL. In another preferred embodiment, a composition comprising LNPs is provided, wherein the LNPs comprise QS-21, and wherein the concentration of the saponin in the composition is about 0.045 mg/mL. In yet another preferred embodiment, a composition comprising LNPs is provided, wherein the LNPs comprise QS-21, and wherein the concentration of the QS-21 in the composition is about 0.1 mg/mL.

### LNP Components

In an embodiment, a mRNA vaccine comprises a LNP formulation comprising lipids and mRNA (RNA-LNPs). The lipids encapsulate the mRNA in the form of a lipid nanoparticle (LNP) to aid cell entry and stability of the RNA-LNPs.

Lipid nanoparticles may include a lipid component and one or more additional components, such as a therapeutic and/or prophylactic. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combinations of elements. The efficacy and tolerability of a LNP formulation may be affected by the stability of the formulation.

Lipid nanoparticles may be designed for one or more specific applications or targets. For example, a LNP may be designed to deliver a therapeutic and/or prophylactic such as an RNA to a particular cell, tissue, organ, or system or group thereof in a mammal's body. The LNP delivery system may have adjuvant effects which enhance the immunogenicity of an encoded antigen and/or other antigens in the composition.

Physiochemical properties of lipid nanoparticles may be altered to increase selectivity for particular bodily targets. For instance, particle sizes may be adjusted based on the fenestration sizes of different organs. The therapeutic and/or prophylactic included in a LNP may also be selected based on the desired delivery target or targets. For example, a therapeutic and/or prophylactic may be selected for a particular indication, condition, disease, or disorder and/or for delivery to a particular cell, tissue, organ, or system or group thereof (e.g., localized or specific delivery). In certain embodiments, a LNP may include an mRNA encoding a polypeptide of interest capable of being translated within a cell to produce the polypeptide of interest. Such a composition may be designed to be specifically delivered to a particular organ. In some embodiments, a composition may be designed to be specifically delivered to a mammalian liver. In some embodiments, a composition may be designed to be specifically delivered to a lymph node. In some embodiments, a composition may be designed to be specifically delivered to a mammalian spleen.

Lipid nanoparticles may include a lipid component and one or more additional components, such as a therapeutic and/or prophylactic, such as a nucleic acid. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combination of elements. The efficacy and tolerability of a LNP formulation may be affected by the stability of the formulation.

In some embodiments, for example, a polymer may be included in and/or used to encapsulate or partially encapsulate or be conjugated to a lipid (polymer-conjugated lipid or polymer lipid) in a LNP. A polymer may be biodegradable and/or biocompatible. A polymer may be selected from, but is not limited to, polyamines, polyethers, polyamides, polyesters, poly carbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(gly colic acid) (PGA), poly(lactic acid-co-gly colic acid) (PLGA), poly(L-lactic acid-co-gly colic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl (meth)acrylate) (PMMA), poly(ethyl (meth)acrylate), poly(butyl (meth)acrylate), poly(isobutyl (meth)acrylate), poly(hexyl (meth)acrylate), poly(isodecyl (meth)acrylate), poly(lauryl (meth)acrylate), poly(phenyl (meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho) esters, poly(butyric acid), poly(valeric acid), poly(lactide-co-caprolactone), trimethylene carbonate, poly(N-acryloylmorpholine) (PACM), poly(2-methyl-2-oxazoline) (PMOX), poly(2-ethyl-2-oxazoline) (PEOZ), poly(2-oxazoline) (POZ), which is a synthetic, water-soluble, and low-viscosity polymer, and polyglycerol.

In some embodiments, the amount of polymer-lipid in the lipid composition of a pharmaceutical composition disclosed herein ranges from about 0.1 mol % to about 5 mol %, from about 0.5 mol % to about 5 mol %, from about 1 mol % to about 5 mol %, from about 1.5 mol % to about 5 mol %, from about 2 mol % to about 5 mol %, from about 0.1 mol % to about 4 mol %, from about 0.5 mol % to about 4 mol %, from about 1 mol % to about 4 mol %, from about 1.5 mol % to about 4 mol %, from about 2 mol % to about 4 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 0.1 mol % to about 2 mol %, from about 0.5 mol % to about 2 mol %, from about 1 mol % to about 2 mol %, from about 1.5 mol % to about 2 mol %, from about 0.1 mol % to about 1.5 mol %, from about 0.5 mol % to about 1.5 mol %, or from about 1 mol % to about 1.5 mol %.

In some embodiments, the amount of polymer-lipid in the lipid composition disclosed herein is about 2 mol %. In some embodiments, the amount of polymer-lipid in the lipid composition disclosed herein is about 1.5 mol %. In some embodiments, the amount of polymer-lipid in the lipid composition disclosed herein is at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 mol %. In a preferred embodiment, the amount of polymer-lipid (or polymer-conjugated lipid) in the lipid composition is 1.8 mol %.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A LNP may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, surface active agents, buffering agents, preservatives, and other species.

Surface active agents and/or emulsifiers may include, but are not limited to, natural emulsifiers (e.g., acacia, alginic acid, sodium alginate, cholesterol, and lecithin), sorbitan fatty acid esters (e.g., polyoxy ethylene sorbitan monolaurate [TWEEN®20], polyoxy ethylene sorbitan [TWEEN® 60], polyoxy ethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

Examples of preservatives may include, but are not limited to, antioxidants, chelating agents, free radical scavengers, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxy toluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxy toluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®. An exemplary free radical scavenger includes butylated hydroxytoluene (BHT or butylhydroxytoluene) or deferoxamine.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, Tris buffer, and/or combinations thereof.

In some embodiments, the formulation including a LNP may further include a salt, such as a chloride salt. In some embodiments, the formulation including a LNP may further includes a sugar such as a disaccharide. In some embodiments, the formulation further includes a sugar but not a salt, such as a chloride salt. In some embodiments, a LNP may further include one or more small hydrophobic molecules such as a vitamin (e.g., vitamin A or vitamin E) or a sterol. Carbohydrates may include simple sugars (e.g., glucose) and polysaccharides (e.g., glycogen and derivatives and analogs thereof).

The characteristics of a LNP may depend on the components thereof. For example, a LNP including cholesterol as a structural lipid may have different characteristics than a LNP that includes a different structural lipid. As used herein, the term “structural lipid” refers to sterols and also to lipids containing sterol moieties. As defined herein, “sterols” are a subgroup of steroids consisting of steroid alcohols. In some embodiments, the structural lipid is a steroid. In some embodiments, the structural lipid is cholesterol. In some embodiments, the structural lipid is an analog of cholesterol.

The lipid nanoparticle compositions may include a mixture of structural lipids. Incorporation of structural lipids in the lipid nanoparticle may help mitigate aggregation of other lipids in the particle. Structural lipids can be selected from the group including but not limited to, cholesterol and cholesterol analogs such as fecosterol, sitosterol, β-sitosterol, ergosterol, campesterol, stigmasterol, brassicasterol, tomatidine, tomatine, ursolic acid, alpha-tocopherol, hopanoids, sitostanol, phytosterols, steroids, and mixtures thereof. In some embodiments, the structural lipid is a sterol. In some embodiments, the structural lipid is a steroid. In some embodiments, the structural lipid is cholesterol. In certain embodiments, the structural lipid is an analog of cholesterol. In some preferred embodiments, the sterol comprises

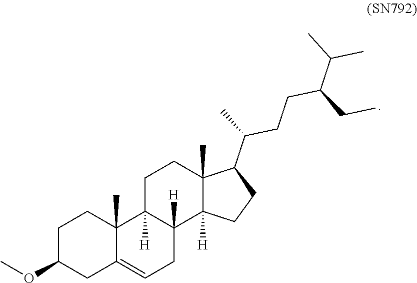

In some preferred embodiments, the sterol comprises

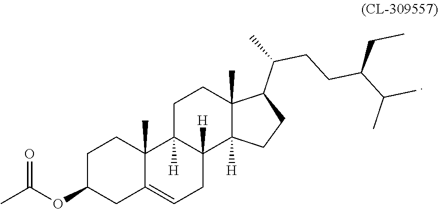

In some preferred embodiments, the sterol comprises stigmasterol.

In some preferred embodiments, the sterol comprises β-sitosterol

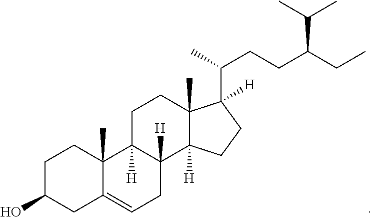

In some embodiments, the structural lipid is a sitosterol, a stigmasterol, a campesterol, a sitostanol, a campestanol, a brassicasterol, a fucosterol, beta-sitosterol, stigmastanol, beta-sitostanol, ergosterol, lupeol, cycloartenol, Δ5-avenasterol, Δ7-avenasterol or a Δ7-stigmasterol, including analogs, salts or esters thereof, alone or in combination. In some embodiments, the sterol component of a LNP of the disclosure is a single phytosterol. In some embodiments, the phytosterol component of a LNP of the disclosure is a mixture of different phytosterols (e.g. 2, 3, 4, 5 or 6 different phytosterols). In some embodiments, the phytosterol component of an LNP of the disclosure is a blend of one or more phytosterols and one or more zoosterols, such as a blend of a phytosterol (e.g., a sitosterol, such as beta-sitosterol) and cholesterol. In some embodiments, the phytosterol is β-sitosterol, campesterol, sigmastanol, or any combination thereof. In some embodiments, the phytosterol is β-sitosterol. In some embodiments, the cholesterol analog comprises β-sitosterol, campesterol, and stigmasterol. In some embodiments, an LNP disclosed herein comprises 1, 2, 3, or more structural lipids.

In some embodiments, an LNP formulation disclosed herein comprises cholesterol and β-sitosterol. In some embodiments, an LNP formulation disclosed herein comprises cholesterol and β-sitosterol, wherein the ratio of cholesterol to β-sitosterol is between about 6:1 and about 1:6. In some embodiments, an LNP formulation disclosed herein comprises cholesterol and β-sitosterol, wherein the ratio of cholesterol to β-sitosterol is between about 6:1 and about 1:1. In some embodiments, an LNP formulation disclosed herein comprises cholesterol and β-sitosterol, wherein the ratio of cholesterol to β-sitosterol is about 6:5, about 6:4, about 6:3, about 6:2, or about 6:1.

In some embodiments, an LNP formulation disclosed herein comprises cholesterol and β-sitosterol. In some embodiments, an LNP formulation disclosed herein comprises cholesterol and β-sitosterol, wherein the ratio of cholesterol to β-sitosterol is between about 1:9 and about 9:1. In some embodiments, an LNP formulation disclosed herein comprises cholesterol and β-sitosterol, wherein the ratio of cholesterol to β-sitosterol is about 1:9, about 2:8, about 8:2, or about 9:1.

In a preferred embodiment, an LNP formulation disclosed herein comprises cholesterol and β-sitosterol, wherein the ratio of cholesterol to β-sitosterol is about 6:4. In another preferred embodiment, an LNP formulation disclosed herein comprises cholesterol and β-sitosterol, wherein the ratio of cholesterol to β-sitosterol is about 4:6.

In some embodiments, an LNP formulation disclosed herein comprises β-sitosterol, stigmasterol, and campesterol. In some embodiments, an LNP formulation disclosed herein comprises structural lipids β-sitosterol, stigmasterol, and campesterol, wherein the mixture of structural lipids comprises about 35% to about 85% of β-sitosterol, about 5% to about 35% stigmasterol, and about 5% to about 35% of campesterol. In some embodiments, an LNP formulation disclosed herein comprises structural lipids β-sitosterol, stigmasterol, and campesterol, wherein the mixture of structural lipids comprises about 35% to about 45% of β-sitosterol, about 20% to about 30% stigmasterol, and about 20% to about 30% of campesterol. In some embodiments, an LNP formulation disclosed herein comprises structural lipids β-sitosterol, stigmasterol, and campesterol, wherein the mixture of structural lipids comprises about 65% to about 75% of β-sitosterol, about 5% to about 15% stigmasterol, and about 5% to about 15% of campesterol.

In some embodiments, an LNP formulation disclosed herein comprises structural lipids β-sitosterol and stigmasterol, wherein the mixture of structural lipids comprises about 35% to about 85% of β-sitosterol and about 5% to about 35% stigmasterol. In some embodiments, an LNP formulation disclosed herein comprises structural lipids β-sitosterol and stigmasterol, wherein the mixture of structural lipids comprises about 35% to about 45% of β-sitosterol, and about 20% to about 30% stigmasterol.

In some embodiments, an LNP formulation disclosed herein comprises cholesterol, β-sitosterol, and stigmasterol, wherein the mixture of structural lipids comprises about 10% to about 30% of cholesterol, about 10% to about 30% β-sitosterol, and about 10% to about 30% stigmasterol. In some embodiments, an LNP formulation disclosed herein comprises about 30-50% cationic lipid and about 5-25% phospholipid.

In some embodiments, the mol % of structural lipids out of total lipids in an LNP formulation disclosed herein is between about 30 mol % and about 60 mol %. In some embodiments, the mol % of structural lipids out of total lipids in an LNP formulation disclosed herein is between about 40 mol % and about 60 mol %. In some embodiments, the mol % of structural lipids out of total lipids in an LNP formulation disclosed herein is between about 30 mol % and about 50 mol %. In some embodiments, the mol % of structural lipids out of total lipids in an LNP formulation disclosed herein is between about 40 mol % and about 50 mol %. In some embodiments, the mol % of structural lipids out of total lipids in an LNP formulation disclosed herein is about 35 mol %, about 36 mol %, about 37 mol %, about 38 mol %, about 39 mol %, about 40 mol %, about 41 mol %, about 42 mol %, about 43 mol %, about 44 mol %, or about 45 mol %. In a preferred embodiment, the mol % of structural lipids out of total lipids in an LNP formulation disclosed herein is between about 40 mol % and about 41 mol %. In a preferred embodiment, the mol % of structural lipids out of total lipids in an LNP formulation disclosed herein is about 40.7 mol %.

In another preferred embodiment, an LNP formulation disclosed herein comprises cholesterol and β-sitosterol, and the mol % of cholesterol and β-sitosterol out of total lipids in the LNP formulation is between about 40 mol % and about 41 mol %. In another preferred embodiment, an LNP formulation disclosed herein comprises cholesterol and β-sitosterol, and the mol % of cholesterol and β-sitosterol out of total lipids in the LNP formulation is about 40.7 mol %

In some embodiments, the characteristics of a LNP may depend on the absolute or relative amounts of its components. For instance, a LNP including a higher molar fraction of a phospholipid may have different characteristics than a LNP including a lower molar fraction of a phospholipid. Characteristics may also vary depending on the method and conditions of preparation of the lipid nanoparticle. In general, phospholipids comprise a phospholipid moiety and one or more fatty acid moieties. A phospholipid moiety can be selected, for example, from the non-limiting group consisting of phosphatidyl choline, phosphatidyl ethanolamine, phosphatidyl glycerol, phosphatidyl serine, phosphatidic acid, and 2-lysophosphatidyl choline.

Further examples of a phospholipid moiety for the lipid nanoparticle include a lipid that is selected from the group consisting of distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, I-stearoyl-2-oleoyl-phosphatidyethanolamine (SOPE), hydrogenated soy phosphatidylcholine (HSPC), egg phosphatidylcholine (EPC), dioleoylphosphatidyl serine (DOPS), sphingomyelin (SM), dimyristoyl phosphatidylcholine (DMPC), dimyristoyl phosphatidylglycerol (DMPG), distearoylphosphatidylglycerol (DSPG), diemcoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, cephalin, cardiolipin, phosphatidicacid, cerebrosides, dicetylphosphate, lysophosphatidylcholine, dilinoleoylphosphatidylcholine, and mixtures thereof.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Particular phospholipids can facilitate fusion to a membrane. In some embodiments, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue. Non-natural phospholipid species including natural species with modifications and substitutions including branching, oxidation, cyclization, and alkynes are also contemplated. In some embodiments, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye). Phospholipids include, but are not limited to, glycerophospholipids such as phosphatidylcholines, phosphatidyl-ethanolamines, phosphatidylserines, phosphatidylinositols, phosphatidy glycerols, and phosphatidic acids. In some embodiments, a phospholipid useful or potentially useful in the present invention is an analog or variant of DSPC.

Lipid nanoparticles may be characterized by a variety of methods. For example, microscopy (e.g., transmission electron microscopy or scanning electron microscopy) may be used to examine the morphology and size distribution of a LNP. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a LNP, such as particle size, polydispersity index, and zeta potential.

The term “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PDI), which is dimensionless (Koppel, D., J. Chem. Phys. 57, 1972, pp 4814-4820, ISO 13321). Here, “mean diameter,” “diameter,” “size” or “mean size” for particles is used synonymously with this value of the Z-average. In some aspects, an LNP formulation disclosed herein comprises LNPs having a mean diameter between about 1 and about 500 nm. In some aspects, an LNP formulation disclosed herein comprises LNPs having a mean diameter between about 30 nm and about 150 nm, between about 40 nm and about 150 nm, between about 50 nm and about 150 nm, between about 60 nm and about 130 nm, between about 70 nm and about 110 nm, between about 70 nm and about 100 nm, between about 80 nm and about 100 nm, between about 90 nm and about 100 nm, between about 70 and about 90 nm, between about 80 nm and about 90 nm, between about 70 nm and about 80 nm, between about 60 nm and about 70 nm, or at least, at most, exactly, or between any two of 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In a particular embodiment, an LNP formulation disclosed herein comprises LNPs having a mean diameter between about 60 nm and about 140 nm.

In some embodiments, an LNP formulation is provided herein wherein the LNPs comprise a TLR 7/8 modulating molecule, and the average size of the LNPs in the formulation is within 20%, 15%, 10%, 5%, 4%, 3%, 2%, or 1% of the average size of LNPs in a formulation wherein the LNPs do not comprise a TLR 7/8 modulating molecule. In a preferred embodiment, an LNP formulation is provided herein wherein the LNPs comprise a TLR 7/8 modulating molecule and the average size of the LNPs in the formulation is within 10% of the average size of LNPs in a formulation wherein the LNPs do not comprise a TLR 7/8 modulating molecule. In another preferred embodiment, an LNP formulation is provided herein wherein the LNPs comprise a TLR 7/8 modulating molecule and the average size of the LNPs in the formulation is within 5% of the average size of LNPs in a formulation wherein the LNPs do not comprise a TLR 7/8 modulating molecule.

An LNP formulation disclosed herein may be relatively homogenous. A polydispersity index (PDI) may be used to indicate the homogeneity of an LNP formulation, e.g., the particle size distribution of the lipid nanoparticles. The PDI is, in some aspects, calculated based on dynamic light scattering measurements by the so-called cumulant analysis as mentioned in the definition of the “average diameter.” Under certain prerequisites, it may be taken as a measure of the size distribution of an ensemble of nanoparticles. A small (e.g., less than 0.3) PDI generally indicates a narrow particle size distribution. An LNP formulation disclosed herein may have a PDI from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. An LNP formulation disclosed herein may exhibit a PDI from about 0.10 to about 0.20. An LNP formulation disclosed herein may exhibit a PDI less than about 0.5, less than about 0.4, less than about 0.3, less than about 0.2, or less than about 0.1.

In a preferred embodiment, an LNP formulation disclosed herein has a PDI less than about 0.2. In another preferred embodiment, an LNP formulation disclosed herein has a PDI between about 0.05 and about 0.2.

In some embodiments, in an LNP formulation wherein the LNPs comprise a TLR 7/8 modulating molecule, the PDI of the LNPs in the formulation is within 20%, 15%, 10%, 5%, 4%, 3%, 2%, or 1% of the PDI of LNPs in a formulation wherein the LNPs do not comprise a TLR 7/8 modulating molecule. In a preferred embodiment, in an LNP formulation wherein the LNPs comprise a TLR 7/8 modulating molecule, the PDI of the LNPs in the formulation is within 10% of the PDI of LNPs in a formulation wherein the LNPs do not comprise a TLR 7/8 modulating molecule. In another preferred embodiment, in an LNP formulation wherein the LNPs comprise a TLR 7/8 modulating molecule, the PDI of the LNPs in the formulation is within 5% of the PDI of LNPs in a formulation wherein the LNPs do not comprise a TLR 7/8 modulating molecule.

The zeta potential of a LNP may be used to indicate the electrokinetic potential of the composition. For example, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

A LNP may optionally comprise one or more coatings. For example, a LNP may be formulated in a capsule, film, or tablet having a coating. A capsule, film, or tablet including a composition described herein may have any useful size, tensile strength, hardness, or density.

Formulations comprising amphiphilic polymers and lipid nanoparticles may be formulated in whole or in part as pharmaceutical compositions. Pharmaceutical compositions may include one or more amphiphilic polymers and one or more lipid nanoparticles. For example, a pharmaceutical composition may include one or more amphiphilic polymers and one or more lipid nanoparticles including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21st Edition, A. R. Gennaro; Lippincott, Williams & Wilkins, Baltimore, MD, 2006. Conventional excipients and accessory ingredients may be used in any pharmaceutical composition, except insofar as any conventional excipient or accessory ingredient may be incompatible with one or more components of a LNP or the one or more amphiphilic polymers in the formulation of the disclosure. An excipient or accessory ingredient may be incompatible with a component of a LNP or the amphiphilic polymer of the formulation if its combination with the component or amphiphilic polymer may result in any undesirable biological effect or otherwise deleterious effect.

In some embodiments, one or more excipients or accessory ingredients may make up greater than 50% of the total mass or volume of a pharmaceutical composition including a LNP. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia. Relative amounts of the one or more amphiphilic polymers, the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more lipid nanoparticles. As another example, a pharmaceutical composition may comprise between 0.1% and 15% (wt/vol) of one or more amphiphilic polymers (e.g., 0.5%, 1%, 2.5%, 5%, 10%, or 12.5% w/V).

The chemical properties of the LNP, LNP suspension, lyophilized LNP composition, or LNP formulation of the present disclosure may be characterized by a variety of methods. In some embodiments, fragment analyzer, electrophoresis (e.g., capillary electrophoresis) or chromatography (e.g., reverse phase liquid chromatography) may be used to examine the mRNA integrity.

In certain embodiments, the lipid nanoparticles and/or pharmaceutical compositions of the disclosure are refrigerated or frozen for storage and/or shipment (e.g., being stored at a temperature of about 5° C. or lower, such as a temperature between about −150° C. and about 5° C. or between about −150° C. and about 0° C. or between about −80° C. and about −20° C. (e.g., about 5° C., 0° C., −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more amphiphilic polymers and one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles by adding an effective amount of an amphiphilic polymer and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.).

### RNA Encapsulation

In one aspect RNA is encapsulated in an LNP disclosed herein to produce lipid nanoparticle (LNP)-encapsulated RNA (RNA-LNPs). Without intending to be bound by any theory, it is believed that the cationic or cationically ionizable lipid or lipid-like material and/or the cationic polymer combine together with the nucleic acid to form aggregates, and this aggregation results in colloidally stable particles.

A lipid may be a naturally occurring lipid or a synthetic lipid. However, a lipid is usually a biological substance. Biological lipids are well known in the art, and include for example, neutral fats, phospholipids, phosphoglycerides, steroids, terpenes, lysolipids, glycosphingolipids, glucolipids, sulphatides, lipids with ether and ester-linked fatty acids and polymerizable lipids, and combinations thereof. A lipid is a substance that is insoluble in water and extractable with an organic solvent. Compounds other than those specifically described herein are understood by one of skill in the art as lipids and are encompassed by the compositions and methods of the present disclosure. A lipid component and a non-lipid may be attached to one another, either covalently or non-covalently.

In some aspects, LNPs may be designed to protect RNA molecules (e.g., saRNA, mRNA) from extracellular Rnases and/or may be engineered for systemic delivery of the RNA to target cells. In some aspects, such LNPs may be particularly useful to deliver RNA molecules (e.g., saRNA, mRNA) when RNA molecules are intravenously administered to a subject in need thereof. In some aspects, such LNPs may be particularly useful to deliver RNA molecules (e.g., saRNA, mRNA) when RNA molecules are intramuscularly administered to a subject in need thereof. In some aspects, such LNPs may be particularly useful to deliver RNA molecules (e.g., saRNA, mRNA) when RNA molecules are intradermally administered to a subject in need thereof. In some aspects, such LNPs may be particularly useful to deliver RNA molecules (e.g., saRNA, mRNA) when RNA molecules are intranasally administered to a subject in need thereof.

The “efficiency of encapsulation” of a therapeutic and/or prophylactic (e.g., RNA or TLR 7/8 modulating compound) describes the amount of therapeutic and/or prophylactic (e.g., RNA or TLR 7/8 modulating compound) that is encapsulated or otherwise associated with an LNP after preparation, relative to the initial amount provided. The encapsulation efficiency is desirably high (e.g., close to 100%). The encapsulation efficiency may be measured, for example, by comparing the amount of therapeutic and/or prophylactic (e.g., RNA or TLR 7/8 modulating compound) in a solution containing the lipid nanoparticle before and after breaking up the lipid nanoparticle with one or more organic solvents or detergents. Fluorescence may be used to measure the amount of free therapeutic and/or prophylactic (e.g., RNA or TLR 7/8 modulating compound) in a solution. For example, if 97 mg of mRNA are encapsulated in a composition out of a total 100 mg of mRNA initially provided to the composition, the encapsulation efficiency may be given as 97%. For the lipid nanoparticle formulations described herein, the encapsulation efficiency of the RNA may be at least 50%, for example at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100%. In a preferred embodiment of the lipid nanoparticle formulations described herein, the encapsulation efficiency of the RNA is at least 60%. In another preferred embodiment of the lipid nanoparticle formulations described herein, the encapsulation efficiency of the RNA is at least 70%. In yet another preferred embodiment of the lipid nanoparticle formulations described herein, the encapsulation efficiency of the RNA is at least 80%.

For the lipid nanoparticle formulations described herein, the encapsulation efficiency of the TLR 7/8 modulating molecule may be at least 40%, for example at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100%. In a preferred embodiment of the lipid nanoparticle formulations described herein, the encapsulation efficiency of the TLR 7/8 modulating molecule is at least 50%. In another preferred embodiment of the lipid nanoparticle formulations described herein, the encapsulation efficiency of the TLR 7/8 modulating molecule is at least 60%. In yet another preferred embodiment of the lipid nanoparticle formulations described herein, the encapsulation efficiency of the TLR 7/8 modulating molecule is at least 70%.

In some embodiments, in an LNP formulation wherein the LNPs comprise a TLR 7/8 modulating molecule, the encapsulation efficiency of the RNA is within 20%, 15%, 10%, 5%, 4%, 3%, 2%, or 1% of the encapsulation efficiency of RNA within an LNP formulation wherein the LNPs do not comprise a TLR 7/8 modulating molecule. In a preferred embodiment, in an LNP formulation wherein the LNPs comprise a TLR 7/8 modulating molecule, the encapsulation efficiency of the RNA is within 10% of the encapsulation efficiency of RNA within an LNP formulation wherein the LNPs do not comprise a TLR 7/8 modulating molecule. In another preferred embodiment, in an LNP formulation wherein the LNPs comprise a TLR 7/8 modulating molecule, the encapsulation efficiency of the RNA is within 5% of the encapsulation efficiency of RNA within an LNP formulation wherein the LNPs do not comprise a TLR 7/8 modulating molecule.

In one aspect, a composition is provided herein wherein the composition comprises RNA-LNPs, and wherein the concentration of RNA in the composition is about 1 mg/mL, or more. For example, a composition is provided herein wherein the composition comprises RNA-LNPs, and wherein the concentration of RNA in the composition is about 1 mg/mL, about 10 mg/mL, about 50 mg/mL, about 75 mg/mL, about 100 mg/mL, about 150 mg/mL, about 200 mg/mL, about 250 mg/mL, about 300 mg/mL, about 400 mg/mL, or more. In one aspect, a composition is provided herein wherein the composition comprises RNA-LNPs, and wherein the concentration of RNA in the composition is <1 mg/mL. In another aspect, a composition is provided herein wherein the composition comprises RNA-LNPs, and wherein the concentration of RNA in the composition is at least about 0.05 mg/mL. In another aspect, a composition is provided herein wherein the composition comprises RNA-LNPs, and wherein the concentration of RNA in the composition is between about 0.05 mg/mL and about 1 mg/mL. In another aspect, a composition is provided herein wherein the composition comprises RNA-LNPs, and wherein the concentration of RNA in the composition is between about 0.05 mg/mL and about 0.5 mg/mL. In some aspects, a composition is provided herein wherein the composition comprises RNA-LNPs, and wherein the concentration of RNA in the composition is at least, at most, exactly, between (inclusive or exclusive) any two of, or about 0.10 mg/mL, 0.11 mg/mL, 0.12 mg/mL, 0.13 mg/mL, 0.14 mg/mL, 0.15 mg/mL, 0.16 mg/mL, 0.17 mg/mL, 0.18 mg/mL, 0.19 mg/mL, 0.20 mg/mL, about 0.21 mg/mL, 0.22 mg/mL, 0.23 mg/mL, 0.24 mg/mL, 0.25 mg/mL, 0.26 mg/mL, 0.27 mg/mL, 0.28 mg/mL, 0.29 mg/mL, 0.30 mg/mL, 0.31 mg/mL, 0.32 mg/mL, 0.33 mg/mL, 0.34 mg/mL, or 0.35 mg/mL.

In a preferred embodiment, a composition is provided herein wherein the composition comprises RNA-LNPs, and wherein the concentration of RNA in the composition is about 0.1 mg/mL.

In one aspect, an LNP formulation is provided, wherein the concentration of TLR 7/8 modulating molecule in the LNP formulation is less than 1 mg/mL. In some embodiments, an LNP formulation is provided, wherein the concentration of TLR 7/8 modulating molecule in the LNP formulation is between about 1 μg/mL and about 1000 μg/mL. In some embodiments, an LNP formulation is provided, wherein the concentration of TLR 7/8 modulating molecule in the LNP formulation is between about 100 μg/mL and about 500 μg/mL, for example, about 100 μg/mL, about 150 μg/mL, about 200 μg/mL, about 250 g/mL, about 300 μg/mL, about 350 μg/mL, about 400 μg/mL, about 450 μg/mL, or about 500 g/mL. In some embodiments, an LNP formulation is provided, wherein the concentration of TLR 7/8 modulating molecule in the LNP formulation is between about 10 μg/mL and about 100 μg/mL, for example, about 10 μg/mL, about 15 μg/mL, about 20 μg/mL, about 25 μg/mL, about 30 μg/mL, about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, about 85 μg/mL, about 90 μg/mL, about 95 μg/mL, or about 100 μg/mL.

In a preferred embodiment, an LNP formulation is provided, wherein the concentration of TLR 7/8 modulating molecule in the LNP formulation is about 17 μg/mL. In another preferred embodiment, an LNP formulation is provided, wherein the concentration of TLR 7/8 modulating molecule in the LNP formulation is about 86 μg/mL. In yet another preferred embodiment, an LNP formulation is provided, wherein the concentration of TLR 7/8 modulating molecule in the LNP formulation is about 344 μg/mL.

The efficacy of a product is dependent on expression of the delivered RNA, which requires a sufficiently intact RNA molecule. RNA integrity is a measure of RNA quality that quantitates intact RNA. The method is also capable of detecting potential degradation products. RNA integrity can be determined by capillary gel electrophoresis or fragment analyzer (FA). The initial specification is set to ensure sufficient RNA integrity in drug product preparations. In some embodiments, the RNA polynucleotides within an LNP formulation have an integrity of at least about 80%, at least about 85%, at least about 90%, at least about 91%, at least about 92%, at least about 93%, at least about 94%, at least about 95%, at least about 96%, at least about 97%, at least about 98%, or at least about 99%. In a preferred embodiment, the RNA polynucleotides within an LNP formulation have an integrity of at least about 80%. In another preferred embodiment, the RNA polynucleotides within an LNP formulation have an integrity of at least about 85%. In yet another preferred embodiment, the RNA polynucleotides within an LNP formulation have an integrity of at least about 90%.

In some embodiments, in an LNP formulation wherein the LNPs comprise a TLR 7/8 modulating molecule, the RNA polynucleotides within the LNP formulation have an integrity within 20%, 15%, 10%, 5%, 4%, 3%, 2%, or 1% of the integrity of RNA polynucleotides within an LNP formulation wherein the LNPs do not comprise a TLR 7/8 modulating molecule. In a preferred embodiment, in an LNP formulation wherein the LNPs comprise a TLR 7/8 modulating molecule, the RNA polynucleotides within the LNP formulation have an integrity within 10% of the integrity of RNA polynucleotides within an LNP formulation wherein the LNPs do not comprise a TLR 7/8 modulating molecule. In another preferred embodiment, in an LNP formulation wherein the LNPs comprise a TLR 7/8 modulating molecule, the RNA polynucleotides within the LNP formulation have an integrity within 5% of the integrity of RNA polynucleotides within an LNP formulation wherein the LNPs do not comprise a TLR 7/8 modulating molecule.

In some embodiments, the LNP integrity of the LNP, LNP suspension, lyophilized LNP composition, or LNP formulation of the present disclosure is about 20% or higher, about 25% or higher, about 30% or higher, about 35% or higher, about 40% or higher, about 45% or higher, about 50% or higher, about 55% or higher, about 60% or higher, about 65% or higher, about 70% or higher, about 75% or higher, about 80% or higher, about 85% or higher, about 90% or higher, about 95% or higher, about 96% or higher, about 97% or higher, about 98% or higher, or about 99% or higher.

In some embodiments, the LNP integrity of the LNP, LNP suspension, lyophilized LNP composition, or LNP formulation of the present disclosure is higher than the LNP integrity of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more, about 10 folds or more, about 20 folds or more, about 30 folds or more, about 40 folds or more, about 50 folds or more, about 100 folds or more, about 200 folds or more, about 300 folds or more, about 400 folds or more, about 500 folds or more, about 1000 folds or more, about 2000 folds or more, about 3000 folds or more, about 4000 folds or more, about 5000 folds or more, or about 10000 folds or more.

In some embodiments, the Txo % of the LNP, LNP suspension, lyophilized LNP composition, or LNP formulation of the present disclosure is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer. As used herein, the term “Tx” refers to the amount of time lasted for the nucleic acid integrity (e.g., mRNA integrity) of a LNP, LNP suspension, lyophilized LNP composition, or LNP formulation to degrade to about X of the initial integrity of the nucleic acid (e.g., mRNA) used for the preparation of the LNP, LNP suspension, lyophilized LNP composition, or LNP formulation. For example, “T8o %” refers to the amount of time lasted for the nucleic acid integrity (e.g., mRNA integrity) of a LNP, LNP suspension, lyophilized LNP composition, or LNP formulation to degrade to about 80% of the initial integrity of the nucleic acid (e.g., mRNA) used for the preparation of the LNP, LNP suspension, lyophilized LNP composition, or LNP formulation. In other words, the time for a LNP, LNP suspension, lyophilized LNP composition, or LNP formulation, to lose 20% of its integrity.

In preferred embodiments, the RNA polynucleotide has a clinical grade purity. In some embodiments, the purity of the RNA polynucleotide is between about 60% and about 100%. In some embodiments, the purity of the RNA polynucleotide is between about 80% and 99%. In some embodiments, the purity of the RNA polynucleotide is between about 90% and about 99%. In some embodiments, wherein the purified mRNA has a clinical grade purity without further purification. In some embodiments, the clinical grade purity is achieved through a method including tangential flow filtration (TFF) purification. In some embodiments, the clinical grade purity is achieved without the further purification selected from high performance liquid chromatography (HPLC) purification, ligand or binding based purification, and/or ion exchange chromatography. In some embodiments, the method of producing the RNA polynucleotides removes long abortive RNA species, double-stranded RNA (dsRNA), residual plasmid DNA residual solvent and/or residual salt. In some embodiments, the short abortive transcript contaminants comprise less than 15 bases. In some embodiments, the short abortive transcript contaminants comprise about 8-12 bases. In some embodiments, the method of the invention also removes RNAse inhibitor.

In some embodiments, the purified RNA polynucleotide comprises 5% or less, 4% or less, 3% or less, 2% or less, 1% or less or is substantially free of protein contaminants as determined by capillary electrophoresis. In some embodiments, the purified RNA polynucleotide comprises less than 5%, less than 4%, less than 3%, less than 2%, less than 1%, or is substantially free of salt contaminants determined by high performance liquid chromatography (HPLC). In some embodiments, the purified RNA polynucleotide comprises 5% or less, 4% or less, 3% or less, 2% or less, 1% or less or is substantially free of short abortive transcript contaminants determined by known methods, such as, e.g., high performance liquid chromatography (HPLC). In some embodiments, the purified RNA polynucleotide has integrity of 60% or greater, 70% or greater, 80% or greater, 81% or greater, 82% or greater, 83% or greater, 84% or greater, 85% or greater, 86% or greater, 87% or greater, 88% or greater, 89% or greater, 90% or greater, 95% or greater, 96% or greater, 97% or greater, 98% or greater, or 99% or greater as determined by a known method, such as, e.g., capillary electrophoresis.

The present disclosure provides for an RNA product solution and a lipid preparation mixture or compositions thereof comprising at least one RNA encoding, e.g., an immunogen complexed with, encapsulated in, and/or formulated with one or more lipids, and forming lipid nanoparticles (LNPs), liposomes, lipoplexes and/or nanoliposomes. In some aspects, the composition comprises a lipid nanoparticle.

A lipid nanoparticle or “LNP” refers to particles of any morphology generated when a cationic lipid and optionally one or more further lipids are combined, e.g., in an aqueous environment and/or in the presence of RNA. In some aspects, lipid nanoparticles are included in a formulation that may be used to deliver an active agent or therapeutic agent, such as a nucleic acid (e.g., mRNA) to a target site of interest (e.g., cell, tissue, organ, tumor, and the like). In some aspects, the lipid nanoparticles of the present disclosure comprise a nucleic acid (e.g., mRNA). Such lipid nanoparticles typically comprise a cationic lipid and one or more excipients, e.g., one or more neutral lipids, charged lipids, steroids, polymer conjugated lipids, or combinations thereof. In some aspects, the LNPs comprise at least one cationic (e.g., ionizable) lipid, at least one neutral (e.g., non-cationic) lipid, at least one structural lipid (e.g., a steroid), and/or at least one polymer conjugated lipid (e.g., a polyethylene glycol (PEG)-modified lipid). In some aspects, 1, 2, 3, or more of the foregoing excipients may be excluded from the LNPs.

In some aspects, an LNP formulation described herein comprises 20-60 mole percent (mol %) cationic (e.g., ionizable) lipid(s) as a percentage of total lipid. For example, an LNP formulation described herein may comprise 20-50 mol %, 20-40 mol %, 20-30 mol %, 30-60 mol %, 30-50 mol %, 30-40 mol %, 40-60 mol %, 40-50 mol %, or 50-60 mol % cationic (e.g., ionizable) lipid(s) as a percentage of total lipid. In some aspects, an LNP formulation comprises 45-55 mol % cationic (e.g., ionizable) lipid(s) as a percentage of total lipid. For example, an LNP formulation may comprise at least, at most, or exactly, any one of 45, 46, 47, 48, 49, 50, 51, 52, 53, 54, or 55 mol % cationic (e.g., ionizable) lipid(s) as a percentage of total lipid. In some embodiments, an LNP formulation may comprise at least, at most, or exactly, any one of 47, 47.1, 47.2, 47.3, 47.4, 47.5, 47.6, 47.7, 47.8, 47.9, or 48 mol % cationic (e.g., ionizable) lipid(s) as a percentage of total lipid.

In a preferred embodiment, an LNP formulation comprises between 47 mol % and 48 mol % cationic (e.g., ionizable) lipid(s) as a percentage of total lipid. In another preferred embodiment, an LNP formulation comprises 47.5 mol % cationic (e.g., ionizable) lipid(s) as a percentage of total lipid.

In a preferred embodiment, an LNP formulation comprises between 47 mol % and 48 mol % ALC-0315 cationic lipid as a percentage of total lipid. In another preferred embodiment, an LNP formulation comprises 47.5 mol % ALC-0315 cationic lipid as a percentage of total lipid.

In some aspects, an LNP formulation comprises 5-25 mol % neutral (e.g., non-cationic) lipid(s) as a percentage of total lipid. For example, an LNP formulation may comprise 5-20 mol %, 5-15 mol %, 5-10 mol %, 10-25 mol %, 10-20 mol %, 10-25 mol %, 15-25 mol %, 15-20 mol %, or 20-25 mol % neutral (e.g., non-cationic) lipid(s) as a percentage of total lipid. In some aspects, an LNP formulation comprises at least, at most, exactly, or between (inclusive or exclusive) any two of 5 mol %, 10 mol %, 15 mol %, 20 mol %, or 25 mol % neutral (e.g., non-cationic) lipid(s) as a percentage of total lipid. In some aspects, an LNP formulation comprises 5 to 15 mol % neutral (e.g., non-cationic) lipid(s) as a percentage of total lipid. For example, an LNP formulation may comprise at least, at most, exactly, or between (inclusive or exclusive) any two of 5, 6, 7, 8, 9, 10, 11, 12, 13, 14, or 15 mol % neutral (e.g., non-cationic) lipid(s) as a percentage of total lipid.

In a preferred embodiment, an LNP formulation comprises 10 mol % neutral (e.g., non-cationic) lipid(s) as a percentage of total lipid. In another preferred embodiment, an LNP formulation comprises 10 mol % DSPC neutral lipid as a percentage of total lipid.

In some aspects, an LNP formulation comprises 25-55 mol % structural lipid(s) (e.g., a steroid) as a percentage of total lipid. For example, an LNP formulation may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % structural lipid(s) (e.g., a steroid) as a percentage of total lipid. In some aspects, an LNP formulation may comprise at least, at most, exactly, or between (inclusive or exclusive) any two of 25 mol %, 30 mol %, 35 mol %, 40 mol %, 45 mol %, 50 mol %, or 55 mol % structural lipid(s) as a percentage of total lipid. In some aspects, an LNP formulation comprises 35 to 45 mol % structural lipid(s) as a percentage of total lipid. For example, an LNP formulation may comprise at least, at most, exactly, or between (inclusive or exclusive) any two of 35, 36, 37, 38, 39, 40, 41, 42, 43, 44, or 45 mol % structural lipid(s) as a percentage of total lipid. In some embodiments, an LNP formulation comprises between about 40 mol % and about 41 mol % structural lipid(s) as a percentage of total lipid. For example, an LNP formulation may comprise at least, at most, exactly, or between (inclusive or exclusive) any two of 40, 40.1, 40.2, 40.3, 40.4, 40.5, 40.6, 40.7, 40.8, 40.9, or 50 mol % structural lipid(s) as a percentage of total lipid.

In a preferred embodiment, an LNP formulation comprises between about 40 mol % and about 41 mol % structural lipid(s) as a percentage of total lipid. In another preferred embodiment, an LNP formulation comprises about 40.7 mol % structural lipid(s) as a percentage of total lipid.

In a preferred embodiment, an LNP formulation comprises between about 40 mol % and about 41 mol % cholesterol as a percentage of total lipid. In another preferred embodiment, an LNP formulation comprises about 40.7 mol % cholesterol as a percentage of total lipid.

In a preferred embodiment, an LNP formulation comprises between about 40 mol % and about 41 mol % structural lipids as a percentage of total lipid, wherein the structural lipids consist of cholesterol and β-Sitosterol. In another preferred embodiment, an LNP formulation comprises about 40.7 mol % structural lipids as a percentage of total lipid, wherein the structural lipids consist of cholesterol and β-Sitosterol.

In some aspects, an LNP formulation comprises 0.5-15 mol % polymer conjugated lipid(s) (e.g., a polyethylene glycol (PEG)-modified lipid) as a percentage of total lipid. For example, an LNP formulation may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol % polymer conjugated lipid(s) as a percentage of total lipid. In some aspects, an LNP formulation comprises at least, at most, exactly, or between (inclusive or exclusive) any two of 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % polymer conjugated lipid(s) as a percentage of total lipid. In some aspects, an LNP formulation comprises 1 to 2 mol % polymer conjugated lipid(s) as a percentage of total lipid.

For example, an LNP formulation may comprise at least, at most, exactly, or between (inclusive or exclusive) any two of 1, 1.5, or 2 mol % polymer conjugated lipid(s) as a percentage of total lipid.

In a preferred embodiment, an LNP formulation comprises between about 1.5 mol % and about 2 mol % polymer conjugated lipid(s) as a percentage of total lipid. In another preferred embodiment, an LNP formulation comprises about 1.8 mol % polymer conjugated lipid(s) as a percentage of total lipid.

In a preferred embodiment, an LNP formulation comprises between about 1.5 mol % and about 2 mol % ALC-0159 polymer conjugated lipid as a percentage of total lipid. In another preferred embodiment, an LNP formulation comprises about 1.8 mol % ALC-0159 polymer conjugated lipid as a percentage of total lipid.

In some aspects, the LNPs comprise 20-75 mol % cationic (e.g., ionizable) lipid(s) (e.g., at least, at most, exactly, or between (inclusive or exclusive) any two of 20%, 25%, 30%, 35%, 40%, 45%, 50%, 55%, 60%, 65%, 70%, and 75%), 0.5-25 mol % neutral (e.g., non-cationic) lipid(s) (e.g., at least, at most, exactly, or between (inclusive or exclusive) of 0.5%, 2.25%, 4%, 5.75%, 7.5%, 9.25%, 10%, 11%, 12.75%, 14.5%, 16.25%, 18%, 19.75%, 21.5%, 23.25%, and 25%), 5-55 mol % structural lipid(s) (e.g., a sterol) e.g., non-cationic) lipid(s) (e.g., at least, at most, exactly, or between (inclusive or exclusive) of 5%, 10%, 15%, 20%, 25%, 30%, 35%, 40%, 45%, 50%, and 55%), and 0.5-20 mol % polymer conjugated lipid(s) (e.g., a polyethylene glycol (PEG)-modified lipid) (e.g., at least, at most, exactly, or between (inclusive or exclusive) of 0.5%, 2%, 3.5%, 5%, 6.5%, 8%, 9.5%, 11%, 12.5%, 14%, 15.5%, 17%, 18.5%, and 20%). In some aspects, 1, 2, 3, or more of the lipids may be excluded from the LNPs.

In some non-limiting aspects, the molar lipid ratio is 50/10/38.5/1.5 (mol % cationic lipid/neutral lipid/structural lipid/polymer conjugated lipid), 60/7.5/31/1.5 (mol % cationic lipid/neutral lipid/structural lipid/polymer conjugated lipid), 57.5/7.5/31.5/3.5 (mol % cationic lipid/neutral lipid/structural lipid/polymer conjugated lipid), 57.2/7.1/34.3/1.4 (mol % cationic lipid/neutral lipid/structural lipid/polymer conjugated lipid), 40/15/40/5 (mol % cationic lipid/neutral lipid/structural lipid/polymer conjugated lipid), 50/10/35/4.5/0.5 (mol % cationic lipid/neutral lipid/structural lipid/polymer conjugated lipid), 50/10/35/5 (mol % cationic lipid/neutral lipid/structural lipid/polymer conjugated lipid), 47.5/10/40.7/1.8 (mol % cationic lipid/neutral lipid/structural lipid/polymer conjugated lipid), 40/10/40/10 (mol % cationic lipid/neutral lipid/structural lipid/polymer conjugated lipid), 35/15/40/10 (mol % cationic lipid/neutral lipid/structural lipid/polymer conjugated lipid), or 52/13/30/5 (mol % cationic lipid/neutral lipid/structural lipid/polymer conjugated lipid).

In some aspects, the active agent or therapeutic agent, such as a nucleic acid (e.g., mRNA), may be encapsulated in the lipid portion of the lipid nanoparticle and/or an aqueous space enveloped by some or all of the lipid portion of the lipid nanoparticle, thereby protecting it from enzymatic degradation or other undesirable effects induced by the mechanisms of the host organism or cells, e.g., an adverse immune response. The nucleic acid (e.g., mRNA) or a portion thereof may also be associated and complexed with the lipid nanoparticle. A lipid nanoparticle may comprise any lipid capable of forming a particle to which the nucleic acids are attached, and/or in which the one or more nucleic acids are encapsulated.

In some embodiments, a polynucleotide includes 200 to 3,000 nucleotides. For example, a polynucleotide may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides.

In some embodiments, a LNP includes one or more RNAs, and the one or more RNAs, lipids, and amounts thereof may be selected to provide a specific N:P ratio. The N:P ratio of the composition refers to the molar ratio of nitrogen atoms in one or more lipids to the phosphate groups in an RNA. In general, a lower N:P ratio is preferred. In one embodiment, the lipid is an ionizable lipid. In another embodiment, the lipid is an ionizable cationic lipid. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1 or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 5.67:1, about 6.0:1, about 6.5:1, or about 7.0:1. For example, the N:P ratio may be about 5.67:1. In a preferred embodiment, the N:P ratio refers to the molar ratio of nitrogen atoms in the cationic lipid to the phosphate groups in an RNA, and the N:P ratio is about 6:1. In a preferred embodiment, the N:P ratio refers to the molar ratio of nitrogen atoms in the cationic lipid to the phosphate groups in an RNA, and the N:P ratio is about 5.67:1.

In some aspects, an LNP of the disclosure comprises a wt/wt ratio of the cationic lipid component to the RNA of or of from about 5:1 to about 100:1, e.g., at least, at most, exactly, or between (inclusive or exclusive) of 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, or 100:1. In some aspects, an LNP of the disclosure comprises a wt/wt ratio of the ionizable cationic lipid component to the RNA of or of about 20:1. In some aspects, an LNP of the disclosure comprises a wt/wt ratio of the ionizable cationic lipid component to the RNA of or of about 10:1.

In certain aspects, nucleic acids (e.g., RNA molecules), when present in provided LNPs, are resistant in aqueous solution to degradation with a nuclease. In some aspects, LNPs are liver-targeting lipid nanoparticles. In some aspects, LNPs are cationic lipid nanoparticles comprising one or more cationic lipids (e.g., those described herein). In some aspects, cationic LNPs may comprise at least one cationic lipid, at least one polymer conjugated lipid, and at least one helper lipid (e.g., at least one neutral lipid).

In certain aspects, the RNA solution and lipid preparation mixture or compositions thereof may have at least, at most, exactly, between (inclusive or exclusive) of, or about 1%, 2%, 3%, 4% 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of a particular lipid, lipid type, or non-lipid component such as lipid-like materials and/or cationic polymers and/or an adjuvant, antigen, peptide, polypeptide, sugar, nucleic acid or other material disclosed herein or as would be known to one of skill in the art.

LNPs described herein can be generated using components, compositions, and methods as are generally known in the art, see, e.g., PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/052117; PCT/US2012/069610; F PCT/US2017/027492; PCT/US2016/059575 and PCT/US2016/069491, all of which are incorporated by reference herein in their entirety. Other non-limiting examples of methods for preparing LNPs can be found in, e.g., WO 2022/032154, the disclosure of which is incorporated by reference herein in its entirety.

For example, methods of preparing LNPs may involve obtaining a colloid from at least one cationic or cationically ionizable lipid or lipid-like material and/or at least one cationic polymer and mixing the colloid with nucleic acid to obtain nucleic acid particles. The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” refers only to the particles in the mixture and not the entire suspension.

For the preparation of colloids comprising at least one cationic or cationically ionizable lipid or lipid-like material and/or at least one cationic polymer, methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media).

In the film hydration method, lipids are first dissolved in a suitable organic solvent and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that subsequently turns into a liposomal suspension.

The term “ethanol injection technique” refers to a process in which an ethanol solution comprising lipids is rapidly injected into an aqueous solution through a needle. This action disperses the lipids throughout the solution and promotes lipid structure formation, for example, lipid vesicle formation such as liposome formation. Generally, the RNA lipoplex particles described herein are obtainable by adding RNA to a colloidal liposome dispersion. Using the ethanol injection technique, such colloidal liposome dispersion is, in some aspects, formed as follows: an ethanol solution comprising lipids, such as cationic lipids and additional lipids, is injected into an aqueous solution under stirring. In some aspects, the RNA lipoplex particles described herein are obtainable without a step of extrusion. The term “extruding” or “extrusion” refers to the creation of particles having a fixed, cross-sectional profile. In particular, it refers to the downsizing of a particle, whereby the particle is forced through filters with defined pores.

Other methods for preparing a colloid having organic solvent free characteristics may also be used according to the present disclosure.

In some aspects, LNP-encapsulated RNA may be produced by rapid mixing of an RNA solution described herein (e.g., the RNA product solution) and a lipid preparation described herein (comprising, e.g., at least one cationic lipid and optionally one or more other lipid components, in an organic solvent) under conditions such that a sudden change in solubility of lipid component(s) is triggered, which drives the lipids towards self-assembly in the form of LNPs. In some aspects, suitable buffering agents comprise tris, histidine, citrate, acetate, phosphate, and/or succinate. In some aspects, 1, 2, 3, or more of the foregoing buffering agents are excluded. The pH of a liquid formulation relates to the pKa of the encapsulating agent (e.g., cationic lipid). The pH of the acidifying buffer may be at least half a pH scale less than the pKa of the encapsulating agent (e.g., cationic lipid), and the pH of the final buffer may be at least half a pH scale greater than the pKa of the encapsulating agent (e.g., cationic lipid). In some aspects, properties of a cationic lipid are chosen such that nascent formation of particles occurs by association with an oppositely charged backbone of a nucleic acid (e.g., RNA). In this way, particles are formed around the nucleic acid, which, for example, in some aspects, may result in much higher encapsulation efficiency than is achieved in the absence of interactions between nucleic acids and at least one of the lipid components. In certain aspects, nucleic acids, when present in the lipid nanoparticles, are resistant in aqueous solution to degradation with a nuclease.

Lipid nanoparticles comprising nucleic acids and their method of preparation are disclosed in, e.g., U.S. Patent Publication Nos. 2004/0142025, 2007/0042031 and PCT Pub. Nos. WO 2013/016058 and WO 2013/086373, the full disclosures of which are herein incorporated by reference in their entirety for all purposes.

Some aspects described herein relate to compositions, methods and uses involving more than one, e.g., 2, 3, 4, 5, 6 or even more nucleic acid species, such as RNA species. In an LNP formulation, it is possible that each nucleic acid species is separately formulated as an individual LNP formulation. In that case, each individual LNP formulation will comprise one nucleic acid species. The individual LNP formulations may be present as separate entities, e.g., in separate containers. Such formulations are obtainable by providing each nucleic acid species separately (typically each in the form of a nucleic acid-containing solution) together with suitable cationic or cationically ionizable lipids or lipid-like materials and cationic polymers that allow the formation of LNPs. Respective particles will contain exclusively the specific nucleic acid species that is being provided when the particles are formed (individual particulate formulations).

In some aspects, a composition such as a pharmaceutical composition comprises more than one individual LNP formulation. Respective pharmaceutical compositions are referred to as mixed LNP formulations. Mixed LNP formulations according to the invention are obtainable by forming, separately, individual LNP formulations, as described above, followed by a step of mixing of the individual LNP formulations. By the step of mixing, a formulation comprising a mixed population of nucleic acid-containing LNPs is obtainable. Individual LNP populations may be together in one container, comprising a mixed population of individual LNP formulations.

Alternatively, it is possible that different nucleic acid species are formulated together as a combined LNP formulation. Such formulations are obtainable by providing a combined formulation (typically combined solution) of different RNA species together with suitable cationic or cationically ionizable lipids or lipid-like materials and cationic polymers that allow the formation of LNPs. As opposed to a mixed LNP formulation, a combined LNP formulation will typically comprise LNPs that comprise more than one RNA species. In a combined LNP composition, different RNA species are typically present together in a single particle.

### Cationic Polymeric Materials

Given their high degree of chemical flexibility, polymeric materials are commonly used for nanoparticle-based delivery. Typically, cationic materials are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic materials useful in some aspects herein. In addition, some investigators have synthesized polymeric materials specifically for nucleic acid delivery. Poly(P-amino esters), in particular, have gained widespread use in nucleic acid delivery owing to their ease of synthesis and biodegradability. In some aspects, such synthetic materials may be suitable for use as cationic materials herein.

A “polymeric material,” as used herein, is given its ordinary meaning, e.g., a molecular structure comprising one or more repeat units (monomers), connected by covalent bonds. In some aspects, such repeat units may all be identical; alternatively, in some cases, there may be more than one type of repeat unit present within the polymeric material. In some cases, a polymeric material is biologically derived, e.g., a biopolymer such as a protein. In some cases, additional moieties may also be present in the polymeric material, for example targeting moieties such as those described herein.

Those skilled in the art are aware that, when more than one type of repeat unit is present within a polymer (or polymeric moiety), then the polymer (or polymeric moiety) is said to be a “copolymer.” In some aspects, a polymer (or polymeric moiety) utilized in accordance with the present disclosure may be a copolymer. Repeat units forming the copolymer may be arranged in any fashion. For example, in some aspects, repeat units may be arranged in a random order; alternatively or additionally, in some aspects, repeat units may be arranged in an alternating order, or as a “block” copolymer, e.g., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks.

In certain aspects, a polymeric material for use in accordance with the present disclosure is biocompatible. Biocompatible materials are those that typically do not result in significant cell death at moderate concentrations. In certain aspects, a biocompatible material is biodegradable, e.g., is able to degrade, chemically and/or biologically, within a physiological environment, such as within the body. In certain aspects, a polymeric material may be or comprise protamine or polyalkyleneimine, in particular protamine.

As those skilled in the art are aware term “protamine” is often used to refer to any of various strongly basic proteins of relatively low molecular weight that are rich in arginine and are found associated especially with DNA in place of somatic histones in the sperm cells of various animals (as fish). In particular, the term “protamine” is often used to refer to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

In some aspects, the term “protamine” as used herein is refers to a protamine amino acid sequence obtained or derived from natural or biological sources, including fragments thereof and/or multimeric forms of said amino acid sequence or fragment thereof, as well as (synthesized) polypeptides which are artificial and specifically designed for specific purposes and cannot be isolated from native or biological sources.

In some aspects, a polyalkyleneimine comprises polyethylenimine and/or polypropylenimine. In some aspects, the polyalkyleneimine is polyethyleneimine (PEI). In some aspects, the polyalkyleneimine is a linear polyalkyleneimine, e.g., linear polyethyleneimine (PEI).

Cationic materials (e.g., polymeric materials, including polycationic polymers) contemplated for use herein include those which are able to electrostatically bind nucleic acid. In some aspects, cationic polymeric materials contemplated for use herein include any cationic polymeric materials with which nucleic acid may be associated, e.g. by forming complexes with the nucleic acid or forming vesicles in which the nucleic acid is enclosed or encapsulated.

In some aspects, particles described herein may comprise polymers other than cationic polymers, e.g., non-cationic polymeric materials and/or anionic polymeric materials. Collectively, anionic and neutral polymeric materials are referred to herein as non-cationic polymeric materials.

### Lipids and Lipid-Like Materials

The terms “lipid” and “lipid-like material” are used herein to refer to molecules which comprise one or more hydrophobic moieties or groups and optionally also one or more hydrophilic moieties or groups. According to the disclosure, lipids and lipid-like materials may be cationic, anionic or neutral. Neutral lipids or lipid-like materials exist in an uncharged or neutral zwitterionic form at a selected pH.

The term “lipid” refers to a group of organic compounds that are characterized by being insoluble in water but soluble in many organic solvents. Generally, lipids may be divided into eight categories: fatty acids and their derivatives (including tri-, di-, monoglycerides, and phospholipids), glycerolipids, glycerophospholipids, sphingolipids, saccharolipids, polyketides, sterol lipids as well as sterol-containing metabolites such as cholesterol, and prenol lipids. Examples of fatty acids include, but are not limited to, fatty esters and fatty amides. Examples of glycerolipids include, but are not limited to, glycosylglycerols and glycerophospholipids (e.g., phosphatidylcholine, phosphatidylethanolamine, phosphatidylserine). Examples of sphingolipids include, but are not limited to, ceramides phosphosphingolipids (e.g., sphingomyelins, phosphocholine), and glycosphingolipids (e.g., cerebrosides, gangliosides). Examples of sterol lipids include, but are not limited to, cholesterol and its derivatives and tocopherol and its derivatives. In some aspects, 1, 2, 3, 4, 5, or more of the lipids may be excluded from the LNPs of the present disclosure.

The term “lipid-like material,” “lipid-like compound,” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids.

In some aspects, the RNA solution and lipid preparation mixture or compositions thereof may comprise cationic lipids, neutral lipids, cholesterol, and/or polymer (e.g., polyethylene glycol) conjugated lipids which form lipid nanoparticles that encompass the RNA molecules. Therefore, in some aspects, the LNP may comprise a cationic lipid and one or more excipients, e.g., one or more neutral lipids, charged lipids, steroids or steroid analogs (e.g., cholesterol), polymer conjugated lipids (e.g. PEG-lipid), or combinations thereof. In some aspects, 1, 2, 3, or more of the foregoing excipients may be excluded from the LNPs of the present disclosure. In some aspects, the lipids are present in a composition in an amount that is effective to form a lipid nanoparticle and deliver a therapeutic agent, e.g., an RNA molecule, for treating a particular disease or condition of interest. In some aspects, the LNPs encompass, or encapsulate, the nucleic acid molecules.

### Cationic Lipids

Cationic or cationically ionizable lipids or lipid-like materials refer to a lipid or lipid-like material capable of being positively charged and able to electrostatically bind nucleic acid. As used herein, a “cationic lipid” or “cationic lipid-like material” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl, or more acyl chains, and the head group of the lipid typically carries the positive charge. Exemplary cationic lipids include one or more amine group(s) which bear the positive charge. Cationic lipids may encapsulate negatively charged RNA.

In some aspects, cationic lipids are ionizable such that they may exist in a positively charged or neutral form depending on pH. The ionization of the cationic lipid affects the surface charge of the lipid nanoparticle under different pH conditions. Without wishing to be bound by theory, this ionizable behavior is thought to enhance efficacy through helping with endosomal escape and reducing toxicity as compared with particles that remain cationic at physiological pH. For purposes of the present disclosure, such “cationically ionizable” lipids or lipid-like materials are comprised by the term “cationic lipid” or “cationic lipid-like material” unless contradicted by the circumstances.

Examples of cationic lipids include, but are not limited to: ((4-hydroxybutyl)azanediyl)bis(hexane-6,1-diyl)bis(2-hexyldecanoate), 1,2-dioleoyl-3-trimethylammonium propane (DOTAP), N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl) cholesterol (DC-Chol), dimethyldioctadecylammonium (DDAB), 1,2-dioleoyl-3-dimethylammonium-propane (DODAP), 1,2-diacyloxy-3-dimethylammonium propanes, 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleoyloxypropyl-3-dimethyl-hydroxyethyl ammonium bromide (DORIE), 2,3-dioleoyloxy-N-[2 (spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-I-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-dilinoleoyloxy-N,N-dimethylpropylamine (DLinDAP), 1,2-N,N′-dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (bAE-DMRIE), N-(4-carboxybenzyl)-N,N-dimethyl-2,3-bis(oleoyloxy)propan-1-aminium (DOBAQ), 2- ({8-[(3b)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-I-yl) 8,8′-((((2 (dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-I-yl)-9-((4-(dimethylaminobutanoyl)oxy)heptadecanedioate (L319), N-dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino) propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid 02-200); C 12-200; or heptadecan-9-yl 8-((2-hydroxyethyl) (6-oxo-6-(undecyloxy) hexyl)amino) octanoate (SM-102). In some aspects, 1, 2, 3, 4, 5, or more of the foregoing cationic lipids may be excluded from the LNPs of the present disclosure.

In some aspects, an ionizable cationic lipid of the disclosure comprises a compound of Formula (I):

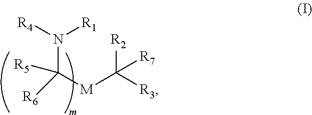

or a pharmaceutically acceptable salt, tautomer, prodrug, or stereoisomer thereof, wherein:
R₁ is a C₅₋₃₀ alkyl, C₅₋₂₀ alkenyl, —R*YR″, —YR″, or —R″M′R′;
R₂ and R₃ are independently a H, C₁₋₁₄ alkyl, C₂₋₁₄ alkenyl, —R*YR″, —YR″, or —R*OR″, and/or R₂ and R₃, together with the atom to which they are attached, form a heterocycle or carbocycle;
R₄ is a C₃₋₆ carbocycle, —(CH₂)ₙQ, —(CH₂)ₙCHQR, —CHQR, —CQ(R)₂, or unsubstituted C₁₋₆ alkyl, where Q is a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, or —C(R)N(R)₂C(O)OR, and/or each n is independently a 1, 2, 3, 4, or 5;
each R₅ is independently a C₁₋₃ alkyl, C₂₋₃ alkenyl, or H;
each R₆ is independently a C₁₋₃ alkyl, C₂₋₃ alkenyl, or H;
M and M′ are independently a —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, or a heteroaryl group;
R₇ is a C₁₋₃ alkyl, C₂₋₃ alkenyl, or H;
R₈ is a C₃₋₆ carbocycle or heterocycle;
R₉ is a H, CN, NO₂, C₁₋₆ alkyl, —OR, —S(O)₂R, —S(O)₂N(R)₂, C₂₋₆ alkenyl, C₃₋₆ carbocycle, or heterocycle;
each R is a C₁₋₃ alkyl, C₂₋₃ alkenyl, or H;
each R′ is a C₁₋₁₈ alkyl, C₂₋₁₈ alkenyl, —R*YR″, —YR″, or H;
each R″ is a C₃₋₁₄ alkyl or C₃₋₁₄ alkenyl;
each R* is independently a C₁₋₁₂ alkyl or C₂₋₁₂ alkenyl;
each Y is independently a C₃₋₆ carbocycle;
each X is independently a F, Cl, Br, or I; and
m is a 5, 6, 7, 8, 9, 10, 11, 12, or 13.

In some aspects, a subset of compounds of Formula (I) includes those in which when R₄ is —(CH₂)ₙQ, —(CH₂)ₙCHQR, —CHQR, or —CQ(R)₂, then (i) Q is not —N(R)₂ when n is 1, 2, 3, 4, or 5, or (ii) Q is not 5, 6, or 7-membered heterocycloalkyl when n is 1 or 2.

In some aspects, another subset of compounds of Formula (I) includes those in which R₁ is a C₅₋₃₀ alkyl, C₅₋₂₀ alkenyl, —R*YR″, —YR″, or —R″M′R′;
R₂ and R₃ are independently an H, C₁₋₁₄ alkyl, C₂₋₁₄ alkenyl, —R*YR″, —YR″, or —R*OR″, and/or R2 and R3, together with the atom to which they are attached, form a heterocycle or carbocycle;
R₄ is a C₃₋₆ carbocycle, —(CH₂)ₙQ, —(CH₂)ₙCHQR, —CHQR, —CQ(R)₂, or unsubstituted C₁₋₆ alkyl, where Q is a C₃₋₆ carbocycle, a 5- to 14-membered heteroaryl having one or more heteroatoms comprising N, O, or S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, or a 5- to 14-membered heterocycloalkyl having one or more heteroatoms comprising N, O, and S which is substituted with one or more substituents comprising oxo (═O), OH, amino, mono- or di-alkylamino, or C₁₋₃ alkyl, and/or each n is independently 1, 2, 3, 4, or 5;
each R₅ is independently a C₁₋₃ alkyl, C₂₋₃ alkenyl, or H;
each R₆ is independently a C₁₋₃ alkyl, C₂₋₃ alkenyl, or H;
M and M′ are independently a —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, or a heteroaryl group;
R₇ is a C₁₋₃ alkyl, C₂₋₃ alkenyl, or H;
R₈ is a C₃₋₆ carbocycle or heterocycle;
R₉ is a H, CN, NO₂, C₁₋₆ alkyl, —OR, —S(O)₂R, —S(O)₂N(R)₂, C₂₋₆ alkenyl, C₃₋₆ carbocycle or heterocycle;
each R is independently a C₁₋₃ alkyl, C₂₋₃ alkenyl, or H;
each R′ is independently a C₁₋₁₈ alkyl, C₂₋₁₈ alkenyl, —R* YR″, —YR″, or H;
each R″ is independently a C₃₋₁₄ alkyl or C₃₋₁₄ alkenyl;
each R* is independently a C₁₋₁₂ alkyl or C₂₋₁₂ alkenyl;
each Y is independently a C₃₋₆ carbocycle;
each X is independently a F, Cl, Br, or I; and
m is 5, 6, 7, 8, 9, 10, 11, 12, or 13, and/or pharmaceutically acceptable salts, tautomers, prodrugs, or stereoisomers thereof.

In some aspects, another subset of compounds of Formula (I) includes those in which:
R₁ is a C₅₋₃₀ alkyl, C₅₋₂₀ alkenyl, —R*YR″, —YR″, or —R″M′R′;
R₂ and R₅ are independently an H, C₁₋₁₄ alkyl, C₂₋₁₄ alkenyl, —R*YR″, —YR″, or —R*OR″, and/or R₂ and R₃, together with the atom to which they are attached, form a heterocycle or carbocycle;
R₄ is a C₃₋₆ carbocycle, —(CH₂)ₙQ, —(CH₂)ₙCHQR, —CHQR, —CQ(R)₂, or unsubstituted C₁₋₆ alkyl, where Q is a C₃₋₆ carbocycle, a 5- to 14-membered heterocycle having one or more heteroatoms comprising N, O, or S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, or —C(═NR₉)N(R)₂, and/or each n is independently 1, 2, 3, 4, or 5; and/or when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently a C₁₋₃ alkyl, C₂₋₃ alkenyl, or H;
each R₆ is independently a C₁₋₃ alkyl, C₂₋₃ alkenyl, or H;
M and M′ are independently a —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, or a heteroaryl group;
R₇ is a C₁₋₃ alkyl, C₂₋₃ alkenyl, or H;
R₈ is C₃₋₆ carbocycle or heterocycle;
R₉ is H, CN, NO₂, C₁₋₆ alkyl, —OR, —S(O)₂R, —S(O)₂N(R)₂, C₂₋₆ alkenyl, C₃₋₆ carbocycle, or heterocycle;
each R is independently a C₁₋₃ alkyl, C₂₋₃ alkenyl, or H;
each R′ is independently a C₁₋₁₈ alkyl, C₂₋₁₈ alkenyl, —R*YR″, —YR″, or H;
each R″ is independently a C₃₋₁₄ alkyl or C₃₋₁₄ alkenyl;
each R* is independently a C₁₋₁₂ alkyl or C₂₋₁₂ alkenyl;
each Y is independently a C₃₋₆ carbocycle;
each X is independently a F, Cl, Br, or I; and
m is 5, 6, 7, 8, 9, 10, 11, 12, or 13, and/or pharmaceutically acceptable salts, tautomers, prodrugs, or stereoisomers thereof.

In some aspects, another subset of compounds of Formula (I) includes those in which:
R₁ is a C₅₋₃₀ alkyl, C₅₋₂₀ alkenyl, —R*YR″, —YR″, or —R″M′R′;
R₂ and R₃ are independently an H, C₁₋₁₄ alkyl, C₂₋₁₄ alkenyl, —R*YR″, —YR″, or —R*OR″, and/or R₂ and R₃, together with the atom to which they are attached, form a heterocycle or carbocycle;
R₄ is a C₃₋₆ carbocycle, —(CH₂)ₙQ, —(CH₂)ₙCHQR, —CHQR, —CQ(R)₂, or unsubstituted C₁₋₆ alkyl, where Q is a C₃₋₆ carbocycle, a 5- to 14-membered heteroaryl having one or more heteroatoms comprising N, O, or S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, or —C(═NR₉)N(R)₂, and/or each n is independently 1, 2, 3, 4, or 5;
each R₅ is independently a C₁₋₃ alkyl, C₂₋₃ alkenyl, or H;
each R₆ is independently a C₁₋₃ alkyl, C₂₋₃ alkenyl, or H;
M and M′ are independently a —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, or a heteroaryl group;
R₇ is a C₁₋₃ alkyl, C₂₋₃ alkenyl, or H;
R₈ is a C₃₋₆ carbocycle or heterocycle;
R₉ is an H, CN, NO₂, C₁₋₆ alkyl, —OR, —S(O)₂R, —S(O)₂N(R)₂, C₂₋₆ alkenyl, C₃₋₆ carbocycle, or heterocycle;
each R is independently a C₁₋₃ alkyl, C₂₋₃ alkenyl, or H;
each R′ is independently a C₁₋₁₈ alkyl, C₂₋₁₈ alkenyl, —R*YR″, —YR″, or H;
each R″ is independently a C₃₋₁₄ alkyl or C₃₋₁₄ alkenyl;
each R* is independently a C₁₋₁₂ alkyl or C₂₋₁₂ alkenyl;
each Y is independently a C₃₋₆ carbocycle;
each X is independently a F, Cl, Br, or I; and
m is 5, 6, 7, 8, 9, 10, 11, 12, or 13, and/or pharmaceutically acceptable salts, tautomers, prodrugs, or stereoisomers thereof.

In some aspects, another subset of compounds of Formula (I) includes those in which:
R₁ is a C₅₋₃₀ alkyl, C₅₋₂₀ alkenyl, —R*YR″, —YR″, or —R″M′R′;
R₂ and R₅ are independently an H, C₂₋₁₄ alkyl, C₂₋₁₄ alkenyl, —R*YR″, —YR″, or —R*OR″, and/or R₂ and R₃, together with the atom to which they are attached, form a heterocycle or carbocycle;
R₄ is —(CH₂)ₙQ or —(CH₂)ₙCHQR, where Q is —N(R)₂, and/or n is 3, 4, or 5;
each R₅ is independently a C₁₋₃ alkyl, C₂₋₃ alkenyl, or H;
each R₆ is independently a C₁₋₃ alkyl, C₂₋₃ alkenyl, or H;
M and M′ are independently a —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, or a heteroaryl group;
R₇ is a C₁₋₃ alkyl, C₂₋₃ alkenyl, or H;
each R is independently a C₁₋₃ alkyl, C₂₋₃ alkenyl, or H;
each R′ is independently a C₁₋₁₈ alkyl, C₂₋₁₈ alkenyl, —R*YR″, —YR″, or H;
each R″ is independently a C₃₋₁₄ alkyl or C₃₋₁₄ alkenyl;
each R* is independently a C₁₋₁₂ alkyl or C₁₋₁₂ alkenyl;
each Y is independently a C₃₋₆ carbocycle;
each X is independently a F, Cl, Br, or I; and
m is 5, 6, 7, 8, 9, 10, 11, 12, or 13, and/or pharmaceutically acceptable salts, tautomers, prodrugs, or stereoisomers thereof.

In some aspects, another subset of compounds of Formula (I) includes those in which:
R₁ is a C₅₋₃₀ alkyl, C₅₋₂₀ alkenyl, —R*YR″, —YR″, or —R″M′R′;
R₂ and R₃ are independently a C₁₋₁₄ alkyl, C₂₋₁₄ alkenyl, —R*YR″, —YR″, or —R*OR″, and/or R₂ and R₃, together with the atom to which they are attached, form a heterocycle or carbocycle;
R₄ is a —(CH₂)ₙQ, —(CH₂)ₙCHQR, —CHQR, or —CQ(R)₂, where Q is —N(R)₂, and/or n is 1, 2, 3, 4, or 5;
each R₅ is independently a C₁₋₃ alkyl, C₂₋₃ alkenyl, or H;
each R₆ is independently a C₁₋₃ alkyl, C₂₋₃ alkenyl, or H;
M and M′ are independently a —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, —S—S—, an aryl group, or a heteroaryl group;
R₇ is a C₁₋₃ alkyl, C₂₋₃ alkenyl, or H;
each R is independently a C₁₋₃ alkyl, C₂₋₃ alkenyl, or H;
each R′ is independently a C₁₋₁₈ alkyl, C₂₋₁₈ alkenyl, —R*YR″, —YR″, or H;
each R″ is independently a C₃₋₁₄ alkyl or C₃₋₁₄ alkenyl;
each R* is independently a C₁₋₁₂ alkyl or C₁₋₁₂ alkenyl;
each Y is independently a C₃₋₆ carbocycle;
each X is independently a F, Cl, Br, or I; and
m is 5, 6, 7, 8, 9, 10, 11, 12, or 13, and/or pharmaceutically acceptable salts, tautomers, prodrugs, or stereoisomers thereof.

In some aspects, a subset of compounds of Formula (I) includes those of Formula (IA):

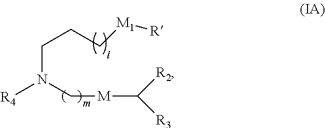

or a pharmaceutically acceptable salt, tautomer, prodrug, or stereoisomer thereof, wherein I is 1, 2, 3, 4, or 5; m is 5, 6, 7, 8, or 9; M₁ is a bond or M′; R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently a —C(O)O—, —OC(O)—, —C(O)N(R′)—, —P(O)(OR′)O—, —S—S—, an aryl group, or a heteroaryl group; and R₂ and R₃ are independently a H, C₁₋₁₄ alkyl, or C₂₋₁₄ alkenyl.

In some aspects, a subset of compounds of Formula (I) includes those of Formula (II):

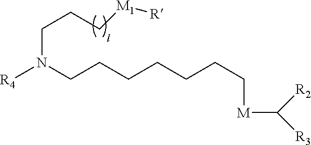

or a pharmaceutically acceptable salt, tautomer, prodrug, or stereoisomer thereof, wherein I is 1, 2, 3, 4, or 5; M₁ is a bond or M′; R₄ is unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently a —C(O)O—, —OC(O)—, —C(O)N(R′)—, —P(O)(OR′)O—, —S—S—, an aryl group, or a heteroaryl group; and R₂ and R₃ are independently a H, C₁₋₁₄ alkyl, or C₂₋₁₄ alkenyl. In some aspects, a subset of compounds of Formula (I) includes those of Formula (IIa), (IIb), (IIc), or (IIe):

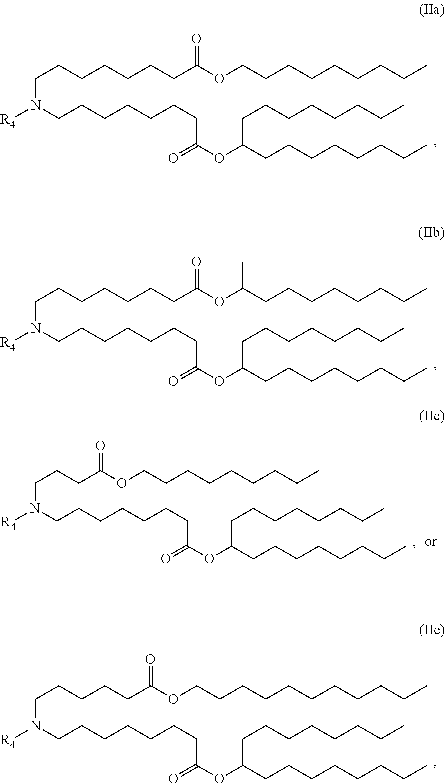

or a pharmaceutically acceptable salt, tautomer, prodrug, or stereoisomer thereof, wherein R₄ is as described herein.

In some aspects, a subset of compounds of Formula (I) includes those of Formula (IId):

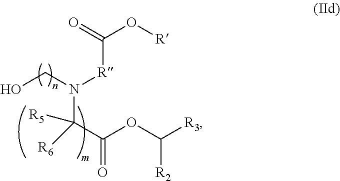

or a pharmaceutically acceptable salt, tautomer, prodrug, or stereoisomer thereof, wherein n is 2, 3, or 4; and m, R′, R″, and R₂ through R₆ are as described herein. For example, each of R₂ and R₃ may be independently a C₅₋₁₄ alkyl or C₅₋₁₄ alkenyl.

In some aspects, an ionizable cationic lipid of the disclosure comprises a compound having structure:

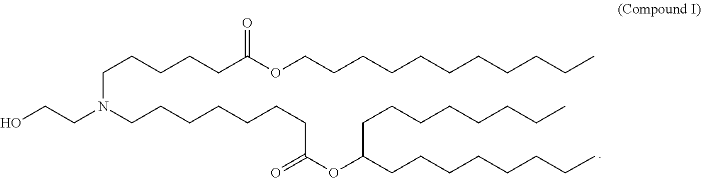 [SMILES: CCCCCCCCCCCOC(=O)CCCCCN(CCO)CCCCCCCC(=O)OC(CCCCCCCC)CCCCCCCC]

In some aspects, an ionizable cationic lipid of the disclosure comprises a compound having structure:

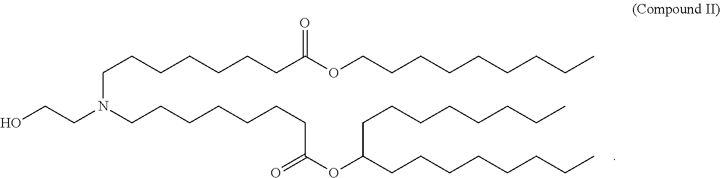 [SMILES: CCCCCCCCCOC(=O)CCCCCCCN(CCO)CCCCCCCC(=O)OC(CCCCCCCC)CCCCCCCC]

LNPs described herein can also be generated using the cationic lipids and compositions comprising them as are known in the art, see, e.g., PCT Publication Nos. WO2015/199952, WO2017/004143, WO2017/075531, WO2017/117528, WO2016/176330, WO2018/191657, WO2018/081480, WO2018/107026, WO2018/200943, WO2018/078053, WO2019/036000, WO2019/036028, WO2019/036030, WO2019/036008, WO2020/061426, WO2020/081938, WO2020/146805, WO2021/030701, WO2022/016070, WO2023/114944, WO2023/114937, WO2023/114943, WO2024/054843, and WO2023/250427, all of which are incorporated by reference herein in their entirety for all purposes.

In some aspects, an ionizable cationic lipid of the disclosure comprises a compound having structure:

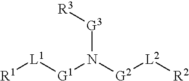

or a pharmaceutically acceptable salt, tautomer, prodrug, or stereoisomer thereof, wherein:
   one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)═NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or ^{a} direct bond;
   G¹ and G² are each independently unsubstituted C₁-C₁₂ alkylene or C₁-C₁₂ alkenylene; G is C₁-C₂₄ alkylene, C₁-C₂₄ alkenylene, C₃-C₈ cycloalkylene, C₃-C₈ cycloalkenylene;
   R^{a} is H or C₁-C₁₂ alkyl;
   R¹ and R² are each independently C₆-C₂₄ alkyl or C₆-C₂₄ alkenyl;
   R³ is H, OR⁵, CN, —C(═O)OR⁴, —OC(═O)R⁴ or —NR⁵C(═O)R⁴;
   R⁴ is C₁-C₁₂ alkyl;
   R⁵ is H or C₁-C₆ alkyl; and
   x is 0, 1, or 2.

In some of the foregoing aspects, the ionizable cationic lipid comprises a compound having one of the following structures:

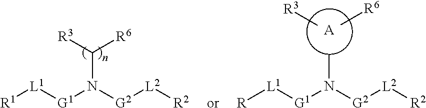

or a pharmaceutically acceptable salt, tautomer, prodrug, or stereoisomer thereof, wherein:
   A is a 3 to 8-membered cycloalkyl or cycloalkylene ring;
   R⁶ is, at each occurrence, independently H, OH or C₁-C₂₄ alkyl; and
   n is an integer ranging from 1 to 15.

In some of the foregoing aspects, the ionizable cationic lipid comprises a compound having one of the following structures:

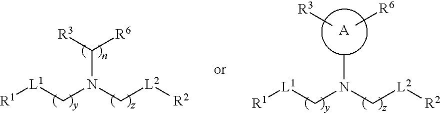

or a pharmaceutically acceptable salt, tautomer, prodrug, or stereoisomer thereof, wherein y and z are each independently integers ranging from 1 to 12.

In any of the foregoing aspects, one of L¹ or L² is —OCCO)—. For example, in some aspects, each of L¹ and L² are —O(C═O)—. In some aspects of any of the foregoing, L¹ and L² are each independently —(C═O)O— or —O(C═O)—. For example, in some aspects, each of L¹ and L² is —(C═O)O—.

In some of the foregoing aspects, the ionizable cationic lipid comprises a compound having one of the following structures:

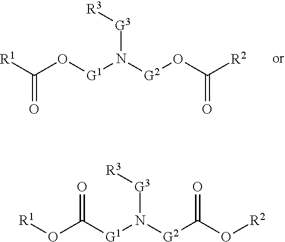

or a pharmaceutically acceptable salt, tautomer, prodrug, or stereoisomer thereof.

In some of the foregoing aspects, the ionizable cationic lipid comprises a compound having one of the following structures:

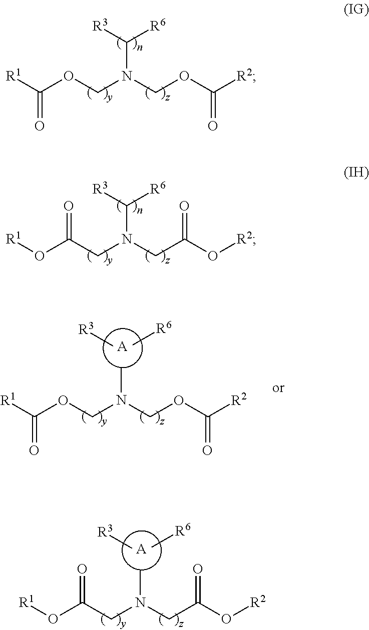

Or a pharmaceutically acceptable salt, tautomer, prodrug, or stereoisomer thereof.

In some of the foregoing aspects, n is an integer ranging from 2 to 12, for example from 2 to 8 or from 2 to 4. For example, in some aspects, n is 3, 4, 5, or 6. In some aspects, n is 3. In some aspects, n is 4. In some aspects, n is 5. In some aspects, n is 6.

In some of the foregoing aspects, y and z are each independently an integer ranging from 2 to 10. For example, in some aspects, y and z are each independently an integer ranging from 4 to 9 or from 4 to 6.

In some of the foregoing aspects, R6 is H. In other of the foregoing embodiments, R⁶ is C₁-C₂₄ alkyl. In other aspects, R⁶ is OH. In some embodiments, G is unsubstituted. In other aspects, G³ is substituted. In various different aspects, G³ is linear C₁-C₂₄ alkylene or linear C₁-C₂₄ alkenylene.

In some other foregoing embodiments, R¹ or R², or both, is C₆-C₂₄ alkenyl. For example, in some embodiments, R¹ and R² each, independently have the following structure:

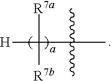

wherein:
   R^{7a} and R^{7b} are, at each occurrence, independently H or C₁-C₁₂ alkyl; and a is an integer from 2 to 12,
   wherein R^{7a}, R^{7b}, and a are each selected such that R¹ and R² each independently comprise from 6 to 20 carbon atoms. For example, in some embodiments a is an integer ranging from 5 to 9 or from 8 to 12.

In some of the foregoing aspects, at least one occurrence of R^{7a} is H. For example, in some aspects, R^{7a} is H at each occurrence. In other different aspects of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl, or n-octyl.

In different aspects, R¹ or R², or both, has one of the following:

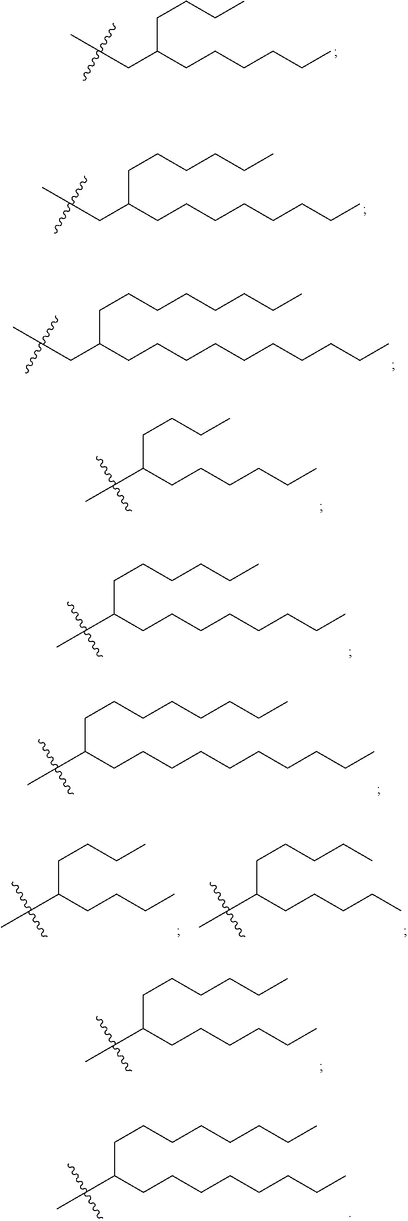

In some of the foregoing aspects, R is OH, CN, —C(═O)OR⁴—OC(═O)R⁴ or —NHC(═O)R⁴. In some aspects, R4 is methyl or ethyl.

It is understood that any aspect of the compounds set forth above, and any specific substituent and/or variable in the compounds set forth above, may be independently combined with other aspects and/or substituents and/or variables of compounds to form aspects of the inventions not specifically set forth above. In addition, in the event that a list of substituents and/or variables is listed for any particular substituent and/or variable in a particular embodiment and/or claim, it is understood that each individual substituent and/or variable may be deleted from the particular aspect and/or claim and that the remaining list of substituents and/or variables will be considered to be within the scope of the disclosure. It is understood that in the present description, combinations of substituents and/or variables of the depicted formulae are permissible only if such contributions result in stable compounds.

In some embodiments, the cationic lipid is

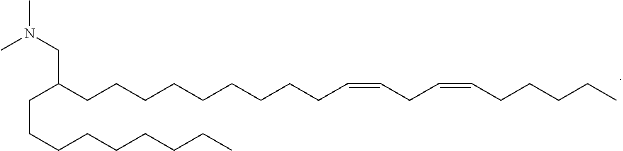 [SMILES: CCCCC/C=C\C/C=C\CCCCCCCCCC(CCCCCCCCC)CN(C)C]

In some embodiments, the cationic lipid is

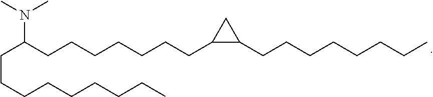 [SMILES: CCCCCCCCCC(CCCCCCCC1CC1CCCCCCCC)N(C)C]

In some aspects, the lipid nanoparticles comprise one or more cationic lipids. In one aspect, the lipid nanoparticles comprise (4-hydroxybutyl)azanediyl)bis(hexane-6,1-diyl)bis(2-hexyldecanoate) (ALC-0315), having the formula:

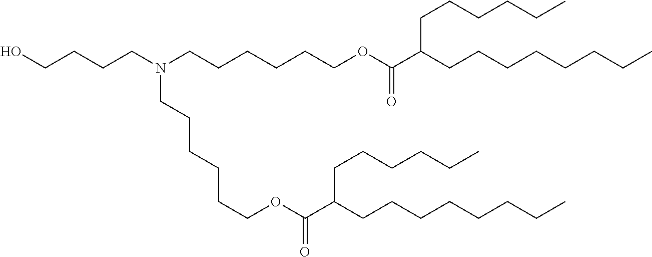 [SMILES: CCCCCCCCC(CCCCCC)C(=O)OCCCCCCN(CCCCO)CCCCCCOC(=O)C(CCCCCC)CCCCCCCC]

Exemplary cationic lipids are disclosed in, e.g., U.S. Pat. No. 10,166,298, the full disclosure of which is herein incorporated by reference in its entirety for all purposes. Representative cationic lipids include:

| No. | Structure |
|---|---|
| 1 | 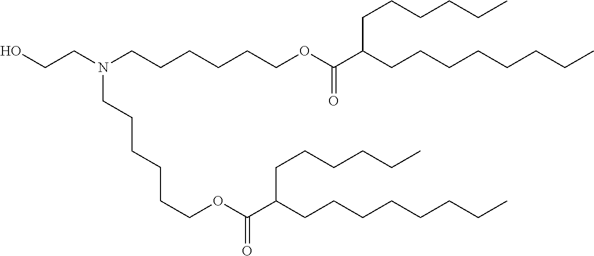 [SMILES: CCCCCCCCC(CCCCCC)C(=O)OCCCCCCN(CCO)CCCCCCOC(=O)C(CCCCCC)CCCCCCCC] |
| 2 | 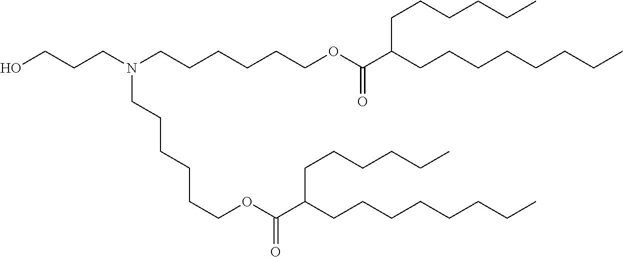 [SMILES: CCCCCCCCC(CCCCCC)C(=O)OCCCCCCN(CCCO)CCCCCCOC(=O)C(CCCCCC)CCCCCCCC] |
| 3 | 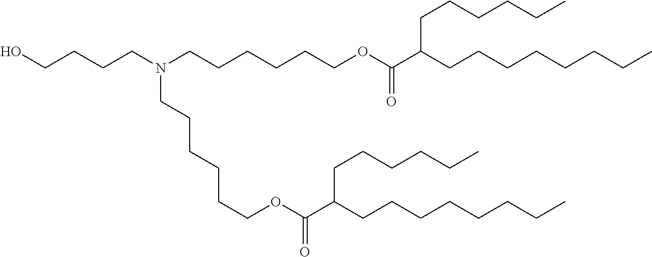 [SMILES: CCCCCCCCC(CCCCCC)C(=O)OCCCCCCN(CCCCO)CCCCCCOC(=O)C(CCCCCC)CCCCCCCC] |
| 4 | 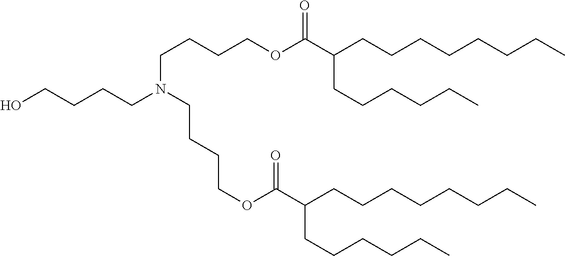 [SMILES: CCCCCCCCC(CCCCCC)C(=O)OCCCCN(CCCCO)CCCCOC(=O)C(CCCCCC)CCCCCCCC] |
| 5 | 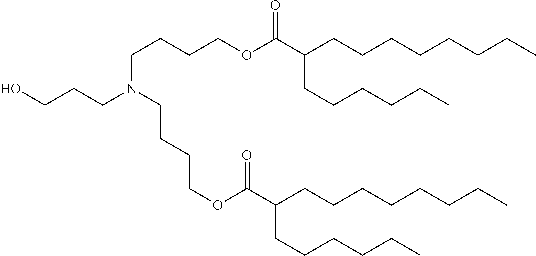 [SMILES: CCCCCCCCC(CCCCCC)C(=O)OCCCCN(CCCO)CCCCOC(=O)C(CCCCCC)CCCCCCCC] |
| 6 | 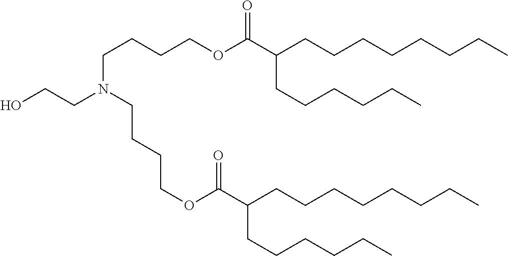 [SMILES: CCCCCCCCC(CCCCCC)C(=O)OCCCCN(CCO)CCCCOC(=O)C(CCCCCC)CCCCCCCC] |
| 7 | 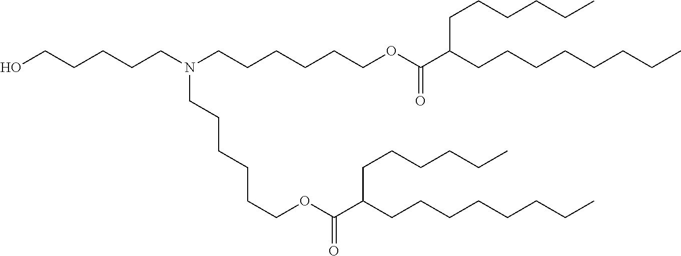 [SMILES: CCCCCCCCC(CCCCCC)C(=O)OCCCCCCN(CCCCCO)CCCCCCOC(=O)C(CCCCCC)CCCCCCCC] |
| 8 | 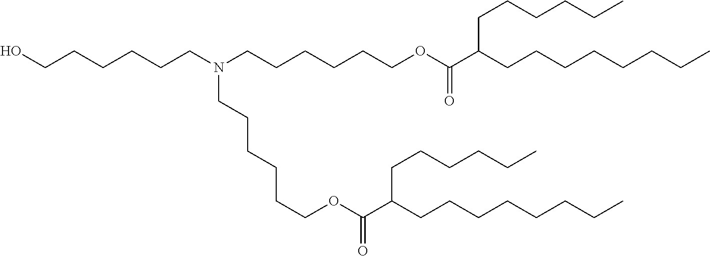 [SMILES: CCCCCCCCC(CCCCCC)C(=O)OCCCCCCN(CCCCCCO)CCCCCCOC(=O)C(CCCCCC)CCCCCCCC] |
| 9 | 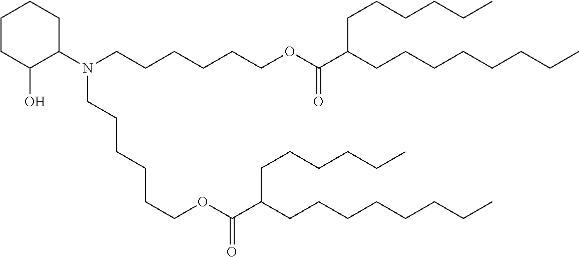 [SMILES: CCCCCCCCC(CCCCCC)C(=O)OCCCCCCN(CCCCCCOC(=O)C(CCCCCC)CCCCCCCC)C1CCCCC1O] |
| 10 | 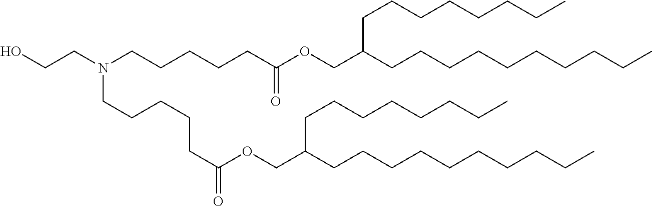 [SMILES: CCCCCCCCCCC(CCCCCCCC)COC(=O)CCCCCN(CCO)CCCCCC(=O)OCC(CCCCCCCC)CCCCCCCCCC] |
| 11 | 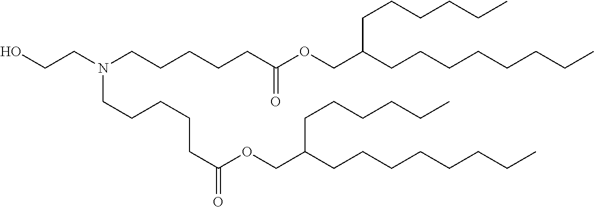 [SMILES: CCCCCCCCC(CCCCCC)COC(=O)CCCCCN(CCO)CCCCCC(=O)OCC(CCCCCC)CCCCCCCC] |
| 12 | 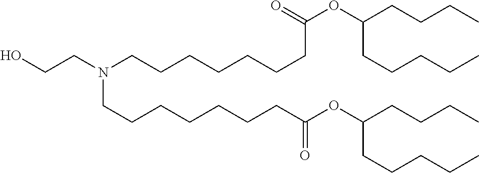 [SMILES: CCCCCC(CCCC)OC(=O)CCCCCCCN(CCO)CCCCCCCC(=O)OC(CCCC)CCCCC] |
| 13 | 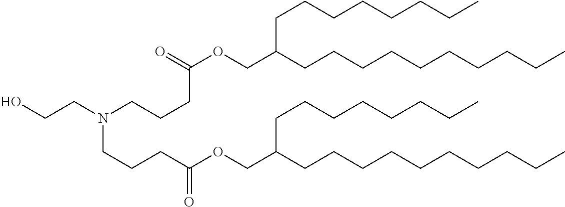 [SMILES: CCCCCCCCCCC(CCCCCCCC)COC(=O)CCCN(CCO)CCCC(=O)OCC(CCCCCCCC)CCCCCCCCCC] |
| 14 | 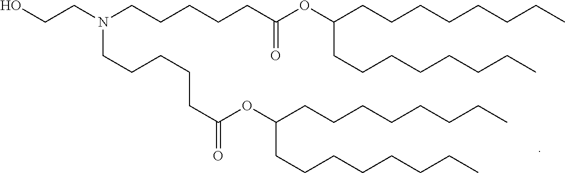 [SMILES: CCCCCCCCC(CCCCCCCC)OC(=O)CCCCCN(CCO)CCCCCC(=O)OC(CCCCCCCC)CCCCCCCC] |
| 15 | 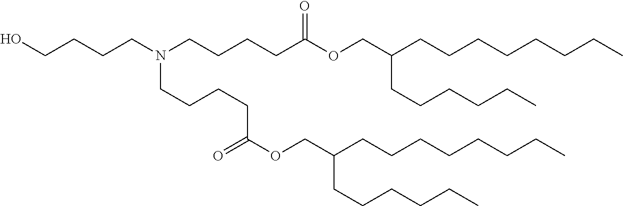 [SMILES: CCCCCCCCC(CCCCCC)COC(=O)CCCCN(CCCCO)CCCCC(=O)OCC(CCCCCC)CCCCCCCC] |
| 16 | 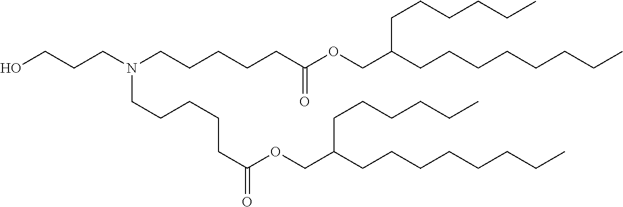 [SMILES: CCCCCCCCC(CCCCCC)COC(=O)CCCCCN(CCCO)CCCCCC(=O)OCC(CCCCCC)CCCCCCCC] |
| 17 | 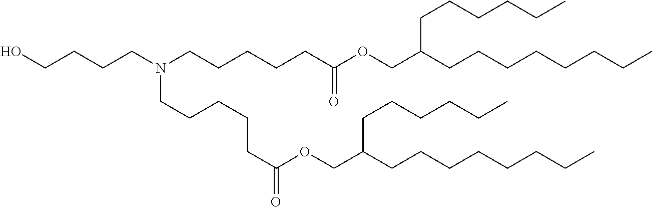 [SMILES: CCCCCCCCC(CCCCCC)COC(=O)CCCCCN(CCCCO)CCCCCC(=O)OCC(CCCCCC)CCCCCCCC] |
| 18 | 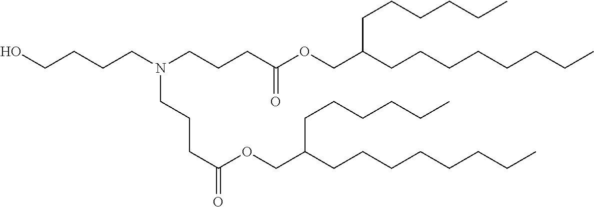 [SMILES: CCCCCCCCC(CCCCCC)COC(=O)CCCN(CCCCO)CCCC(=O)OCC(CCCCCC)CCCCCCCC] |
| 19 | 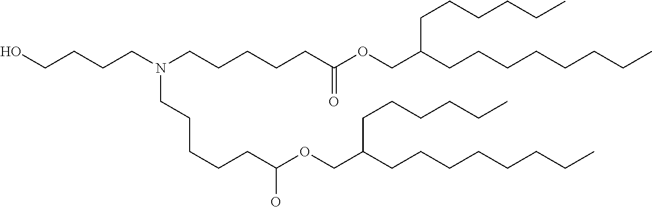 [SMILES: CCCCCCCCC(CCCCCC)COC(=O)CCCCCN(CCCCO)CCCCCC(O)OCC(CCCCCC)CCCCCCCC] |
| 20 | 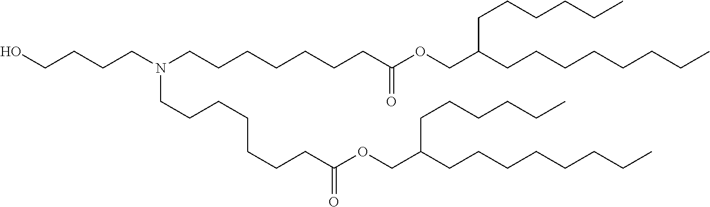 [SMILES: CCCCCCCCC(CCCCCC)COC(=O)CCCCCCCN(CCCCO)CCCCCCCC(=O)OCC(CCCCCC)CCCCCCCC] |
| 21 | 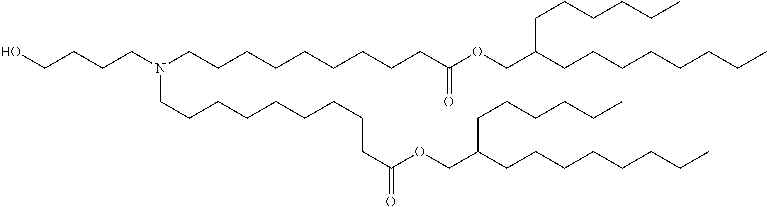 [SMILES: CCCCCCCCC(CCCCCC)COC(=O)CCCCCCCCCN(CCCCO)CCCCCCCCCC(=O)OCC(CCCCCC)CCCCCCCC] |
| 22 | 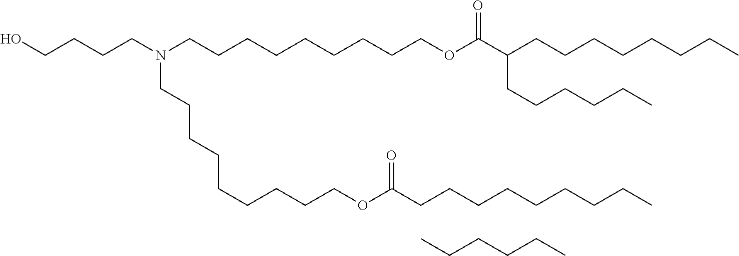 |
| 23 | 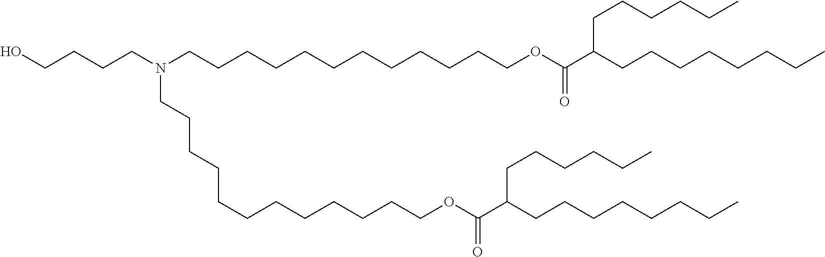 [SMILES: CCCCCCCCC(CCCCCC)C(=O)OCCCCCCCCCCCCN(CCCCO)CCCCCCCCCCCCOC(=O)C(CCCCCC)CCCCCCCC] |
| 24 | 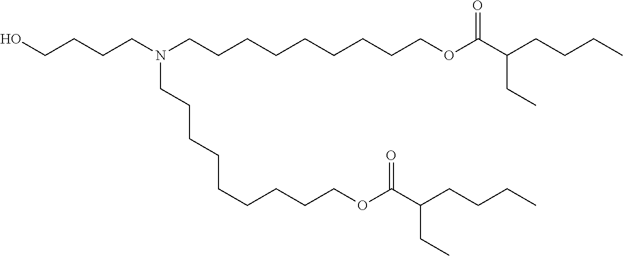 [SMILES: CCCCC(CC)C(=O)OCCCCCCCCCN(CCCCO)CCCCCCCCCOC(=O)C(CC)CCCC] |
| 25 | 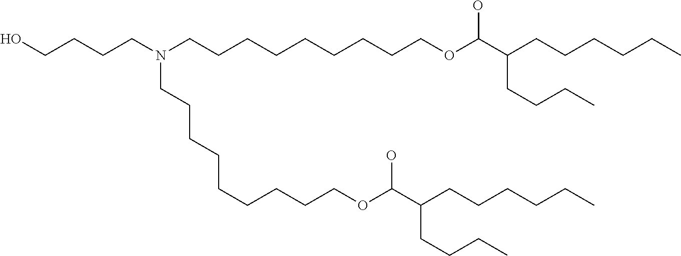 [SMILES: CCCCCCC(CCCC)C(O)OCCCCCCCCCN(CCCCO)CCCCCCCCCOC(O)C(CCCC)CCCCCC] |
| 26 | 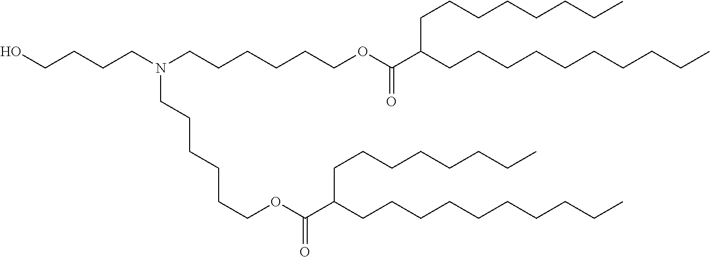 [SMILES: CCCCCCCCCCC(CCCCCCCC)C(=O)OCCCCCCN(CCCCO)CCCCCCOC(=O)C(CCCCCCCC)CCCCCCCCCC] |
| 27 | 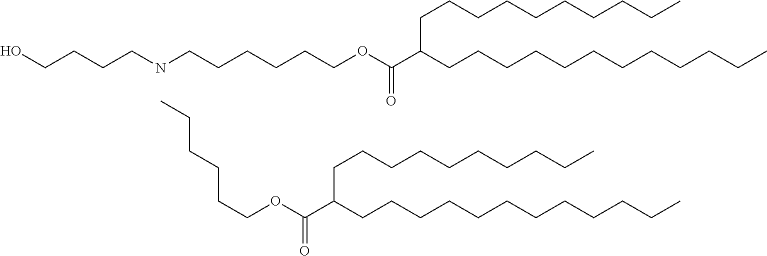 |
| 28 | 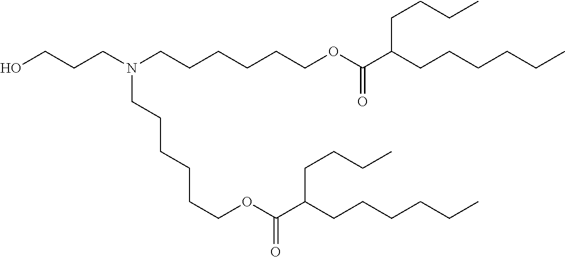 [SMILES: CCCCCCC(CCCC)C(=O)OCCCCCCN(CCCO)CCCCCCOC(=O)C(CCCC)CCCCCC] |
| 29 | 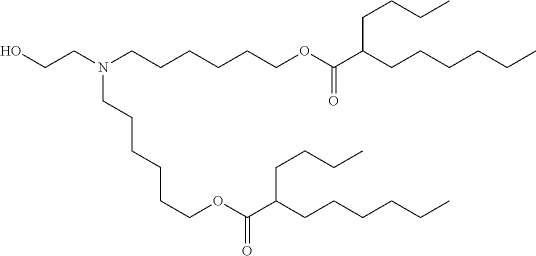 [SMILES: CCCCCCC(CCCC)C(=O)OCCCCCCN(CCO)CCCCCCOC(=O)C(CCCC)CCCCCC] |
| 30 | 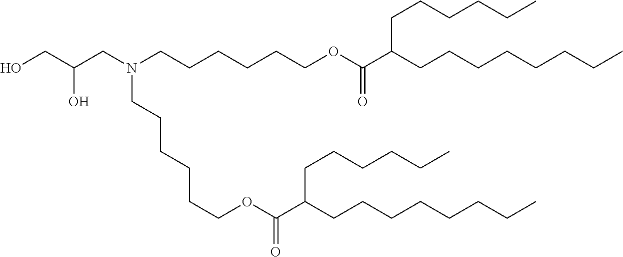 [SMILES: CCCCCCCCC(CCCCCC)C(=O)OCCCCCCN(CCCCCCOC(=O)C(CCCCCC)CCCCCCCC)CC(O)CO] |
| 31 | 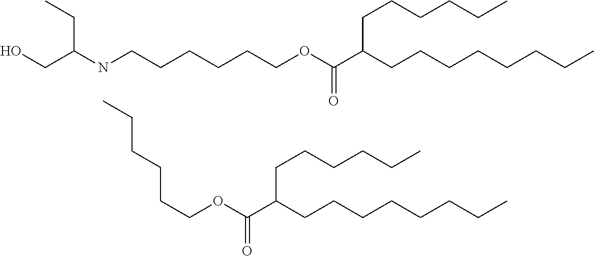 |
| 32 | 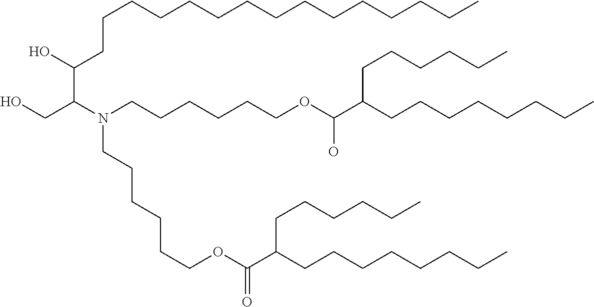 [SMILES: CCCCCCCCCCCCCCCC(O)C(CO)N(CCCCCCOC(=O)C(CCCCCC)CCCCCCCC)CCCCCCOC(O)C(CCCCCC)CCCCCCCC] |
| 33 | 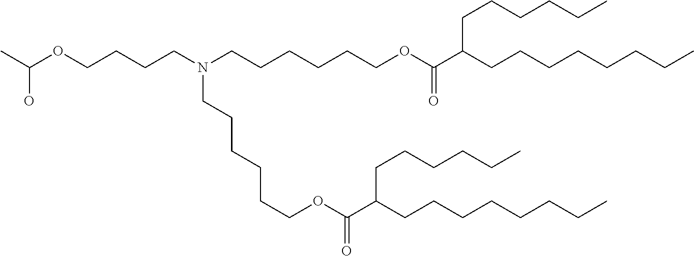 [SMILES: CCCCCCCCC(CCCCCC)C(=O)OCCCCCCN(CCCCCCOC(=O)C(CCCCCC)CCCCCCCC)CCCCOC(C)O] |
| 34 | 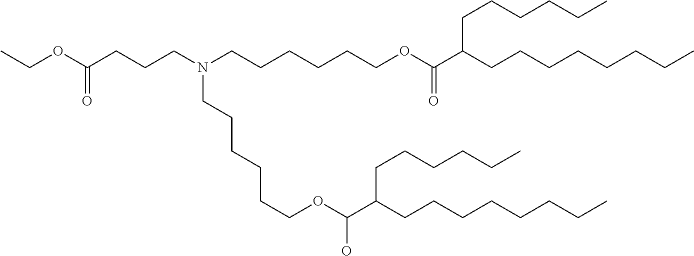 [SMILES: CCCCCCCCC(CCCCCC)C(=O)OCCCCCCN(CCCCCCOC(O)C(CCCCCC)CCCCCCCC)CCCC(=O)OCC] |
| 35 | 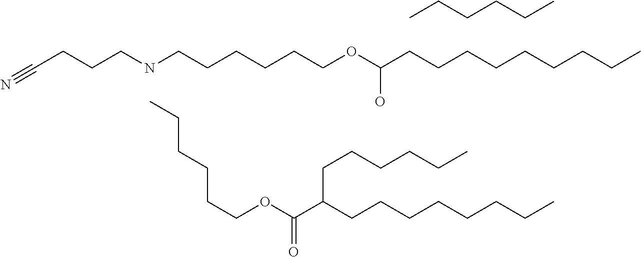 |
| 36 | 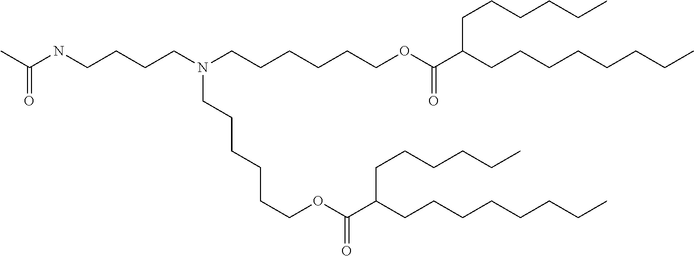 [SMILES: CCCCCCCCC(CCCCCC)C(=O)OCCCCCCN(CCCCCCOC(=O)C(CCCCCC)CCCCCCCC)CCCCNC(C)=O] |

In yet another aspect, the ionizable cationic lipid is described in PCT Publication No. WO2015/199952, the contents of which is hereby incorporated herein by reference in its entirety for all purposes. In one embodiment therein, an ionizable cationic lipid of the disclosure comprises a compound of Formula (I):

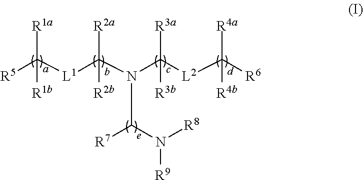

or a pharmaceutically acceptable salt, tautomer, prodrug or stereoisomer thereof, wherein:
L¹ and L² are each independently —O(C═O)—, —(C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R2b and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl;
R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24;
b and c are each independently an integer from 1 to 24; and
e is 1 or 2.

In other embodiments, the lipid compounds have the following Structure (Ia):

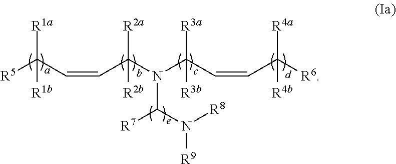

In another embodiment, the lipid compounds have the following Structure (Ib):

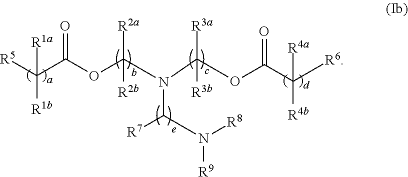

In yet other embodiments, the lipid compounds have the following Structure (Ic):

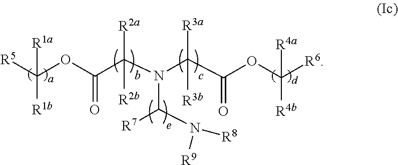

Compound Nos. 1-41 as specifically exemplified in Table 1 of PCT Publication No. WO2015/199952, and falling within the generic scope of Structures (I), (Ia), (Ib) or (Ic) described above, are hereby incorporated herein by reference for all purposes.

In yet another aspect, the ionizable cationic lipid is described in PCT Publication No. WO2016/176330, the contents of which is hereby incorporated herein by reference in its entirety for all purposes. In one embodiment therein, an ionizable cationic lipid of the disclosure comprises a compound of Formula (I):

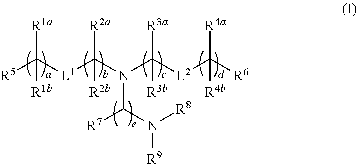

or a pharmaceutically acceptable salt, tautomer, prodrug or stereoisomer thereof, wherein:
L¹ and L² are each independently —O(C═O)—, —(C═O)O— or a carbon carbon double bond;
   R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
   R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
   R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
   R^{4a} and R^{4b} are, at each occurrence, independently either (a) H or C₁-C₁₂ alkyl, or (b) R4a is H or C₁-C₁₂ alkyl, and R^{4b} together with the carbon atom to which it is bound is taken together with an adjacent R^{4b} and the carbon atom to which it is bound to form a carbon-carbon double bond;
   R⁵ and R⁶ are each independently methyl or cycloalkyl;
   R⁷ is, at each occurrence, independently H or C₁-C₁₂ alkyl;
   R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;
   a and d are each independently an integer from 0 to 24;
   b and c are each independently an integer from 1 to 24; and e is 1 or 2.

In one embodiment, the ionizable cationic lipid comprises a compound having a structure of

### Formula (II):

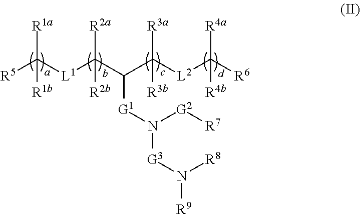

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}, —OC(═O)NR^{a}—, —NR^{a}C(═O)O—, or a direct bond;
G¹ is C₁-C₂ alkylene, —(C═O)—, —O(C═O)—, —SC(═O)—, —NR^{a}C(═O)— or a direct bond;
G² is —C(═O)—, —(C═O)O—, —C(═O)S—, —C(═O)NR^{a} or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either: (a) H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4B} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R8 and R9, together with the nitrogen atom to which they are attached, form a 5, 6 or 7-membered heterocyclic ring;
a, b, c and d are each independently an integer from 1 to 24; and x is 0, 1 or 2.

In one embodiment, the ionizable cationic lipid comprises a compound having a structure of Formula (III):

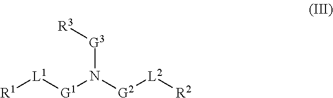

or a pharmaceutically acceptable salt, tautomer, prodrug or stereoisomer thereof, wherein:
one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl; R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

Compound Nos. I-1 to I-41 as specifically exemplified in Table 1, Compound Nos. II-1 to II-34 as specifically exemplified in Table 2, and Compound Nos. III-1 to III-36 as specifically exemplified in Table 3, of PCT Publication No. WO2016/176330, and falling within the generic scope of Structures (I), (II) or (III) described above, are hereby incorporated herein by reference for all purposes.

In yet another aspect, the ionizable cationic lipid is described in PCT Publication No. WO2017/004143, the contents of which is hereby incorporated herein by reference in its entirety for all purposes. In one embodiment therein, an ionizable cationic lipid of the disclosure comprises a compound of Formula (I):

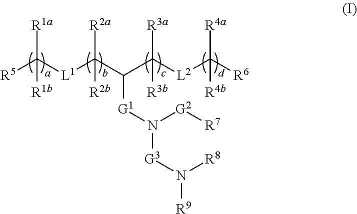

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}—, —OC(═O)R^{a}—, —NR^{a}C(═O)O— or a direct bond;
G¹ is C₁-C₂ alkylene, —(C═O)—, —O(C═O)—, —SC(═O)—, —NR^{a}C(═O)— or a direct bond;
G² is —C(═O)—, —(C═O)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R1b and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R2b and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R4b and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and x is 0, 1 or 2.

In one embodiment, the cationic lipid comprises a compound having a structure of Formula (IA), (IB), (IC) or (ID):

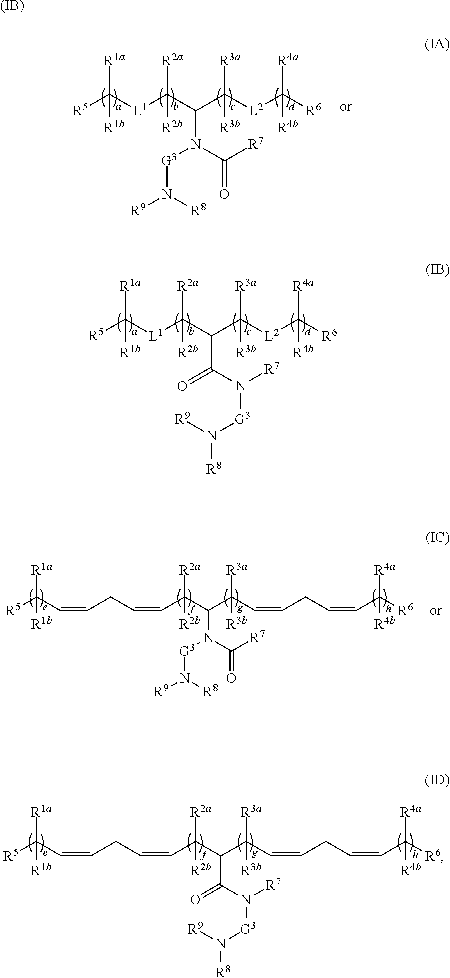

wherein e, f, g and h are each independently an integer from 1 to 12.

In different aspects, R^{b} is branched C₁-C₁₅ alkyl. For example, in some embodiments R^{b} has one of the following structures:

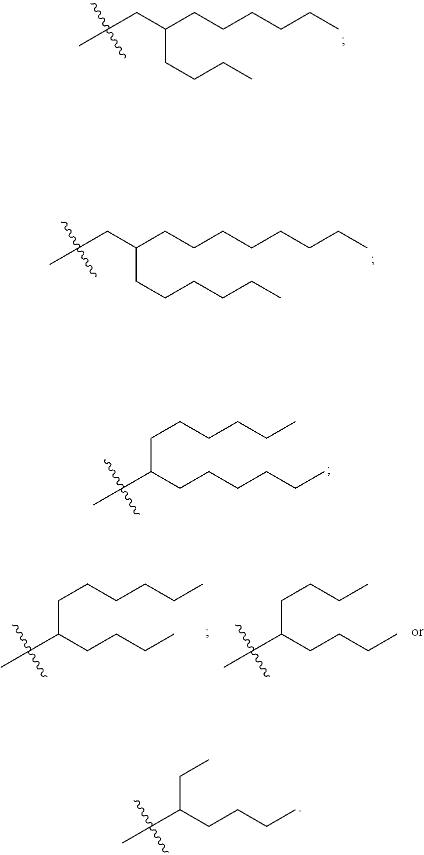

Compound Nos. 1-46 as specifically exemplified in Table 1 of PCT Publication No. WO2017/004143 described above, are hereby incorporated herein by reference for all purposes.

In another aspect, the cationic lipid is described in PCT Publication No. WO2017/117528, the contents of which is hereby incorporated herein by reference in its entirety for all purposes. In one embodiment therein, a cationic lipid of the disclosure comprises a compound of Formula (I):

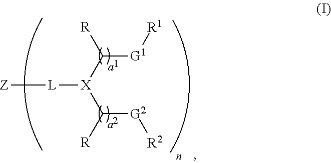

or a pharmaceutically acceptable salt, prodrug or stereoisomer thereof.

Compound Nos. 1-3 as specifically exemplified in Table 1 of PCT Publication No. WO2017/117528 described above, are hereby incorporated herein by reference for all purposes.

In another aspect, the cationic lipid is described in PCT Publication No. WO2018/191657, the contents of which is hereby incorporated herein by reference in its entirety for all purposes. In one embodiment therein, a cationic lipid of the disclosure comprises a compound of Formula (I):

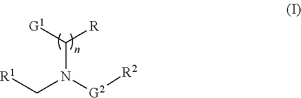

or a pharmaceutically acceptable salt, prodrug or stereoisomer thereof, wherein:
G¹ is —OH, —R³R⁴, —(C═O)NR⁵ or —NR³(C═O)R⁵;
G² is —CH₂— or —(C═O)—;
R is, at each occurrence, independently H or OH;
R¹ and R² are each independently optionally substituted branched, saturated or unsaturated C₁₂-C₃₆ alkyl;
R³ and R⁴ are each independently H or optionally substituted straight or branched, saturated or unsaturated C₁-C₆ alkyl;
R⁵ is optionally substituted straight or branched, saturated or unsaturated C₁-C₆ alkyl; and
n is an integer from 2 to 6.

In one embodiment, the cationic lipid comprises a compound having a structure of Formula (IA):

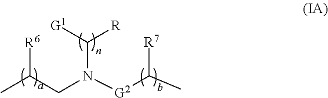

wherein:
R⁶ and R⁷ are, at each occurrence, independently H or straight or branched, saturated or unsaturated C₁-C₁₄ alkyl;
a and b are each independently an integer ranging from 1 to 15, provided that R⁶ and a, and R⁷ and b, are each independently selected such that R¹ and R², respectively, are each independently branched, saturated or unsaturated C₁₂-C₃₆ alkyl.

In one embodiment, the cationic lipid comprises a compound having a structure of Formula (IB):

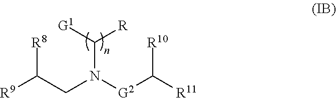

wherein R⁸, R⁹, R¹⁰ and R¹¹ are each independently straight or branched, saturated or unsaturated C₄-C₁₂ alkyl, provided that R⁸ and R⁹, and R¹⁰ and R¹¹, are each independently selected such that R¹ and R², respectively, are each independently branched, saturated or unsaturated C₁₂-C₃₆ alkyl.

In different aspects, R¹ and R² have the following structure:

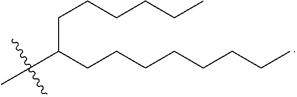 [SMILES: *C(CCCCCC)CCCCCCCC]

Compound Nos. 1-17 as specifically exemplified in Table 1 of PCT Publication No. WO2018/191657 described above, are hereby incorporated herein by reference for all purposes.

In yet another aspect, the cationic lipid is described in PCT Publication No. WO2018/081480, the contents of which is hereby incorporated herein by reference in its entirety for all purposes. In one embodiment therein, a cationic lipid of the disclosure comprises a compound of Structure (I), (II), (III), (IV) or (IV), or a pharmaceutically acceptable salt, prodrug or stereoisomer thereof.

Compound Nos. I-1 to I-41 as specifically exemplified in Table 1, Compound Nos. II-1 to II-46 as specifically exemplified in Table 2, Compound Nos. III-1 to III-49 as specifically exemplified in Table 3, and Compound Nos. IV-1 to IV-3 as specifically exemplified in Table 4 of PCT Publication No. WO2018/081480 described above, are hereby incorporated herein by reference for all purposes.

In yet another aspect, the cationic lipid is described in PCT Publication No. WO2018/107026, the contents of which is hereby incorporated herein by reference in its entirety for all purposes. In one embodiment therein, a cationic lipid of the disclosure is selected from compounds having the following Formulas (I, II, III and IV):

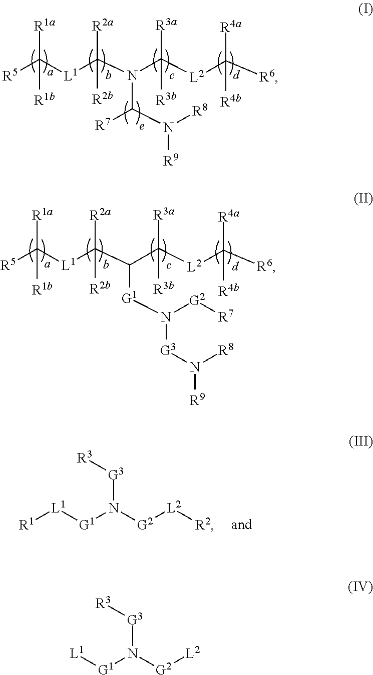

or a pharmaceutically acceptable salt, tautomer or stereoisomer thereof, wherein, for Formula (I):
L¹ and L² are each independently —O(C═O)—, (C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R1b and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R2b and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R4b and the carbon atom to which it is bound to form a carbon-carbon double bond;
R⁵ and R⁶ are each independently methyl or cycloalkyl; R⁷ is, at each occurrence, independently H or C₁-C₁₂ alkyl;
R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom; a and d are each independently an integer from 0 to 24; b and c are each independently an integer from 1 to 24; and e is 1 or 2, for Formula (II):
L¹ and L² are each independently —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;
G¹ is C₁-C₂ alkylene, —(C═O)—, —O(C═O)—, —SC(═O)—, —NR^{a}C(═O)— or a direct bond;
G² is —C(═O)—, —(C═O)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R2b and the carbon atom to which it is bound to form a carbon-carbon double bond;
R^{3a} and R^{3b} are, at each occurrence, independently either: (a) H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and
x is 0, 1 or 2;

### for Formula (III):

one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— —R^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)R^{a}—, —OC(═O)R^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2; and

### for Formula (IV):

one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}—, —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, —NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In one embodiment, the cationic lipid is selected from any one of Compound Nos. I-1 to I-41 as specifically exemplified in Table 1, Compound Nos. II-1 to II-36 as specifically exemplified in Table 2, Compound Nos. III-1 to III-49 as specifically exemplified in Table 3, and Compound Nos. 1 to 17 as specifically exemplified in Table 4, of PCT Publication No. WO2018/107026, which Tables and compounds listed therein are hereby incorporated herein by reference for all purposes.

In yet another aspect, the cationic lipid is described in PCT Publication No. WO2018/200943, the contents of which is hereby incorporated herein by reference in its entirety for all purposes. In one embodiment therein, a cationic lipid of the disclosure is selected from compounds having the following Structures (I) or (II):

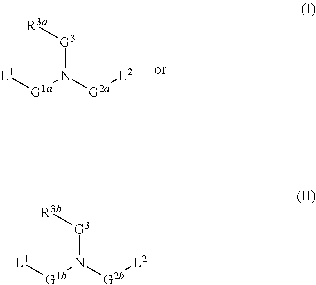

or a pharmaceutically acceptable salt, prodrug or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)R^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G^{1a} and G^{2a} are each independently C₂-C₁₂ alkylene or C₂-C₁₂ alkenylene;
G^{1b} and G^{2b} are each independently C₁-C₁₂ alkylene or C₂-C₁₂ alkenylene;
G3 is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₂-C₁₂ alkenyl;
R^{c} and R^{f} are each independently C₁-C₁₂ alkyl or C₂-C₁₂ alkenyl;
R¹ and R² are each independently branched C₁-C₂₄ alkyl or branched C₂-C₂₄ alkenyl;
R^{3a} is —C(═O)N(R^{4a})R^{5a} or —C(═O)OR⁶;
R^{3b} is —NR^{4b}C(═O)R^{5b};
R^{4a} is C₁-C₁₂ alkyl;
R^{4b} is H, C₁-C₁₂ alkyl or C₂-C₁₂ alkenyl;
R^{5a} is H, C₁-C₈ alkyl or C₂-C₈ alkenyl;
R^{5b} is C₂-C₁₂ alkyl or C₂-C₁₂ alkenyl when R^{4b} is H; or R^{5b} is C1-C12 alkyl or C₂-C₁₂ alkenyl when R^{4b} is C₁-C₁₂ alkyl or C₂-C₁₂ alkenyl;
R⁶ is H, aryl or aralkyl; and
x is 0, 1 or 2, and
wherein each alkyl, alkenyl, alkylene, alkenylene, cycloalkylene, cycloalkenylene, aryl and aralkyl is independently substituted or unsubstituted.

In one embodiment, a cationic lipid of the disclosure is selected from compounds having the following Structures (IA) or (IIA):

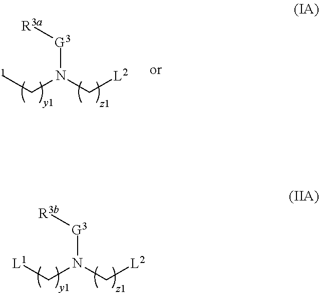

wherein y1 and z1 are each independently integers ranging from 2 to 12, and y2 and z2 are each independently integers ranging from 1 to 12.

In another embodiment, a cationic lipid of the disclosure is selected from compounds having the following Structures (IB), (IC), (ID), (IE), (IIB) or (IIC):

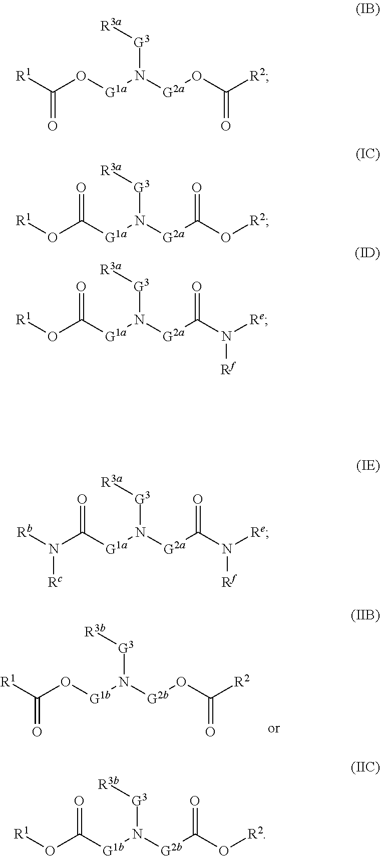

In another embodiment, a cationic lipid of the disclosure is selected from compounds having the following Structures (IF), (IG), (IH), (IJ), (IID) or (IIE):

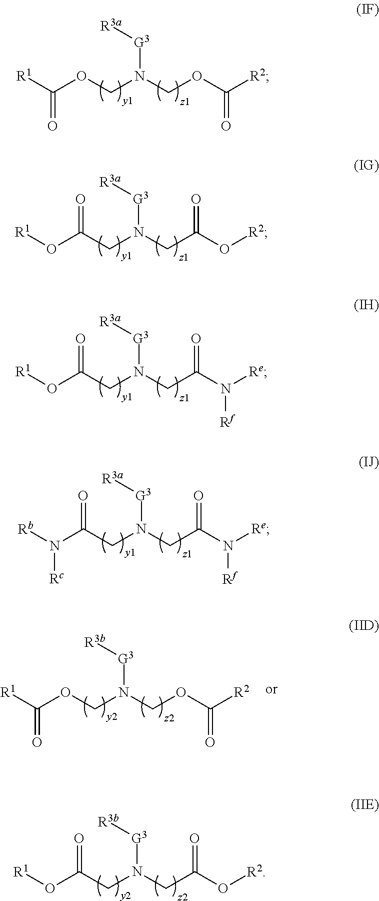

In different aspects, R1 or R2, or both, independently has one of the following structures:

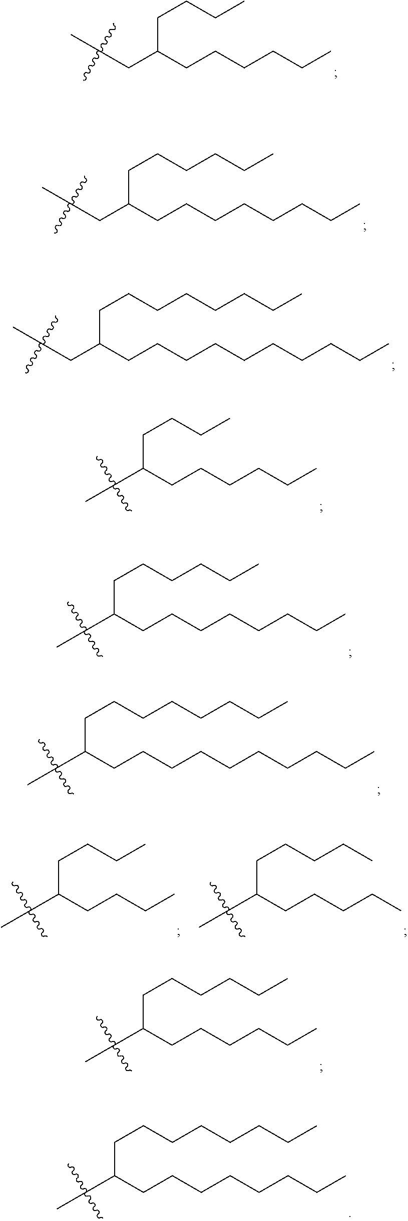

In a further embodiment, the cationic lipid is selected from any one of Compound Nos. I-1 to I-19 as specifically exemplified in Table 1, or Compound Nos. II-1 to II-20 as specifically exemplified in Table 2, of PCT Publication No. WO2018/200943, which Tables and compounds listed therein are hereby incorporated herein by reference for all purposes.

In yet another aspect, the cationic lipid is described in PCT Publication No. WO2018/078053, the contents of which is hereby incorporated herein by reference in its entirety for all purposes. In one embodiment therein, a cationic lipid of the disclosure is a compound having Formula (I):

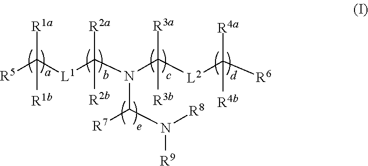

or a pharmaceutically acceptable salt, tautomer or stereoisomer thereof, wherein:
L¹ and L² are each independently —O(C═O)—, —(CO)O— or a carbon-carbon double bond;
R^{1a} and R^{1b} are, at each occurrence, independently either (a) H or C₁-C₁₂, alkyl, or (b) R^{1a} is H or C₁-C₁₂, alkyl, and R^{1b} together with the carbon atom to which it is bound is taken together with an adjacent R1b and the carbon atom to which it is bound to form a carbon-carbon double bond;
R^{2a} and R^{2b} are, at each occurrence, independently either (a) H or C₁-C₁₂, alkyl, or (b) R^{2a} is H or C₁-C₁₂, alkyl, and R^{2b} together with the carbon atom to which it is bound is taken together with an adjacent R2b and the carbon atom to which it is bound to form a carbon-carbon double bond;
R^{1a} and R^{3b} are, at each occurrence, independently either (a) H or C₁-C₁₂, alkyl, or (b) R^{1a} is H or C₁-C₁₂, alkyl, and R^{3b} together with the carbon atom to which it is bound is taken together with an adjacent R30 and the carbon atom to which it is bound to form a carbon-carbon double bond;
R^{4a} and R^{4b} are, at each occurrence, independently either (a) H or C₁-C₁₂, alkyl, or (b) R^{4a} is H or C₁-C₁₂, alkyl, and R^{4b} together with the carbon atom to which it is bound is taken together with an adjacent R4b and the carbon atom to which it is bound to form a carbon-carbon double bond;
R⁵ and R⁶ are each independently methyl or cycloalkyl;
R⁷ is, at each occurrence, independently H or C₁-C₁₂, alkyl;
R⁸ and R⁹ are each independently C₁-C₁₂, alkyl; or R⁸ and R⁹, together with the nitrogen atom to which they are attached, form a 5, 6 or 7-membered heterocyclic ring comprising one nitrogen atom;
a and d are each independently an integer from 0 to 24;
b and c are each independently an integer from 1 to 24; and
e is 1 or 2.

In another embodiment, a cationic lipid of the disclosure is a compound having Formula (II):

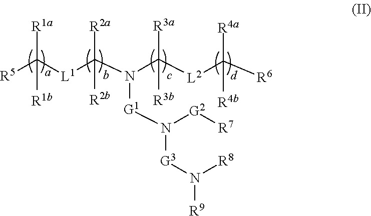

or a pharmaceutically acceptable salt, tautomer or stereoisomer thereof, wherein:
L¹ and L² are each independently —O(C═O)—, —(C═O)O—, —(C═O)—, —O—, —S—S—, —C(═O)S—, —SC(═O)—, —NR^{a}C(═O)—, —C(═O)NR^{a}—, —NR^{a}C(═O)NR^{a}—; —OC(═O)NR^{a}—, —NR^{a}C(═O)O—, or a direct bond;
G¹ is C₁-C₂ alkylene, —(C═)—, —O(CO)—, —SC(═O)—, —NR^{a}C(═O)− or a direct bond
G² is —C(═O)—, —(C═O)O—, —C(═O)S—, —C(═O)NR^{a}— or a direct bond; G3 is C₁-C₆ alkylene;
R^{a} is, at each occurrence, independently H or C₁-C₁₂ alkyl;
R^{1a} and R¹⁶ are, at each occurrence, independently either: (a) H or C₁-C₁₂ alkyl; or (b) R^{1a} is H or C₁-C₁₂, alkyl, and R¹⁵ together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂, alkyl; or (b) R^{2a} is H or C₁-C₁₂, alkyl, and R^{2b} together with the carbon atom to which it is bound is taken together with an adjacent R^{2b} and the carbon atom to which it is bound to form a carbon-carbon double bond;
R^{1a} and R^{3b} are, at each occurrence, independently either: (a) H or C₁-C₁₂, alkyl; or (b) R^{1a} is H or C₁-C₁₂, alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C₁-C₁₂, alkyl; or (b) R^{4a} is H or C₁-C₁₂, alkyl, and R^{4b} together with the carbon atom to which it is bound is taken together with an adjacent R4b and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂, alkyl; or R⁸ and R⁹, together with the nitrogen atom to which they are attached, form a 5, 6 or 7-membered heterocyclic ring;
a, b, c and d are each independently an integer from 1 to 24; and
x is 0, 1 or 2.

In another embodiment, a cationic lipid of the disclosure is a compound having Formula (III):

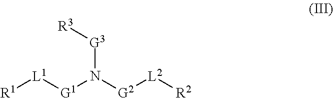

or a pharmaceutically acceptable salt, tautomer or stereoisomer thereof, wherein:
L¹ and L² are each independently —O(C═O)—, —(C═O)O—, —C(═O)—, S(O)ₓ—, —S—S—, —C(═O)—, —SC(═O)—, —NR^{a}C(═O)—, —C(═O)NR^{a}—, —NR^{a}C(═O)NR^{a}—, —OC(═O)NR^{a}— or —NR^{a}C(═O)O—;
G¹ and G² are each independently unsubstituted C₁-C₁₂ alkylene or C₁-C₁₂ alkenylene;
G³ is C₁-C₂₄ alkylene, C₁-C₂₄ alkenylene, C₅-C₈cycloalkylene, or C₁-C₁₂ cycloalkenylene;
R^{a} is, at each occurrence, independently H or C₁-C₁₂, alkyl;
R¹ and R² are each independently C₆-C₂₄ alkyl or C₆-C₂₄ alkenyl;
R³ is OR⁵, CN, —C(═O)OR⁴, —OC(═O)R⁴ or —NR⁵C(═O)R⁴;
R⁴ is C₁-C₁₂ alkyl;
R⁵ is H or C₁-C₁₂ alkyl; and
x is 0, 1 or 2.

In a further embodiment, the cationic lipid is selected from any one of Compound Nos. I-1 to I-41 as specifically exemplified in Table 7, or Compound Nos. II-1 to II-36 as specifically exemplified in Table 8, or Compound Nos. III-1 to III-36 as specifically exemplified in Table 9, of PCT Publication No. WO2018/078053, which Tables and compounds listed therein are hereby incorporated herein by reference for all purposes.

In yet another aspect, the cationic lipid is described in PCT Publication No. WO2019/036000, the contents of which is hereby incorporated herein by reference in its entirety for all purposes. In one embodiment therein, an ionizable cationic lipid of the disclosure comprises a compound of Formula (I):

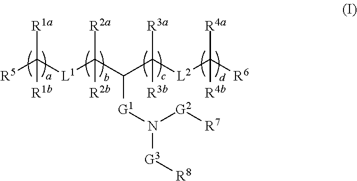

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;
G¹ is C₁-C₂ alkylene, —(C═O)—, —O(C═O)—, —SC(═O)—, —NR^{a}C(═O)— or a direct bond;
G² is —C(═O)—, —(C═O)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R′b and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is H or C₁-C₂₀ alkyl;
R⁸ is OH, —N(R⁹)(C═O)R¹⁰, —(C═O)NR⁹R¹⁰, —NR⁹R¹⁰, —(C═O)OR¹¹ or —O(C═O)R¹¹, provided that G³ is C₄-C₆ alkylene when R⁸ is —NR⁹R¹⁰,
R⁹ and R¹⁰ are each independently H or C₁-C₁₂ alkyl;
R¹¹ is aralkyl;
a, b, c and d are each independently an integer from 1 to 24; and
x is 0, 1 or 2,
wherein each alkyl, alkylene and aralkyl is optionally substituted.

In another embodiment, a cationic lipid of the disclosure is a compound having the following structures (IA) or (IB):

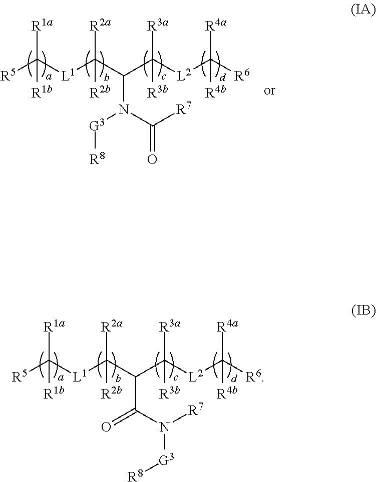

In another embodiment, a cationic lipid of the disclosure is a compound having the following structures (IC) or (ID):

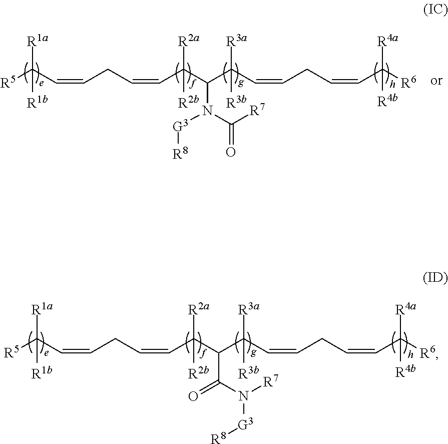

wherein e, f, g and h are each independently an integer from 1 to 12.

In a further embodiment, the cationic lipid is selected from any one of Compound Nos. 1-37 as specifically exemplified in Table 1 of PCT Publication No. WO2019/036000, which Table and compounds listed therein are hereby incorporated herein by reference for all purposes.

In yet another aspect, the cationic lipid is described in PCT Publication No. WO2019/036028, the contents of which is hereby incorporated herein by reference in its entirety for all purposes. In one embodiment therein, an ionizable cationic lipid of the disclosure comprises a compound of structure (I):

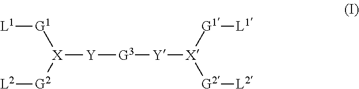

or a pharmaceutically acceptable salt or stereoisomer thereof, wherein:
X and X′ are each independently N or CR;
Y and Y′ are each independently absent, —O(C═O)—, —(C═O)O— or NR, provided that:
a) Y is absent when X is N;
b) Y′ is absent when X′ is N;
c) Y is —O(C═O)—, —(C═O)O— or NR when X is CR; and
d) Y′ is —O(C═O)—, —(C═O)O— or NR when X′ is CR,
L¹ and L^{1′} are each independently —O(C═O)R¹, —(C═O)OR¹, —C(═O)R¹, —OR¹, —S(O)₂R¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² and L^{2′} are each independently —O(C═O)R², —(C═O)OR², —C(═O)R², —OR², —S(O)_{z}R², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G¹, G^{1′}, G² and G^{2′} are each independently C₄-C₈ alkylene or C₂-C₈ alkenylene;
G³ is C₂-C₂₄ heteroalkylene or C₂-C₂₄ heteroalkenylene;
R^{a}, R^{b}, R^{d} and R^{e} are, at each occurrence, independently H, C₁-C₁₂ alkyl or C₂-C₁₂ alkenyl;
R^{e} and R^{f} are, at each occurrence, independently C₁-C₁₂ alkyl or C₂-C₁₂ alkenyl;
R is, at each occurrence, independently H or C₁-C₁₂ alkyl;
R¹ and R² are, at each occurrence, independently branched C₆-C₂₄ alkyl or branched C₆-C₂₄ alkenyl;
z is 0, 1 or 2, and
wherein each alkyl, alkenyl, alkylene, alkenylene, heteroalkylene and heteroalkenylene is independently substituted or unsubstituted unless otherwise specified.

In another embodiment, a cationic lipid of the disclosure is a compound having any one of the following structures (IA), (IB), (IC), (ID), (IE), (IF), (IG) or (IH):

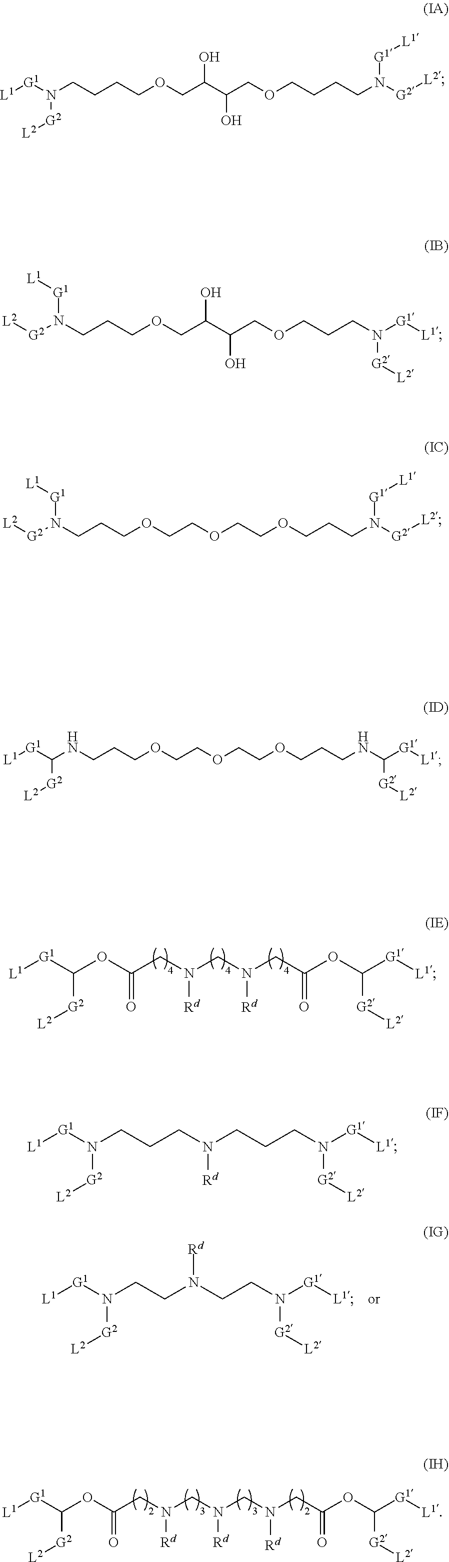

wherein R^{d} is, at each occurrence, independently H or optionally substituted C₁-C₆ alkyl.

In a further embodiment, the cationic lipid is selected from any one of Compound Nos. 1-11 as specifically exemplified in Table 1 of PCT Publication No. WO2019/036028, which Table and compounds listed therein are hereby incorporated herein by reference for all purposes.

In yet another aspect, the cationic lipid is described in PCT Publication No. WO2019/036030, the contents of which is hereby incorporated herein by reference in its entirety for all purposes. In one embodiment therein, an ionizable cationic lipid of the disclosure comprises a compound of structure (I):

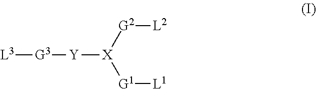

or a pharmaceutically acceptable salt, prodrug or stereoisomer thereof, wherein:
one of L¹ or L² is —O(C═O)—, —(C═O)O—, —C(═O)—, —O—, —S—S—, —C(═O)S—, —SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR S, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In another embodiment, a cationic lipid of the disclosure is a compound having any one of the following structures (IA), (IB), (IC) or (ID):

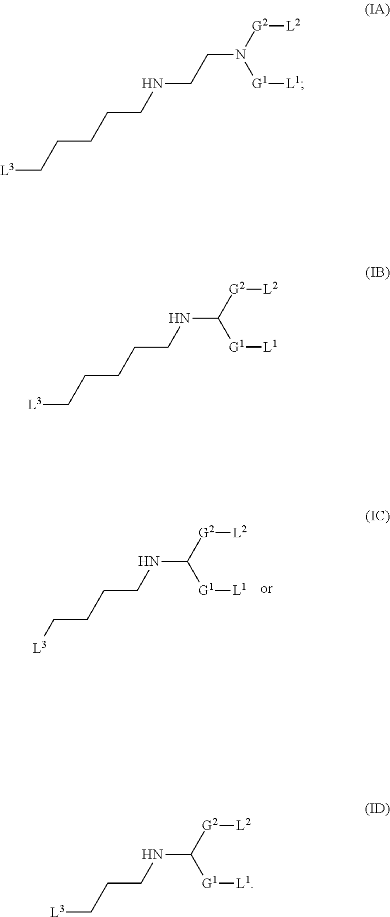

In a further embodiment, the cationic lipid is selected from any one of Compound Nos. 1-12 as specifically exemplified in Table 1 of PCT Publication No. WO2019/036030, which Table and compounds listed therein are hereby incorporated herein by reference for all purposes.

In yet another aspect, the cationic lipid is described in PCT Publication No. WO2019/036008, the contents of which is hereby incorporated herein by reference in its entirety for all purposes. In one embodiment therein, an ionizable cationic lipid of the disclosure comprises a compound of structure (I):

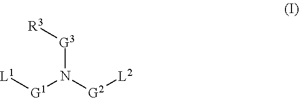

or a pharmaceutically acceptable salt or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{a}C(—O)NR^{e}R^{f}, —OC(═O)NR^{e}R^{f}; —NR^{a}C(═O)OR² or a direct bond to R²;
G¹ and G² are each independently C₂-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H, C₁-C₁₂ alkyl or C₁-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R³ is —N(R⁴)R⁵;
R⁴ is C₁-C₁₂ alkyl;
R⁵ is substituted C₁-C₁₂ alkyl, wherein R⁵ is substituted with one or more substituents selected from the group consisting of —OR^{g}, —NR^{g}C(═O)R^{h}, —C(═O)NR^{g}R^{h}, —C(═O)R^{h}, —OC(═O)R^{h}, —C(═O)OR^{h} and —ORⁱOH, wherein:
R^{g} is, at each occurrence independently H or C₁-C₆ alkyl;
R^{h} is at each occurrence independently C₁-C₆ alkyl; and
Rⁱ is, at each occurrence independently C₁-C₆ alkylene; and
x is 0, 1 or 2, and
wherein each alkyl, alkenyl, alkylene, alkenylene, cycloalkylene and cycloalkenylene is independently substituted or unsubstituted unless otherwise specified.

In another embodiment, a cationic lipid of the disclosure is a compound having the following structures (IA),

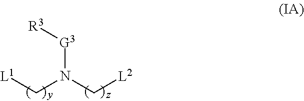

wherein y and z are each independently integers ranging from 2 to 12.

In another embodiment, a cationic lipid of the disclosure is a compound having any one of the following structures (IB), (IC), (ID) or (IE):

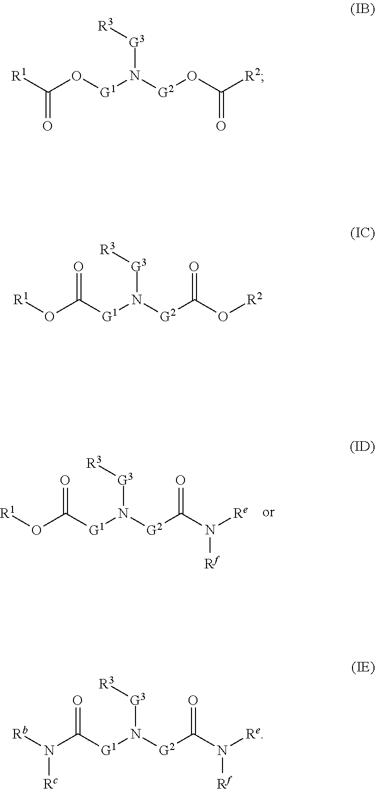

In another embodiment, a cationic lipid of the disclosure is a compound having any one of the following structures (IF), (IG), (IH) or (IJ):

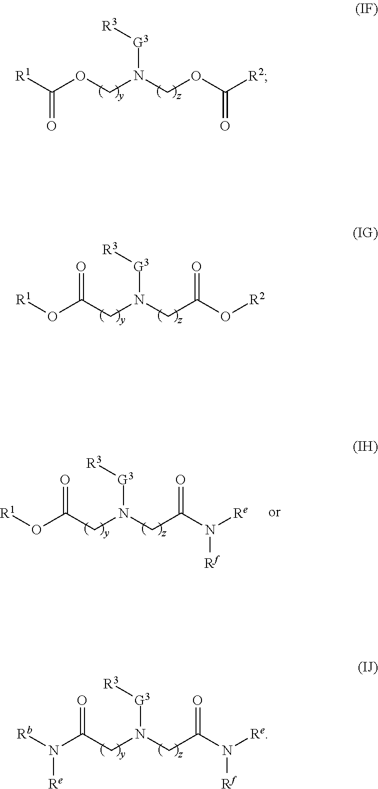

wherein y and z are each independently integers ranging from 2-12.

In a further embodiment, the cationic lipid is selected from any one of Compound Nos. 1-18 as specifically exemplified in Table 1 of PCT Publication No. WO2019/036008, which Table and compounds listed therein are hereby incorporated herein by reference for all purposes.

In yet another aspect, the cationic lipid is described in PCT Publication No. WO2020/081938, the contents of which is hereby incorporated herein by reference in its entirety for all purposes. In one embodiment therein, an ionizable cationic lipid of the disclosure comprises a compound of structure (I):

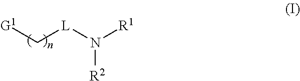

or a pharmaceutically acceptable salt, tautomer, or stereoisomer thereof, wherein:
G¹ is —N(R³)R⁴ or —OR′;
R¹ is optionally substituted branched, saturated or unsaturated C₁₂-C₃₆ alkyl;
R² is optionally substituted branched or unbranched, saturated or unsaturated C₁₂-C₃₆ alkyl when L is —C(═O)—; or R2 is optionally substituted branched or unbranched, saturated or unsaturated C₄-C₃₆ alkyl when L is C₆-C₁₂ alkylene, C₆-C₁₂ alkenylene, or C₂-C₆ alkynylene;
R³ and R⁴ are each independently H, optionally substituted branched or unbranched, saturated or unsaturated C₁-C₆ alkyl; or R³ and R⁴ are each independently optionally substituted branched or unbranched, saturated or unsaturated C₁-C₆ alkyl when L is C₆-C₁₂ alkylene, C₆-C₁₂ alkenylene, or C₂-C₆ alkynylene; or R³ and R⁴, together with the nitrogen to which they are attached, join to form a heterocyclyl;
R⁵ is H or optionally substituted C₁-C₆ alkyl;
L is —C(═O)—, C₆-C₁₂ alkylene, C₆-C₁₂ alkenylene, or C₂-C₆ alkynylene; and
n is an integer from 1 to 12.

In another embodiment, a cationic lipid of the disclosure is a compound having the following structure (IA):

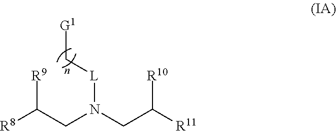

wherein
R⁸ and R⁹ are each independently H or optionally substituted branched or unbranched, saturated or unsaturated C₂-C₁₂ alkyl, provided that R⁸ and R⁹ are each independently selected such that R¹ is optionally substituted branched, saturated or unsaturated C₁₂-C₃₆ alkyl; and
R¹⁰ and R¹¹ are each independently H or optionally substituted branched or unbranched, saturated or unsaturated C₂-C₁₂ alkyl, provided that R¹⁰ and R¹¹ are each independently selected such that: R² is optionally substituted branched or unbranched, saturated or unsaturated C₁₂-C₃₆ alkyl when L is —C(═O)—; and R² is optionally substituted branched or unbranched, saturated or unsaturated C₄-C₃₆ alkyl when L is C₆-C₁₂ alkylene, C₆-C₁₂ alkenylene, or C₂-C₆ alkynylene.

In a further embodiment, the cationic lipid is selected from any one of Compound Nos. I-1 to I-22 as specifically exemplified in Table 1 of PCT Publication No. WO2020/081938, which Table and compounds listed therein are hereby incorporated herein by reference for all purposes.

In yet another aspect, the cationic lipid is described in PCT Publication No. WO2020/146805, the contents of which is hereby incorporated herein by reference in its entirety for all purposes. In one embodiment therein, an ionizable cationic lipid of the disclosure comprises a compound of structure (I):

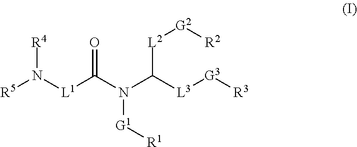

or a pharmaceutically acceptable salt, tautomer, or stereoisomer thereof, wherein:
R¹ is optionally substituted C1-C24 alkyl or optionally substituted C₂-C₂₄ alkenyl;
R² and R³ are each independently optionally substituted C₁-C₃₆ alkyl;
R⁴ and R⁵ are each independently optionally substituted C₁-C₆ alkyl, or R4 and R⁵ join, along with the N to which they are attached, to form a heterocyclyl or heteroaryl;
L¹, L², and L³ are each independently optionally substituted C_{I}-C_{IX} alkylene;
G¹ is a direct bond, —(CH₂)ₙO(C═O)—, —(CH₂)ₙ(C═O)O—, or —(C═O)—;
G² and G³ are each independently —(C═O)O— or —O(C═O)—; and n is an integer greater than 0.

In another embodiment, a cationic lipid of the disclosure is a compound having the following structure (IA):

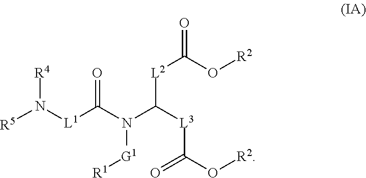

In another embodiment, a cationic lipid of the disclosure is a compound having the following structure (IB):

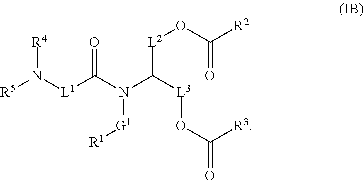

In a further embodiment, the cationic lipid is selected from any one of Compound Nos. I-1 to I-40 as specifically exemplified in Table 1 of PCT Publication No. WO2020/146805, which Table and compounds listed therein are hereby incorporated herein by reference for all purposes.

In yet another aspect, the cationic lipid is described in PCT Publication No. WO2022/016070, the contents of which is hereby incorporated herein by reference in its entirety for all purposes. In one embodiment therein, an ionizable cationic lipid of the disclosure comprises a compound of structure (I):

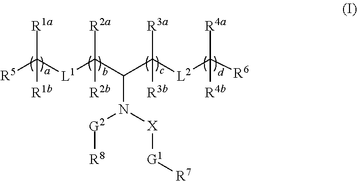

or a pharmaceutically acceptable salt, tautomer or stereoisomer thereof, wherein:
G¹ and G² are each independently C₁-C₆ alkylene;
L¹ and L² are each independently —O(C═O)— or —(C═O)O—;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is —O(C═O)R¹⁰, —(C═O)OR¹⁰, —NR^{a}(C═O)R¹⁰ or —(C═O)NR⁹R¹⁰;
R⁸ is OH, —N(R¹¹)(C═O)R¹², —(CO)NR¹¹R¹², —NR¹¹R¹², —(C═O)OR² or —O(C═O)R′²;
R⁹ is H or C₁-C₁₅ alkyl;
R¹⁰ is C₁-C₁₅ alkyl;
R¹¹ is H or C₁-C₆ alkyl;
R¹² is C₁-C₆ alkyl;
X is —(C═O)— or a direct bond; and
a, b, c and d are each independently an integer from 1 to 24;
wherein each methyl, alkyl and alkylene is independently optionally substituted.

In another embodiment, a cationic lipid of the disclosure is a compound having any one of the following structures (IA) or (IB):

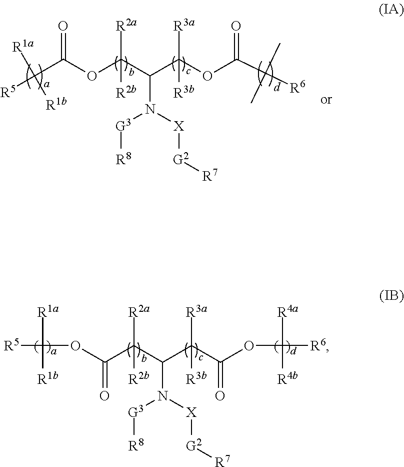

or a pharmaceutically acceptable salt, tautomer or stereoisomer thereof.

In a further embodiment, the cationic lipid is selected from any one of Compound Nos. I-1 to I-23 as specifically exemplified in Table 1 of PCT Publication No. WO2022/016070, which Table and compounds listed therein are hereby incorporated herein by reference for all purposes.

In yet another aspect, the cationic lipid is described in PCT Publication No. WO2023/114944, the contents of which is hereby incorporated herein by reference in its entirety for all purposes. In one embodiment therein, an ionizable cationic lipid of the disclosure comprises a compound of structure (I):

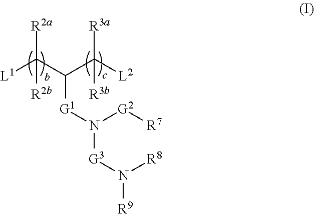

or a pharmaceutically acceptable salt, tautomer, or stereoisomer thereof, wherein: L¹ is —O(C═O)R^{1a}, —(C═O)OR^{1a}, —C(═O)R^{1a}, —OR^{1a}, —S(O)ₓR^{1a}, —S—SR^{1a}, —C(═O)SR^{1a}, —SC(═O)R^{1a}, —NR^{a}C(═O)R^{1a}, —C(═O)NR^{a}R^{1a}, —NR^{a}C(═O)NR^{a}R^{1a}, —OC(═O)NR^{a}R^{1a}, —NR^{a}C(═O)OR^{1a} or R^{1b}; L² is —O(C═O)R^{4a}, —(C═O)OR^{4a}, —C(═O)R^{4a}, —OR^{4a}, —S(O)ₓR^{4a}, —S—SR^{4a}, —C(═O)SR^{4a}, —SC(═O)R^{4a}, —NR^{a}C(═O)R₄ₐ, —C(═O)NR^{a}R^{4a}, —NR^{a}C(═O)NR^{a}R^{4a}, —OC(═O)NR^{a}R^{4a}, —NR^{a}C(═O)OR^{4a}, or R^{4b}; G¹ is C₁-C₂ alkylene, —(C═O)—, —O(C═O)—, —SC(═O)—, —NR^{a}C(═O)— or a direct bond; G² is —C(═O)—, —(C═O)O—, —C(═O)S—, —C(═O)NR^{a}— or a direct bond; G³ is C₁-C₆ alkylene; R^{a} is H or C₁-C₁₂ alkyl; R^{1a} and R^{4a} are each independently branched C₆-C₂₄ alkyl, branched C₆-C₂₄ alkenyl, branched C₆-C₂₄ fluoroalkyl, branched C₆-C₂₄ fluoroalkenyl, C₆-C₂₄ alkylacetal or C₆-C₂₄ fluoroalkylacetal; R^{1b} and R^{4b} are each independently —CH(OR)(OR), wherein each R is independently linear or branched C₆-C₁₈ alkyl, linear or branched C₆-C₁₈ alkenyl, linear or branched C₆-C₁₈ fluoroalkyl, or linear or branched C₆-C₁₈ fluoroalkenyl R^{2a} and R^{2b} are, at each occurrence, independently H, F, C₁-C₁₂ alkyl, or C₁-C₁₂ fluoroalkyl; R^{3a} and R^{3b} are, at each occurrence, independently H, F, C₁-C₁₂ alkyl, or C₁-C₁₂ fluoroalkyl; R7 is H, C₄-C₂₀ alkyl, or C₂-C₁₀ fluoroalkyl; R⁸ and R⁹ are each independently C₁-C₁₂ alkyl; or R⁸ and R⁹, together with the nitrogen atom to which they are attached, form a 5, 6 or 7-membered heterocyclic ring; b and c are each independently an integer from 1 to 24; and wherein at least one of R^{2a}, R^{2b}, R^{3a}, and R^{3b} is F or C₁-C₁₂ fluoroalkyl; at least one of R^{1a} and R^{4a} is present and selected from branched C₆-C₂₄ fluoroalkyl, branched C₆-C₂₄ fluoroalkenyl and C₆-C₂₄ fluoroalkylacetal; at least one of R^{1b} and R^{4b} is present and selected from linear or branched C₆-C₁₈ fluoroalkyl and linear or branched C₆-C₁₈ fluoroalkenyl; G³ is C₁-C₆ fluoroalkylene; and/or R⁷ is C₂-C₁₀ fluoroalkyl.

In another embodiment, a cationic lipid of the disclosure is a compound having any one of the following structures (IA) or (IB):

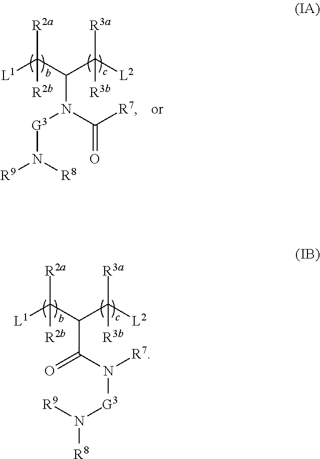

In a further embodiment, the cationic lipid is selected from any one of Compound Nos. I-1 to I-25 as specifically exemplified in Table 1 of PCT Publication No. WO2023/114944, which Table and compounds listed therein are hereby incorporated herein by reference for all purposes.

In yet another aspect, the cationic lipid is described in PCT Publication No. WO2023/114937, the contents of which is hereby incorporated herein by reference in its entirety for all purposes. In one embodiment therein, an ionizable cationic lipid of the disclosure comprises a compound of structure (I):

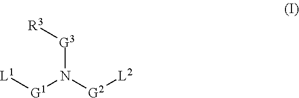

or a pharmaceutically acceptable salt or stereoisomer thereof, wherein:
L¹ is —O(C═O)R^{Ia}, —(C═O)OR^{Ia}, —C(═O)R^{Ia}, —OR^{Ia}, —S(O)ₓR^{Ia}, —S—SR^{Ia}, —C(═O)SR^{Ia}, —SC(═O)R^{Ia}, —NR^{a}C(═O)R^{Ia}, —C(═O)NR^{a}R^{Ia}, —NR^{a}C(═O)NR^{a}R^{Ia}, —OC(═O)NR^{a}R^{Ia}, —NR^{a}C(═O)OR^{Ia} or R^{Ib};
L² is —O(C═O)R^{2a}, —(C═O)OR^{2a}, —C(═O)R^{2a}, —OR^{2a}, —S(O)ₓR^{2a}, —S—SR^{2a}, —C(═O)SR^{2a}, —SC(═O)R^{2a}, —NR^{a}C(═O)R^{2a}, —C(═O)NR^{a}R^{2a}, —NR^{a}C(═O)NR^{a}R^{2a}, —OC(═O)NR^{a}R^{2a}, —NR^{a}C(═O)OR^{2a}, or R^{2b};
G¹ and G² are each independently linear or branched C₁-C₁₂ alkylene or linear or branched C₁-C₁₂ fluoroalkylene;
G³ is linear or branched C₁-C₁₂ alkylene or linear or branched C₁-C₁₂ fluoroalkylene; each R^{a} is independently H or C₁-C₁₂ alkyl;
R^{Ia} and R^{2a} are each independently branched C₆-C₂₄ alkyl, branched C₆-C₂₄ alkenyl, branched C₆-C₂₄ fluoroalkyl, branched C₆-C₂₄ fluoroalkenyl, C₆-C₂₄ alkylacetal or C₆-C₂₄ fluoroalkylacetal;
R^{1b} and R^{2b} are each independently —CH(OR)(OR), wherein each R is independently linear or branched Ce-Cis alkyl, linear or branched Ce-Cis alkenyl, linear or branched Ce-Cis fluoroalkyl, or linear or branched Ce-Cis fluoroalkenyl;
R³ is H, —OR⁵, —CN, —C(═O)OR⁴, —OC(═O)R⁴, —N(R⁵)N⁴, —C(═O)N(R⁴)R⁵, or —NR⁵C(═O)R⁴; and
R⁴ is H, C₁-C₁₂ alkyl, or aryl, and R⁵ is H or Ci-Ce alkyl; or R⁴ and R⁵, together with the nitrogen atom to which they are attached, form a 5, 6 or 7-membered heterocyclic ring, and 66 wherein at least one of G¹ and G² is linear or branched C₁-C₁₂ fluoroalkylene;
G³ is linear or branched C1-C12 fluoroalkylene; at least one of R^{Ia} and R^{2a} is present and selected from branched C6-C24 fluoroalkyl, branched C6-C24 fluoroalkenyl and C6-C24 fluoroalkylacetal; and/or at least one of R^{Ib} and R^{2b} is present and selected from linear or branched Ce-Cis fluoroalkyl and linear or branched Ce-Cis fluoroalkenyl.

In another embodiment, a cationic lipid of the disclosure is a compound having the following structure (IA):

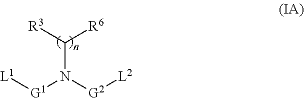

wherein:
R⁶ is, at each occurrence, independently H, F, OH or C1-C24 alkyl; and n is an integer ranging from 1 to 15.

In another embodiment, a cationic lipid of the disclosure is a compound having the following structure (IB):

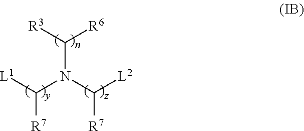

wherein: y and z are each independently integers ranging from 1 to 12; and R⁷ is, at each occurrence, independently H or F.

In another embodiment, a cationic lipid of the disclosure is a compound having any one of the following structures (IC) or (ID):

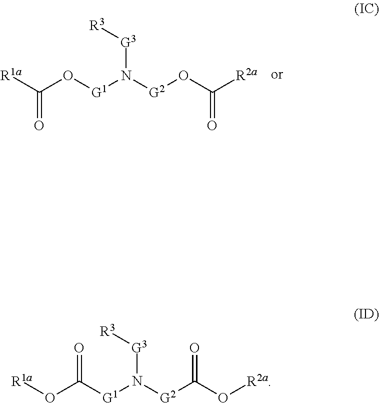

In another embodiment, a cationic lipid of the disclosure is a compound having any one of the following structures (IE) or (IF):

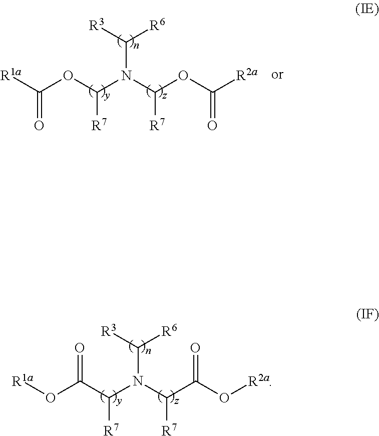

In another embodiment, a cationic lipid of the disclosure is a compound having any one of the following structures (IG) or (IH):

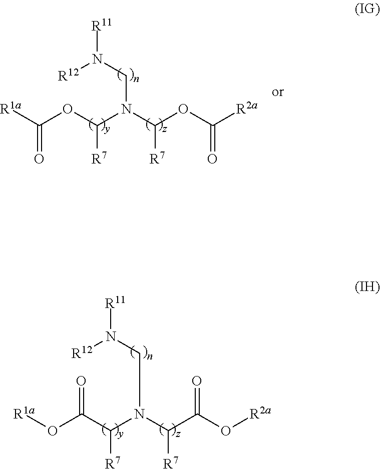

wherein R¹¹ and R¹² are each independently C₁-C₁₂ alkyl; or R11 and R12, together with the nitrogen atom to which they are attached, form a 5, 6 or 7-membered heterocyclic ring comprising one nitrogen atom.

In a further embodiment, the cationic lipid is selected from any one of Compound Nos. I-1 to I-10 as specifically exemplified in Table 1 of PCT Publication No. WO2023/114937, which Table and compounds listed therein are hereby incorporated herein by reference for all purposes.

In yet another aspect, the cationic lipid is described in PCT Publication No. WO2023/114943, the contents of which is hereby incorporated herein by reference in its entirety for all purposes. In one embodiment therein, an ionizable cationic lipid of the disclosure comprises a compound of structure (I):

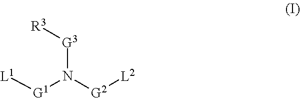

or a pharmaceutically acceptable salt, prodrug or stereoisomer thereof, wherein: L¹ is —NRaC(═O)R1 or —C(═O)NRbRc; L2 is —NRdC(═O)R2 or —C(═O)NReRf; G1 and G2 are each independently C2-C12 alkylene, or C2-C12 alkenylene; G3 is C1-C24 alkylene, or C2-C24 alkenylene; Ra, Rb, Rd and Re are each independently H, C1-C16 alkyl or C2-C16 alkenyl; Rc and Rf are each independently C1-C16 alkyl or C2-C16 alkenyl; R1 and R2 are each independently C6-C24 alkyl or C6-C24 alkenyl; R3 is H, —OH, CN, —N(R4)R5; —C(═O)N(R4) R5; —N(R4)C(═O)R5; —N(R4)C(═O)OR5; —C(═O)OR6, —OC(═O)R6, —OR7, heteroaryl or aryl; R4 and R5 are each independently is H, C1-C12 alkyl, C3-C6 cycloalkyl or C3-C6 cycloalkenyl, or R4 and R5, together with the nitrogen or carbon atom to which they are bound, form a 5 to 7-membered heterocyclic ring; R6 is H, C₁-C₁₂ alkyl, C₂-C₁₂ alkenyl or aralkyl; R7 is C₁-C₁₂ alkyl optionally substituted with hydroxyl or alkoxy; and wherein each alkyl, alkenyl, alkylene, alkenylene, aryl and aralkyl is independently substituted or unsubstituted unless otherwise specified.

In another embodiment, a cationic lipid of the disclosure is a compound having the following structure (IA):

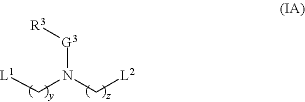

wherein y and z are each independently integers ranging from 2 to 12.

In another embodiment, a cationic lipid of the disclosure is a compound having any one of the following structures (IB) or (IC):

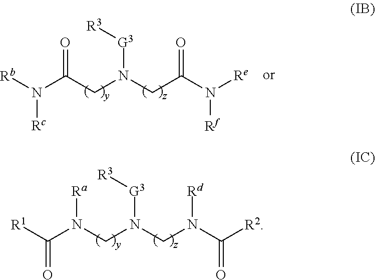

In a further embodiment, the cationic lipid is selected from any one of Compound Nos. I-1 to I-85 as specifically exemplified in Table 1 of PCT Publication No. WO2023/114943, which Table and compounds listed therein are hereby incorporated herein by reference for all purposes.

In yet another aspect, the ionizable cationic lipid is described in PCT Publication No. WO2024/054843, the contents of which is hereby incorporated herein by reference in its entirety for all purposes. In one embodiment therein, an ionizable cationic lipid of the disclosure comprises a compound of Structure (I):

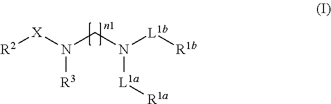

or a pharmaceutically acceptable salt, tautomer, or stereoisomer thereof, wherein:
   L^{1a} and L^{1b} are each independently optionally substituted C₃-C₁₂ alkyl;
   R^{1a} is —C(═O)OR^{4a} or —O(C═O)R^{4a};
   R^{1b} is —C(═O)OR^{4b} or —O(C═O)R^{4b};
   R² is —NR⁶(C═O)R⁵, —(C═O)N(R⁶)R⁵ or —(C═O)OR⁷;
   R³ and R⁶ are each independently hydrogen or optionally substituted C₁-C₆ alkyl;
   R^{4a}, R^{4b}, and R⁵ are each independently optionally substituted alkyl;
   R⁷ is optionally substituted arylalkyl;
   n1 is 2, 3, 4, 5, or 6; and
   X is C₂-C₆ alkylene or C₄-C₂₀ alkyleneoxide.

One embodiment provides a compound having Structure (I):

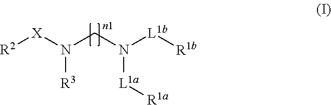

or a pharmaceutically acceptable salt, tautomer, or stereoisomer thereof, wherein:
L^{1a} and L^{1b} are each independently optionally substituted C₃-C₁₂ alkyl;
R^{1a} is —C(═O)OR^{4a} or —O(C═O)R^{4a},
R^{1b} is —C(═O)OR^{4b} or —O(C═O)R^{4b};
R² is —NR⁶(C═O)R⁵, —(C═O)N(R⁶)R⁵ or —(C═O)OR⁷;
R³ and R⁶ are each independently hydrogen or optionally substituted C₁-C₁₂ alkyl;
R^{4a}, R^{4b}, and R⁵ are each independently optionally substituted alkyl;
R⁷ is optionally substituted C₁-C₆ alkyl or optionally substituted arylalkyl;
n1 is 2, 3, 4, 5, or 6; and
X is C₂-C₆ alkylene or C₄-C₂₀ alkyleneoxide.

In some embodiments, wherein X is:

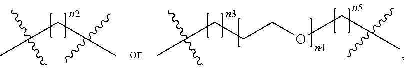

wherein:
n2 is 2, 3, 4, 5, or 6;
n3 is 0, 1, 2, 3, or 4;
n4 is 2, 3, or 4; and
n5 is 2, 3, 4, or 5.

In some embodiments, L^{1a} is C₅-C₉ alkyl. In certain embodiments, L^{1b} is C₅-C₉ alkyl. In some embodiments, L^{1a} is C₅-, C₆-, C₇-, or C₈-alkyl. In certain embodiments, L^{1b} is C₅-, C₆-, C₇-, or C₈-alkyl. In some embodiments, L^{1a} is C₅-alkyl. In certain embodiments, L^{1a} is C₆-alkyl. In some embodiments, L^{1a} is C₇-alkyl. In certain embodiments, L^{1a} is C₈-alkyl. In some embodiments, L^{1b} is C₅-alkyl. In certain embodiments, L^{1b} is C₆-alkyl. In some embodiments, L^{1b} is C₇-alkyl. In certain embodiments, L^{1b} is C₈-alkyl. In some embodiments, L^{1a} is unsubstituted. In certain embodiments, L^{1b} is unsubstituted. In some embodiments, L^{1a} is unbranched. In certain embodiments, L^{1b} is unbranched.

In some embodiments, one of R^{1a} is —O(C═O)R^{4a}. In certain embodiments, one of R^{1a} is —(C═O)OR^{4a}. In some embodiments, R^{1b} is —O(C═O)R^{4b}. In certain embodiments, one of R¹⁵ is —(C═O)OR^{4b}.

In some embodiments, R^{4a} is C₈-C₂₄-alkyl. In certain embodiments, R^{4a} is C₁₀-C₁₈-alkyl. In certain embodiments, R^{4a} is C₁₁-C₁₆-alkyl. In some embodiments, R^{4a} is C₁₁-alkyl. In certain embodiments, R^{4a} is C₁₅-alkyl. In some embodiments, R^{4a} is C₁₆-alkyl. In certain embodiments, R^{4b} is C₈-C₂₄-alkyl. In some embodiments, R^{4b} is C₁₀-C₁₈-alkyl. In certain embodiments, R^{4b} is C₁₁-C₁₆-alkyl. In some embodiments, R^{4b} is C₁₁-alkyl. In certain embodiments, R^{4b} is C₁₅-alkyl. In some embodiments, R^{4b} is C₁₆-alkyl.

In certain embodiments, R^{4a} is branched. In some embodiments, R^{4b} is branched. In certain embodiments, R^{4a} is unsubstituted. In some embodiments, R^{4b} is unsubstituted. In certain embodiments, R^{4a} has one of the following structures:

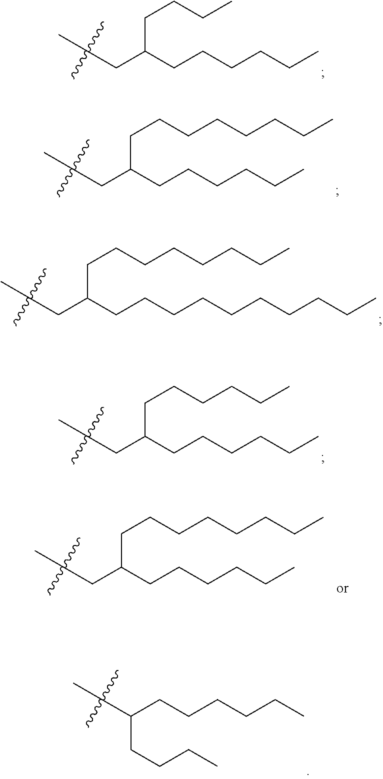 [SMILES: *CC(CCCC)CCCCCC ; *CC(CCCCCC)CCCCCCCC ; *CC(CCCCCCCC)CCCCCCCCCC ; *CC(CCCCCC)CCCCCC ; *CC(CCCCCC)CCCCCCCC ; *C(CCCC)CCCCCC]

In some embodiments, R^{4b} has one of the following structures:

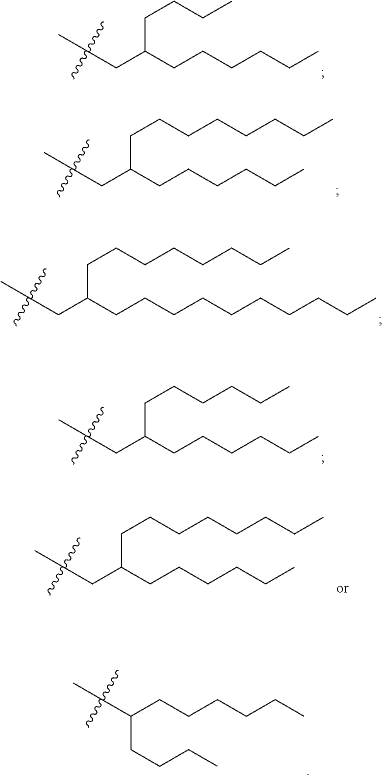 [SMILES: *CC(CCCC)CCCCCC ; *CC(CCCCCC)CCCCCCCC ; *CC(CCCCCCCC)CCCCCCCCCC ; *CC(CCCCCC)CCCCCC ; *CC(CCCCCC)CCCCCCCC ; *C(CCCC)CCCCCC]

In some embodiments, R² is —NR⁶(C═O)R⁵. In certain embodiments, R² is —(C═O)N(R⁶)R⁵. In some embodiments, R⁵ is C₂-C₁₆-alkyl. In certain embodiments, R⁵ is C₄-C₁₃-alkyl. In some embodiments, R⁵ is C₄-, C₇-, C₈-, C₁₀-, or C₁₃-alkyl. In certain embodiments, R⁵ is unsubstituted. In some embodiments, R⁵ is substituted with hydroxyl. In some embodiments, R⁵ is branched. In certain embodiments, R⁵ is unbranched. In some embodiments, R⁵ has one of the following structures:

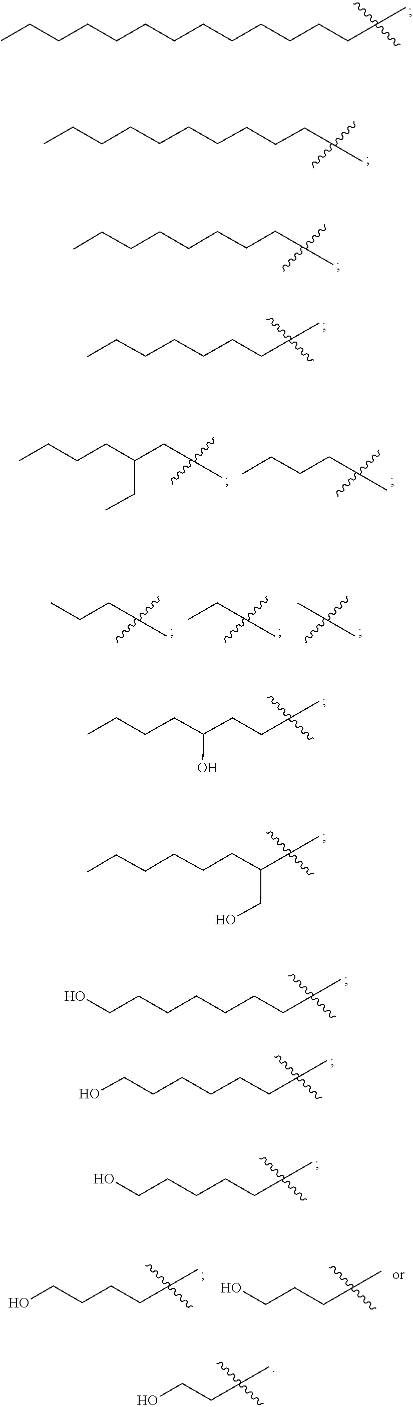

In certain embodiments, R⁵ is unbranched. In some embodiments, R⁵ has one of the following structures:

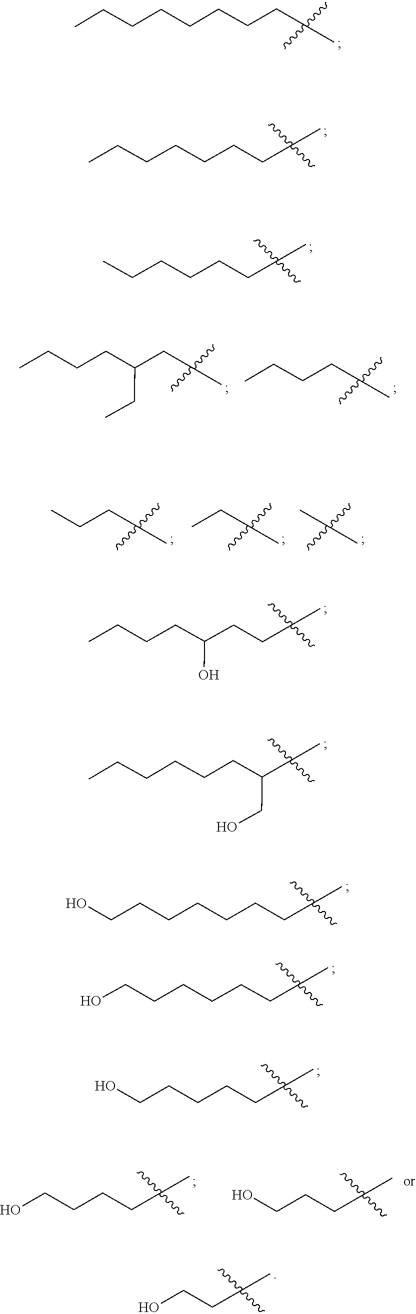

In some embodiments, R⁶ is C₁-C₆ alkyl. In some embodiments, R⁶ is C₁-C₁₀ alkyl. In certain embodiments, R⁶ is C₁-C₄-alkyl. In some embodiments, R⁶ is C₁-, C₂-, C₃-, C₆-, C₈-, or C₁₀-alkyl. In certain embodiments, methyl, ethyl, n-butyl, n-hexyl, n-octyl, or n-decyl. In some embodiments, R⁶ is unbranched. In certain embodiments, R⁶ is methyl or n-butyl. In some embodiments, R⁶ is unsubstituted. In some embodiments, R⁶ is substituted. In some embodiments, R⁶ is C₁-C₆ alkyl substituted with one or more hydroxyl. In some embodiments, R⁶ is C₂-, C₃-, C₄-, or C₆-alkyl substituted with one or more hydroxyl. In certain embodiments, R⁶ is hydrogen. In some embodiments, R² is —(C═O)OR⁷.

In some embodiments, R⁷ is C₁-C3 alkyl or C7-C₁₆ arylalkyl. In certain embodiments, R⁷ is C₇-C₁₆ arylalkyl. In some embodiments, R⁷ is C₁-C₃ alkyl. In some embodiments, R⁷ is unsubstituted.

In certain embodiments, R⁷ is —CH₃ or has the following structure:

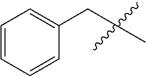 [SMILES: *Cc1ccccc1]

In certain embodiments, R⁷ has the following structure:

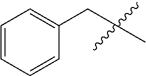 [SMILES: *Cc1ccccc1]

In some embodiments, R³ is optionally substituted C₁-C₆ alkyl. In certain embodiments, R³ is optionally substituted methyl, ethyl, n-propyl, iso-propyl, n-butyl, iso-butyl, tert-butyl, n-hexyl. In some embodiments, R³ is optionally substituted methyl. In some embodiments, R³ is C₁-C₆ alkyl substituted with one or more hydroxyl. In some embodiments, R³ is C₂- or C₄-alkyl substituted with one or more hydroxyl. In certain embodiments, R³ is unsubstituted. In some embodiments, R³ is hydrogen.

In some embodiments, X is

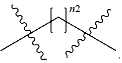

For example, in certain aspects the compound has the following Structure (II):

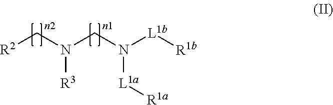

or a pharmaceutically acceptable salt, tautomer, or stereoisomer thereof. For example, in some of these embodiments n2 is 3, 4, or 5.

In other embodiments X is

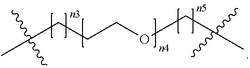

In some such embodiments, the compound has the following Structure (III):

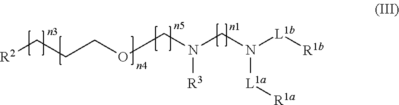

or a pharmaceutically acceptable salt, tautomer, or stereoisomer thereof. In different of these embodiments, n3 is 0 or 1. In other embodiments, n4 is 2 or 3. In some other different embodiments, n5 is 3.

In some embodiments, n1 is 3, 4, or 5. In certain embodiments, n1 is 2.

In some embodiments, the compound has one of the structures set forth in the Table below or a pharmaceutically acceptable salt, tautomer, or stereoisomer thereof:

In yet another aspect, the cationic lipid is described in PCT Publication No. WO2019/036028, the contents of which is hereby incorporated herein by reference in its entirety for all purposes. In one embodiment therein, an ionizable cationic lipid of the disclosure comprises a compound of structure (I):

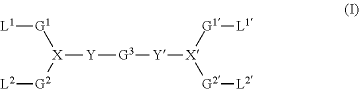

or a pharmaceutically acceptable salt or stereoisomer thereof, wherein:
X and X′ are each independently N or CR;
Y and Y′ are each independently absent, —O(C═O)—, —(C═O)O— or NR, provided that:
a) Y is absent when X is N;
b) Y′ is absent when X′ is N;
c) Y is —O(C═O)—, —(C═O)O— or NR when X is CR; and
d) Y′ is —O(C═O)—, —(C═O)O— or NR when X′ is CR,
L¹ and L^{1′} are each independently —O(C═O)R¹, —(C═O)OR¹, —C(═O)R¹, —OR¹, —S(O)_{z}R¹, —S—SR¹, —C(═O)SR¹, —SC(═O)R¹, —NR^{a}C(═O)R¹, —C(—O)NR^{b}R^{c}, —NR^{a}C(═O)NR^{b}R^{c}, —OC(═O)NR^{b}R^{c} or —NR^{a}C(═O)OR¹;
L² and L^{2′} are each independently —O(C═O)R², —(C═O)OR², —C(═O)R², —OR², —S(O)_{z}R², —S—SR², —C(═O)SR², —SC(═O)R², —NR^{d}C(—O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G¹, G^{1′}, G² and G^{2′} are each independently C₄-C₈ alkylene or C₂-C₈ alkenylene;
G³ is C₂-C₂₄ heteroalkylene or C₂-C₂₄ heteroalkenylene;
R^{a}, R^{b}, R^{d} and R^{e} are, at each occurrence, independently H, C₁-C₁₂ alkyl or C₂-C₁₂ alkenyl;
R^{e} and R^{f} are, at each occurrence, independently C₁-C₁₂ alkyl or C₂-C₁₂ alkenyl;
R is, at each occurrence, independently H or C₁-C₁₂ alkyl;
R¹ and R² are, at each occurrence, independently branched C₆-C₂₄ alkyl or branched C₆-C₂₄ alkenyl;
z is 0, 1 or 2, and
wherein each alkyl, alkenyl, alkylene, alkenylene, heteroalkylene and heteroalkenylene is independently substituted or unsubstituted unless otherwise specified.

In another embodiment, a cationic lipid of the disclosure is a compound having any one of the following structures (IA), (IB), (IC), (ID), (IE), (IF), (IG) or (IH):

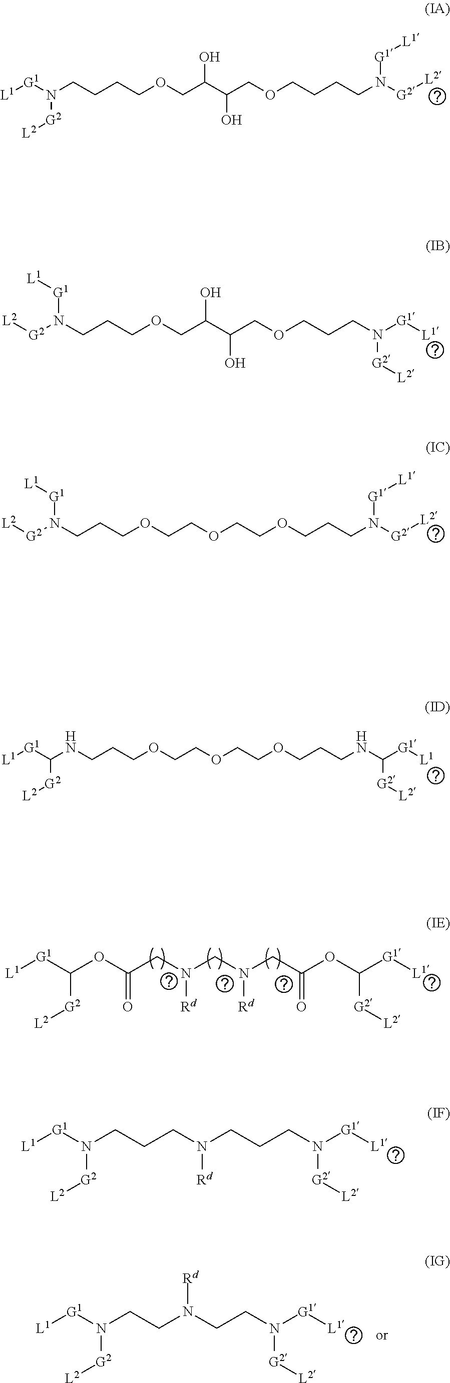

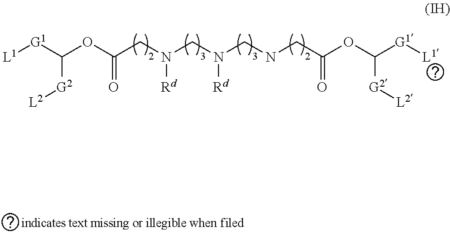

wherein R^{d} is, at each occurrence, independently H or optionally substituted C₁-C₆ alkyl.

In a further embodiment, the cationic lipid is selected from any one of Compound Nos. 1-11 as specifically exemplified in Table 1 of PCT Publication No. WO2019/036028, which Table and compounds listed therein are hereby incorporated herein by reference for all purposes.

In yet another aspect, the cationic lipid is described in PCT Publication No. WO2019/036030, the contents of which is hereby incorporated herein by reference in its entirety for all purposes. In one embodiment therein, an ionizable cationic lipid of the disclosure comprises a compound of structure (I):

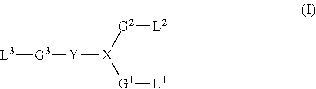

or a pharmaceutically acceptable salt, prodrug or stereoisomer thereof, wherein:
one of L¹ or L² is —O(C═O)—, —(C═O)O—, —C(═O)—, —O—, —S—S—, —C(═O)S—, —SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR S, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In another embodiment, a cationic lipid of the disclosure is a compound having any one of the following structures (IA), (IB), (IC) or (ID):

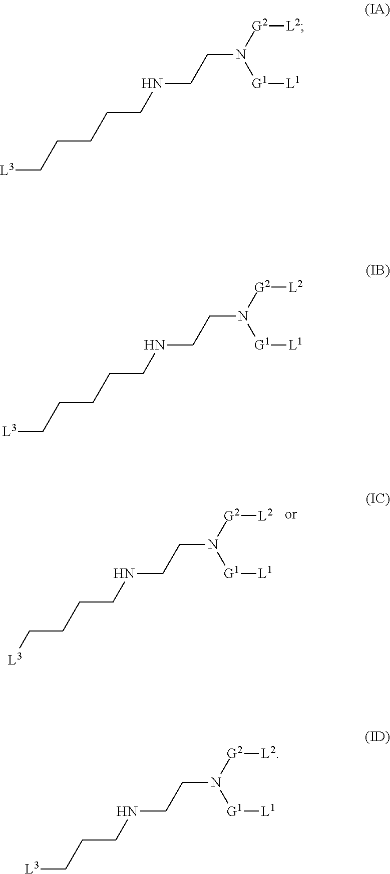

In a further embodiment, the cationic lipid is selected from any one of Compound Nos. 1-12 as specifically exemplified in Table 1 of PCT Publication No. WO2019/036030, which Table and compounds listed therein are hereby incorporated herein by reference for all purposes.

In yet another aspect, the cationic lipid is described in PCT Publication No. WO2019/036008, the contents of which is hereby incorporated herein by reference in its entirety for all purposes. In one embodiment therein, an ionizable cationic lipid of the disclosure comprises a compound of structure (I):

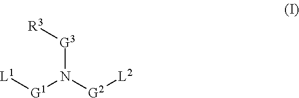

or a pharmaceutically acceptable salt or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{a}C(—O)R², —C(═O)NR^{e}R^{f}, —NR^{a}C(—O)NR^{e}R^{f}, —OC(═O)NR^{e}R^{f}; —NR^{a}C(═O)OR² or a direct bond to R²;
G¹ and G² are each independently C₂-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H, C₁-C₁₂ alkyl or C₁-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R³ is —N(R⁴)R⁵;
R⁴ is C₁-C₁₂ alkyl;
R⁵ is substituted C₁-C₁₂ alkyl, wherein R⁵ is substituted with one or more substituents selected from the group consisting of —OR^{g}, —NR^{g}C(—O)R^{h}, —C(═O)NR^{g}R^{h}, —C(═O)R^{h}, —OC(═O)R^{h}, —C(═O)OR^{h} and —ORⁱOH, wherein:
R^{g} is, at each occurrence independently H or C₁-C₆ alkyl;
R^{h} is at each occurrence independently C₁-C₆ alkyl; and
Rⁱ is, at each occurrence independently C₁-C₆ alkylene; and
x is 0, 1 or 2, and
wherein each alkyl, alkenyl, alkylene, alkenylene, cycloalkylene and cycloalkenylene is independently substituted or unsubstituted unless otherwise specified.

In another embodiment, a cationic lipid of the disclosure is a compound having the following structures (IA),

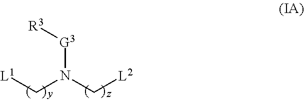

wherein y and z are each independently integers ranging from 2 to 12.

In another embodiment, a cationic lipid of the disclosure is a compound having any one of the following structures (IB), (IC), (ID) or (IE):

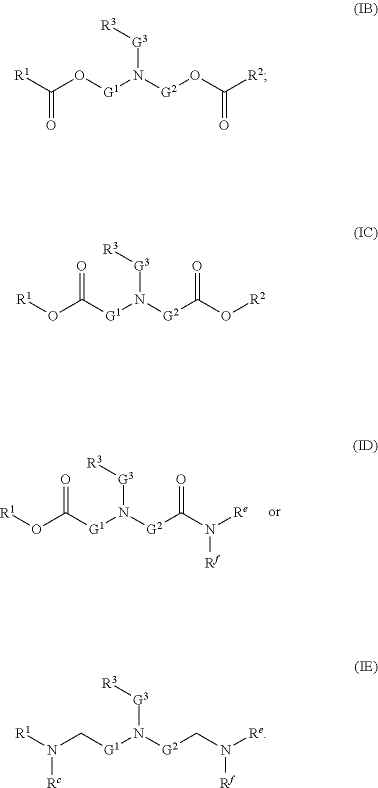

In another embodiment, a cationic lipid of the disclosure is a compound having any one of the following structures (IF), (IG), (IH) or (IJ):

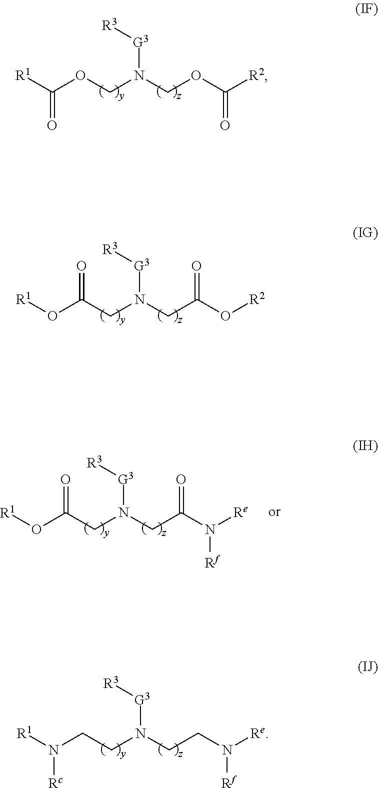

wherein y and z are each independently integers ranging from 2-12.

In a further embodiment, the cationic lipid is selected from any one of Compound Nos. 1-18 as specifically exemplified in Table 1 of PCT Publication No. WO2019/036008, which Table and compounds listed therein are hereby incorporated herein by reference for all purposes.

In yet another aspect, the cationic lipid is described in PCT Publication No. WO2020/081938, the contents of which is hereby incorporated herein by reference in its entirety for all purposes. In one embodiment therein, an ionizable cationic lipid of the disclosure comprises a compound of structure (I):

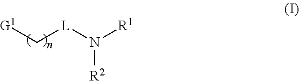

or a pharmaceutically acceptable salt, tautomer, or stereoisomer thereof, wherein:
G¹ is —N(R³)R⁴ or —OR′;
R¹ is optionally substituted branched, saturated or unsaturated C₁₂-C₃₆ alkyl;
R² is optionally substituted branched or unbranched, saturated or unsaturated C₁₂-C₃₆ alkyl when L is —C(═O)—; or R² is optionally substituted branched or unbranched, saturated or unsaturated C₄-C₃₆ alkyl when L is C₆-C₁₂ alkylene, C₆-C₁₂ alkenylene, or C₂-C₆ alkynylene;
R³ and R⁴ are each independently H, optionally substituted branched or unbranched, saturated or unsaturated C₁-C₆ alkyl; or R³ and R⁴ are each independently optionally substituted branched or unbranched, saturated or unsaturated C₁-C₆ alkyl when L is C₆-C₁₂ alkylene, C₆-C₁₂ alkenylene, or C₂-C₆ alkynylene; or R³ and R⁴, together with the nitrogen to which they are attached, join to form a heterocyclyl;
R⁵ is H or optionally substituted C₁-C₆ alkyl;
L is —C(═O)—, C₆-C₁₂ alkylene, C₆-C₁₂ alkenylene, or C₂-C₆ alkynylene; and
n is an integer from 1 to 12.

In another embodiment, a cationic lipid of the disclosure is a compound having the following structure (IA):

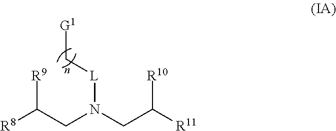

wherein
R⁸ and R⁹ are each independently H or optionally substituted branched or unbranched, saturated or unsaturated C₂-C₁₂ alkyl, provided that R⁸ and R⁹ are each independently selected such that R¹ is optionally substituted branched, saturated or unsaturated C₁₂-C₃₆ alkyl; and
R¹⁰ and R¹¹ are each independently H or optionally substituted branched or unbranched, saturated or unsaturated C₂-C₁₂ alkyl, provided that R¹⁰ and R¹¹ are each independently selected such that: R² is optionally substituted branched or unbranched, saturated or unsaturated C₁₂-C₃₆ alkyl when L is —C(═O)—; and R² is optionally substituted branched or unbranched, saturated or unsaturated C₄-C₃₆ alkyl when L is C₆-C₁₂ alkylene, C₆-C₁₂ alkenylene, or C₂-C₆ alkynylene.

In a further embodiment, the cationic lipid is selected from any one of Compound Nos. I-1 to I-22 as specifically exemplified in Table 1 of PCT Publication No. WO2020/081938, which Table and compounds listed therein are hereby incorporated herein by reference for all purposes.

In yet another aspect, the cationic lipid is described in PCT Publication No. WO2020/146805, the contents of which is hereby incorporated herein by reference in its entirety for all purposes. In one embodiment therein, an ionizable cationic lipid of the disclosure comprises a compound of structure (I):

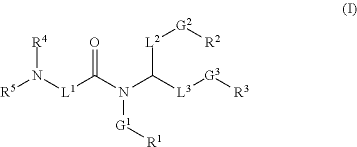

or a pharmaceutically acceptable salt, tautomer, or stereoisomer thereof, wherein:
R¹ is optionally substituted C₁-C₂₄ alkyl or optionally substituted C₂-C₂₄ alkenyl;
R² and R³ are each independently optionally substituted C₁-C₃₆ alkyl;
R⁴ and R⁵ are each independently optionally substituted C₁-C₆ alkyl, or R⁴ and R⁵ join, along with the N to which they are attached, to form a heterocyclyl or heteroaryl;
L¹, L², and L³ are each independently optionally substituted C_{I}-C_{IX} alkylene;
G¹ is a direct bond, —(CH₂)ₙO(C═O)—, —(CH₂)ₙ(C═O)O—, or —(C═O)—;
G² and G³ are each independently —(C═O)O— or —O(C═O)—; and n is an integer greater than 0.

In another embodiment, a cationic lipid of the disclosure is a compound having the following structure (IA):

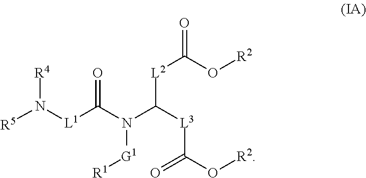

In another embodiment, a cationic lipid of the disclosure is a compound having the following structure (IB):

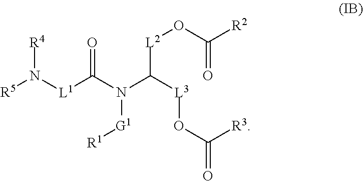

In a further embodiment, the cationic lipid is selected from any one of Compound Nos. I-1 to I-40 as specifically exemplified in Table 1 of PCT Publication No. WO2020/146805, which Table and compounds listed therein are hereby incorporated herein by reference for all purposes.

In yet another aspect, the cationic lipid is described in PCT Publication No. WO2022/016070, the contents of which is hereby incorporated herein by reference in its entirety for all purposes. In one embodiment therein, an ionizable cationic lipid of the disclosure comprises a compound of structure (I):

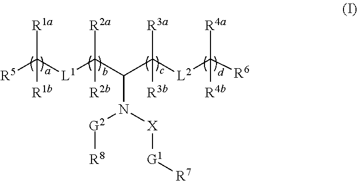

or a pharmaceutically acceptable salt, tautomer or stereoisomer thereof, wherein:
G¹ and G² are each independently C₁-C₆ alkylene;
L¹ and L² are each independently —O(C═O)— or —(C═O)O—;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is —O(C═O)R¹⁰, —(C═O)OR¹⁰, —NR⁹(C═O)R¹⁰ or —(C═O)NR⁹R¹⁰;
R⁸ is OH, —N(R¹¹)(C═O)R¹², —(C═O)NR¹¹R¹², —NR¹¹R¹², —(C═O)OR² or —O(C═O)R²;
R⁹ is H or C₁-C₁₅ alkyl;
R¹⁰ is C₁-C₁₅ alkyl;
R¹¹ is H or C₁-C₆ alkyl;
R¹² is C₁-C₆ alkyl;
X is —(C═O)— or a direct bond; and
a, b, c and d are each independently an integer from 1 to 24;
wherein each methyl, alkyl and alkylene is independently optionally substituted.

In another embodiment, a cationic lipid of the disclosure is a compound having any one of the following structures (IA) or (IB):

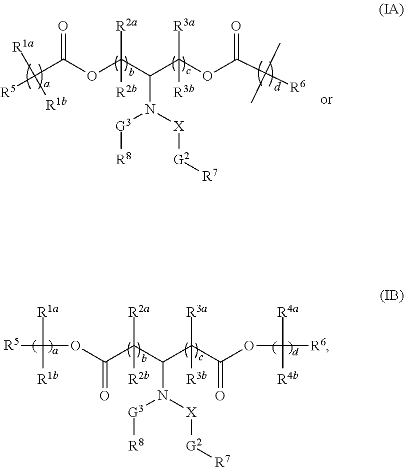

or a pharmaceutically acceptable salt, tautomer or stereoisomer thereof.

In a further embodiment, the cationic lipid is selected from any one of Compound Nos. I-1 to I-23 as specifically exemplified in Table 1 of PCT Publication No. WO2022/016070, which Table and compounds listed therein are hereby incorporated herein by reference for all purposes.

In yet another aspect, the cationic lipid is described in PCT Publication No. WO2023/114944, the contents of which is hereby incorporated herein by reference in its entirety for all purposes. In one embodiment therein, an ionizable cationic lipid of the disclosure comprises a compound of structure (I):

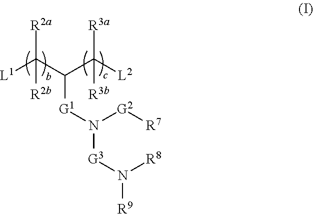

or a pharmaceutically acceptable salt, tautomer, or stereoisomer thereof, wherein: L¹ is —O(C═O)R^{1a}, —(C═O)OR^{1a}, —C(═O)R^{1a}, —OR^{1a}, —S(O)ₓR^{1a}, —S—SR^{1a}, —C(═O)SR^{1a}, —SC(═O)R^{1a}, —NR^{a}C(═O)R^{1a}, —C(═O)NR^{a}R^{1a}, —NR^{a}C(═O)NR^{a}R^{1a}, —OC(═O)NR^{a}R^{1a}, —NR^{a}C(═O)OR^{1a} or R^{1b}, L² is —O(C═O)R^{4a}, —(C═O)OR^{4a}, —C(═O)R^{4a}, —OR^{4a}, —S(O)ₓR^{4a}, —S—SR^{4a}, —C(═O)SR^{4a}, —SC(═O)R^{4a}, —NR^{a}C(═O)R^{4a}, —C(═O)NR^{a}R^{4a}, —NR^{a}C(═O)NR^{a}R^{4a}, —OC(═O)NR^{a}R^{4a}, —NR^{a}C(═O)OR^{4a}, or R^{4b}; G¹ is C₁-C₂ alkylene, —(C═O)—, —O(C═O)—, —SC(═O)—, —NR^{a}C(═O)— or a direct bond; G² is —C(═O)—, —(C═O)O—, —C(═O)S—, —C(═O)NR^{a}— or a direct bond; G³ is C₁-C₆ alkylene; R^{a} is H or C₁-C₁₂ alkyl; R^{1a} and R^{4a} are each independently branched C₆-C₂₄ alkyl, branched C₆-C₂₄ alkenyl, branched C₆-C₂₄ fluoroalkyl, branched C₆-C₂₄ fluoroalkenyl, C₆-C₂₄ alkylacetal or C₆-C₂₄ fluoroalkylacetal; R^{1b} and R^{4b} are each independently —CH(OR)(OR), wherein each R is independently linear or branched C₆-C₁₈ alkyl, linear or branched C₆-C₁₈ alkenyl, linear or branched C₆-C₁₈ fluoroalkyl, or linear or branched C₆-C₁₈ fluoroalkenyl R^{2a} and R^{2b} are, at each occurrence, independently H, F, C₁-C₁₂ alkyl, or C₁-C₁₂ fluoroalkyl; R^{3a} and R^{3b} are, at each occurrence, independently H, F, C₁-C₁₂ alkyl, or C₁-C₁₂ fluoroalkyl; R⁷ is H, C₄-C₂₀ alkyl, or C₂-C₁₀ fluoroalkyl; R⁸ and R⁹ are each independently C₁-C₁₂ alkyl; or R⁸ and R⁹, together with the nitrogen atom to which they are attached, form a 5, 6 or 7-membered heterocyclic ring; b and c are each independently an integer from 1 to 24; and wherein at least one of R^{2a}, R^{2b}, R^{3a}, and R^{3b} is F or C₁-C₁₂ fluoroalkyl; at least one of R^{1a} and R^{4a} is present and selected from branched C₆-C₂₄ fluoroalkyl, branched C₆-C₂₄ fluoroalkenyl and C₆-C₂₄ fluoroalkylacetal; at least one of R^{1b} and R^{4b} is present and selected from linear or branched C₆-C₁₈ fluoroalkyl and linear or branched C₆-C₁₈ fluoroalkenyl; G³ is C₁-C₆ fluoroalkylene; and/or R⁷ is C₂-C₁₀ fluoroalkyl.

In another embodiment, a cationic lipid of the disclosure is a compound having any one of the following structures (IA) or (IB):

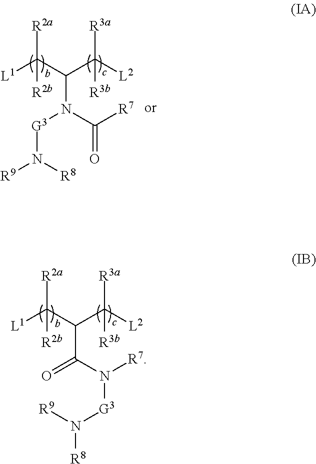

In a further embodiment, the cationic lipid is selected from any one of Compound Nos. I-1 to I-25 as specifically exemplified in Table 1 of PCT Publication No. WO2023/114944, which Table and compounds listed therein are hereby incorporated herein by reference for all purposes.

In yet another aspect, the cationic lipid is described in PCT Publication No. WO2023/114937, the contents of which is hereby incorporated herein by reference in its entirety for all purposes. In one embodiment therein, an ionizable cationic lipid of the disclosure comprises a compound of structure (I):

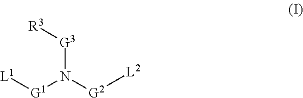

or a pharmaceutically acceptable salt or stereoisomer thereof, wherein:
L¹ is —O(C═O)R^{Ia}, —(C═O)OR^{Ia}, —C(═O)R^{Ia}, —OR^{Ia}, —S(O)ₓR^{Ia}, —S—SR^{Ia}, —C(═O)SR^{Ia}, —SC(═O)R^{Ia}, —NR^{a}C(═O)R^{Ia}, —C(═O)NR^{a}R^{Ia}, —NR^{a}C(═O)NR^{a}R^{Ia}, —OC(═O)NR^{a}R^{Ia}, —NR^{a}C(═O)OR^{Ia} or R^{Ib};
L² is —O(C═O)R^{2a}, —(C═O)OR^{2a}, —C(═O)R^{2a}, —OR^{2a}, —S(O)ₓR^{2a}, —S—SR^{2a}, —C(═O)SR^{2a}, —SC(═O)R^{2a}, —NR^{a}C(═O)R^{2a}, —C(═O)NR^{a}R^{2a}, —NR^{a}C(═O)NR^{a}R^{2a}, —OC(═O)NR^{a}R^{2a}, NR^{a}C(═O)OR^{2a}, or R^{2b};
G¹ and G² are each independently linear or branched C1-C12 alkylene or linear or branched C1-C12 fluoroalkylene;
G³ is linear or branched C1-C12 alkylene or linear or branched C1-C12 fluoroalkylene; each R^{a} is independently H or C1-C12 alkyl;
R^{Ia} and R^{2a} are each independently branched C6-C24 alkyl, branched C6-C24 alkenyl, branched C6-C24 fluoroalkyl, branched C6-C24 fluoroalkenyl, C6-C24 alkylacetal or C6-C24 fluoroalkylacetal;
R^{Ib} and R^{2b} are each independently —CH(OR)(OR), wherein each R is independently linear or branched Ce-Cis alkyl, linear or branched Ce-Cis alkenyl, linear or branched Ce-Cis fluoroalkyl, or linear or branched Ce-Cis fluoroalkenyl;
R³ is H, —OR⁵, —CN, —C(═O)OR⁴, —OC(═O)R⁴, —N(R⁵)N⁴, —C(═O)N(R⁴)R⁵, or —NR⁵C(═O)R⁴; and
R⁴ is H, C1-C12 alkyl, or aryl, and R⁵ is H or Ci-Ce alkyl; or R⁴ and R⁵, together with the nitrogen atom to which they are attached, form a 5, 6 or 7-membered heterocyclic ring, and
66 wherein at least one of G1 and G2 is linear or branched C₁-C₁₂ fluoroalkylene;
G³ is linear or branched C1-C12 fluoroalkylene; at least one of R^{Ia} and R^{2a} is present and selected from branched C6-C24 fluoroalkyl, branched C6-C24 fluoroalkenyl and C6-C24 fluoroalkylacetal; and/or at least one of R^{Ib} and R^{2b} is present and selected from linear or branched Ce-Cis fluoroalkyl and linear or branched Ce-Cis fluoroalkenyl.

In another embodiment, a cationic lipid of the disclosure is a compound having the following structure (IA):

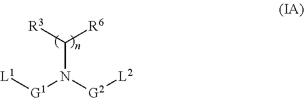

wherein:
R⁶ is, at each occurrence, independently H, F, OH or C1-C24 alkyl; and n is an integer ranging from 1 to 15.

In another embodiment, a cationic lipid of the disclosure is a compound having the following structure (IB):

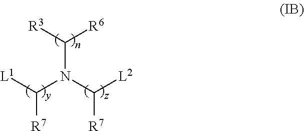

wherein: y and z are each independently integers ranging from 1 to 12; and R⁷ is, at each occurrence, independently H or F.

In another embodiment, a cationic lipid of the disclosure is a compound having any one of the following structures (IC) or (ID):

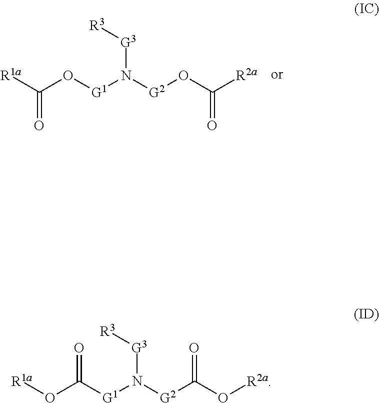

In another embodiment, a cationic lipid of the disclosure is a compound having any one of the following structures (IE) or (IF):

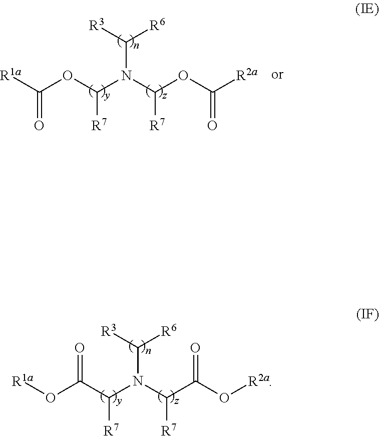

In another embodiment, a cationic lipid of the disclosure is a compound having any one of the following structures (IG) or (IH):

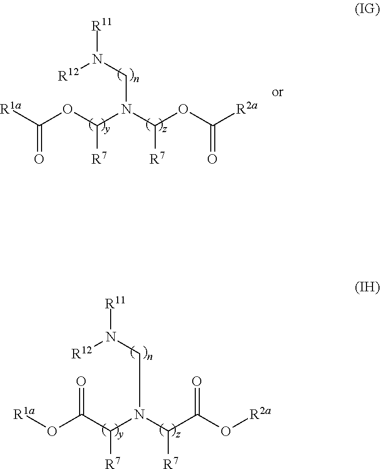

wherein R¹¹ and R¹² are each independently C1-C12 alkyl; or R¹¹ and R¹², together with the nitrogen atom to which they are attached, form a 5, 6 or 7-membered heterocyclic ring comprising one nitrogen atom.

In a further embodiment, the cationic lipid is selected from any one of Compound Nos. I-1 to I-10 as specifically exemplified in Table 1 of PCT Publication No. WO2023/114937, which Table and compounds listed therein are hereby incorporated herein by reference for all purposes.

In yet another aspect, the cationic lipid is described in PCT Publication No. WO2023/114943, the contents of which is hereby incorporated herein by reference in its entirety for all purposes. In one embodiment therein, an ionizable cationic lipid of the disclosure comprises a compound of structure (I):

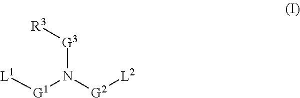

or a pharmaceutically acceptable salt, prodrug or stereoisomer thereof, wherein: L1 is —NRaC(═O)R1 or —C(═O)NRbRc; L2 is —NR^{d}C(═O)R2 or —C(═O)NReRf; G1 and G2 are each independently C2-C12 alkylene, or C2-C12 alkenylene; G3 is C1-C24 alkylene, or C2-C24 alkenylene; Ra, Rb, Rd and Re are each independently H, C1-C16 alkyl or C2-C16 alkenyl; Rc and Rf are each independently C1-C16 alkyl or C2-C16 alkenyl; R1 and R2 are each independently C6-C24 alkyl or C6-C24 alkenyl; R3 is H, —OH, CN, —N(R4)R5; —C(═O)N(R4)R5; —N(R4)C(═O)R5; —N(R4)C(═O)OR5; —C(═O)OR6, —OC(═O)R6, —OR7, heteroaryl or aryl; R4 and R5 are each independently is H, C₁-C₁₂ alkyl, C₃-C₆ cycloalkyl or C₃-C₆ cycloalkenyl, or R₄ and R5, together with the nitrogen or carbon atom to which they are bound, form a 5 to 7-membered heterocyclic ring; R6 is H, C1-C12 alkyl, C2-C12 alkenyl or aralkyl; R7 is C1-C12 alkyl optionally substituted with hydroxyl or alkoxy; and wherein each alkyl, alkenyl, alkylene, alkenylene, aryl and aralkyl is independently substituted or unsubstituted unless otherwise specified.

In another embodiment, a cationic lipid of the disclosure is a compound having the following structure (IA):

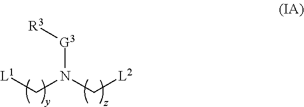

wherein y and z are each independently integers ranging from 2 to 12.

In another embodiment, a cationic lipid of the disclosure is a compound having any one of the following structures (IB) or (IC):

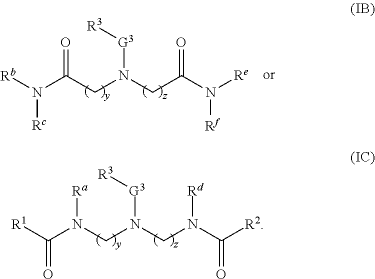

In a further embodiment, the cationic lipid is selected from any one of Compound Nos. I-1 to I-85 as specifically exemplified in Table 1 of PCT Publication No. WO2023/114943, which Table and compounds listed therein are hereby incorporated herein by reference for all purposes.

In yet another aspect, the ionizable cationic lipid is described in PCT Publication No. WO2024/054843, the contents of which is hereby incorporated herein by reference in its entirety for all purposes. In one embodiment therein, an ionizable cationic lipid of the disclosure comprises a compound of Structure (I):

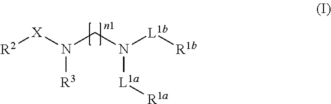

or a pharmaceutically acceptable salt, tautomer, or stereoisomer thereof, wherein:
   L^{1a} and L^{1b} are each independently optionally substituted C₃-C₁₂ alkyl;
   R^{1a} is —C(═O)OR^{4a} or —O(C═O)R^{4a};
   R^{1b} is —C(═O)OR^{4b} or —O(C═O)R^{4b};
   R² is —NR⁶(C═O)R⁵, —(C═O)N(R⁶)R⁵ or —(C═O)OR⁷;
   R³ and R⁶ are each independently hydrogen or optionally substituted C₁-C₆ alkyl;
   R^{4a}, R^{4b}, and R⁵ are each independently optionally substituted alkyl;
   R⁷ is optionally substituted arylalkyl;
   n1 is 2, 3, 4, 5, or 6; and
   X is C₂-C₆ alkylene or C₄-C₂₀ alkyleneoxide.

One embodiment provides a compound having Structure (I):

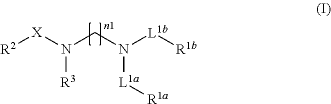

or a pharmaceutically acceptable salt, tautomer, or stereoisomer thereof, wherein:
L^{1a} and L^{1b} are each independently optionally substituted C₃-C₁₂ alkyl;
R^{1a} is —C(═O)OR^{4a} or —O(C═O)R^{4a};
R^{1b} is —C(═O)OR^{4b} or —O(C═O)R^{4b};
R² is —NR⁶(C═O)R⁵, —(C═O)N(R⁶)R⁵ or —(C═O)OR⁷;
R³ and R⁶ are each independently hydrogen or optionally substituted C₁-C₁₂ alkyl;
R^{4a}, R^{4b}, and R⁵ are each independently optionally substituted alkyl;
R⁷ is optionally substituted C₁-C₆ alkyl or optionally substituted arylalkyl;
n1 is 2, 3, 4, 5, or 6; and
X is C₂-C₆ alkylene or C₄-C₂₀ alkyleneoxide.

In some embodiments, wherein X is:

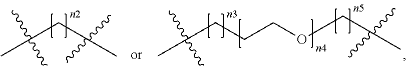

wherein:
n2 is 2, 3, 4, 5, or 6;
n3 is 0, 1, 2, 3, or 4;
n4 is 2, 3, or 4; and
n5 is 2, 3, 4, or 5.

In some embodiments, L^{1a} is C₅-C₉ alkyl. In certain embodiments, L^{1b} is C₅-C₉ alkyl. In some embodiments, L^{1a} is C₅-, C₆-, C₇-, or C₉-alkyl. In certain embodiments, L^{1b} is C₅-, C₆-, C₇-, or C₉-alkyl. In some embodiments, L^{1a} is C₅-alkyl. In certain embodiments, L^{1a} is C₆-alkyl. In some embodiments, L^{1a} is C₇-alkyl. In certain embodiments, L^{1a} is C₉-alkyl. In some embodiments, L^{1b} is C₅-alkyl. In certain embodiments, L^{1b} is C₆-alkyl. In some embodiments, L^{1b} is C₇-alkyl. In certain embodiments, L^{1b} is C₉-alkyl. In some embodiments, L^{1a} is unsubstituted. In certain embodiments, L^{1b} is unsubstituted. In some embodiments, L^{1a} is unbranched. In certain embodiments, L^{1b} is unbranched.

In some embodiments, one of R^{1a} is —O(C═O)R^{4a}. In certain embodiments, one of R^{1a} is —(C═O)OR^{4a}. In some embodiments, R^{1b} is —O(C═O)R^{4b}. In certain embodiments, one of R^{1b} is —(C═O)OR^{4b}.

In some embodiments, R^{4a} is C₅-C₂₄-alkyl. In certain embodiments, R^{4a} is C₁₀-C₁₈-alkyl. In certain embodiments, R^{4a} is C₁₁-C₁₆-alkyl. In some embodiments, R^{4a} is C₁₁-alkyl. In certain embodiments, R^{4a} is C₁₅-alkyl. In some embodiments, R^{4a} is C₁₆-alkyl. In certain embodiments, R^{4b} is C₈-C₂₄-alkyl. In some embodiments, R^{4b} is C₁₀-C₁₈-alkyl. In certain embodiments, R^{4b} is C₁₁-C₁₆-alkyl. In some embodiments, R^{4b} is C₁₁-alkyl. In certain embodiments, R^{4b} is C₁₅-alkyl. In some embodiments, R^{4b} is C₁₆-alkyl.

In certain embodiments, R^{4a} is branched. In some embodiments, R^{4b} is branched. In certain embodiments, R^{4a} is unsubstituted. In some embodiments, R^{4b} is unsubstituted. In certain embodiments, R^{4a} has one of the following structures:

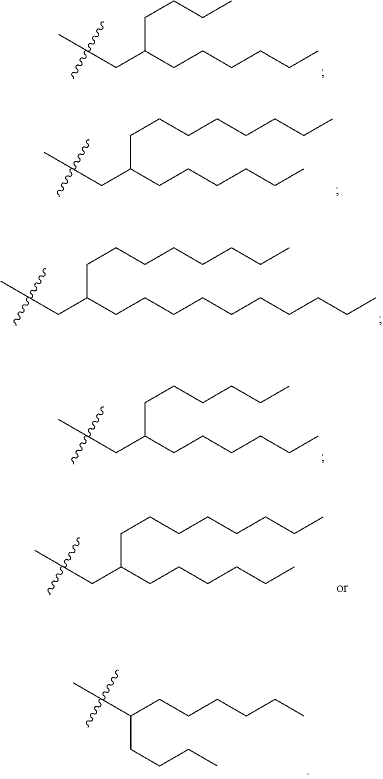 [SMILES: *CC(CCCC)CCCCCC ; *CC(CCCCCC)CCCCCCCC ; *CC(CCCCCCCC)CCCCCCCCCC ; *CC(CCCCCC)CCCCCC ; *CC(CCCCCC)CCCCCCCC ; *C(CCCC)CCCCCC]

In some embodiments, R^{4b} has one of the following structures:

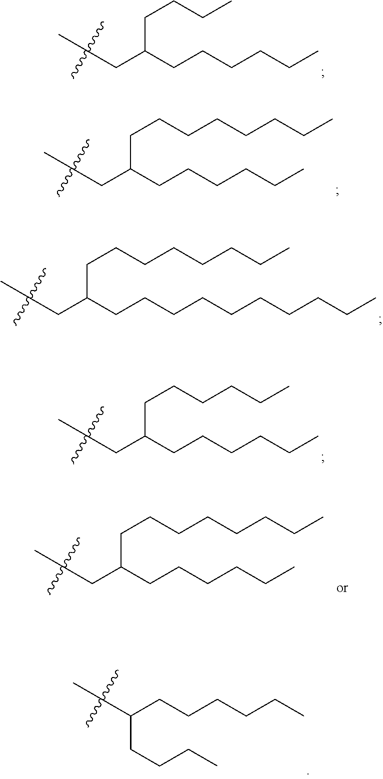 [SMILES: *CC(CCCC)CCCCCC ; *CC(CCCCCC)CCCCCCCC ; *CC(CCCCCCCC)CCCCCCCCCC ; *CC(CCCCCC)CCCCCC ; *CC(CCCCCC)CCCCCCCC ; *C(CCCC)CCCCCC]

In some embodiments, R² is —NR⁶(C═O)R⁵. In certain embodiments, R² is —(C═O)N(R⁶)R⁵. In some embodiments, R⁵ is C₂-C₁₆-alkyl. In certain embodiments, R⁵ is C₄-C₁₃-alkyl. In some embodiments, R⁵ is C₄-, C₇-, C₉-, C₁₀-, or C₁₃-alkyl. In certain embodiments, R⁵ is unsubstituted. In some embodiments, R⁵ is substituted with hydroxyl. In some embodiments, R⁵ is branched. In certain embodiments, R⁵ is unbranched. In some embodiments, R⁵ has one of the following structures:

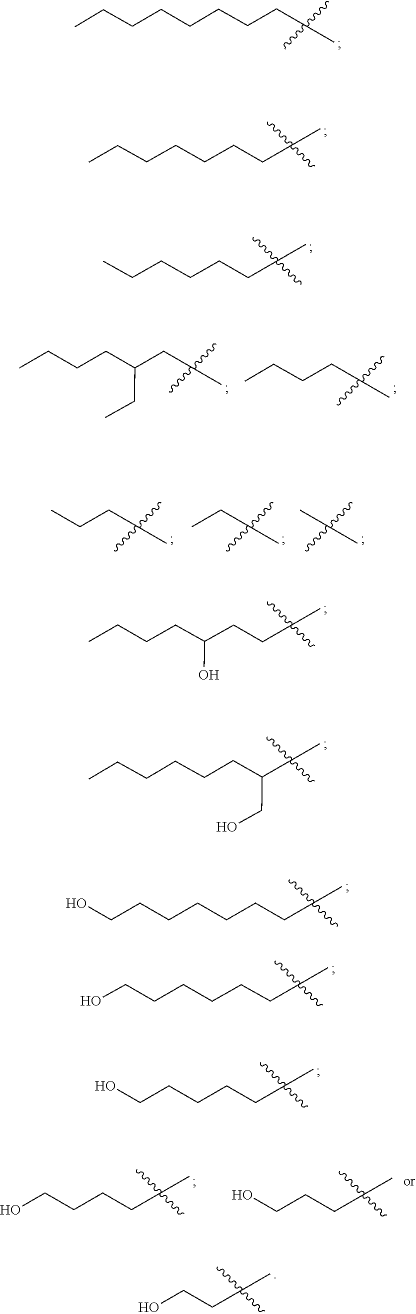

In certain embodiments, R⁵ is unbranched. In some embodiments, R⁵ has one of the following structures:

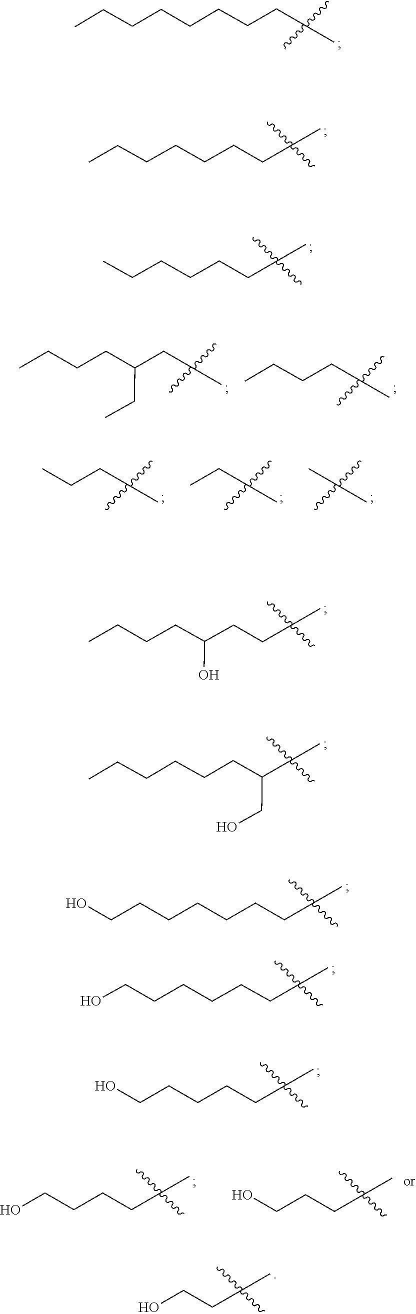

In some embodiments, R⁶ is C₁-C₆ alkyl. In some embodiments, R⁶ is C₁-C₁₀ alkyl. In certain embodiments, R⁶ is C₁-C₄-alkyl. In some embodiments, R⁶ is C₁-, C₂-, C₃-, C₆-, C₈-, or C₁₀-alkyl. In certain embodiments, methyl, ethyl, n-butyl, n-hexyl, n-octyl, or n-decyl. In some embodiments, R⁶ is unbranched. In certain embodiments, R⁶ is methyl or n-butyl. In some embodiments, R⁶ is unsubstituted. In some embodiments, R⁶ is substituted. In some embodiments, R⁶ is C₁-C₆ alkyl substituted with one or more hydroxyl. In some embodiments, R⁶ is C₂-, C₃-, C₄-, or C₆-alkyl substituted with one or more hydroxyl. In certain embodiments, R⁶ is hydrogen. In some embodiments, R² is —(C═O)OR⁷.

In some embodiments, R⁷ is C₁-C₃ alkyl or C₇-C₁₆ arylalkyl. In certain embodiments, R⁷ is C₇-C₁₆ arylalkyl. In some embodiments, R⁷ is C₁-C₃ alkyl. In some embodiments, R⁷ is unsubstituted.

In certain embodiments, R⁷ is —CH₃ or has the following structure:

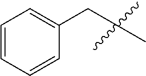 [SMILES: *Cc1ccccc1]

In certain embodiments, R⁷ has the following structure:

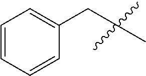 [SMILES: *Cc1ccccc1]

In some embodiments, R³ is optionally substituted C₁-C₆ alkyl. In certain embodiments, R³ is optionally substituted methyl, ethyl, n-propyl, iso-propyl, n-butyl, iso-butyl, tert-butyl, n-hexyl. In some embodiments, R³ is optionally substituted methyl. In some embodiments, R³ is C₁-C₆ alkyl substituted with one or more hydroxyl. In some embodiments, R³ is C₂- or C₄-alkyl substituted with one or more hydroxyl. In certain embodiments, R³ is unsubstituted. In some embodiments, R³ is hydrogen.

In some embodiments, X is

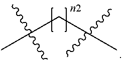

For example, in certain aspects the compound has the following Structure (II):

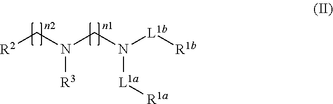

or a pharmaceutically acceptable salt, tautomer, or stereoisomer thereof. For example, in some of these embodiments n2 is 3, 4, or 5.

In other embodiments X is

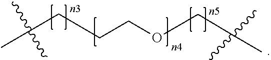

In some such embodiments, the compound has the following Structure (III):

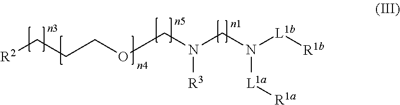

or a pharmaceutically acceptable salt, tautomer, or stereoisomer thereof. In different of these embodiments, n3 is 0 or 1. In other embodiments, n4 is 2 or 3. In some other different embodiments, n5 is 3.

In some embodiments, n1 is 3, 4, or 5. In certain embodiments, n1 is 2.

In some embodiments, the compound has one of the structures set forth in the Table below or a pharmaceutically acceptable salt, tautomer, or stereoisomer thereof:

| No. | Structure |
|---|---|
| I- 1 | 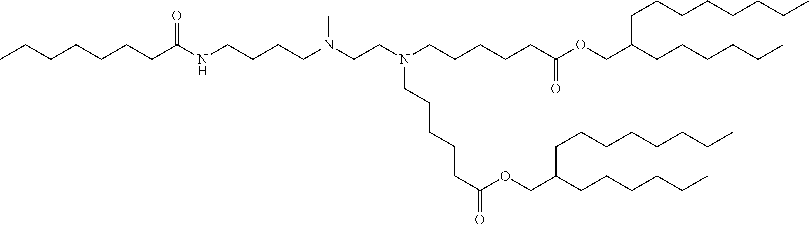 [SMILES: CCCCCCCCC(CCCCCC)COC(=O)CCCCCN(CCCCCC(=O)OCC(CCCCCC)CCCCCCCC)CCN(C)CCCCNC(=O)CCCCCCC] |
| I- 2 | 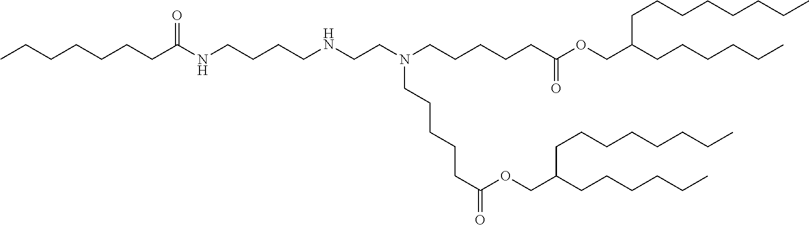 [SMILES: CCCCCCCCC(CCCCCC)COC(=O)CCCCCN(CCCCCC(=O)OCC(CCCCCC)CCCCCCCC)CCNCCCCNC(=O)CCCCCCC] |
| I- 3 | 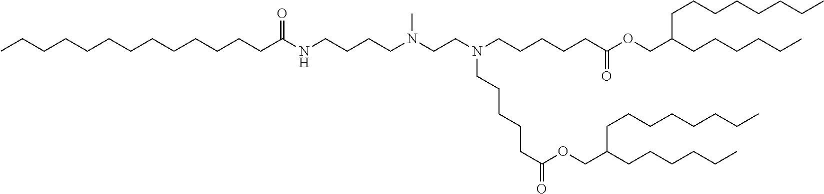 [SMILES: CCCCCCCCCCCCCC(=O)NCCCCN(C)CCN(CCCCCC(=O)OCC(CCCCCC)CCCCCCCC)CCCCCC(=O)OCC(CCCCCC)CCCCCCCC] |
| I- 4 | 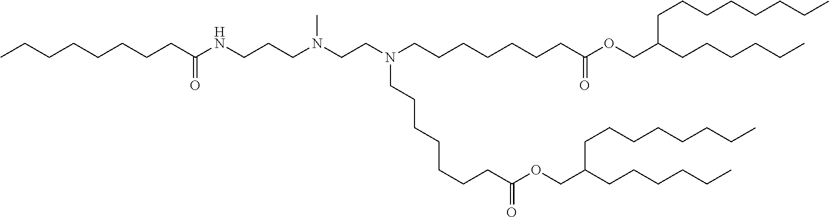 [SMILES: CCCCCCCCC(=O)NCCCN(C)CCN(CCCCCCCC(=O)OCC(CCCCCC)CCCCCCCC)CCCCCCCC(=O)OCC(CCCCCC)CCCCCCCC] |
| I- 5 | 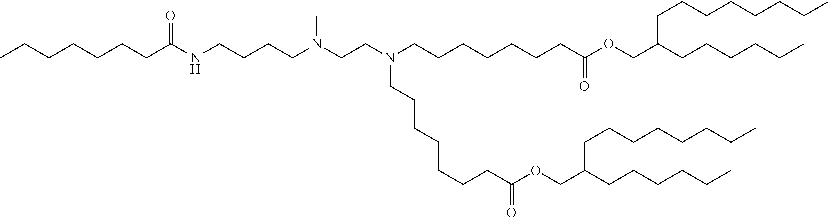 [SMILES: CCCCCCCCC(CCCCCC)COC(=O)CCCCCCCN(CCCCCCCC(=O)OCC(CCCCCC)CCCCCCCC)CCN(C)CCCCNC(=O)CCCCCCC] |
| I- 6 | 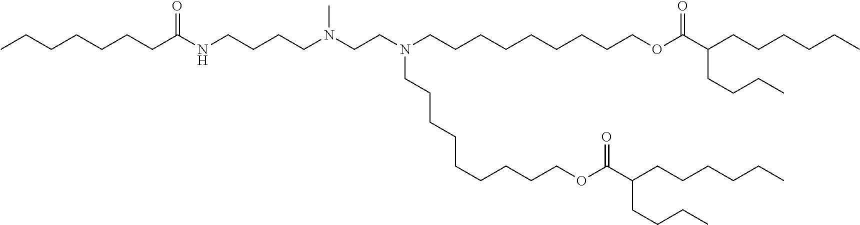 [SMILES: CCCCCCCC(=O)NCCCCN(C)CCN(CCCCCCCCCOC(=O)C(CCCC)CCCCCC)CCCCCCCCCOC(=O)C(CCCC)CCCCCC] |
| I- 7 | 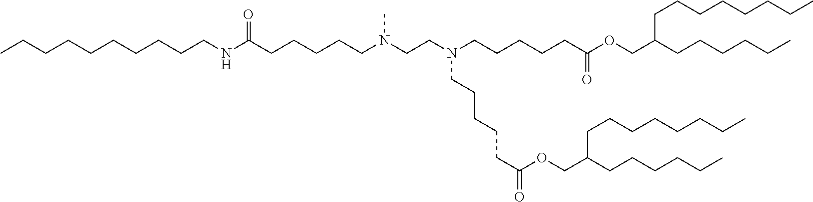 |
| I- 8 | 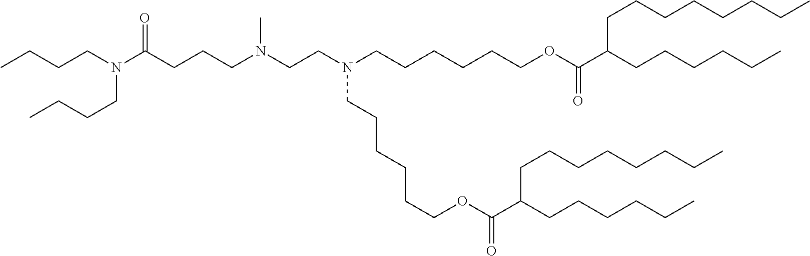 |
| I- 9 | 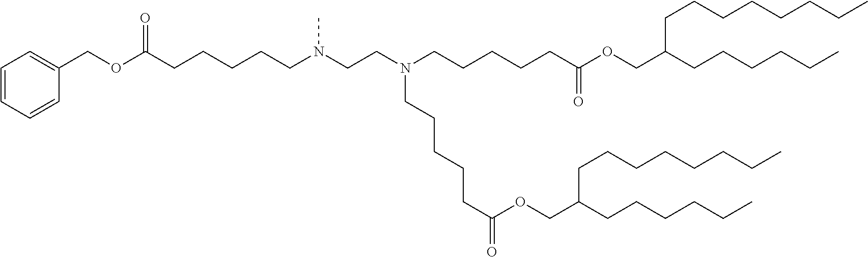 |
| I- 10 | 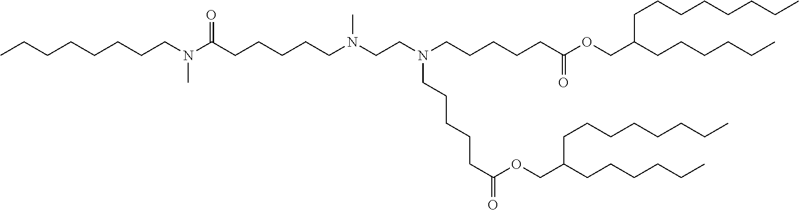 [SMILES: CCCCCCCCC(CCCCCC)COC(=O)CCCCCN(CCCCCC(=O)OCC(CCCCCC)CCCCCCCC)CCN(C)CCCCCC(=O)N(C)CCCCCCCC] |
| I- 11 | 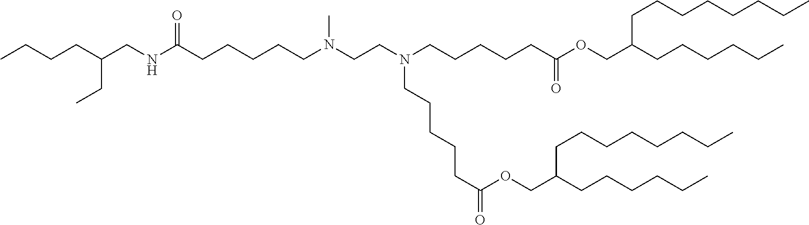 [SMILES: CCCCCCCCC(CCCCCC)COC(=O)CCCCCN(CCCCCC(=O)OCC(CCCCCC)CCCCCCCC)CCN(C)CCCCCC(=O)NCC(CC)CCCC] |
| I- 12 | 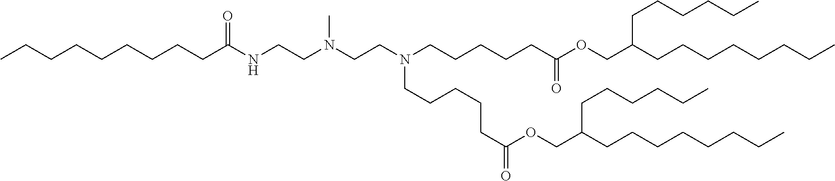 [SMILES: CCCCCCCCCC(=O)NCCN(C)CCN(CCCCCC(=O)OCC(CCCCCC)CCCCCCCC)CCCCCC(=O)OCC(CCCCCC)CCCCCCCC] |
| I- 13 | 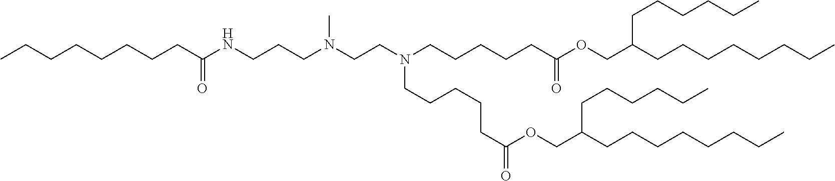 [SMILES: CCCCCCCCC(=O)NCCCN(C)CCN(CCCCCC(=O)OCC(CCCCCC)CCCCCCCC)CCCCCC(=O)OCC(CCCCCC)CCCCCCCC] |
| I- 14 | 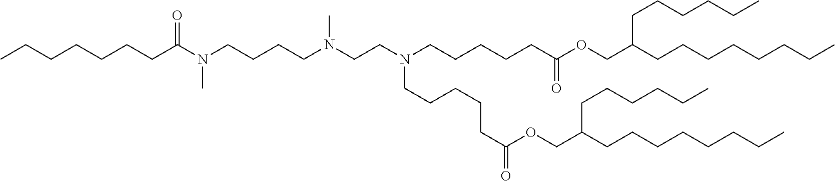 [SMILES: CCCCCCCCC(CCCCCC)COC(=O)CCCCCN(CCCCCC(=O)OCC(CCCCCC)CCCCCCCC)CCN(C)CCCCN(C)C(=O)CCCCCCC] |
| I- 15 | 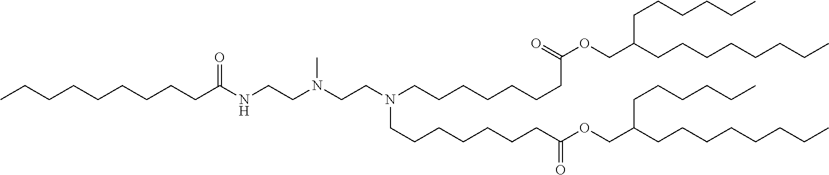 [SMILES: CCCCCCCCCC(=O)NCCN(C)CCN(CCCCCCCC(=O)OCC(CCCCCC)CCCCCCCC)CCCCCCCC(=O)OCC(CCCCCC)CCCCCCCC] |
| I- 16 | 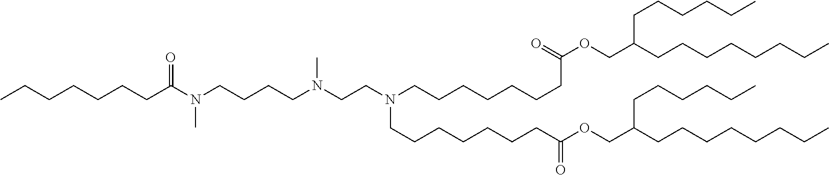 [SMILES: CCCCCCCCC(CCCCCC)COC(=O)CCCCCCCN(CCCCCCCC(=O)OCC(CCCCCC)CCCCCCCC)CCN(C)CCCCN(C)C(=O)CCCCCCC] |
| I- 17 | 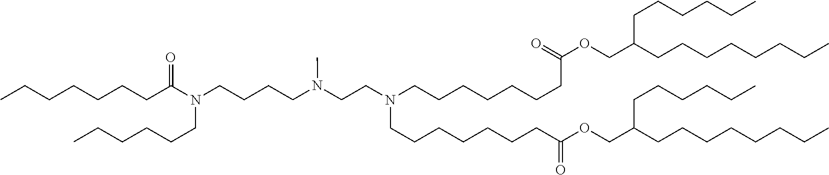 [SMILES: CCCCCCCCC(CCCCCC)COC(=O)CCCCCCCN(CCCCCCCC(=O)OCC(CCCCCC)CCCCCCCC)CCN(C)CCCCN(CCCCCC)C(=O)CCCCCCC] |
| I- 18 | 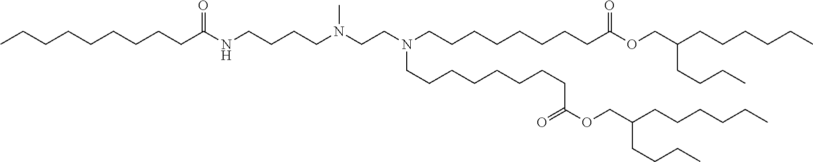 [SMILES: CCCCCCCCCC(=O)NCCCCN(C)CCN(CCCCCCCCC(=O)OCC(CCCC)CCCCCC)CCCCCCCCC(=O)OCC(CCCC)CCCCCC] |
| I- 19 | 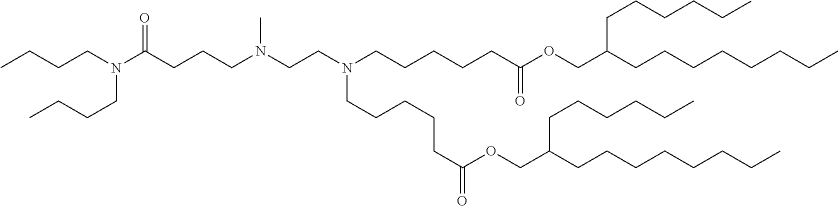 [SMILES: CCCCCCCCC(CCCCCC)COC(=O)CCCCCN(CCCCCC(=O)OCC(CCCCCC)CCCCCCCC)CCN(C)CCCC(=O)N(CCCC)CCCC] |
| I- 20 | 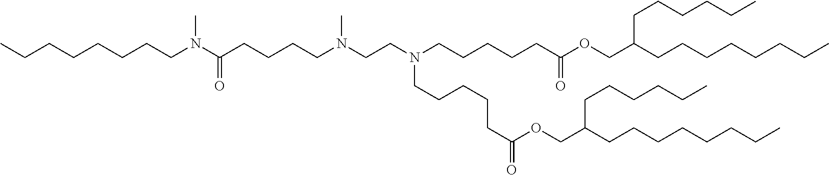 [SMILES: CCCCCCCCC(CCCCCC)COC(=O)CCCCCN(CCCCCC(=O)OCC(CCCCCC)CCCCCCCC)CCN(C)CCCCC(=O)N(C)CCCCCCCC] |
| I- 21 | 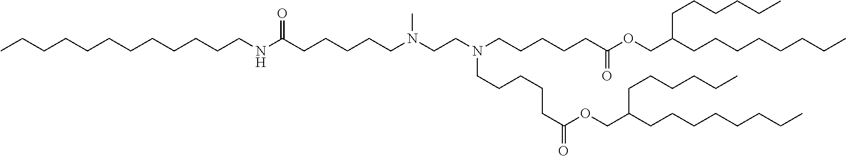 [SMILES: CCCCCCCCCCCCNC(=O)CCCCCN(C)CCN(CCCCCC(=O)OCC(CCCCCC)CCCCCCCC)CCCCCC(=O)OCC(CCCCCC)CCCCCCCC] |
| I- 22 | 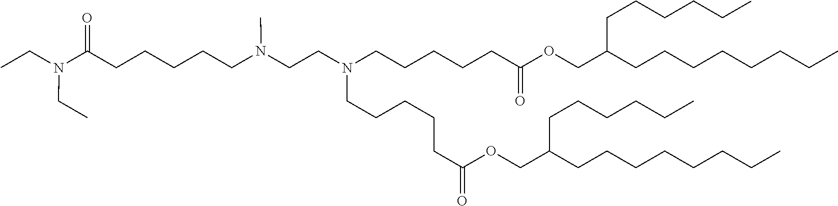 [SMILES: CCCCCCCCC(CCCCCC)COC(=O)CCCCCN(CCCCCC(=O)OCC(CCCCCC)CCCCCCCC)CCN(C)CCCCCC(=O)N(CC)CC] |
| I- 23 | 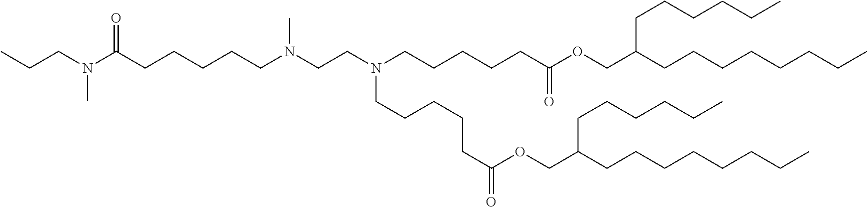 [SMILES: CCCCCCCCC(CCCCCC)COC(=O)CCCCCN(CCCCCC(=O)OCC(CCCCCC)CCCCCCCC)CCN(C)CCCCCC(=O)N(C)CCC] |
| I- 24 | 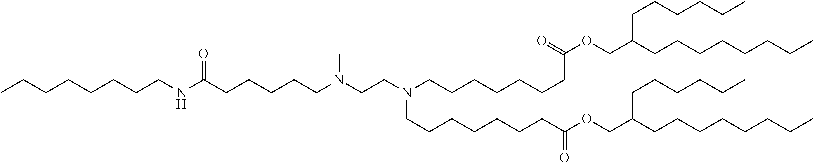 [SMILES: CCCCCCCCNC(=O)CCCCCN(C)CCN(CCCCCCCC(=O)OCC(CCCCCC)CCCCCCCC)CCCCCCCC(=O)OCC(CCCCCC)CCCCCCCC] |
| I- 25 | 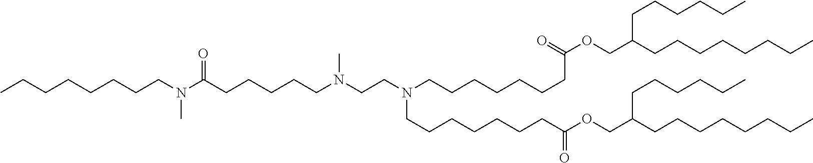 [SMILES: CCCCCCCCC(CCCCCC)COC(=O)CCCCCCCN(CCCCCCCC(=O)OCC(CCCCCC)CCCCCCCC)CCN(C)CCCCCC(=O)N(C)CCCCCCCC] |
| I- 26 | 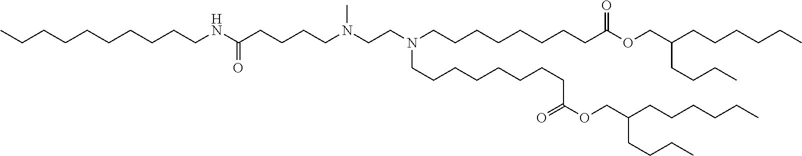 [SMILES: CCCCCCCCCCNC(=O)CCCCN(C)CCN(CCCCCCCCC(=O)OCC(CCCC)CCCCCC)CCCCCCCCC(=O)OCC(CCCC)CCCCCC] |
| I- 27 | 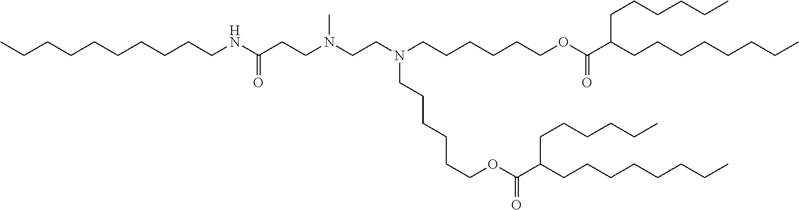 [SMILES: CCCCCCCCCCNC(=O)CCN(C)CCN(CCCCCCOC(=O)C(CCCCCC)CCCCCCCC)CCCCCCOC(=O)C(CCCCCC)CCCCCCCC] |
| I- 28 | 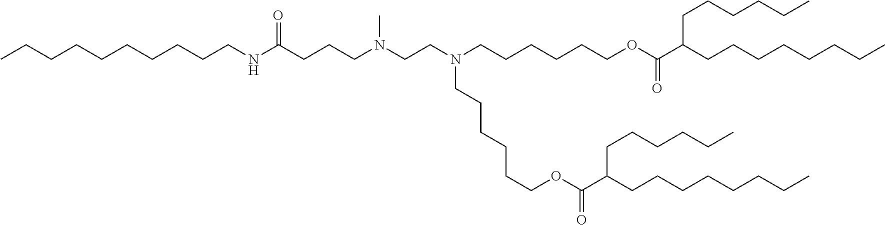 [SMILES: CCCCCCCCCCNC(=O)CCCN(C)CCN(CCCCCCOC(=O)C(CCCCCC)CCCCCCCC)CCCCCCOC(=O)C(CCCCCC)CCCCCCCC] |
| I- 29 | 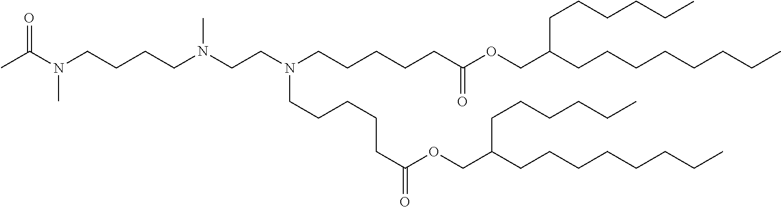 [SMILES: CCCCCCCCC(CCCCCC)COC(=O)CCCCCN(CCCCCC(=O)OCC(CCCCCC)CCCCCCCC)CCN(C)CCCCN(C)C(C)=O] |
| I- 30 | 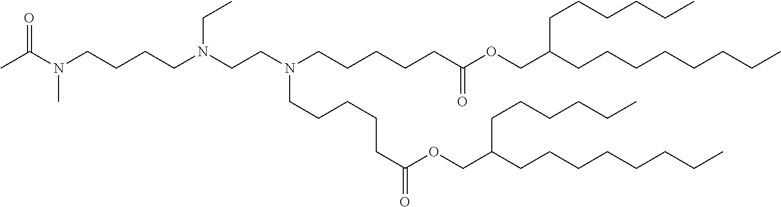 [SMILES: CCCCCCCCC(CCCCCC)COC(=O)CCCCCN(CCCCCC(=O)OCC(CCCCCC)CCCCCCCC)CCN(CC)CCCCN(C)C(C)=O] |
| I- 31 | 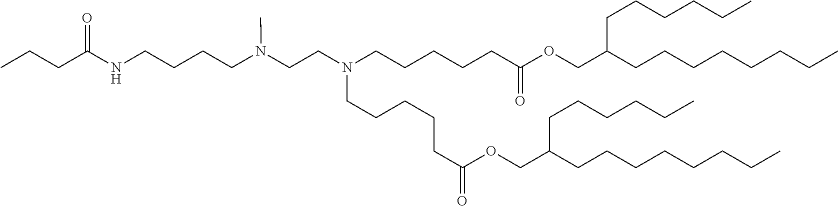 [SMILES: CCCCCCCCC(CCCCCC)COC(=O)CCCCCN(CCCCCC(=O)OCC(CCCCCC)CCCCCCCC)CCN(C)CCCCNC(=O)CCC] |
| I- 32 | 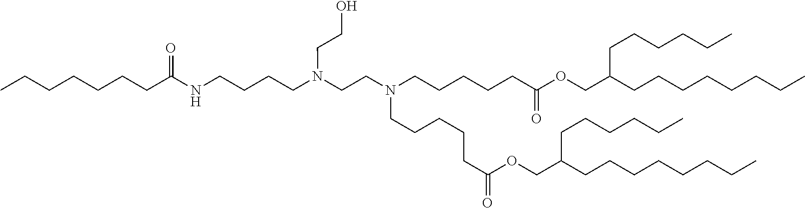 [SMILES: CCCCCCCCC(CCCCCC)COC(=O)CCCCCN(CCCCCC(=O)OCC(CCCCCC)CCCCCCCC)CCN(CCO)CCCCNC(=O)CCCCCCC] |
| I- 33 | 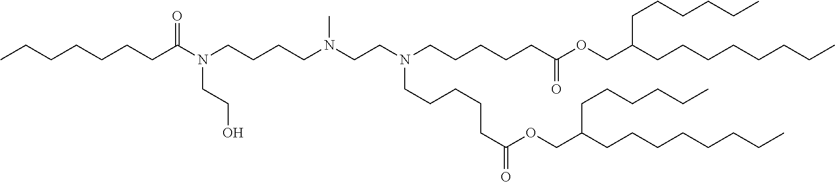 [SMILES: CCCCCCCCC(CCCCCC)COC(=O)CCCCCN(CCCCCC(=O)OCC(CCCCCC)CCCCCCCC)CCN(C)CCCCN(CCO)C(=O)CCCCCCC] |
| I- 34 | 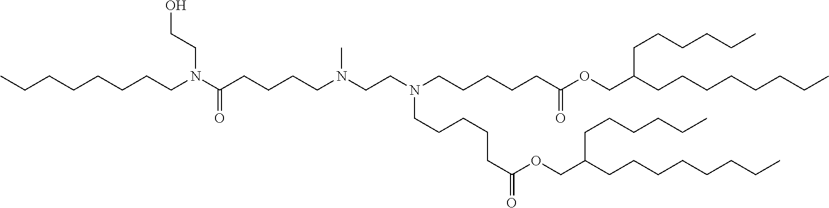 [SMILES: CCCCCCCCC(CCCCCC)COC(=O)CCCCCN(CCCCCC(=O)OCC(CCCCCC)CCCCCCCC)CCN(C)CCCCC(=O)N(CCO)CCCCCCCC] |
| I- 35 | 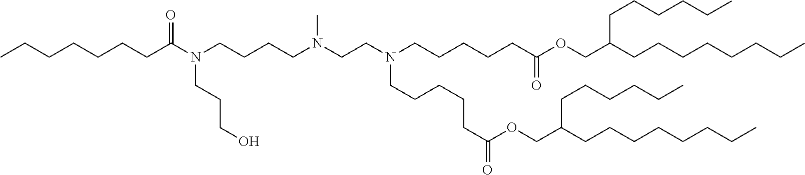 [SMILES: CCCCCCCCC(CCCCCC)COC(=O)CCCCCN(CCCCCC(=O)OCC(CCCCCC)CCCCCCCC)CCN(C)CCCCN(CCCO)C(=O)CCCCCCC] |
| I- 36 | 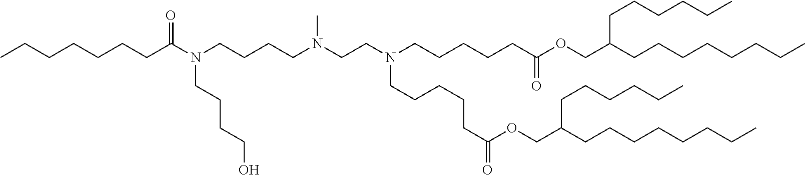 [SMILES: CCCCCCCCC(CCCCCC)COC(=O)CCCCCN(CCCCCC(=O)OCC(CCCCCC)CCCCCCCC)CCN(C)CCCCN(CCCCO)C(=O)CCCCCCC] |
| I- 37 | 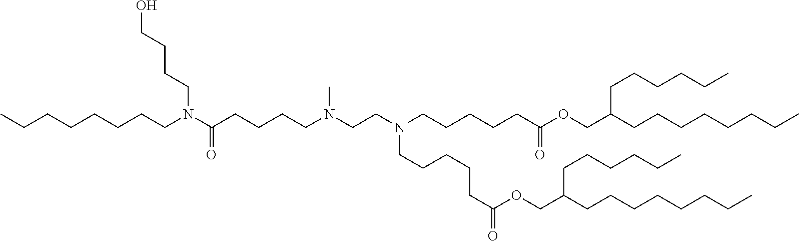 [SMILES: CCCCCCCCC(CCCCCC)COC(=O)CCCCCN(CCCCCC(=O)OCC(CCCCCC)CCCCCCCC)CCN(C)CCCCC(=O)N(CCCCO)CCCCCCCC] |
| I- 38 | 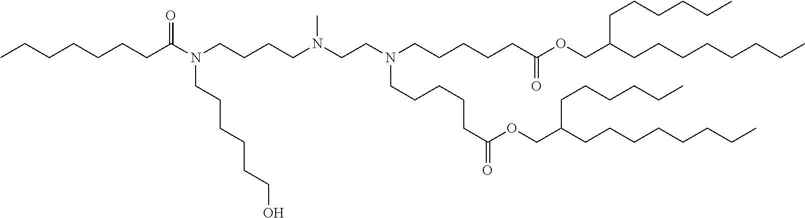 [SMILES: CCCCCCCCC(CCCCCC)COC(=O)CCCCCN(CCCCCC(=O)OCC(CCCCCC)CCCCCCCC)CCN(C)CCCCN(CCCCCCO)C(=O)CCCCCCC] |
| I- 39 | 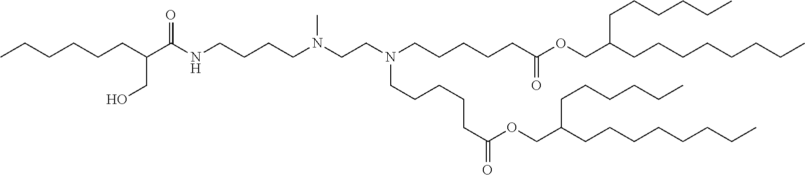 [SMILES: CCCCCCCCC(CCCCCC)COC(=O)CCCCCN(CCCCCC(=O)OCC(CCCCCC)CCCCCCCC)CCN(C)CCCCNC(=O)C(CO)CCCCCC] |
| I- 40 | 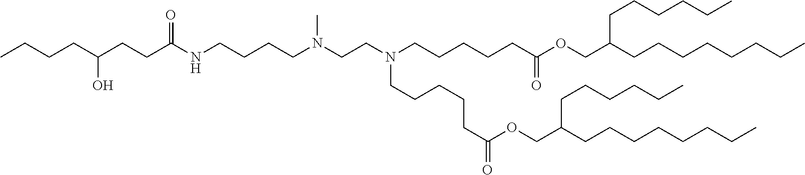 [SMILES: CCCCCCCCC(CCCCCC)COC(=O)CCCCCN(CCCCCC(=O)OCC(CCCCCC)CCCCCCCC)CCN(C)CCCCNC(=O)CCC(O)CCCC] |
| I- 41 | 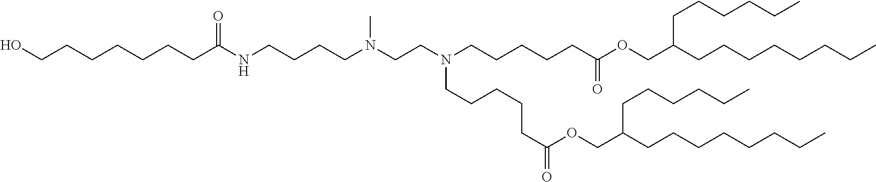 [SMILES: CCCCCCCCC(CCCCCC)COC(=O)CCCCCN(CCCCCC(=O)OCC(CCCCCC)CCCCCCCC)CCN(C)CCCCNC(=O)CCCCCCCO] |
| I- 42 | 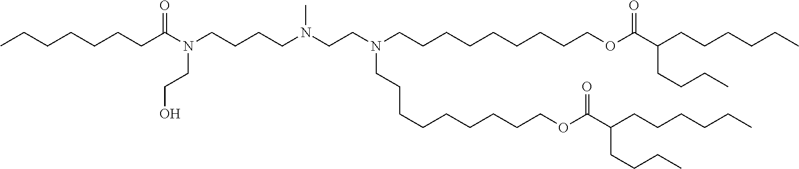 [SMILES: CCCCCCCC(=O)N(CCO)CCCCN(C)CCN(CCCCCCCCCOC(=O)C(CCCC)CCCCCC)CCCCCCCCCOC(=O)C(CCCC)CCCCCC] |
| I- 43 | 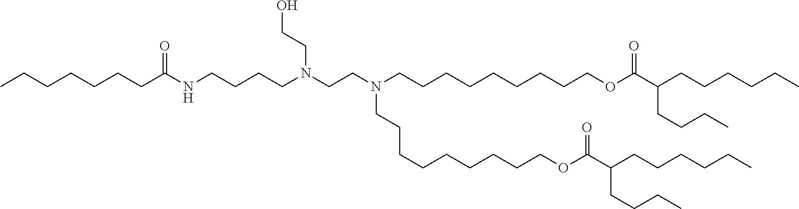 [SMILES: CCCCCCCC(=O)NCCCCN(CCO)CCN(CCCCCCCCCOC(=O)C(CCCC)CCCCCC)CCCCCCCCCOC(=O)C(CCCC)CCCCCC] |
| I- 44 | 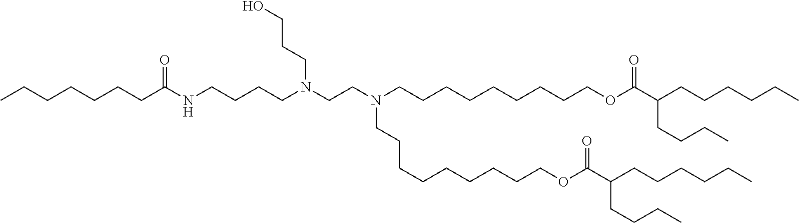 [SMILES: CCCCCCCC(=O)NCCCCN(CCCO)CCN(CCCCCCCCCOC(=O)C(CCCC)CCCCCC)CCCCCCCCCOC(=O)C(CCCC)CCCCCC] |
| I- 45 | 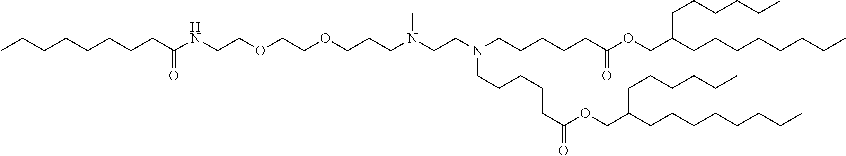 [SMILES: CCCCCCCCC(=O)NCCOCCOCCCN(C)CCN(CCCCCC(=O)OCC(CCCCCC)CCCCCCCC)CCCCCC(=O)OCC(CCCCCC)CCCCCCCC] |
| I- 46 | 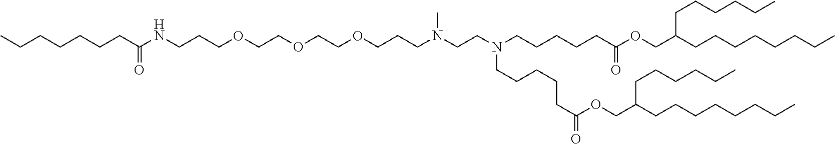 [SMILES: CCCCCCCCC(CCCCCC)COC(=O)CCCCCN(CCCCCC(=O)OCC(CCCCCC)CCCCCCCC)CCN(C)CCCOCCOCCOCCCNC(=O)CCCCCCC] |
| I- 47 | 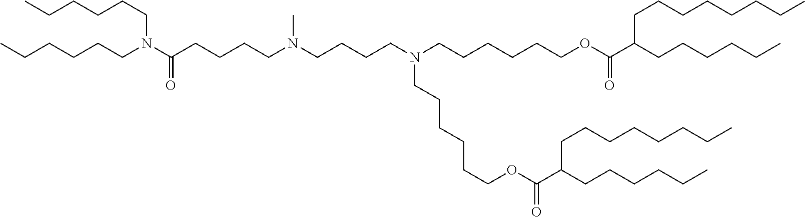 [SMILES: CCCCCCCCC(CCCCCC)C(=O)OCCCCCCN(CCCCCCOC(=O)C(CCCCCC)CCCCCCCC)CCCCN(C)CCCCC(=O)N(CCCCCC)CCCCCC] |
| I- 48 | 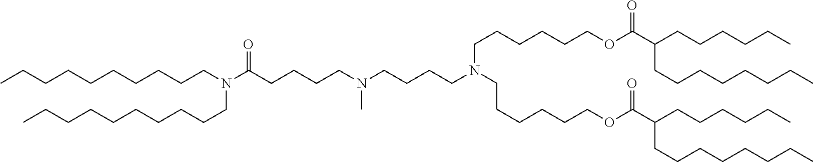 [SMILES: CCCCCCCCCCN(CCCCCCCCCC)C(=O)CCCCN(C)CCCCN(CCCCCCOC(=O)C(CCCCCC)CCCCCCCC)CCCCCCOC(=O)C(CCCCCC)CCCCCCCC] |
| I- 49 | 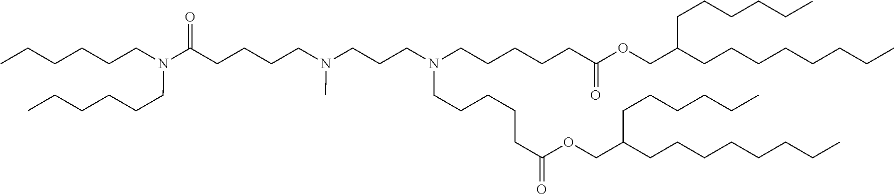 [SMILES: CCCCCCCCC(CCCCCC)COC(=O)CCCCCN(CCCCCC(=O)OCC(CCCCCC)CCCCCCCC)CCCN(C)CCCCC(=O)N(CCCCCC)CCCCCC] |
| I- 50 | 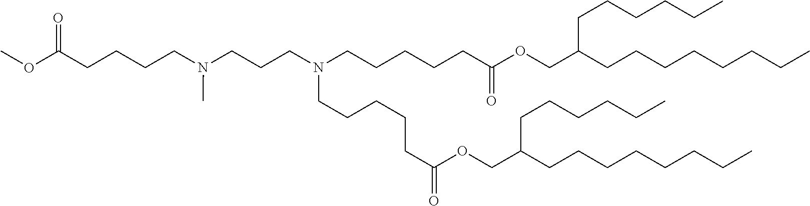 [SMILES: CCCCCCCCC(CCCCCC)COC(=O)CCCCCN(CCCCCC(=O)OCC(CCCCCC)CCCCCCCC)CCCN(C)CCCCC(=O)OC] |
| I- 51 | 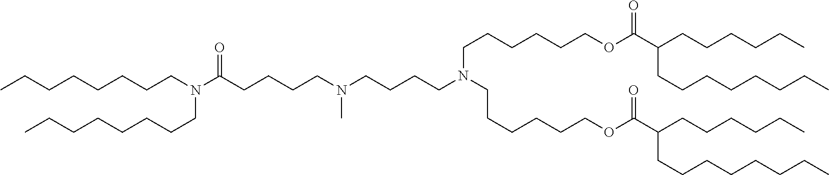 [SMILES: CCCCCCCCC(CCCCCC)C(=O)OCCCCCCN(CCCCCCOC(=O)C(CCCCCC)CCCCCCCC)CCCCN(C)CCCCC(=O)N(CCCCCCCC)CCCCCCCC] |
| I- 52 | 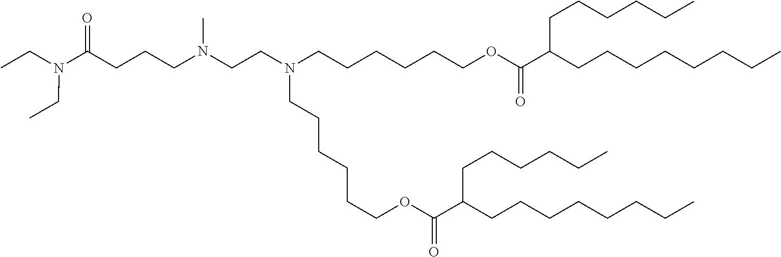 [SMILES: CCCCCCCCC(CCCCCC)C(=O)OCCCCCCN(CCCCCCOC(=O)C(CCCCCC)CCCCCCCC)CCN(C)CCCC(=O)N(CC)CC] |
| I- 53 | 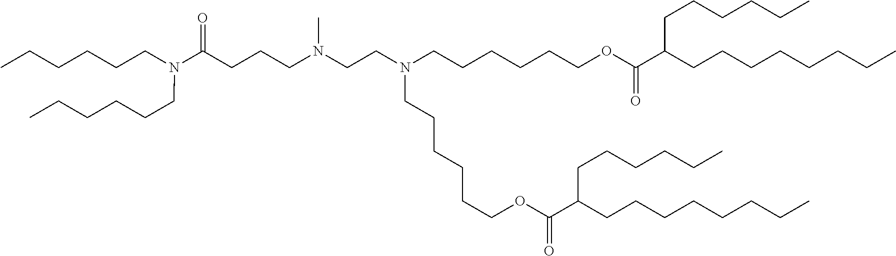 [SMILES: CCCCCCCCC(CCCCCC)C(=O)OCCCCCCN(CCCCCCOC(=O)C(CCCCCC)CCCCCCCC)CCN(C)CCCC(=O)N(CCCCCC)CCCCCC] |

The names of exemplary Compounds I-1 to I-53 set forth in the Table above are listed below:

| Compound | |
|---|---|
| Number | Name |
| I-1 | bis(2-hexyldecyl) 6,6′-((2-(methyl(4- |
| | octanamidobutyl)amino)ethyl)azanediyl)dihexanoate |
| I-2 | bis(2-hexyldecyl) 6,6′-((2-((4- |
| | octanamidobutyl)amino)ethyl)azanediyl)dihexanoate |
| I-3 | bis(2-hexyldecyl) 6,6′-((2-(methyl(4- |
| | tetradecanamidobutyl)amino)ethyl)azanediyl)dihexanoate |
| I-4 | bis(2-hexyldecyl) 8,8′-((2-(methyl(3- |
| | nonanamidopropyl)amino)ethyl)azanediyl)dioctanoate |
| I-5 | bis(2-hexyldecyl) 8,8′-((2-(methyl(4- |
| | octanamidobutyl)amino)ethyl)azanediyl)dioctanoate |
| I-6 | ((2-(methyl(4- |
| | octanamidobutyl)amino)ethyl)azanediyl)bis(nonane-9,1- |
| | diyl) bis(2-butyloctanoate) |
| I-7 | bis(2-hexyldecyl) 6,6′-((2-((6-(decylamino)-6- |
| | oxohexyl)(methyl)amino)ethyl)azanediyl)dihexanoate |
| I-8 | ((2-((4-(dibutylamino)-4- |
| | oxobutyl)(methyl)amino)ethyl)azanediyl)bis(hexane- |
| | 6,1-diyl) bis(2-hexyldec anoate) |
| I-9 | bis(2-hexyldecyl) 6,6′-((2-((6-(benzyloxy)-6- |
| | oxohexyl)(methyl)amino)ethyl)azanediyl)dihexanoate |
| I-10 | bis(2-hexyldecyl) 6,6′-((2-(methyl(6-(methyl(octyl)amino)- |
| | 6-oxohexyl)amino)ethyl)azanediyl)dihexanoate |
| I-11 | bis(2-hexyldecyl) 6,6′-((2-((6-((2-ethylhexyl)amino)- |
| | 6-oxohexyl)(methyl)amino)ethyl)azanediyl)dihexanoate |
| I-47 | ((4-((5-(dihexylamino)-5- |
| | oxopentyl)(methyl)amino)butyl)azanediyl)bis(hexane- |
| | 6,1-diyl) bis(2-hexyldecanoate) |
| I-48 | ((4-((5-(didecylamino)-5- |
| | oxopentyl)(methyl)amino)butyl)azanediyl)bis(hexane- |
| | 6,1-diyl) bis(2-hexyldecanoate) |
| I-49 | bis(2-hexyldecyl) 6,6′-((3-((5-(dihexylamino)-5- |
| | oxopentyl)(methyl)amino)propyl)azanediyl)dihexanoate |
| I-50 | bis(2-hexyldecyl) 6,6′-((3-((5-methoxy-5- |
| | oxopentyl)(methyl)amino)propyl)azanediyl)dihexanoate |
| I-51 | ((4-((5-(dioctylamino)-5- |
| | oxopentyl)(methyl)amino)butyl)azanediyl)bis(hexane- |
| | 6,1-diyl) bis(2-hexyldecanoate) |
| I-52 | 6-((2-((3-(a,a-diethylcarbamoyl)propyl)-n- |
| | methylamino)ethyl)(6- |
| | (1-hexylnonylcarbonyloxy)hexyl)amino)hexyl |
| | 2-hexyldecanoate |
| I-53 | 6-((2-((3-(7v,7v-dihexylcarbamoyl)propyl)-n- |
| | methylamino)ethyl)(6- |
| | (1-hexylnonylcarbonyloxy)hexyl)amino)hexyl |
| | 2-hexyldecanoate |

In a further aspect, the ionizable cationic lipid is described in PCT Publication No. WO2024/233387, the contents of which is hereby incorporated herein by reference in its entirety for all purposes. In one embodiment therein, an ionizable cationic lipid of the disclosure comprises a compound having the following structure (I):

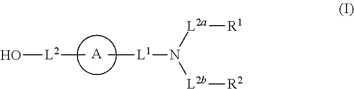

or a pharmaceutically acceptable salt or stereoisomer thereof, wherein:
A is a 3-10 membered carbocyclic or oxygen-containing heterocyclic ring optionally substituted with one or more fluoro, hydroxyl, C₁-C₆ alkyl, or C₁-C₆ alkylhydroxyl substituents;
R¹ is —NR^{a}C(═O)R³ or —C(═O)NR^{b}R^{c};
R² is —NR^{a}C(═O)R⁴ or —C(═O)NR^{e}R^{f};
R³ and R⁴ are each independently C6-C24 alkyl or C6-C24 alkenyl;
R^{a}, R^{b}, R^{d} and R^{e} are each independently H, C1-C20 alkyl or C2-C20 alkenyl;
R^{c} and R^{f} are each independently C1-C20 alkyl or C2-C20 alkenyl;
L¹ and L² are each independently a direct bond or C1-C6 alkylene; and
L^{2a} and L^{2b} are each independently C4-C12 alkylene;
wherein each alkyl, alkylene, and alkenyl is optionally substituted with one or more fluoro.

In another embodiment, the cationic lipid is selected from any one of Compound Nos. I-1 to I-63 as specifically exemplified in Table 1 of PCT Publication No. WO2024/233387, including any racemic mixtures of enantiomer pairs of such compounds as described therein, which Table, compounds and racemic mixtures thereof listed therein are hereby incorporated herein by reference for all purposes.

In another embodiment, the cationic lipid is selected from any compound having a structure set forth in Formula (I), (Ia), (Ib), (Ic), or (Id), and Compound Nos. I-1 to 1-21 as specifically exemplified in Table 1 of PCT Publication No. WO2024/259315, including any racemic mixtures of enantiomer pairs of such compounds as described therein, which Formulas and Table 1 compounds and racemic mixtures thereof listed therein are hereby incorporated herein by reference for all purposes.

In another embodiment, the cationic lipid is selected from any compound having a structure set forth in Formula (I), (Ia) or (Ib), and Compound Nos. I-1 to I-25 as specifically exemplified in Table 1 of PCT Publication No. WO2024/259322, including any racemic mixtures of enantiomer pairs of such compounds as described therein, which Formulas and Table 1 compounds and racemic mixtures thereof listed therein are hereby incorporated herein by reference for all purposes.

In another embodiment, the cationic lipid is selected from any compound having a structure set forth in Formula (I), and Compound Nos. I-1 to I-52 as specifically exemplified in Table 1 of PCT Publication No. WO2024/259356, including any racemic mixtures of enantiomer pairs of such compounds as described therein, which Formulas and Table 1 compounds and racemic mixtures thereof listed therein are hereby incorporated herein by reference for all purposes.

In another embodiment, the cationic lipid is selected from any compound having a structure set forth in Formula (I), (Ia), (Ib), (Ic), and Compound Nos. 1-71 as specifically exemplified in Examples 1-71 of PCT Publication No. WO2024/095179, including any racemic mixtures of enantiomer pairs of such compounds as described therein, which formulas and compounds and racemic mixtures thereof described therein are hereby incorporated herein by reference for all purposes.

In another embodiment, the cationic lipid is selected from any compound having a structure set forth in Formula (I), (Ia), (Ib), and Compound Nos. 1-8 as specifically exemplified in Examples 1-8 of PCT Publication No. WO2024/161249, including any racemic mixtures of enantiomer pairs of such compounds as described therein, which formulas and compounds and racemic mixtures thereof described therein are hereby incorporated herein by reference for all purposes.

In another embodiment, the cationic lipid is selected from any compound having a structure set forth in Formula (I), (Ia), and Compound Nos. 1-11 as specifically exemplified in Examples 1-11 of PCT Application No. PCT/IB2024/062593, including any racemic mixtures of enantiomer pairs of such compounds as described therein, which formulas and compounds and racemic mixtures thereof described therein are hereby incorporated herein by reference for all purposes.

In a further embodiment, the cationic lipid is selected from any compound having a structure set forth in Formula (I), (Ia), (Ib), and Compound Nos. 1-11 as specifically exemplified in Examples 1-11 of PCT Application No. PCT/IB2024/062587, including any racemic mixtures of enantiomer pairs of such compounds as described therein, which formulas and compounds and racemic mixtures thereof described therein are hereby incorporated herein by reference for all purposes.

In some aspects, 1, 2, 3, 4, 5, or more of the foregoing cationic lipids may be excluded from the LNPs of the present disclosure.

In some aspects, the immunogenic compositions comprise a cationic lipid, a RNA molecule as described herein and one or more of neutral lipids, steroids, pegylated lipids, or combinations thereof. If more than one cationic lipid is incorporated within the LNP, such percentages apply to the combined cationic lipids. In one aspect, the cationic lipid is present in the LNP in an amount such as at least, at most, exactly, or between (inclusive or exclusive) of, or about 40, 41, 42, 43, 44, 45, 46, 47, 48, 49, 50, 51, 52, 53, 54, 55, 56, 57, 58, 59 or 60 mole percent (mol %). In some aspects, two or more cationic lipids are incorporated within the LNP. If more than one cationic lipid is incorporated within the LNP, the foregoing percentages apply to the combined cationic lipids.

In some aspects of the disclosure the LNP comprises a combination or mixture of any the lipids described above.

### Polymer Conjugated Lipid

In some aspects, the LNPs comprise a polymer conjugated lipid. The term “polymer conjugated lipid” refers to a molecule comprising both a lipid portion and a polymer portion. An example of a polymer conjugated lipid is a pegylated lipid (e.g., polyethylene glycol-lipid, PEG-lipid). In certain aspects, the LNP comprises an additional, stabilizing lipid that is a pegylated lipid. The term “pegylated lipid” refers to a molecule comprising both a lipid portion and a polyethylene glycol portion.

Pegylated lipids are known in the art and include, but are not limited to, PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols, 2-[(polyethylene glycol)-2000]-N,N-ditetradecylacetamide, and mixtures thereof. Representative polyethylene glycol-lipids include PEG-c-DOMG, PEG-c-DMA, PEG-DSG, PEG-DPG, and PEG-s-DMG (1-(monomethoxy-polyethyleneglycol)-2,3-dimyristoylglycerol). In one aspect, the polyethylene glycol-lipid is N-[(methoxy polyethylene glycol) 2000) carbamoyl]-1,2-dimyristyloxlpropyl-3-amine (PEG-c-DMA). In one aspect, the polyethylene glycol-lipid is PEG-2000-DMG. In one aspect, the polyethylene glycol-lipid is PEG-c-DOMG. In other aspects, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanolamine (PEG-PE), a PEG succinate diacylglycerol (PEG-S-DAG) such as 4-O-(2′,3′-di(tetradecanoyloxy)propyl-1-O—((O-methoxy (polyethoxy)ethyl) butanedioate (PEG-S-DMG), a PEGylated ceramide (PEG-cer), or a PEG dialkoxypropylcarbamate such as co-methoxy (polyethoxy)ethyl-N-(2,3di(tetradecanoxy)propyl)carbamate or 2,3-di(tetradecanoxy)propyl-N-(ω-methoxy (polyethoxy)ethyl)carbamate. PEG-lipids are disclosed in, e.g., U.S. Pat. No. 9,737,619, the full disclosures of which is herein incorporated by reference in its entirety for all purposes. In some aspects, 1, 2, 3, 4, 5, or more of the foregoing pegylated lipids may be excluded from the LNPs of the present disclosure.

In some aspects, the composition comprises a pegylated lipid having the following structure:

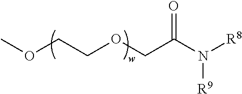

or a pharmaceutically acceptable salt, tautomer, or stereoisomer thereof, wherein:
   R⁸ and R⁹ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In some aspects, R⁸ and R⁹ are each independently straight, saturated alkyl chains containing from 12 to 16 carbon atoms. In some aspects, w has a mean value ranging from 43 to 53. In other aspects, the average w is or is about 45. In other different embodiments, the average w is or is about 49.

In some aspects, the lipid nanoparticles comprise a polymer conjugated lipid. In one aspect, the lipid nanoparticle comprises 2-[(polyethylene glycol)-2000]-N,N-ditetradecylacetamide (ALC-0159), having the formula:

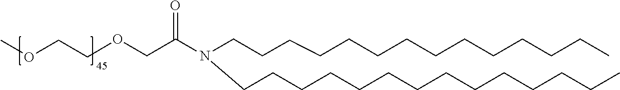

In various aspects, the molar ratio of the cationic lipid to the pegylated lipid ranges from or from about 100:1 to about 20:1, e.g., 20:1, 25:1, 30:1, 35:1, 40:1, 45:1, 50:1, 55:1, 60:1, 65:1, 70:1, 75:1, 80:1, 85:1, 90:1, 95:1, or 100:1, or any range or value derivable therein.

In certain aspects, the PEG-lipid is or is not present in the LNP in an amount from or from about 1 to about 10 mole percent (mol %) (e.g., at least, at most, exactly, or between (inclusive or exclusive) of 1, 2, 3, 4, 5, 6, 7, 8, 9, or 10 mol %), relative to the total lipid content of the nanoparticle.

In some aspects, the ratio of PEG in the lipid nanoparticle formulations may be increased or decreased and/or the carbon chain length of the PEG lipid may be modified to alter the pharmacokinetics and/or biodistribution of the lipid nanoparticle formulations.

### Additional Lipids

In certain aspects, the LNP comprises one or more additional lipids or lipid-like materials that stabilize particles during their formation. Suitable stabilizing or structural lipids include non-cationic lipids, e.g., neutral lipids and anionic lipids. Without being bound by any theory, optimizing the formulation of LNPs by addition of other hydrophobic moieties, such as cholesterol and lipids, in addition to an ionizable/cationic lipid or lipid-like material may enhance particle stability and efficacy of nucleic acid delivery.

As used herein, an “anionic lipid” refers to any lipid that is negatively charged at a selected pH. The term “neutral lipid” refers to any one of a number of lipid species that exist in either an uncharged or neutral zwitterionic form at physiological pH. In some aspects, additional lipids comprise one of the following neutral lipid components: (1) a phospholipid, (2) cholesterol or a derivative thereof; or (3) a mixture of a phospholipid and cholesterol or a derivative thereof.

Representative neutral lipids include phosphatidylcholines, phosphatidylethanolamines, phosphatidylglycerols, phosphatidic acids, phosphatidylserines, ceramides, sphingomyelins, dihydro-sphingomyelins, cephalins, and cerebrosides. Exemplary phospholipids include, for example, phosphatidylcholines, e.g., diacylphosphatidylcholines, such as (DSPC), dioleoylphosphatidylcholine (DOPC), distearoylphosphatidylcholine dimyristoylphosphatidylcholine (DMPC), dipentadecanoylphosphatidylcholine, dilauroylphosphatidylcholine, dipalmitoylphosphatidylcholine (DPPC), dioleoylphosphatidylglycerol (DOPG), dipalmitoylphosphatidylglycerol (DPPG), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), and 1-hexadecyl-sn-glycero-3-phosphocholine (C16 Lyso PC); and phosphatidylethanolamines, e.g., diacylphosphatidylethanolamines, such as dioleoyl-phosphatidylethanolamine (DOPE), 1,2-diundecanoyl-sn-glycero-phosphocholine (DUPC), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), dilauroyl-phosphatidylethanolamine (DLPE), distearoyl-phosphatidylethanolamine (DSPE), 1-phytanoyl-phosphatidylethanolamine (DpyPE), 16-O-monomethyl PE, 16-O-dimethyl PE, 18-1-trans PE, 1-stearoyl-2-oleoylphosphatidyethanolamine (SOPE), 1,2-dielaidoyl-sn-glycero-3-phosphoethanolamine (transDOPE), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In some aspects, 1, 2, 3, 4, 5, or more of the foregoing neutral lipids may be excluded from the LNPs of the present disclosure.

In one aspect, the neutral lipid is 1,2-distearoyl-sn-glycero-3-phosphocholine (DSPC), having the formula:

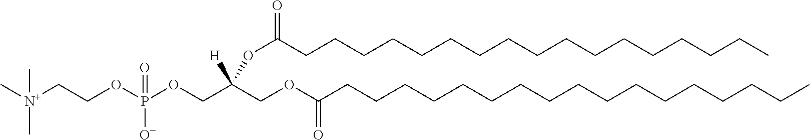 [SMILES: CCCCCCCCCCCCCCCCCC(=O)OC[C@H](COP(=O)([O-])OCC[N+](C)(C)C)OC(=O)CCCCCCCCCCCCCCCCC]

In some aspects, the LNPs comprise a neutral lipid, and the neutral lipid comprises one or more of DSPC, DPPC, DMPC, DOPC, POPC, DOPE, and/or SM. In some aspects, 1, 2, 3, 4, 5, or more of the foregoing neutral lipids may be excluded from the LNPs of the present disclosure.

In various aspects, the LNPs further comprise a steroid or steroid analogue. A “steroid” is a compound comprising the following carbon skeleton:

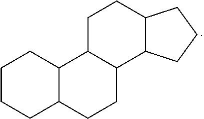 [SMILES: C1CCC2C(C1)CCC1C3CCCC3CCC21]

In certain aspects, the steroid or steroid analogue is cholesterol, fecosterol, sitosterol, ergosterol, campesterol, stigmasterol, brassicasterol, tomatidine, ursolic acid, alpha-tocopherol, and mixtures thereof. In some aspects, 1, 2, 3, 4, 5, or more of the foregoing steroid or steroid analogues may be excluded from the LNPs of the present disclosure. In certain aspects, the steroid or steroid analogue is cholesterol. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof. In some aspects, 1, 2, 3, 4, 5, or more of the foregoing cholesterol derivatives may be excluded from the LNPs of the present disclosure. In one aspect, the cholesterol has the formula:

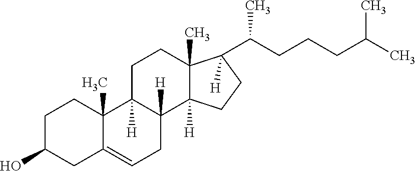

Without being bound by any theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some aspects, the molar ratio of the cationic lipid to the neutral lipid ranges from or from about 2:1 to about 8:1, or from or from about 10:0 to about 1:9, about 4:1 to about 1:2, or about 3:1 to about 1:1. In another aspect, the molar ratio of the cationic lipid to cholesterol ranges from or from about 2:1 to 1:1. In a further aspect, the molar ratio of the cationic lipid to the pegylated lipid ranges from or from about 100:1 to about 10:1 or from about 100:1 to about 25.1.

mRNA Vaccines

The present disclosure relates to mRNA vaccines in general. Several mRNA vaccine platforms are available in the prior art. The basic structure of in vitro transcribed (IVT) mRNA closely resembles “mature” eukaryotic mRNA and includes (i) a protein-encoding open reading frame (ORF), flanked by (ii) 5′ and 3′ untranslated regions (UTRs), and at the end sides (iii) a 7-methyl guanosine 5′ cap structure and (iv) a 3′ poly(A) tail. The non-coding structural features play important roles in the pharmacology of mRNA and can be individually optimized to modulate the mRNA stability, translation efficiency, and immunogenicity. By incorporating modified nucleosides, mRNA transcripts referred to as “nucleoside-modified mRNA” or “modRNA” can be produced with reduced immunostimulatory activity, and therefore an improved safety profile can be obtained. In addition, modified nucleosides allow the design of mRNA vaccines with strongly enhanced stability and translation capacity, as they can avoid the direct antiviral pathways that are induced by type IFNs and are programmed to degrade and inhibit invading mRNA. For instance, the replacement of uridine with pseudouridine in IVT of mRNA reduces the activity of 2′-5′-oligoadenylate synthetase, which regulates the mRNA cleavage by RNase L. In addition, lower activities are measured for protein kinase R, an enzyme that is associated with the inhibition of the mRNA translation process.

Besides the incorporation of modified nucleotides, other approaches have been validated to increase the translation capacity and stability of mRNA. One example is the development of “sequence-engineered mRNA”. Here, mRNA expression can be strongly increased by sequence optimizations in the ORF and UTRs of mRNA, for instance by enriching the GC content, or by selecting the UTRs of long-lived mRNA molecules.

A “5′ untranslated region” (UTR) refers to a region of an mRNA that is directly upstream (e.g., 5′) from the start codon (e.g., the first codon of an mRNA transcript translated by a ribosome) that does not encode a polypeptide of an immunogen of interest. 5′ UTRs may contain various regulatory elements, e.g., 5′ cap structure, stem-loop structure, and an internal ribosome entry site (IRES), which may play a role in the control of translation initiation.

A “3′ untranslated region” (UTR) refers to a region of an mRNA that is directly downstream (e.g., 3′) from the stop codon (e.g., the codon of an mRNA transcript that signals a termination of translation) that does not encode a polypeptide of an immunogen of interest. The 3′ UTR may be involved in regulatory processes including transcript cleavage, stability and polyadenylation, translation, and mRNA localization.

In some embodiments, the UTR is derived from an mRNA that is naturally abundant in a specific tissue (e.g., lymphoid tissue), to which the mRNA expression is targeted. In some embodiments, the UTR increases protein synthesis. Without being bound by mechanism or theory, the UTR may increase protein synthesis by increasing the time that the mRNA remains in translating polysomes (message stability) and/or the rate at which ribosomes initiate translation on the message (message translation efficiency). According, the UTR sequence may prolong protein synthesis in a tissue-specific manner. In some embodiments, the 5′ UTR and the 3′ UTR sequences are computationally derived. In some embodiments, the 5′ UTR and the 3′ UTRs are derived from a naturally abundant mRNA in a tissue. The tissue may be, for example, liver, a stem cell, or lymphoid tissue. The lymphoid tissue may include, for example, any one of a lymphocyte (e.g., a B-lymphocyte, a helper T-lymphocyte, a cytotoxic T-lymphocyte, a regulatory T-lymphocyte, or a natural killer cell), a macrophage, a monocyte, a dendritic cell, a neutrophil, an eosinophil and a reticulocyte. In some embodiments, the 5′ UTR and the 3′ UTR are derived from an alphavirus. In some embodiments, the 5′ UTR and the 3′ UTR are from a wild-type alphavirus. Examples of alphaviruses are described below.

In some embodiments, the first RNA molecule includes a 5′ UTR and the 3′ UTR derived from a naturally abundant mRNA in a tissue. In some embodiments, the first RNA molecule includes a 5′ UTR and the 3′ UTR derived from an alphavirus. In some embodiments, the second mRNA or the saRNA molecule includes a 5′ UTR and the 3′ UTR derived from an alphavirus. In some embodiments, the second mRNA or the saRNA molecule includes a 5′ UTR and the 3′ UTR from a wild-type alphavirus. In some embodiments, the RNA molecule includes a 5′ cap.

In some embodiments, the RNA molecule includes a 5′UTR or 3′UTR as described in WO2024/154061, which is hereby incorporated herein by reference in its entirety, including all sequences set forth therein.

In some embodiments, the RNA molecule comprises a 5′ UTR having the sequence of any one of SEQ ID NOs: 4-11. In some embodiments, the RNA molecule comprises a 3′ UTR having the sequence of any one of SEQ ID NOs: 12-17.

In some embodiments, the mRNA or saRNA molecule described herein includes a 5′ cap. In some embodiments, the 5′-cap moiety is a natural 5′-cap. A “natural 5′-cap” is defined as a cap that includes 7-methylguanosine connected to the 5′ end of an mRNA molecule through a 5′ to 5′ triphosphate linkage. In some embodiments, the 5′-cap moiety is a 5′-cap analog. In some embodiments, the 5′ end of the RNA is capped with a modified ribonucleotide with the structure m7G (5′) ppp (5′) N (cap 0 structure) or a derivative thereof, which may be incorporated during RNA synthesis (e.g., co-transcriptional capping) or may be enzymatically engineered after RNA transcription (e.g., post-transcriptional capping), wherein “N” is any ribonucleotide. In some embodiments, the 5′ end of the RNA molecule is capped with a modified ribonucleotide via an enzymatic reaction after RNA transcription.

In some embodiments, the RNA molecule includes a 5′ cap as described in WO2024/256962, which is hereby incorporated herein by reference in its entirety.

Also, several modifications have been implemented at the end structures of mRNA. Anti-reverse cap (ARCA) modifications can ensure the correct cap orientation at the 5′ end, which yields almost complete fractions of mRNA that can efficiently bind the ribosomes. Other cap modifications, such as phosphorothioate cap analogs, can further improve the affinity towards the eukaryotic translation initiation factor 4E, and increase the resistance against the RNA decapping complex.

Conversely, by modifying its structure, the potency of mRNA to trigger innate immune responses can be further improved, but to the detriment of translation capacity. By stabilizing the mRNA with either a phosphorothioate backbone, or by its precipitation with the cationic protein protamine, antigen expression can be diminished, but stronger immune-stimulating capacities can be obtained.

In one aspect the disclosure relates to an immunogenic composition comprising an mRNA molecule that encodes one or more polypeptides or fragments thereof of an immunogen.

In some embodiments, the mRNA molecule comprises a nucleoside-modified mRNA. mRNA useful in the disclosure typically include a first region of linked nucleosides encoding a polypeptide of interest (e.g., a coding region), a first flanking region located at the 5′-terminus of the first region (e.g., a 5′-UTR), a second flanking region located at the 3′-terminus of the first region (e.g., a 3′-UTR), at least one 5′-cap region, and a 3′-stabilizing region. In some embodiments, the mRNA of the disclosure further includes a poly-A region or a Kozak sequence (e.g., in the 5′-UTR). In some cases, mRNA of the disclosure may contain one or more intronic nucleotide sequences capable of being excised from the polynucleotide. In some embodiments, mRNA of the disclosure may include a 5′ cap structure, a chain terminating nucleotide, a stem loop, a poly A sequence, and/or a polyadenylation signal. Any one of the regions of a nucleic acid may include one or more alternative components (e.g., an alternative nucleoside). For example, the 3′-stabilizing region may contain an alternative nucleoside such as an L-nucleoside, an inverted thymidine, or a 2′-O-methyl nucleoside and/or the coding region, 5′-UTR, 3′-UTR, or cap region may include an alternative nucleoside such as a 5-substituted uridine (e.g., 5-methoxyuridine), a 1-substituted pseudouridine (e.g., 1-methyl-pseudouridine), and/or a 5-substituted cytidine (e.g., 5-methyl-cytidine).

The 5′ and 3′ UTRs may be operably linked to an open reading frame (ORF), which may be a sequence of codons derived from a gene of interest that is capable of being translated into a polypeptide of interest. As stated above, the RNA molecule may include one (monocistronic), two (bicistronic) or more (multicistronic) open reading frames (ORFs).

An “open reading frame” is a continuous stretch of DNA beginning with a start codon (e.g., methionine (ATG)), and ending with a stop codon (e.g., TAA, TAG or TGA) and encodes a polypeptide of an immunogen of interest.

In some embodiments, the ORF encodes a non-structural viral gene. In some embodiments, the ORF further includes one or more subgenomic promoters. In some embodiments, the RNA molecule includes a subgenomic promoter operably linked to the ORF. In some embodiments, the subgenomic promoter comprises a cis-acting regulatory element. In some embodiments, the cis-acting regulatory element is immediately downstream (5′-3′) of B². In some embodiments, the cis-acting regulatory element is immediately downstream (5′-3′) of a guanine that is immediately downstream of B². In some embodiments, the cis-acting regulatory element is an AU-rich element. In some embodiments, the AU-rich element is au, auaaaagau, auaaaaagau, auag, auauauauau, auauauau, auauauauauau, augaugaugau, augau, auaaaagaua, or auaaaagaug. In some embodiments, the second mRNA or the saRNA molecule may include (i) an ORF encoding a replicase which may transcribe RNA from the second mRNA or the saRNA molecule and (ii) an ORF encoding at least one an antigen or polypeptide of interest. The polymerase may be an alphavirus replicase e.g., including any one of the non-structural alphavirus proteins nsP1, nsP2, nsP3 and nsP4, or a combination thereof. In some embodiments, the RNA molecule includes alphavirus nonstructural protein nsP1. In some embodiments, the RNA molecule includes alphavirus nonstructural protein nsP2. In some embodiments, the RNA molecule includes alphavirus nonstructural protein nsP3. In some embodiments, the RNA molecule includes alphavirus nonstructural protein nsP4. In some embodiments, the RNA molecule includes alphavirus nonstructural proteins nsP1, nsP2, and nsP3. In some embodiments, the RNA molecule includes alphavirus nonstructural proteins nsP1, nsP2, nsP3, and nsP4. In some embodiments, the RNA molecule includes any combination of nsP1, nsP2, nsP3, and nsP4. In some embodiments, the RNA molecule does not include nsP4.

In some embodiments, an open reading frame of an RNA (e.g., modified mRNA or saRNA) composition is codon-optimized. In some embodiments, the open reading frame from which the polypeptide or fragment thereof is encoded is codon-optimized.

The compositions described herein comprise at least one RNA polynucleotide, such as a mRNA (e.g., modified mRNA). mRNA, for example, is transcribed in vitro from template DNA, referred to as an “in vitro transcription template.” In some embodiments, an in vitro transcription template encodes a 5′ untranslated (UTR) region, contains an open reading frame (ORF), and encodes a 3′ UTR and a polyA tail. The particular nucleic acid sequence composition and length of an in vitro transcription template will depend on the mRNA encoded by the template.

A “polyA tail” is a region of mRNA that is downstream, e.g., directly downstream (e.g., 3′), from the 3′ UTR which contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, export of the mRNA from the nucleus and translation.

In preferred embodiments, the 3′ polyadenylation tail comprises about 80 adenosine monophosphates (e.g. 80A).

In some embodiments, the RNA molecule includes a polyA tail as described in WO2024/256962, which is hereby incorporated herein by reference in its entirety.

In some embodiments, the composition comprises a second LNP, wherein the second LNP does not encapsulate a polynucleotide as described in WO2023057930, the entirety of which is hereby incorporated herein by reference.

In some aspects, the compositions and methods described herein relate to frozen or lyophilized lipid nanoparticles encapsulating or associated with RNA in the presence of a cryoprotectant, preferably a carbohydrate cryoprotectant, and/or further in the presence of lipid nanoparticles that are devoid of nucleic acid, (e.g., not encapsulating and not associated with RNA (also referred herein as “blank” LNPs), or liposomes, or a higher cryoprotectant concentration) resulting in a composition comprising LNPs encapsulating RNA or associated with RNA that is characterized by, among other things, an improved integrity of the RNA after completion of the respective freezing or lyophilization process and which is further characterized by increased storage stability, such as, for example, with respect to storage for extended periods and/or under non-cooling conditions, as compared to a composition comprising lipid nanoparticles encapsulating or associated with RNA in the absence of the blank LNPs, or liposomes, or a higher cryoprotectant concentration when assessed under identical conditions. In other words, in some aspects, the compositions include a mixture of a first lipid nanoparticle encapsulating or associated with RNA, a second lipid nanoparticle that is devoid of nucleic acid, and a cryoprotectant that results in improved characteristics of the encapsulated RNA after freezing or lyophilization processes, preferably for use as a pharmaceutical composition, such as, for example, an immunogenic composition or vaccine. In some aspects, the compositions include a mixture of a first lipid nanoparticle encapsulating or associated with RNA and an increased cryoprotectant concentration that results in improved characteristics of the encapsulated RNA after freezing or lyophilization processes, preferably for use as a pharmaceutical composition, such as, for example, an immunogenic composition or vaccine. In some aspects, the compositions include a mixture of a first lipid nanoparticle encapsulating or associated with RNA and a second lipid nanoparticle that is devoid of nucleic acid that results in improved characteristics of the encapsulated RNA after freezing or lyophilization processes, preferably for use as a pharmaceutical composition, such as, for example, an immunogenic composition or vaccine. In some aspects, the compositions include a mixture of a first lipid nanoparticle encapsulating or associated with RNA and a liposome that results in improved characteristics of the encapsulated RNA after freezing or lyophilization processes, preferably for use as a pharmaceutical composition, such as, for example, an immunogenic composition or vaccine. In some aspects, the compositions include a mixture of a first lipid nanoparticle encapsulating or associated with RNA, a liposome, and an increased cryoprotectant concentration that result in improved characteristics of the encapsulated RNA after freezing or lyophilization processes, preferably for use as a pharmaceutical composition, such as, for example, an immunogenic composition or vaccine.

In some aspects, the compositions and methods described herein relate to liquid (never frozen), frozen or lyophilized lipid nanoparticles encapsulating or associated with RNA in the presence of cholesterol or a cholesterol analog, and further in the presence of a fatty acid or derivative or salt thereof resulting in a composition comprising LNPs encapsulating RNA or associated with RNA that is characterized by, among other things, an improved integrity of the RNA and quality of the lipid nanoparticle after completion of the respective freezing and thawing process or lyophilization process, and which is also characterized by increased storage stability, such as, for example, with respect to storage for extended periods and/or under non-cooling conditions, and is further characterized by a decrease in lipid adduct formation, as compared to a composition comprising lipid nanoparticles encapsulating or associated with RNA in the absence of the cholesterol analog and the fatty acid or derivative or salt thereof when assessed under identical conditions, preferably for use as a pharmaceutical composition, such as, for example, an immunogenic composition or vaccine. In addition, the composition and methods described herein result in a lyophilized, frozen, or liquid lipid nanoparticle drug product capable of combination with other product modalities, including but not limited to for example, subunit protein vaccines, conjugate vaccines or adjuvants, while maintaining the quality attributes of the original LNP particle, and which is also characterized by increased storage stability preferably for use as a pharmaceutical composition, such as, for example, an immunogenic composition or vaccine.

Advantageously, the compositions and methods thereof described herein are suitable for use at an industrial scale. The methods described herein may be used to produce, for example, a liquid (never frozen), frozen or lyophilized composition comprising LNPs encapsulating or associated with RNA having the above-mentioned properties in a reproducible and cost-effective manner. The composition comprising LNPs encapsulating or associated with RNA may advantageously be stored, shipped and applied, e.g., as a vaccine, without a cold chain, while the integrity and the biological activity of the RNA in the composition remain unexpectedly high.

mRNA of the disclosure may include one or more naturally occurring components, including any of the canonical nucleotides A (adenosine), G (guanosine), C (cytosine), U (uridine), or T (thymidine). In one embodiment, all or substantially all of the nucleotides comprising (a) the 5′-UTR, (b) the open reading frame (ORF), (c) the 3′-UTR, (d) the poly A tail, and any combination of (a, b, c, or d above) comprise naturally occurring canonical nucleotides A (adenosine), G (guanosine), C (cytosine), U (uridine), or T (thymidine).

mRNA of the disclosure may include one or more alternative components, as described herein, which impart useful properties including increased stability and/or the lack of a substantial induction of the innate immune response of a cell into which the polynucleotide is introduced. For example, a modRNA may exhibit reduced degradation in a cell into which the modRNA is introduced, relative to a corresponding unaltered mRNA. These alternative species may enhance the efficiency of protein production, intracellular retention of the polynucleotides, and/or viability of contacted cells, as well as possess reduced immunogenicity.

mRNA of the disclosure may include one or more modified (e.g., altered or alternative) nucleobases, nucleosides, nucleotides, or combinations thereof. The mRNA useful in a LNP can include any useful modification or alteration, such as to the nucleobase, the sugar, or the internucleoside linkage (e.g., to a linking phosphate/to a phosphodiester linkage/to the phosphodiester backbone). In certain embodiments, alterations (e.g., one or more alterations) are present in each of the nucleobase, the sugar, and the internucleoside linkage. Alterations according to the present disclosure may be alterations of ribonucleic acids (RNAs), e.g., the substitution of the 2′-OH of the ribofuranosyl ring to 2′-H, threose nucleic acids (TNAs), glycol nucleic acids (GNAs), peptide nucleic acids (PNAs), locked nucleic acids (LNAs), or hybrids thereof. Additional alterations are described herein.

mRNA of the disclosure may or may not be uniformly altered along the entire length of the molecule. For example, one or more or all types of nucleotide (e.g., purine or pyrimidine, or any one or more or all of A, G, U, C) may or may not be uniformly altered in a mRNA, or in a given predetermined sequence region thereof. In some instances, all nucleotides X in a mRNA (or in a given sequence region thereof) are altered, wherein X may any one of nucleotides A, G, U, C, or any one of the combinations A+G, A+U, A+C, G+U, G+C, U+C, A+G+U, A+G+C, G+U+C or A+G+C.

Different sugar alterations and/or internucleoside linkages (e.g., backbone structures) may exist at various positions in a polynucleotide. One of ordinary skill in the art will appreciate that the nucleotide analogs or other alteration(s) may be located at any position(s) of a polynucleotide such that the function of the polynucleotide is not substantially decreased. An alteration may also be a 5′- or 3′-terminal alteration. In some embodiments, the polynucleotide includes an alteration at the 3′-terminus. The polynucleotide may contain from about 1% to about 100% alternative nucleotides (either in relation to overall nucleotide content, or in relation to one or more types of nucleotide, e.g., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of a canonical nucleotide (e.g., A, G, U, or C).

Polynucleotides may contain at a minimum zero and at maximum 100% alternative nucleotides, or any intervening percentage, such as at least 5% alternative nucleotides, at least 10% alternative nucleotides, at least 25% alternative nucleotides, at least 50% alternative nucleotides, at least 80% alternative nucleotides, or at least 90% alternative nucleotides. For example, polynucleotides may contain an alternative pyrimidine such as an alternative uracil or cytosine. In some embodiments, at least 5%, at least 10%, at least 25%, at least 50%, at least 80%, at least 90% or 100% of the uracil in a polynucleotide is replaced with an alternative uracil (e.g., a 5-substituted uracil). The alternative uracil can be replaced by a compound having a single unique structure or can be replaced by a plurality of compounds having different structures (e.g., 2, 3, 4 or more unique structures). In some instances, at least 5%, at least 10%, at least 25%, at least 50%, at least 80%, at least 90% or 100% of the cytosine in the polynucleotide is replaced with an alternative cytosine (e.g., a 5-substituted cytosine). The alternative cytosine can be replaced by a compound having a single unique structure or can be replaced by a plurality of compounds having different structures (e.g., 2, 3, 4 or more unique structures).

In some instances, nucleic acids do not substantially induce an innate immune response of a cell into which the polynucleotide (e.g., mRNA) is introduced. Features of an induced innate immune response include 1) increased expression of pro-inflammatory cytokines, 2) activation of intracellular PRRs (RIG-I, MDA5, etc., and/or 3) termination or reduction in protein translation.

The mRNA may include a 5′-cap structure. The 5′-cap structure of a polynucleotide is involved in nuclear export and increasing polynucleotide stability and binds the mRNA Cap Binding Protein (CBP), which is responsible for polynucleotide stability in the cell and translation competency through the association of CBP with poly-A binding protein to form the mature cyclic mRNA species. The cap further assists the removal of 5′-proximal introns removal during mRNA splicing.

Endogenous polynucleotide molecules may be 5′-end capped generating a 5′-ppp-5′-triphosphate linkage between a terminal guanosine cap residue and the 5′-terminal transcribed sense nucleotide of the polynucleotide. This 5′-guanylate cap may then be methylated to generate an N7-methyl-guanylate residue. The ribose sugars of the terminal and/or anteterminal transcribed nucleotides of the 5′end of the polynucleotide may optionally also be 2′-O-methylated. 5′-decapping through hydrolysis and cleavage of the guanylate cap structure may target a polynucleotide molecule, such as an mRNA molecule, for degradation.

Alterations to polynucleotides may generate a non-hydrolyzable cap structure preventing decapping and thus increasing polynucleotide half-life. Because cap structure hydrolysis requires cleavage of 5′-ppp-5′ phosphorodiester linkages, alternative nucleotides may be used during the capping reaction. For example, a Vaccinia Capping Enzyme from New England Biolabs (Ipswich, MA) may be used with a-thio-guanosine nucleotides according to the manufacturer's instructions to create a phosphorothioate linkage in the 5′-ppp-5′ cap.

Additional alternative guanosine nucleotides may be used such as a-methylphosphonate and seleno-phosphate nucleotides. Additional alterations include, but are not limited to, 2′-O-methylation of the ribose sugars of 5′-terminal and/or 5′-anteterminal nucleotides of the polynucleotide (as mentioned above) on the 2′-hydroxy group of the sugar. Multiple distinct 5′-cap structures can be used to generate the 5′-cap of an mRNA molecule.

Cap analogs, which herein are also referred to as synthetic cap analogs, chemical caps, chemical cap analogs, or structural or functional cap analogs, differ from natural (e.g., endogenous, wild-type, or physiological) 5′-caps in their chemical structure, while retaining cap function. Cap analogs may be chemically (e.g., non-enzymatically) or enzymatically synthesized and/linked to a polynucleotide. For example, the Anti-Reverse Cap Analog (ARCA) cap contains two guanosines linked by a 5′-5′-triphosphate group, wherein one guanosine contains an N7-methyl group as well as a 3′-O-methyl group (e.g., N7, ‘-O-dimethyl-guanosine-5’-triphosphate-5 ‘-guanosine, m7G-3’mppp-G, which may equivalently be designated 3′ O-Me-m7G(5′)ppp(5′)G). The 3′-O atom of the other, unaltered, guanosine becomes linked to the 5′-terminal nucleotide of the capped polynucleotide (e.g., an mRNA). The N7- and 3′-O-methylated guanosine provides the terminal moiety of the capped polynucleotide (e.g., mRNA). Another exemplary cap is mCAP, which is similar to ARCA but has a 2′-O-methyl group on guanosine (e.g., N7,2′-O-dimethyl-guanosine-5′-triphosphate-5′-guanosine, m7Gm-ppp-G).

A cap may be a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the cap structures of which are herein incorporated by reference.

Alternatively, a cap analog may be a N7-(4-chlorophenoxy ethyl) substituted dinucleotide cap analog known in the art and/or described herein. Non-limiting examples of N7-(4-chlorophenoxy ethyl) substituted dinucleotide cap analogs include a N7-(4-chlorophenoxyethyl)-G(5)ppp(5′)G and a N7-(4-chlorophenoxyethyl)-m3′-OG(5)ppp(5′)G cap analog (see, e.g., the various cap analogs and the methods of synthesizing cap analogs described in Kore et al. Bioorganic & Medicinal Chemistry 2013 21:4570-4574; the cap structures of which are herein incorporated by reference). In other instances, a cap analog useful in the polynucleotides of the present disclosure is a 4-chloro/bromophenoxy ethyl analog.

While cap analogs allow for the concomitant capping of a polynucleotide in an in vitro transcription reaction, up to 20% of transcripts remain uncapped. This, as well as the structural differences of a cap analog from endogenous 5′-cap structures of polynucleotides produced by the endogenous, cellular transcription machinery, may lead to reduced translational competency and reduced cellular stability.

Alternative polynucleotides may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function, and/or structure as compared to synthetic features or analogs of the prior art, or which outperforms the corresponding endogenous, wild-type, natural, or physiological feature in one or more respects.

Non-limiting examples of more authentic 5′-cap structures useful in the polynucleotides of the present disclosure are those which, among other things, have enhanced binding of cap binding proteins, increased half-life, reduced susceptibility to 5′-endonucleases, and/or reduced 5′-decapping, as compared to synthetic 5′-cap structures known in the art (or to a wild-type, natural or physiological 5′-cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanosine cap nucleotide wherein the cap guanosine contains an N7-methylation and the 5′-terminal nucleotide of the polynucleotide contains a 2′-O-methyl. Such a structure is termed the CapI structure. This cap results in a higher translational-competency, cellular stability, and a reduced activation of cellular pro-inflammatory cytokines, as compared, e.g., to other 5′cap analog structures known in the art. Other exemplary cap structures include 7mG(5′)ppp(5′)N,pN2p (Cap 0), 7mG(5′)ppp(5′)NImpNp (Cap 1), 7mG(5′)-ppp(5′)NImpN2mp (Cap 2), and m(7)Gpppm(3)(6,6,2′)Apm(2′)Apm(2′)Cpm(2)(3,2′)Up (Cap 4).

Because the alternative polynucleotides may be capped post-transcriptionally, and because this process is more efficient, nearly 100% of the mRNA may be capped. This is in contrast to −80% when a cap analog is linked to a polynucleotide in the course of an in vitro transcription reaction. 5′-terminal caps may include endogenous caps or cap analogs. A 5′-terminal cap may include a guanosine analog. Useful guanosine analogs include inosine, N1-methyl-guanosine, 2′-fluoro-guanosine, 7-deaza-guanosine, 8-oxo-guanosine, 2-amino-guanosine, LNA-guanosine, and 2-azido-guanosine. In some cases, a polynucleotide contains a modified 5′-cap. A modification on the 5′-cap may increase the stability of polynucleotide, increase the half-life of the polynucleotide, and could increase the polynucleotide translational efficiency. The modified 5′-cap may include, but is not limited to, one or more of the following modifications: modification at the 2′- and/or 3′-position of a capped guanosine triphosphate (GTP), a replacement of the sugar ring oxygen (that produced the carbocyclic ring) with a methylene moiety (CH2), a modification at the triphosphate bridge moiety of the cap structure, or a modification at the nucleobase (G) moiety.

A 5′-UTR may be provided as a flanking region to the mRNA. A 5′-UTR may be homologous or heterologous to the coding region found in a polynucleotide. Multiple 5′-UTRs may be included in the flanking region and may be the same or of different sequences. Any portion of the flanking regions, including none, may be codon optimized and any may independently contain one or more different structural or chemical alterations, before and/or after codon optimization.

In one embodiment, an ORF encoding an antigen is codon optimized. Codon optimization methods are known in the art. For example, an ORF of any one or more of the sequences provided herein or incorporated by reference herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art-non-limiting examples include services from GeneArt (Life Technologies), DNA2.0 (Menlo Park Calif.) and/or proprietary methods. In some embodiments, the open reading frame (ORF) sequence is optimized using optimization algorithms. To alter one or more properties of an mRNA, 5′-UTRs which are heterologous to the coding region of an mRNA may be engineered. The mRNA may then be administered to cells, tissue or organisms and outcomes such as protein level, localization, and/or half-life may be measured to evaluate the beneficial effects the heterologous 5′-UTR may have on the mRNA. Variants of the 5′-UTRs may be utilized wherein one or more nucleotides are added or removed to the termini, including A, T, C or G. 5′-UTRs may also be codon-optimized, or altered in any manner described herein.

mRNAs may include a stem loop such as, but not limited to, a histone stem loop. The stem loop may be a nucleotide sequence that is about 25 or about 26 nucleotides in length. The histone stem loop may be located 3′-relative to the coding region (e.g., at the 3′-terminus of the coding region). As a non-limiting example, the stem loop may be located at the 3′-end of a polynucleotide described herein. In some cases, an mRNA includes more than one stem loop (e.g., two stem loops). A stem loop may be located in a second terminal region of a polynucleotide. As a non-limiting example, the stem loop may be located within an untranslated region (e.g., 3′-UTR) in a second terminal region. In some cases, a mRNA which includes the histone stem loop may be stabilized by the addition of a 3′-stabilizing region (e.g., a 3′-stabilizing region including at least one chain terminating nucleoside). Not wishing to be bound by theory, the addition of at least one chain terminating nucleoside may slow the degradation of a polynucleotide and thus can increase the half-life of the polynucleotide. In other cases, a mRNA, which includes the histone stem loop may be stabilized by an alteration to the 3′-region of the polynucleotide that can prevent and/or inhibit the addition of oligio(U). In yet other cases, a mRNA, which includes the histone stem loop may be stabilized by the addition of an oligonucleotide that terminates in a 3′-deoxynucleoside, 2′,3′-dideoxynucleoside 3′-O-methylnucleosides, 3-O-ethylnucleosides, 3′-arabinosides, and other alternative nucleosides known in the art and/or described herein. In some instances, the mRNA of the present disclosure may include a histone stem loop, a poly-A region, and/or a 5′-cap structure. The histone stem loop may be before and/or after the poly-A region. The polynucleotides including the histone stem loop and a poly-A region sequence may include a chain terminating nucleoside described herein. In other instances, the polynucleotides of the present disclosure may include a histone stem loop and a 5′-cap structure. The 5′-cap structure may include, but is not limited to, those described herein and/or known in the art. In some cases, the conserved stem loop region may include a miR sequence described herein. As a non-limiting example, the stem loop region may include the seed sequence of a miR sequence described herein. In another non-limiting example, the stem loop region may include a miR-122 seed sequence. mRNA may include at least one histone stem-loop and a poly-A region or polyadenylation signal. In certain cases, the polynucleotide encoding for a histone stem loop and a poly-A region or a polyadenylation signal may code for a pathogen antigen or fragment thereof. In other cases, the polynucleotide encoding for a histone stem loop and a poly-A region or a polyadenylation signal may code for a therapeutic protein. In some cases, the polynucleotide encoding for a histone stem loop and a poly-A region or a polyadenylation signal may code for a tumor antigen or fragment thereof. In other cases, the polynucleotide encoding for a histone stem loop and a poly-A region or a polyadenylation signal may code for an allergenic antigen or an autoimmune self-antigen.

An mRNA may include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of a nucleic acid. During RNA processing, a long chain of adenosine nucleotides (poly-A region) is normally added to messenger RNA (mRNA) molecules to increase the stability of the molecule. Immediately after transcription, the 3′-end of the transcript is cleaved to free a 3′-hydroxy. Then poly-A polymerase adds a chain of adenosine nucleotides to the RNA. The process, called polyadenylation, adds a poly-A region that is between 100 and 250 residues long. Unique poly-A region lengths may provide certain advantages to the alternative polynucleotides of the present disclosure. Generally, the length of a poly-A region of the present disclosure is at least 30 nucleotides in length. In another embodiment, the poly-A region is at least 35 nucleotides in length. In another embodiment, the length is at least 40 nucleotides. In another embodiment, the length is at least 45 nucleotides. In another embodiment, the length is at least 55 nucleotides. In another embodiment, the length is at least 60 nucleotides. In another embodiment, the length is at least 70 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1700 nucleotides. In another embodiment, the length is at least 1800 nucleotides. In another embodiment, the length is at least 1900 nucleotides. In another embodiment, the length is at least 2000 nucleotides. In another embodiment, the length is at least 2500 nucleotides. In another embodiment, the length is at least 3000 nucleotides. In some instances, the poly-A region may be 80 nucleotides, 120 nucleotides, 160 nucleotides in length on an alternative polynucleotide molecule described herein. In other instances, the poly-A region may be 20, 40, 80, 100, 120, 140 or 160 nucleotides in length on an alternative polynucleotide molecule described herein. In some cases, the poly-A region is designed relative to the length of the overall alternative polynucleotide. This design may be based on the length of the coding region of the alternative polynucleotide, the length of a particular feature or region of the alternative polynucleotide (such as mRNA) or based on the length of the ultimate product expressed from the alternative polynucleotide. When relative to any feature of the alternative polynucleotide (e.g., other than the mRNA portion which includes the poly-A region) the poly-A region may be 10, 20, 30, 40, 50, 60, 70, 80, 90 or 100% greater in length than the additional feature. The poly-A region may also be designed as a fraction of the alternative polynucleotide to which it belongs. In this context, the poly-A region may be 10, 20, 30, 40, 50, 60, 70, 80, or 90% or more of the total length of the construct or the total length of the construct minus the poly-A region.

In certain cases, engineered binding sites and/or the conjugation of mRNA for poly-A binding protein may be used to enhance expression. The engineered binding sites may be sensor sequences which can operate as binding sites for ligands of the local microenvironment of the mRNA. As a non-limiting example, the mRNA may include at least one engineered binding site to alter the binding affinity of poly-A binding protein (PABP) and analogs thereof. The incorporation of at least one engineered binding site may increase the binding affinity of the PABP and analogs thereof.

Additionally, multiple distinct mRNA may be linked together to the PABP (poly-A binding protein) through the 3′-end using alternative nucleotides at the 3′-terminus of the poly-A region. Transfection experiments can be conducted in relevant cell lines at and protein production can be assayed by ELISA at 12 hours, 24 hours, 48 hours, 72 hours, and day 7 post-transfection. As a non-limiting example, the transfection experiments may be used to evaluate the effect on PABP or analogs thereof binding affinity as a result of the addition of at least one engineered binding site. In certain cases, a poly-A region may be used to modulate translation initiation. While not wishing to be bound by theory, the poly-A region recruits PABP which in turn can interact with translation initiation complex and thus may be essential for protein synthesis. In some cases, a poly-A region may also be used in the present disclosure to protect against 3′-5′-exonuclease digestion. In some instances, an mRNA may include a polyA-G Quartet. The G-quartet is a cyclic hydrogen bonded array of four guanosine nucleotides that can be formed by G-rich sequences in both DNA and RNA. In this embodiment, the G-quartet is incorporated at the end of the poly-A region. The resultant mRNA may be assayed for stability, protein production and other parameters including half-life at various time points. It has been discovered that the polyA-G quartet results in protein production equivalent to at least 75% of that seen using a poly-A region of 120 nucleotides alone. In some cases, mRNA may include a poly-A region and may be stabilized by the addition of a 3′-stabilizing region. The mRNA with a poly-A region may further include a 5′-cap structure. In other cases, mRNA may include a poly-A-G Quartet. The mRNA with a poly-A-G Quartet may further include a 5′-cap structure. In some cases, the 3′-stabilizing region which may be used to stabilize mRNA includes a poly-A region or poly-A-G Quartet. In other cases, the 3′-stabilizing region which may be used with the present disclosure include a chain termination nucleoside such as 3′-deoxyadenosine (cordycepin), 3′-deoxyuridine, 3′-deoxycytosine, 3′-deoxyguanosine, 3′-deoxy thymine, 2′,3′-dideoxynucleosides, such as 2′,3′-dideoxyadenosine, 2′,3′-dideoxyuridine, 2′,3′-dideoxycytosine, 2′, 3′-dideoxyguanosine, 2′,3′-dideoxythymine, a 2′-deoxynucleoside, or an O-methylnucleoside. In other cases, mRNA which includes a polyA region or a poly-A-G Quartet may be stabilized by an alteration to the 3′-region of the polynucleotide that can prevent and/or inhibit the addition of oligio (U). In yet other instances, mRNA which includes a poly-A region or a poly-A-G Quartet may be stabilized by the addition of an oligonucleotide that terminates in a 3′-deoxynucleoside, 2′,3′-dideoxynucleoside 3-O-methylnucleosides, 3′-O-ethylnucleosides, 3′-arabinosides, and other alternative nucleosides known in the art and/or described herein.

The modRNA immunogenic composition is based on a modRNA platform technology. The single stranded, 5′-capped modRNA contains an open reading frame encoding the vaccine antigen of interest and features structural elements optimized for high efficacy of the RNA. The modRNA also contains a substitution of 1-methyl-pseudouridine for each uridine to decrease recognition of the vaccine RNA by innate immune sensors, such as TLRs 7 and 8, resulting in decreased innate immune activation and increased protein translation. The modRNA is encapsulated in a LNP for delivery into target cells.

### Modified Nucleobases

In some embodiments, an LNP formulation described herein comprises an encapsulated RNA polynucleotide comprising one or more alternative nucleosides or nucleotides. The alternative nucleosides and nucleotides can include an alternative nucleobase. A nucleobase of a nucleic acid is an organic base such as a purine or pyrimidine or a derivative thereof. A nucleobase may be a canonical base (e.g., adenine, guanine, uracil, thymine, and cytosine). These nucleobases can be altered or wholly replaced to provide polynucleotide molecules having enhanced properties, e.g., increased stability such as resistance to nucleases. Non-canonical or modified bases may include, for example, one or more substitutions or modifications including but not limited to alkyl, aryl, halo, oxo, hydroxyl, alkyloxy, and/or thio substitutions; one or more fused or open rings; oxidation; and/or reduction.

In some embodiments, the nucleobase is an alternative uracil. Exemplary nucleobases and nucleosides having an alternative uracil include pseudouridine (ψ), pyridin-4-one ribonucleoside, 5-aza-uracil, 6-aza-uracil, 2-thio-5-aza-uracil, 2-thio-uracil (s2U), 4-thio-uracil (s4U), 4-thio-pseudouridine, 2-thio-pseudouridine, 5-hydroxy-uracil (ho5U), 5-aminoallyl-uracil, 5-halo-uracil (e.g., 5-iodo-uracil or 5-bromo-uracil), 3-methyl-uracil (m U), 5-methoxy-uracil (mo5U), uracil 5-oxyacetic acid (cmo5U), uracil 5-oxyacetic acid methyl ester (mcmo5U), 5-carboxymethyl-uracil (cm5U), 1-carboxymethyl-pseudouridine, 5-carboxyhydroxymethyl-uracil (chm5U), 5-carboxyhydroxymethyl-uracil methyl ester (mchm5U), 5-methoxycarbonylmethyl-uracil (mcm5U), 5-methoxycarbonylmethyl-2-thio-uracil (mcm5s2U), 5-aminomethyl-2-thio-uracil (nmVu), 5-methylaminomethyl-uracil (mnm5U), 5-methylaminomethyl-2-thio-uracil (mnmVu), 5-methylaminomethyl-2-seleno-uracil (mnm5se2U), 5-carbamoylmethyl-uracil (ncm5U), 5-carboxymethylaminomethyl-uracil (cmnm5U), 5-carboxymethylaminomethyl-2-thio-uracil (cmnmVu), 5-propynyl-uracil, 1-propynyl-pseudouracil, 5-taurinomethyl-uracil (xm5U), 1-taurinomethyl-pseudouridine, 5-taurinomethyl-2-thio-uracil (xm5s2U), 1-taurinomethyl-4-thio-pseudouridine, 5-methyl-uracil (m5U, e.g., having the nucleobase deoxythymine), 1-methyl-pseudouridine (mV), 5-methyl-2-thio-uracil (m5s2U), I-methyl-4-thio-pseudouridine (m xj/), 4-thio-1-methyl-pseudouridine, 3-methyl-pseudouridine (m \|/), 2-thio-1-methyl-pseudouridine, 1-methyl-1-deaza-pseudouridine, 2-thio-I-methyl-1-deaza-pseudouridine, dihydrouracil (D), dihydropseudouridine, 5,6-dihydrouracil, 5-methyl-dihydrouracil (m5D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, N1-methyl-pseudouridine, 3-(3-amino-3-carboxypropyl) uracil (acp U), I-methyl-3-(3-amino-3-carboxypropyl) pseudouridine (acp ψ), 5-(isopentenylaminomethyl) uracil (inm5U), 5-(isopentenylaminomethyl)-2-thio-uracil (inm5s2U), 5,2′-O-dimethyl-uridine (m5Um), 2-thio-2′-O-methyl-uridine (s2Um), 5-methoxycarbonylmethyl-2′-O-methyl-uridine (mem Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m Um), and 5-(isopentenylaminomethyl)-2′-O-methyl-uridine (inm5Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(I-E-propenylamino)]uracil.

In some embodiments, the nucleobase is an alternative cytosine. Exemplary nucleobases and nucleosides having an alternative cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocytidine, 4-thio-1-methy 1-pseudoisocytidine, 4-thio-1-methyl-1-deaza-pseudoisocytidine, 1-methyl-1-deaza-pseudoisocytidine, zebularine, 5-aza-zebularine, 5-methy 1-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytosine, 5-hydroxy-cytosine, 5-(3-azidopropyl)-cytosine, and 5-(2-azidoethyl)-cytosine.

In some embodiments, the nucleobase is an alternative adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-azaadenine, 7-deaza-2-amino-purine, 7-deaza-8-aza-2-amino-purine, 7-deaza-2,6-diaminopurine, 7-deaza-8-aza-2,6-diaminopurine, 1-methy 1-adenine (ml A), 2-methyl-adenine (m2A), N6-methyl-adenine (m6A), 2-methylthio-N6-methyl-adenine (ms2m6A), N6-isopentenyl-adenine (16A), 2-methylthio-N6-isopentenyl-adenine (ms2i6A), N6-(cis-hydroxyisopentenyl) adenine (io6A), 2-methylthio-N6-(cis-hydroxyisopentenyl) adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms296A), N6, N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-O-dimethyl-adenosine (m6Am), N6, N6,2′-O-trimethyl-adenosine (m62Am), I,2′-O-dimethyl-adenosine (ml Am), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some embodiments, the nucleobase is an alternative guanine. Exemplary nucleobases and nucleosides having an alternative guanine include inosine (I), 1-methyl-inosine (miI), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQO), 7-aminomethyl-7-deaza-guanine (preQI), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (mIG), N2-methyl-guanine (m2G), N2,N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2, N2-dimethyl-6-thio-guanine, N2-methyl-2′-O-methyl-guanosine (m2Gm), N2, N2-dimethyl-2′-O-methyl-guanosine (m22Gm), 1-methyl-2′-O-methyl-guanosine (mlGm), N2,7-dimethyl-2′-O-methyl-guanosine (m2,7Gm), 2′-O-methyl-inosine (Im), 1,2′-O-dimethyl-inosine (mllm), 1-thio-guanine, and O-6-methyl-guanine.

The alternative nucleobase of a nucleotide can be independently a purine, a pyrimidine, a purine or pyrimidine analog. For example, the nucleobase can be an alternative to adenine, cytosine, guanine, uracil, or hypoxanthine. In another embodiment, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine. When the nucleotides are depicted using the shorthand A, G, C, T or U, each letter refers to the representative base and/or derivatives thereof, e.g., A includes adenine or adenine analogs, e.g., 7-deaza adenine).

Additional exemplary modified nucleotides include any one of N-1-methylpseudouridine; pseudouridine, N6-methyladenosine, 5-methylcytidine, and 5-methyluridine. In some embodiments, the modified nucleotide is N-1-methylpseudouridine. In some embodiments, the modified nucleotide is N1-Methylpseudourodine-5′-triphosphate (m1Y′TP).

In some embodiments, the RNA molecule may include phosphoramidate, phosphorothioate, and/or methylphosphonate linkages.

In some embodiments, at least 10% of a total population of a particular nucleotide in the RNA molecule has been replaced with one or more modified or unnatural nucleotides. In some embodiments, at least 25% of a total population of a particular nucleotide in the molecule has been replaced with one or more modified or unnatural nucleotides. In some embodiments, at least 50% of a total population of a particular nucleotide in the molecule has been replaced with one or more modified or unnatural nucleotides. In some embodiments, at least 75% of a total population of a particular nucleotide in the molecule has been replaced with one or more modified or unnatural nucleotides. In some embodiments, essentially all of the particular nucleotide population in the molecule has been replaced with one or more modified or unnatural nucleotides.

In some embodiments, at least a portion, or all of a total population of a particular nucleotide in the RNA molecule has been replaced with two modified or unnatural nucleotides. In some embodiments, the two modified or unnatural nucleotides are provided in a ratio equal to any one of, at least any one of, at most any one of, or between any two of 1:99 to 99:1, including 1:99; 2:98; 3:97; 4:96; 5:95; 6:94; 7:93; 8:92; 9:91; 10:90; 11:89; 12:88; 13:87; 14:86; 15:85; 16:84; 17:83; 18:82, 19:81; 20:80; 21:79; 22:78; 23:77; 24:76; 25:75; 26:74; 27:73; 28:72; 29:71; 30:70; 31:69; 32:68; 33:67; 34:66; 35:65; 36:64; 37:63; 38:62; 39:61; 40:60; 41:59; 42:58; 43:57; 44:56; 45:55; 46:54; 47:53; 48:52; 49:51; 50:50; 51:49; 52:48; 53:47; 54:46; 55:45; 56:44; 57:43; 58:42; 59:41; 60:40; 61:39; 62:38; 63:37; 64:36; 65:35; 66:34; 67:33; 68:32; 69:31; 70:30; 71:29; 72:28; 73:27; 74:26; 75:25; 76:24; 77:23; 78:22; 79:21; 80:20; 81:19; 82:18; 83:17; 84:16; 85:15; 86:14; 87:13; 88:12; 89:11; 90:10; 91:9; 92:8; 93:7; 94:6; 95:5; 96:4; 97:3; 98:2; and 99:1, or any range derivable therein.

In some embodiments, at least 10% of a total population of a first particular nucleotide in a RNA molecule as disclosed herein has been replaced with one or more modified or unnatural nucleotides, and at least 10% of a total population of a second particular nucleotide in the molecule has been replaced with one or more modified or unnatural nucleotides. In some embodiments, at least 10% of a total population of a first particular nucleotide in the molecule has been replaced with one or more modified or unnatural nucleotides, and at least 25% of a total population of a second particular nucleotide in the molecule has been replaced with one or more modified or unnatural nucleotides. In some embodiments, at least 10% of a total population of a first particular nucleotide in the molecule has been replaced with one or more modified or unnatural nucleotides, and at least 50% of a total population of a second particular nucleotide in the molecule has been replaced with one or more modified or unnatural nucleotides. In some embodiments, at least 10% of a total population of a first particular nucleotide in the molecule has been replaced with one or more modified or unnatural nucleotides, and at least 75% of a total population of a second particular nucleotide in the molecule has been replaced with one or more modified or unnatural nucleotides. In some embodiments, at least 10% of a total population of a first particular nucleotide in the molecule has been replaced with one or more modified or unnatural nucleotides, and essentially all of a total population of a second particular nucleotide in the molecule has been replaced with one or more modified or unnatural nucleotides. In some embodiments, at least 25% of a total population of a first particular nucleotide in the molecule has been replaced with one or more modified or unnatural nucleotides, and at least 25% of a total population of a second particular nucleotide in the molecule has been replaced with one or more modified or unnatural nucleotides. In some embodiments, at least 25% of a total population of a first particular nucleotide in the molecule has been replaced with one or more modified or unnatural nucleotides, and at least 50% of a total population of a second particular nucleotide in the molecule has been replaced with one or more modified or unnatural nucleotides. In some embodiments, at least 25% of a total population of a first particular nucleotide in the molecule has been replaced with one or more modified or unnatural nucleotides, and at least 75% of a total population of a second particular nucleotide in the molecule has been replaced with one or more modified or unnatural nucleotides. In some embodiments, at least 25% of a total population of a first particular nucleotide in the molecule has been replaced with one or more modified or unnatural nucleotides, and essentially all of a total population of a second particular nucleotide in the molecule has been replaced with one or more modified or unnatural nucleotides. In some embodiments, at least 50% of a total population of a first particular nucleotide in the molecule has been replaced with one or more modified or unnatural nucleotides, and at least 50% of a total population of a second particular nucleotide in the molecule has been replaced with one or more modified or unnatural nucleotides. In some embodiments, at least 50% of a total population of a first particular nucleotide in the molecule has been replaced with one or more modified or unnatural nucleotides, and at least 75% of a total population of a second particular nucleotide in the molecule has been replaced with one or more modified or unnatural nucleotides. In some embodiments, at least 50% of a total population of a first particular nucleotide in the molecule has been replaced with one or more modified or unnatural nucleotides, and essentially all of a total population of a second particular nucleotide in the molecule has been replaced with one or more modified or unnatural nucleotides. In some embodiments, at least 75% of a total population of a first particular nucleotide in the molecule has been replaced with one or more modified or unnatural nucleotides, and at least 75% of a total population of a second particular nucleotide in the molecule has been replaced with one or more modified or unnatural nucleotides. In some embodiments, at least 75% of a total population of a first particular nucleotide in the molecule has been replaced with one or more modified or unnatural nucleotides, and essentially all of a total population of a second particular nucleotide in the molecule has been replaced with one or more modified or unnatural nucleotides. In some embodiments, essentially all of a total population of a first particular nucleotide in the molecule has been replaced with one or more modified or unnatural nucleotides, and essentially all of a total population of a second particular nucleotide in the molecule has been replaced with one or more modified or unnatural nucleotides.

In some embodiments, at least 25% of a total population of uridine nucleotides in the RNA molecule has been replaced with N1-methylpseudouridine. In some embodiments, at least 50% of a total population of uridine nucleotides in the molecule has been replaced with N1-methylpseudouridine. In some embodiments, at least 75% of a total population of uridine nucleotides in the molecule has been replaced with N1-methylpseudouridine. In some embodiments, essentially all uridine nucleotides in the molecule have been replaced with N1-methylpseudouridine. In some embodiments, at least 50% of a total population of uridine nucleotides in the molecule has been replaced with 5-methoxyuridine. In some embodiments, essentially all uridine nucleotides in the molecule have been replaced with 5-methoxyuridine. In some embodiments, at least 50% of a total population of uridine nucleotides in the molecule has been replaced with 5-methyluridine. In some embodiments, essentially all uridine nucleotides in the molecule have been replaced with 5-methyluridine. In some embodiments, at least 50% of a total population of cytosine nucleotides in the molecule has been replaced with 5-methylcytosine. In some embodiments, essentially all cytosine nucleotides in the molecule have been replaced with 5-methylcytosine. In some embodiments, at least 50% of a total population of uridine nucleotides in the molecule has been replaced with 2-thiouridine. In some embodiments, essentially all uridine nucleotides in the molecule have been replaced with 2-thiouridine.

In some embodiments, at least 50% of a total population of uridine nucleotides in the molecule has been replaced with N1-methylpseudouridine and essentially all cytosine nucleotides in the molecule have been replaced with 5-methylcytosine. In some embodiments, at least 50% of a total population of uridine nucleotides in the molecule has been replaced with 5-methoxyuridine and essentially all cytosine nucleotides in the molecule have been replaced with 5-methylcytosine. In some embodiments, at least 50% of a total population of uridine nucleotides in the molecule has been replaced with 5-methyluridine and essentially all cytosine nucleotides in the molecule have been replaced with 5-methylcytosine.

In some embodiments, essentially all uridine nucleotides in the molecule have been replaced with about 50% 5-methoxyuridine and about 50% N1-methylpseudouridine. In some embodiments, essentially all uridine nucleotides in the molecule have been replaced with about 75% 5-methoxyuridine and about 25% N1-methylpseudouridine. In some embodiments, essentially all uridine nucleotides in the molecule have been replaced with about 25% 5-methoxyuridine and about 75% N1-methylpseudouridine.

### RNA Transcription

In some aspects, the RNA disclosed herein is produced by in vitro transcription or chemical synthesis. In the context of the present disclosure, the term “transcription” relates to a process, wherein the genetic code in a DNA sequence is transcribed into RNA. Subsequently, the RNA may be translated into peptide or protein.

According to the present disclosure, “transcription” comprises “in vitro transcription” or “IVT,” which refers to the process whereby transcription occurs in vitro in a non-cellular system to produce a synthetic RNA product for use in various applications, including, e.g., production of protein or polypeptides. The methodology for in vitro transcription of mRNA is well known in the art. (see, e.g., Losick, R. 1972. In vitro transcription, Ann Rev Biochem, 41 409-46; Kamakaka, R. T. and Kraus, W. L. 2001. In vitro Transcription, Current Protocols in Cell Biology, 2:11.6:11.6.1-11.6.17; Beckert, B. And Masquida, B. 2010. Synthesis of RNA by In vitro Transcription in RNA, Methods in Molecular Biology, 703 (Neilson, H. Ed), New York, N.Y. Humana Press, 2010; Brunelle, J. L. and Green, R., 2013, Chapter Five—In vitro transcription from plasmid or PCR-amplified DNA, Methods in Enzymology 530:101-114; all of which are incorporated herein by reference).

Cloning vectors may be applied for the generation of transcripts. These cloning vectors are generally designated as transcription vectors and are according to the present invention encompassed by the term “vector.” According to specific aspects, the RNA used is in vitro transcribed RNA (IVT-RNA) and may be obtained by in vitro transcription of an appropriate DNA template. Template DNA can be prepared for in vitro transcription from a number of sources with appropriate techniques which are well known in the art including, but not limited to, plasmid DNA and polymerase chain reaction amplification (see Linpinsel, J. L and Conn, G. L., General protocols for preparation of plasmid DNA template, and Bowman, J. C., Azizi, B., Lenz, T. K., Ray, P., and Williams, L. D. in RNA in vitro transcription and RNA purification by denaturing PAGE in Recombinant and in vitro RNA syntheses, Methods 941 Conn G.L. (ed), New York, N.Y. Humana Press, 2012, each incorporated herein by reference). The promoter for controlling transcription may be any promoter for any RNA polymerase. Particular examples of RNA polymerases are the T7, T3, and SP6 RNA polymerases. Preferably, the in vitro transcription according to the invention is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

Synthetic IVT RNA products may be translated in vitro or introduced directly into cells, where they may be translated. With respect to RNA, the term “expression” or “translation” relates to the process in the ribosomes of a cell by which a strand of mRNA directs the assembly of a sequence of amino acids to make a peptide or protein. Such synthetic RNA products include but are not limited to, e.g., mRNA molecules, saRNA molecules, antisense RNA molecules, shRNA molecules, long non-coding RNA molecules, ribozymes, aptamers, guide RNA molecules (e.g., for CRISPR), ribosomal RNA molecules, small nuclear RNA molecules, small nucleolar RNA molecules, and the like. In some aspects, 1, 2, 3, 4, 5, or more of the foregoing synthetic RNA products may be excluded. An IVT reaction typically utilizes a DNA template (e.g., a linear DNA template) as described and/or utilized herein, ribonucleotides (e.g., non-modified ribonucleotide triphosphates or modified ribonucleotide triphosphates), and an appropriate RNA polymerase.

In some aspects, an mRNA is produced by in vitro transcription using a DNA template where DNA refers to a nucleic acid that contains deoxyribonucleotides. In some aspects, an RNA disclosed herein is in vitro transcribed RNA (IVT-RNA) and may be obtained by in vitro transcription of an appropriate DNA template. The promoter for controlling transcription may be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In some aspects, starting material for IVT may include linearized DNA template, nucleotides, Rnase inhibitor, pyrophosphatase, and/or a polymerase (e.g., a T7 RNA polymerase). The nucleotides may be manufactured in house, may be obtained from a supplier, or may be synthesized. The nucleotides may be, but are not limited to, those described herein including natural and unnatural (modified) nucleotides. Any number of RNA polymerases or variants may be used, including, but not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. In some aspects, 1, 2, 3, 4, 5, or more of the foregoing RNA polymerases may be excluded from. Some embodiments exclude the use of Dnase.

In some aspects, the IVT process is conducted in a bioreactor. The bioreactor may comprise a mixer. In some aspects, nucleotides may be added into the bioreactor throughout the IVT process.

In some aspects, one or more post-IVT agents are added into the IVT mixture comprising RNA in the bioreactor after the IVT process. Exemplary post-IVT agents may include DNAse I configured to digest the linearized DNA template and/or proteinase K configured to digest DNAse I and T7 RNA polymerase. In some aspects, the post-IVT agents are incubated with the mixture in the bioreactor after IVT. In some aspects, the bioreactor may contain at least, at most, exactly, or between (inclusive or exclusive) any two of 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, and 500 or more liters IVT mixture. The IVT mixture may have an RNA concentration that is or is not at least, at most, exactly, or between (inclusive or exclusive) any two of 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 7.0, 8.0, 9.0, 10, 11, 12, 13, 14, 15, 16, 17, 18, 19, 20, 30, 40, 50, 60, 70, 80, 90, and 100 mg/mL or more RNA.

In some aspects, the IVT mixture may include residual spermidine, residual DNA, residual proteins, peptides, HEPES, EDTA, ammonium sulfate, cations (e.g., Mg²⁺, Na⁺, Ca²⁺), RNA fragments, residual nucleotides, free phosphates, or any combinations thereof. In some aspects, 1, 2, 3, 4, 5, or more of the foregoing can be excluded from the IVT mixture.

Isolation and/or purification of the nucleic acids described herein may include, but is not limited to, phenol/chloroform extraction and/or precipitation with either alcohol (ethanol, isopropanol) in the presence of monovalent cations or lithium chloride for nucleic acid clean-up, quality assurance and quality control. Additional, non-limiting examples of purification procedures include AGENCOURT® beads (Beckman Coulter Genomics, Danvers, MA), poly-T beads, LNA™ oligo-T capture probes (EXIQON® Inc, Vedbaek, Denmark), HPLC based purification methods such as, but not limited to, strong anion exchange HPLC, weak anion exchange HPLC, reverse phase HPLC (RP-HPLC), and hydrophobic interaction HPLC (HIC-HPLC), size exclusion chromatography, and silica-based affinity chromatography and polyacrylamide gel electrophoresis. Purification can be performed using a variety of commercially available kits including, but not limited to SV Total Isolation System (Promega) and In vitro Transcription Cleanup and Concentration Kit (Norgen Biotek). In some aspects, 1, 2, 3, 4, 5, or more of the foregoing purification may be excluded.

The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment and/or purification method.

In some aspects, at least a portion of the IVT mixture is filtered. The IVT mixture may be filtered via ultrafiltration and/or diafiltration to remove at least some impurities from the IVT mixture and/or to change buffer solution for the at least a portion of IVT mixture to produce a concentrated RNA solution as a retentate.

In some aspects, both “ultrafiltration” and “diafiltration” refer to a membrane filtration process. Ultrafiltration typically uses membranes having pore sizes of at least, at most, exactly, or between (inclusive or exclusive) any two of 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, and 0.1 μm. In some aspects, ultrafiltration membranes are typically classified by molecular weight cutoff (MWCO) rather than pore size. For example, the MWCO may be at least, at most, exactly, or between (inclusive or exclusive) any two of 30 kDa, 40 kDa, 50 kDa, 60 kDa, 70 kDa, 80 kDa, 90 kDa, 100 kDa, 110 kDa, 120 kDa, 130 kDa, 140 kDa, 150 kDa, 160 kDa, 170 kDa, 180 kDa, 190 kDa, 200 kDa, 210 kDa, 220 kDa, 230 kDa, 240 kDa, 250 kDa, 260 kDa, 270 kDa, 280 kDa, 290 kDa, 300 kDa, 310 kDa, 320 kDa, 330 kDa, 340 kDa, 350 kDa, 360 kDa, 370 kDa, 380 kDa, 390 kDa, 400 kDa, 500 kDa, 600 kDa, 700 kDa, 800 kDa, 900 kDa, 1000 kDa, 2000 kDa, 3000 kDa, 4000 kDa, 5000 kDa, 6000 kDa, 7000 kDa, 8000 kDa, 9000 kDa, and 10000 kDa. A skilled artisan will understand that filtration membranes may comprise different suitable materials, including, e.g., polymers, cellulose, ceramic, etc., depending upon the application. In some aspects, membrane filtration may be more desirable for large volume purification process.

In some aspects, ultrafiltration and diafiltration of the IVT mixture for purifying RNA may include (1) Direct Flow Filtration (DFF), also known as “dead-end” filtration, that applies a feed stream perpendicular to the membrane face and attempts to pass 100% of the fluid through the membrane, and/or (2) Tangential Flow Filtration (TFF), also known as crossflow filtration, where a feed stream passes parallel to the membrane face as one portion passes through the membrane (permeate) while the remainder (retentate) is retained and/or recirculated back to the feed tank.

In some aspects, the filtering of the IVT mixture is conducted via TFF comprising an ultrafiltration step, a first diafiltration step, and a second diafiltration step. In some aspects, the first diafiltration step is conducted in the presence of ammonium sulfate. The first diafiltration step may be configured to remove a majority of impurities from the IVT mixture. In some aspects, the second diafiltration step is conducted without ammonium sulfate. The second diafiltration step may be configured to transfer the RNA into a DS buffer formulation.

A filtration membrane with an appropriate MWCO may be selected for ultrafiltration in the TFF process. The MWCO of a TFF membrane determines which solutes may pass through the membrane into the filtrate and which are retained in the retentate. The MWCO of a TFF membrane may be selected such that substantially all of the solutes of interest (e.g., desired synthesized RNA species) remain in the retentate, whereas undesired components (e.g., excess ribonucleotides, small nucleic acid fragments such as digested or hydrolyzed DNA template, peptide fragments such as digested proteins and/or other impurities) pass into the filtrate. In some aspects, the retentate comprising desired synthesized RNA species may be re-circulated to a feed reservoir to be re-filtered in additional cycles. In some aspects, a TFF membrane may have a MWCO of at least, at most, exactly, or between (inclusive or exclusive) any two of 30 kDa, 40 kDa, 50 kDa, 60 kDa, 70 kDa, 80 kDa, 90 kDa, or more. In some aspects, a TFF membrane may have a MWCO of at least, at most, exactly, or between (inclusive or exclusive) any two of 100 kDa, 150 kDa, 200 kDa, 250 kDa, 300 kDa, 350 kDa, 400 kDa, or more. In some aspects, a TFF membrane may have a MWCO of or of about 250-350 kDa. In some aspects, a TFF membrane (e.g., a cellulose-based membrane) may have a MWCO of or of about 30-300 kDa; 50-300 kDa, 100-300 kDa, or 200-300 kDa.

Diafiltration may be performed either discontinuously, or alternatively, continuously. For example, in continuous diafiltration, a diafiltration solution may be added to a sample feed reservoir at the same rate as filtrate is generated. In this way, the volume in the sample reservoir remains constant but small molecules (e.g., salts, solvents, etc.) that may freely permeate through a membrane are removed. Using solvent removal as an example, each additional diafiltration volume (DV) reduces the solvent concentration further. In discontinuous diafiltration, a solution is first diluted and then concentrated back to the starting volume. This process is then repeated until the desired concentration of small molecules (e.g., salts, solvents, etc.) remaining in the reservoir is reached. Each additional diafiltration volume (DV) reduces the small molecule (e.g., solvent) concentration further. Continuous diafiltration typically requires a minimum volume for a given reduction of molecules to be filtered. Discontinuous diafiltration, on the other hand, permits fast changes of the retentate condition, such as pH, salt content, and the like. In some aspects, the first diafiltration step is conducted with at least, at most, exactly, or between (inclusive or exclusive) any two of 2, 3, 4, 5, 6, 7, 8, 9, 10, or more diavolumes. In some aspects, the second diafiltration step is conducted with at least, at most, exactly, or between (inclusive or exclusive) any two of 5, 6, 7, 8, 9, 10, 11, 12, 13, 14, 15, 16, 17, 18, 19, 20, or more diavolumes. In some aspects, the first diafiltration step is conducted with 5 diavolumes, and second diafiltration step is conducted with 10 diavolumes.

In some aspects, for ultrafiltration and/or diafiltration, the IVT mixture is filtered at a rate of at least, at most, exactly, or between (inclusive or exclusive) any two of 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 500, 600, 700, 800, 900, or 1000 L/m² of filter area per hour, or more. The concentrated RNA solution may comprise at least, at most, exactly, or between (inclusive or exclusive) any two of 2.0, 2.1, 2.2, 2.3, 2.4, or 2.5 mg/mL single stranded RNA.

The bioburden of the concentrated RNA solution via filtration to obtain an RNA product solution may also be reduced, in some aspects. The filtration for reducing bioburden may be conducted using one or more filters. The one or more filters may include a filter with a pore size that is or is not at least, at most, exactly, or between (inclusive or exclusive) any two of 0.2 μm, 0.45 μm, 0.65 μm, 0.8 μm, or any other pore size configured to remove bioburdens.

As one example, reducing the bioburden may include draining a retentate tank containing retentate obtained from the ultrafiltration and/or diafiltration to obtain the retentate. Reducing the bioburden may include flushing a filtration system for ultrafiltration and/or diafiltration using a wash buffer solution to obtain a wash pool solution comprising residue RNA remaining in the filtration system. The retentate may be filtered to obtain a filtered retentate. The wash pool solution may be filtered using a first 0.2 μm filter to obtain a filtered wash pool solution. The retentate may be filtered using the first 0.2 μm filter or another 0.2 μm filter.

The filtered wash pool solution and the filtered retentate may be combined to form a combined pool solution. The combined pool solution may be filtered using a second 0.2 μm filter to obtain a filtered combined pool solution, which is further filtered using a third 0.2 μm filter to produce an RNA product solution.

A quality assurance and/or quality control check may be conducted using methods such as, but not limited to, gel electrophoresis, UV absorbance, and/or analytical HPLC.

In some aspects, the nucleic acids may be sequenced by methods including, but not limited to reverse-transcriptase-PCR.

In some aspects, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size and/or to assess degradation. Degradation of the nucleic acid may be assessed by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE). In some aspects, 1, 2, 3, 4, 5, or more of the foregoing assessment methods may be excluded.

### Characterization and Analysis of RNA Molecule

The RNA molecule described herein may be analyzed and characterized using various methods. Analysis may be performed before or after capping. Alternatively, analysis may be performed before or after poly-A capture-based affinity purification. In another aspect, analysis may be performed before or after additional purification steps, e.g., anion exchange chromatography and the like. For example, RNA template quality may be determined using Bioanalyzer chip based electrophoresis system. In other aspects, RNA template purity is analyzed using analytical reverse phase HPLC respectively. Capping efficiency may be analyzed using, e.g., total nuclease digestion followed by MS/MS quantitation of the dinucleotide cap species vs. uncapped GTP species. In vitro efficacy may be analyzed by, e.g., transfecting RNA molecule into a human cell line. Protein expression of the polypeptide of interest may be quantified using methods such as ELISA or flow cytometry. Immunogenicity may be analyzed by, e.g., transfecting RNA molecules into cell lines that indicate innate immune stimulation, e.g., PBMCs. Cytokine induction may be analyzed using, e.g., methods such as ELISA to quantify a cytokine, e.g., Interferon-α. Biodistribution may be analyzed, e.g. by bioluminescence measurements.

In some aspects, an RNA polynucleotide disclosed herein is characterized in that, when assessed in an organism administered a composition or medical preparation comprising an RNA polynucleotide, elevated expression of a gene of interest; increased duration of expression (e.g., prolonged expression) of a gene of interest; elevated expression and increased duration of expression (e.g., prolonged expression) of a gene of interest; decreased interaction with IFIT1 of an RNA polynucleotide; increased translation of an RNA polynucleotide; is observed relative to an appropriate reference.

In some aspects, a reference comprises an organism administered an otherwise similar RNA polynucleotide without a m7(3′OMeG)(5′)ppp(5′)(2′OMeAi)pG2 cap. In some aspects, a reference comprises an organism administered an otherwise similar RNA polynucleotide without a cap proximal sequence disclosed herein. In some aspects, a reference comprises an organism administered an otherwise similar RNA polynucleotide with a self-hybridizing sequence.

In some aspects, elevated expression is determined at least 24 hours, at least 48 hours at least 72 hours, at least 96 hours, or at least 120 hours after administration of a composition or medical preparation comprising an RNA polynucleotide. In some aspects, elevated expression is determined at least 24 hours after administration of a composition or medical preparation comprising an RNA polynucleotide. In some aspects, elevated expression is determined at least 48 hours after administration of a composition or medical preparation comprising an RNA polynucleotide. In some aspects, elevated expression is determined at least 72 hours after administration of a composition or medical preparation comprising an RNA polynucleotide. In some aspects, elevated expression is determined at least 96 hours after administration of a composition or medical preparation comprising an RNA polynucleotide. In some aspects, elevated expression is determined at least 120 hours after administration of a composition or medical preparation comprising an RNA polynucleotide.

In some aspects, elevated expression is determined at about 24-120 hours after administration of a composition or medical preparation comprising an RNA polynucleotide. In some aspects, elevated expression is determined at about 24-110 hours, about 24-100 hours, about 24-90 hours, about 24-80 hours, about 24-70 hours, about 24-60 hours, about 24-50 hours, about 24-40 hours, about 24-30 hours, about 30-120 hours, about 40-120 hours, about 50-120 hours, about 60-120 hours, about 70-120 hours, about 80-120 hours, about 90-120 hours, about 100-120 hours, or about 110-120 hours after administration of a composition or medical preparation comprising an RNA polynucleotide.

In some aspects, elevated expression of a gene of interest is at least 2-fold to at least 10-fold. In some aspects, elevated expression of a gene of interest is at least 2-fold. In some aspects, elevated expression of a gene of interest is at least 3-fold. In some aspects, elevated expression of a gene of interest is at least 4-fold. In some aspects, elevated expression of a gene of interest is at least 6-fold. In some aspects, elevated expression of a gene of interest is at least 8-fold. In some aspects, elevated expression of a gene of interest is at least 10-fold.

In some aspects, elevated expression of a gene of interest is about 2-fold to about 50-fold. In some aspects, elevated expression of a gene of interest is about 2-fold to about 45-fold, about 2-fold to about 40-fold, about 2-fold to about 30-fold, about 2-fold to about 25-fold, about 2-fold to about 20-fold, about 2-fold to about 15-fold, about 2-fold to about 10-fold, about 2-fold to about 8-fold, about 2-fold to about 5-fold, about 5-fold to about 50-fold, about 10-fold to about 50-fold, about 15-fold to about 50-fold, about 20-fold to about 50-fold, about 25-fold to about 50-fold, about 30-fold to about 50-fold, about 40-fold to about 50-fold, or about 45-fold to about 50-fold. In some aspects, elevated expression of a gene of interest is at least, at most, exactly, or between any two of 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold, or any range or value derivable therein.

In some aspects, elevated expression (e.g., increased duration of expression) of a gene of interest persists for at least, at most, exactly, or between any two of 24 hours, 48 hours, 72 hours, 96 hours, or 120 hours after administration of a composition or a medical preparation comprising an RNA polynucleotide. In some aspects, elevated expression of a gene of interest persists for at least 24 hours after administration. In some aspects, elevated expression of a gene of interest persists for at least 48 hours after administration. In some aspects, elevated expression of a gene of interest persists for at least 72 hours after administration. In some aspects, elevated expression of a gene of interest persists for at least 96 hours after administration. In some aspects, elevated expression of a gene of interest persists for at least 120 hours after administration of a composition or medical preparation comprising an RNA polynucleotide.

In some aspects, elevated expression of a gene of interest persists for about 24-120 hours after administration of a composition or medical preparation comprising an RNA polynucleotide. In some aspects, elevated expression persists for about 24-110 hours, about 24-100 hours, about 24-90 hours, about 24-80 hours, about 24-70 hours, about 24-60 hours, about 24-50 hours, about 24-40 hours, about 24-30 hours, about 30-120 hours, about 40-120 hours, about 50-120 hours, about 60-120 hours, about 70-120 hours, about 80-120 hours, about 90-120 hours, about 100-120 hours, or about 110-120 hours after administration of a composition or medical preparation comprising an RNA polynucleotide. In some aspects, elevated expression of a gene of interest persists for at least, at most, exactly, or between any two of 24 hours, 36 hours, 48 hours, 60 hours, 72 hours, 84 hours, 96 hours, 108 hours, or 120 hours, or any range or value derivable therein.

### Immunoassays

The present disclosure includes the implementation of serological assays to evaluate whether and to what extent an immune response is induced or evoked by compositions of the disclosure. There are many types of immunoassays that may be implemented. Immunoassays encompassed by the present disclosure include, but are not limited to, those described in U.S. Pat. No. 4,367,110 (double monoclonal antibody sandwich assay) and U.S. Pat. No. 4,452,901 (western blot). Other assays include immunoprecipitation of labeled ligands and immunocytochemistry, both in vitro and in vivo.

Immunoassays generally are binding assays. In some aspects, the immunoassays are the various types of enzyme linked immunosorbent assays (ELISAs) and radioimmunoassays (RIA) known in the art. Immunohistochemical detection using tissue sections is also particularly useful. In one example, antibodies or antigens are immobilized on a selected surface, such as a well in a polystyrene microtiter plate, dipstick, or column support. Then, a test composition suspected of containing the desired antigen or antibody, such as a clinical sample, is added to the wells. After binding and washing to remove non-specifically bound immune complexes, the bound antigen or antibody may be detected. Detection is generally achieved by the addition of another antibody, specific for the desired antigen or antibody, that is linked to a detectable label. This type of ELISA is known as a “sandwich ELISA.” Detection also may be achieved by the addition of a second antibody specific for the desired antigen, followed by the addition of a third antibody that has binding affinity for the second antibody, with the third antibody being linked to a detectable label.

Competition ELISAs are also possible implementations in which test samples compete for binding with known amounts of labeled antigens or antibodies. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal. Irrespective of the format employed, ELISAs have certain features in common, such as coating, incubating or binding, washing to remove non-specifically bound species, and detecting the bound immune complexes.

Antigen or antibodies may also be linked to a solid support, such as in the form of plate, beads, dipstick, membrane, or column matrix, and the sample to be analyzed is applied to the immobilized antigen or antibody. In coating a plate with either antigen or antibody, one will generally incubate the wells of the plate with a solution of the antigen or antibody, either overnight or for a specified period. The wells of the plate will then be washed to remove incompletely-adsorbed material. Any remaining available surfaces of the wells are then “coated” with a nonspecific protein that is antigenically neutral with regard to the test antisera. These include bovine serum albumin (BSA), casein, and solutions of milk powder. The coating allows for blocking of nonspecific adsorption sites on the immobilizing surface and thus reduces the background caused by nonspecific binding of antisera onto the surface.

### Protective Immunity

In some aspects of the disclosure, RNA molecules encoding the immunogen, RNA-LNPs and compositions thereof, confer protective immunity to a subject. Protective immunity refers to a body's ability to mount a specific immune response that protects the subject from developing a particular disease or condition that involves the agent against which there is an immune response. An immunogenically effective amount is capable of conferring protective immunity to the subject. In some aspects, the RNA molecules encoding the immunogen polypeptides, RNA-LNPs and compositions thereof of the present disclosure may be used to induce a balanced immune response against the immunogen comprising both cellular and humoral immunity, without many of the risks associated with attenuated virus vaccination. A “humoral” immune response refers to an immune response mediated by antibody molecules, including, e.g., secretory (IgA) or IgG molecules, while a “cellular” immune response is one mediated by T-lymphocytes (e.g., CD4+ helper and/or CD8+ T cells (e.g., CTLs) and/or other white blood cells.

As used herein the phrase “immune response” or its equivalent “immunological response” refers to the development of a humoral (antibody mediated), cellular (mediated by antigen-specific T cells or their secretion products) or both humoral and cellular response directed against an immunogen. Such a response may be an active response or a passive response. A cellular immune response is elicited by the presentation of polypeptide epitopes in association with Class I or Class II MHC molecules, to activate antigen-specific CD4 (+) T helper cells and/or CD8 (+) cytotoxic T cells. The response may also involve activation of monocytes, macrophages, NK cells, basophils, dendritic cells, astrocytes, microglia cells, eosinophils or other components of innate immunity. As used herein “active immunity” refers to any immunity conferred upon a subject from the production of antibodies in response to the presence of an immunogen encoded by an RNA molecule.

As used herein “passive immunity” includes, but is not limited to, administration of activated immune effectors including cellular mediators or protein mediators (e.g., monoclonal and/or polyclonal antibodies) of an immune response. A monoclonal or polyclonal antibody composition may be used in passive immunization to treat, prevent, or reduce the severity of illness caused by infection by organisms that carry the antigen recognized by the antibody. An antibody composition may include antibodies that bind to a variety of antigens that may in turn be associated with various organisms. The antibody component may be a polyclonal antiserum. In certain aspects the antibody or antibodies are affinity purified from an animal or second subject that has been challenged with an antigen(s). Alternatively, an antibody mixture may be used, which is a mixture of monoclonal and/or polyclonal antibodies to antigens present in the same, related, or different microbes or organisms, such as viruses, including but not limited to the antigen.

Passive immunity may be imparted to a patient or subject by administering to the patient immunoglobulins (lg) and/or other immune factors obtained from a donor or other non-patient source having a known immunoreactivity. In other aspects, an immunogenic composition of the present disclosure may be administered to a subject who then acts as a source or donor for globulin, produced in response to challenge with the immunogenic composition (“hyperimmune globulin”), that contains antibodies directed against a the antigen or other organism. A subject thus treated would donate plasma from which hyperimmune globulin would then be obtained, via conventional plasma-fractionation methodology, and administered to another subject in order to impart resistance against or to treat the antigen infection.

For purposes of this specification and the accompanying claims the terms “epitope” and “antigenic determinant” are used interchangeably to refer to a site on an antigen to which B and/or T cells respond or recognize. B-cell epitopes may be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols (1996). Antibodies that recognize the same epitope may be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope may be identified by in vitro assays that measure antigen-dependent proliferation, as determined by 3H-thymidine incorporation by primed T cells in response to an epitope (Burke et al., 1994), by antigen-dependent killing (cytotoxic T lymphocyte assay, Tigges et al., 1996) or by cytokine secretion.

The presence of a cell-mediated immunological response may be determined by proliferation assays (CD4 (+) T cells) or CTL (cytotoxic T lymphocyte) assays. The relative contributions of humoral and cellular responses to the protective or therapeutic effect of an immunogenic composition may be distinguished by separately isolating IgG and T-cells from an immunized syngeneic animal and measuring protective or therapeutic effect in a second subject.

As used herein, the terms “antibody” or “immunoglobulin” are used interchangeably and refer to any of several classes of structurally related proteins that function as part of the immune response of an animal or recipient, which proteins include IgG, IgD, IgE, IgA, IgM and related proteins. Under normal physiological conditions antibodies are found in plasma and other body fluids and in the membrane of certain cells and are produced by lymphocytes of the type denoted B cells or their functional equivalent.

### Pharmaceutical Compositions

In another embodiment, the disclosure comprises pharmaceutical compositions. For pharmaceutical composition purposes, the compound per se or pharmaceutically acceptable salt thereof will simply be referred to as the compounds of the disclosure.

A “pharmaceutical composition” refers to a mixture of one or more of the compounds of the disclosure, or a pharmaceutically acceptable salt, solvate, hydrate or prodrug thereof as an active ingredient, and at least one pharmaceutically acceptable excipient. In some embodiments, a pharmaceutical composition comprises LNPs or RNA-LNPs disclosed herein that comprise one or more of the compounds of the disclosure, or a pharmaceutically acceptable salt, solvate, hydrate or prodrug thereof.

“Excipient” as used herein describes any ingredient other than the compound(s) of the disclosure. The choice of excipient will to a large extent depend on factors such as the mode of administration, the effect of the excipient on solubility and stability, and the nature of the dosage form.

As used herein, “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugar, sodium chloride, or polyalcohol such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives, or buffers, which enhance the shelf life or effectiveness of the compound.

The compositions of this disclosure may be in a variety of forms. These include, for example, liquid, semi-solid and solid dosage forms, such as liquid solutions (e.g., injectable and infusible solutions), dispersions or suspensions, tablets, capsules, pills, powders, liposomes, lipid nanoparticles and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g., intravenous, subcutaneous, intraperitoneal, intramuscular). In another embodiment, the compound is administered by intravenous infusion or injection. In yet another embodiment, the compound is administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the disclosure. In another embodiment, the oral administration may be in a powder or granule form. In another embodiment, the oral dosage form is sub-lingual, such as, for example, a lozenge. In such solid dosage forms, the compounds of the disclosure are ordinarily combined with one or more adjuvants. Such capsules or tablets may comprise a controlled release formulation. In the case of capsules, tablets, and pills, the dosage forms also may comprise buffering agents or may be prepared with enteric coatings.

In another embodiment, oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

In another embodiment, the disclosure comprises a parenteral dosage form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using one or more of suitable dispersing, wetting agents, or suspending agents.

In another embodiment, the disclosure comprises a topical dosage form. “Topical administration” includes, for example, dermal and transdermal administration, such as via transdermal patches (with or without microneedle-mediated transdermal delivery) or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this disclosure are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated-see, for example, B. C. Finnin and T. M. Morgan, J. Pharm. Sci., vol. 88, pp. 955-958, 1999.

Formulations suitable for topical administration to the eye include, for example, eye drops wherein the compound of this disclosure is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration, the compounds of the disclosure are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the disclosure comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the disclosure may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; Rowe, Raymond C. Handbook of Pharmaceutical Excipients. Chicago, Pharmaceutical Press, 2005; Stahl, P. Heinrich and Camilli G. Wermuth, Eds. Handbook of Pharmaceutical Salts: Properties, Selection, and Use. New York: Wiley-VCH, 2011; and Brittain, Harry G., Ed. Polymorphism in Pharmaceutical Solids. New York: Informa Healthcare USA, Inc., 2016.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5)alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

Liposome containing compounds of the disclosure may be prepared by methods known in the art (See, for example, Chang, H. I.; Yeh, M. K.; Clinical development of liposome-based drugs: formulation, characterization, and therapeutic efficacy; Int J Nanomedicine 2012; 7; 49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Lipid nanoparticles (LNPs) comprising the compounds of the disclosure may be prepared by methods known in the art. Lipid nanoparticles (LNPs) constitute an alternative to other particulate systems, such as emulsions, liposomes, micelles, microparticles and/or polymeric nanoparticles, for the delivery of active ingredients, such as oligonucleotides, RNA and small molecule pharmaceuticals, and the adjuvant compounds of the disclosure. LNPs and their use for the delivery of oligonucleotides have been previously disclosed. See U.S. Pat. Nos. 7,691,405 and 11,406,706, U.S. Patent Application Publication Nos: US 2006/0083780, US 2006/0240554, US 2008/0020058, US 2009/0263407 and US 2009/0285881; and International Patent Application Publication Nos.: WO 2009/086558, WO2009/127060, WO2009/132131, WO2010/042877, WO2010/054384, WO2010/054401, WO2010/054405 and WO2010/054406. See also Semple et al., 2010, Nat. Biotechnol. 28:172-176. LNPs and their use for delivery of RNA vaccines have been previously disclosed. See International Patent Application Publication Nos.: WO2021213924 and WO2023019181. Lipid-based nanoparticles as pharmaceutical drug carriers have also been disclosed. See Puri et al., 2009, Crit. Rev. Ther. Drug Carrier Syst. 26:523-580. Cationic lipids are disclosed in U.S. Patent Application Publication Nos. US 2009/0263407, US 2009/0285881, US 2010/0055168, US 2010/0055169, US 2010/0063135, US 2010/0076055, US 2010/0099738 and US 2010/0104629, and U.S. Pat. No. 10,166,298. Lipid nanoparticle capsules are described in U.S. Patent Application Publication No. 2013/0017239. The compounds of the disclosure may be embedded in the lipid layer of the LNP for targeting of the LNP comprising an active ingredient (i.e. oligonucleotide, RNA, small molecule, etc) to the lymph nodes via the TLR7/8 modulating moiety of the compounds of the disclosure.

Compounds of the disclosure may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, lipid nanoparticles, albumin microspheres, microemulsions, nanoparticles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing a compound of the disclosure, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the disclosure are generally placed into a container having a sterile access port, for example, an intravenous solution bag or vial having a stopper pierceable by a hypodermic injection needle.

Suitable emulsions may be prepared using commercially available fat emulsions, such as a lipid emulsions comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets between 0.1 and 1.0 μm, particularly 0.1 and 0.5 μm, and have a pH in the range of 5.5 to 8.0.

For example, the emulsion compositions may be those prepared by mixing a compound of the disclosure with a lipid emulsions comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

A drug product intermediate (DPI) is a partly processed material that must undergo further processing steps before it becomes bulk drug product. Compounds of the disclosure may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability. In one embodiment, the drug product intermediate contains a compound of the disclosure isolated and stabilized in the amorphous state (for example, amorphous solid dispersions (ASDs)). There are many techniques known in the art to manufacture ASD's that produce material suitable for integration into a bulk drug product, for example, spray dried dispersions (SDD's), melt extrudates (often referred to as HME's), co-precipitates, amorphous drug nanoparticles, and nano-adsorbates. In one embodiment amorphous solid dispersions comprise a compound of the disclosure and a polymer excipient. Other excipients as well as concentrations of said excipients and the compound of the disclosure are well known in the art and are described in standard textbooks. See, for example, “Amorphous Solid Dispersions Theory and Practice” by Navnit Shah et al.

### Immunogenic Compositions Including LNPS

In some aspects, a pharmaceutical composition comprises a compound disclosed herein formulated with a lipid-based delivery system. In some aspects, a pharmaceutical composition comprises a compound disclosed herein and an RNA molecule (e.g., polynucleotide) disclosed herein formulated with a lipid-based delivery system. Thus, some aspects, the composition includes a lipid-based delivery system (e.g., LNPs) (e.g., a lipid-based vaccine), which delivers a nucleic acid molecule to the interior of a cell, where it may then replicate, inhibit protein expression of interest, and/or express the encoded polypeptide of interest. The delivery system may have adjuvant effects which enhance the immunogenicity of an encoded antigen. In some aspects, the composition comprises at least one RNA molecule encoding the antigen polypeptide complexed with, encapsulated in, and/or formulated with one or more lipids, and forming lipid nanoparticles (LNPs), liposomes, lipoplexes and/or nanoliposomes. In some aspects, the composition comprises a lipid nanoparticle. Thus, in certain aspects, the present disclosure concerns compositions comprising one or more lipids associated with a nucleic acid or a polypeptide/peptide.

In one aspect, the disclosure relates to an immunogenic composition may be multivalent, wherein the immunogenic composition comprises (i) a first ribonucleic acid (RNA) polynucleotide having an open reading frame encoding a first antigen, said antigen including at least one antigen polypeptide or an immunogenic fragment thereof, and (ii) a second RNA polynucleotide having an open reading frame encoding a second antigen, said second antigen including at least one polypeptide or an immunogenic fragment thereof, wherein the first and second RNA polynucleotides are formulated in a lipid nanoparticle (LNP). In some embodiments, the first antigen includes an antigen protein from a different subtype to the antigenic polypeptide or an immunogenic fragment thereof of the second antigen. In some embodiments, the first and second RNA polynucleotides are formulated in a lipid nanoparticle. In one aspect, the immunogenic composition is a bivalent immunogenic composition comprising a first molecule encoding an antigen protein and a second molecule encoding an antigen protein. In one aspect, the ratio of the first molecule to the second molecule in the immunogenic composition is 1:1. In another aspect, the multivalent immunogenic composition may be a (i) “pre-mix” formulation wherein two or more RNA polynucleotides are combined in equal ratio followed by co-formulation into LNPs, or (ii) a “post-mix” formulation wherein each RNA polynucleotide is individually formulated into LNPs and the two or more LNPs are combined either in equal ratio or not in equal ratio.

The immunogenic composition including a lipid-based delivery system may further include one or more salts and/or one or more pharmaceutically acceptable surfactants, preservatives, carriers, diluents, and/or excipients, in some cases. In some aspects, the immunogenic composition including a lipid-based delivery system further includes a pharmaceutically acceptable vehicle. In some aspects, each of a buffer, stabilizing agent, and optionally a salt, may be included in the immunogenic composition including a lipid-based delivery system. In other aspects, any one or more of a buffer, stabilizing agent, salt, surfactant, preservative, and excipient may be excluded from the immunogenic composition including a lipid-based delivery system.

In a further aspect, the immunogenic composition including a lipid-based delivery system further comprises a stabilizing agent. In some aspects, the stabilizing agent comprises sucrose, mannose, sorbitol, raffinose, trehalose, mannitol, inositol, sodium chloride, arginine, lactose, hydroxyethyl starch, dextran, polyvinylpyrolidone, glycine, or a combination thereof. In some aspects, 1, 2, 3, 4, 5, or more of the foregoing stabilizing agents may be excluded from the immunogenic compositions disclosed herein. In some aspects, the stabilizing agent is a disaccharide, or sugar. In one aspect, the stabilizing agent is sucrose. In another aspect, the stabilizing agent is trehalose. In a further aspect, the stabilizing agent is a combination of sucrose and trehalose. In some aspects, the total concentration of the stabilizing agent(s) in the composition is or is about 5% to about 10% w/v. For example, the total concentration of the stabilizing agent may or may not be equal to at least, at most, exactly, or between (inclusive or exclusive) of 1%, 2%, 3%, 4%, 5%, 6%, 7%, 8%, 9%, 10%, 11%, 12%, 13%, 14%, 15%, 16%, 17%, 18%, 19%, or 20% w/v or any range or value derivable therein. In some aspects, the stabilizing agent concentration includes, but is not limited to, a concentration of or of about 10 mg/mL to about 400 mg/mL, about 100 mg/mL to about 200 mg/mL, about 100 mg/mL to about 150 mg/mL, about 100 mg/mL to about 140 mg/mL, about 100 mg/mL to about 130 mg/mL, about 100 mg/mL to about 120 mg/mL, about 100 mg/mL to about 110 mg/mL, or about 100 mg/mL to about 105 mg/mL. In some aspects, the concentration of the stabilizing agent is or is not equal to at least, at most, exactly, or between (inclusive or exclusive) of 10 mg/mL, 20 mg/mL, 50 mg/mL, 100 mg/mL, 101 mg/mL, 102 mg/mL, 103 mg/mL, 104 mg/mL, 105 mg/mL, 106 mg/mL, 107 mg/mL, 108 mg/mL, 109 mg/mL, 110 mg/mL, 150 mg/mL, 200 mg/mL, 300 mg/mL, 400 mg/mL, or more.

In a further aspect, the mass amount of the stabilizing agent and the mass amount of the RNA are in a specific ratio. In one aspect, the ratio of the mass amount of the stabilizing agent and the RNA is no greater than 5000. In another aspect, the ratio of the mass amount of the stabilizing agent and the RNA is no greater than 2000. In another aspect, the ratio of the mass amount of the stabilizing agent and the RNA is no greater than 1000. In another aspect, the ratio of the mass amount of the stabilizing agent and the RNA is no greater than 500. In another aspect, the ratio of the mass amount of the stabilizing agent and the RNA is no greater than 100. In another aspect, the ratio of the mass amount of the stabilizing agent and the pharmaceutical substance is no greater than 50. In another aspect, the ratio of the mass amount of the stabilizing agent and the RNA is no greater than 10. In another aspect, the ratio of the mass amount of the stabilizing agent and the RNA is no greater than 1. In another aspect, the ratio of the mass amount of the stabilizing agent and the RNA is no greater than 0.5. In another aspect, the ratio of the mass amount of the stabilizing agent and the RNA is no greater than 0.1. In another aspect, the stabilizing agent and RNA comprise a mass ratio of or of about 200-2000 of the stabilizing agent: 1 of the RNA.

In some aspects, the immunogenic composition including a lipid-based delivery system further comprises a buffer. Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, Tris hydrochloride (HCl), amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. In some aspects, 1, 2, 3, 4, 5, or more of the foregoing buffering agents may be excluded from the immunogenic compositions disclosed herein. In some aspects, the buffer is a HEPES buffer, a Tris buffer, and/or a PBS buffer. In one aspect, the buffer is Tris buffer. In another aspect, the buffer is a HEPES buffer. In a further aspect, the buffer is a PBS buffer. For example, the buffer concentration may or may not be equal to at least, at most, exactly, or between (inclusive or exclusive) of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM, or any range or value derivable therein. The buffer may be at a neutral pH, pH 6.5 to 8.5, pH 7.0 to pH 8.0, or pH 7.2 to pH 7.6. For example, the buffer may or may not be at least, at most, exactly, or between (inclusive or exclusive) of pH 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5, or any range or value derivable therein. In specific aspects, the buffer is at pH 7.4.

In a preferred embodiment, the immunogenic composition comprises a 10 mM Tris buffer. In another preferred embodiment, the immunogenic composition comprises a 10 mM Tris buffer and does not comprise a salt. In another preferred embodiment, the immunogenic composition comprises a 10 mM Tris buffer at pH 7.4. In another preferred embodiment, the immunogenic composition comprises a 10 mM Tris buffer at pH 7.4 and does not comprise a salt.

In some aspects, the immunogenic composition including a lipid-based delivery system may further comprise a salt. Examples of salts include but not limited to sodium salts and/or potassium salts. In one aspect, the salt is a sodium salt. In a specific aspect, the sodium salt is sodium chloride. In one aspect, the salt is a potassium salt. In some aspects, the potassium salt comprises potassium chloride. In some aspects, any one or more of the foregoing salts may be excluded from the immunogenic compositions disclosed herein. The concentration of the salts in the composition may be or be about 70 mM to about 140 mM. For example, the salt concentration may or may not be equal to at least, at most, exactly, or between (inclusive or exclusive) of 50 mM, 60 mM, 70 mM, 80 mM, 90 mM, 100 mM, 120 mM, 130 mM, 140 mM, 150 mM, 160 mM, 170 mM, 180 mM, 190 mM, or 200 mM.

In some aspects, the salt concentration includes, but is not limited to, a concentration of or of about 1 mg/mL to about 100 mg/mL, about 1 mg/mL to about 50 mg/mL, about 1 mg/mL to about 40 mg/mL, about 1 mg/mL to about 30 mg/mL, about 1 mg/mL to about 20 mg/mL, about 1 mg/ml to about 10 mg/mL, or about 1 mg/mL to about 15 mg/mL. In some aspects, the concentration of the salt is or is not equal to at least, at most, exactly, or between (inclusive or exclusive) of 1 mg/mL, 2 mg/ml, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, or more. The salt may be at a neutral pH, pH 6.5 to 8.5, pH 7.0 to pH 8.0, or pH 7.2 to pH 7.6. For example, the salt may or may not be at a pH equal to at least, at most, exactly, or between (inclusive or exclusive) of 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5.

In some aspects, the immunogenic composition including a lipid-based delivery system further comprises a surfactant, a preservative, any other excipient, or a combination thereof. As used herein, “any other excipient” includes, but is not limited to, antioxidants, glutathione, EDTA, methionine, desferal, antioxidants, metal scavengers, and/or free radical scavengers. In one aspect, the surfactant, preservative, excipient or combination thereof is sterile water for injection (sWFI), bacteriostatic water for injection (BWFI), saline, dextrose solution, polysorbates, poloxamers, Triton, divalent cations, Ringer's lactate, amino acids, sugars, polyols, polymers, and/or cyclodextrins. In some aspects, 1, 2, 3, 4, 5, or more of the foregoing excipients may be excluded from the immunogenic compositions disclosed herein.

Further examples of excipients, which refer to ingredients in the immunogenic compositions that are not active ingredients, include but are not limited to carriers, binders, diluents, lubricants, thickeners, surface active agents, preservatives, stabilizers, emulsifiers, buffers, flavoring agents, disintegrants, coatings, plasticizers, compression agents, wet granulation agents, and/or colorants. As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyl oleate), dispersion media, coatings, surfactants, antioxidants, preservatives (e.g., antibacterial and/or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. Diluents, or diluting or thinning agents, include but are not limited to ethanol, glycerol, water, sugars such as lactose, sucrose, mannitol, and sorbitol, and starches derived from wheat, corn rice, and potato; and celluloses such as microcrystalline cellulose. The amount of diluent in the composition may range from or from about 10% to about 90% by weight of the total composition, e.g., from or from about 25% to about 75%, about 30% to about 60% by weight, or about 12% to about 60%. Preservatives for use in the compositions disclosed herein include but are not limited to benzalkonium chloride, chlorobutanol, paraben and thimerosal. In some aspects, 1, 2, 3, 4, 5, or more of the foregoing excipients may be excluded from the immunogenic compositions disclosed herein.

The pH and exact concentration of the various components in the immunogenic composition including a lipid-based delivery system are adjusted according to well-known parameters. The use of such media and agents for pharmaceutical active substances is well known in the art. Except insofar as any conventional media or agent is incompatible with the active ingredients, its use in immunogenic, prophylactic and/or therapeutic compositions is contemplated.

In one aspect, an immunogenic composition comprises a compound disclosed herein and an RNA molecule encoding a polypeptide that is complexed with, encapsulated in, and/or formulated with one or more lipids to form RNA-LNPs. In some aspects, the immunogenic composition is a liquid. In some aspects, the immunogenic composition is frozen. In some aspects, the immunogenic composition is lyophilized. In some aspects, an immunogenic composition comprises a RNA polynucleotide molecule encoding a polypeptide, encapsulated in LNPs with a lipid composition of a cationic lipid, a polymer-conjugated lipid (e.g. PEG-lipid), and one or more structural lipids (e.g., a neutral lipid). In some aspects, any one or more of the foregoing lipids may be excluded from the LNPs of the pharmaceutical compositions disclosed herein.

In some aspects, an immunogenic composition comprises a cationic lipid. The cationic lipid may comprise any one or more cationic lipids disclosed herein.

In specific aspects, the cationic lipid comprises ((4-hydroxybutyl)azanediyl)bis(hexane-6,1-diyl)bis(2-hexyldecanoate) (ALC-0315). In specific aspects, the cationic lipid comprises 2-hexyldecyl6-[(2-{[4-(heptylcarbonylamino)butyl]-N-methylamino}ethyl)[5-(2-hexyldecyloxycarbonyl)pentyl]amino]hexanoate (ALC-0515). In some aspects, the cationic lipid (e.g., ALC-0315 or ALC-0515) is included in the composition at a concentration of at least, at most, between (inclusive or exclusive) of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, or 2.99 ng/μg/mg per mL.

In some aspects, the cationic lipid (e.g., ALC-0315 or ALC-0515) is included in the composition at a concentration of at least, at most, between (inclusive or exclusive) of, or 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, or 2.99 mg/mL. In some aspects, the cationic lipid (e.g., ALC-0315 or ALC-0515) is included in the composition at a concentration of at least 2, at least 2.1, at least 2.2, at least 2.3, at least 2.4, at least 2.5, at least 2.6, at least 2.7, at least 2.8, or at least 2.9 mg/mL. In some aspects, the cationic lipid (e.g., ALC-0315 or ALC-0515) is included in the composition at a concentration of between 2.1 and 2.2, between 2.2 and 2.3, between 2.3 and 2.4, between 2.4 and 2.5, between 2.5 and 2.6, between 2.6 and 2.7, between 2.7 and 2.8, between 2.8 and 2.9, or between 2.9 and 3 mg/mL. In some aspects, the cationic lipid (e.g., ALC-0315 or ALC-0515) is included in the composition at a concentration of between 2 and 2.05, between 2.05 and 2.10, between 2.10 and 2.15, between 2.15 and 2.20, between 2.20 and 2.25, between 2.25 and 2.30, between 2.30 and 2.35, between 2.35 and 2.40, between 2.40 and 2.45, between 2.45 and 2.50, between 2.50 and 2.55, between 2.55 and 2.60, between 2.60 and 2.65, between 2.65 and 2.70, between 2.70 and 2.75, between 2.75 and 2.80, between 2.80 and 2.85, between 2.85 and 2.90, between 2.90 and 2.95 or between 2.95 and 3 mg/mL. In specific aspects, the cationic lipid (e.g., ALC-0315 or ALC-0515) is included in the composition at a concentration of or of about 2.58 mg/mL.

In some aspects, the cationic lipid (e.g., ALC-0315 or ALC-0515) is included in the composition at a concentration of at least, at most, between (inclusive or exclusive) of, or exactly 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 mg/mL. In some aspects, the cationic lipid (e.g., ALC-0315 or ALC-0515) is included in the composition at a concentration of at least 0.4, at least 0.45, at least 0.5, at least 0.55, at least 0.6, at least 0.65, at least 0.7, at least 0.75, at least 0.8, at least 0.85, at least 0.9, at least 0.95, or at least 1 mg/mL. In some aspects, the cationic lipid (e.g., ALC-0315 or ALC-0515) is included in the composition at a concentration of between 0.4 and 0.5, between 0.5 and 0.6, between 0.6 and 0.7, between 0.7 and 0.8, between 0.8 and 0.9, or between 0.9 and 1. In some aspects, the cationic lipid (e.g., ALC-0315 or ALC-0515) is included in the composition at a concentration of between 0.4 and 0.45, between 0.45 and 0.5, between 0.5 and 0.55, between 0.55 and 0.6, between 0.6 and 0.65, between 0.65 and 0.7, between 0.7 and 0.75, between 0.75 and 0.8, between 0.8 and 0.85, between 0.85 and 0.9, between 0.9 and 0.95, or between 0.95 and 1 mg/mL.

In specific aspects, the cationic lipid (e.g., ALC-0315 or ALC-0515) is included in the composition at a concentration of 0.8 to 0.95 mg/mL. In specific aspects, the cationic lipid (e.g., ALC-0315 or ALC-0515) is included in the composition at a concentration of or of about 0.8 to 0.9 mg/mL. In specific aspects, the cationic lipid (e.g., ALC-0315 or ALC-0515) is included in the composition at a concentration of or of about 0.85 to 0.9 mg/mL. In specific aspects, the cationic lipid (e.g., ALC-0315 or ALC-0515) is included in the composition at a concentration of or of at least, at most, exactly, between (inclusive or exclusive) any two of, or about 0.8, 0.81, 0.82, 0.83, 0.84, 0.85, 0.86, 0.87, 0.88, 0.89, 0.9, 0.91, 0.92, 0.93, 0.94, or 0.95 mg/mL. In specific aspects, the cationic lipid (e.g., ALC-0315 or ALC-0515) is included in the composition at a concentration of or of about 0.86 mg/mL. Concentrations for lyophilized compositions are determined post-reconstitution.

In some aspects, an immunogenic composition further comprises a PEGylated lipid (e.g., PEG-lipid).

The PEGylated lipid may comprise any one or more PEGylated lipids disclosed herein. In specific aspects, the PEGylated lipid comprises 2-[(polyethylene glycol)-2000]-N,N-ditetradecylacetamide (ALC-0159). In some aspects, the PEGylated lipid (e.g., ALC-0159) is included in the composition at a concentration of at least, at most, between (inclusive or exclusive) of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/ug/mg per mL.

In some aspects, the PEGylated lipid (e.g., ALC-0159) is included in the composition at a concentration of at least, at most, between (inclusive or exclusive) of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the PEGylated lipid (e.g., ALC-0159) is included in the composition at a concentration of at least 0.01, at least 0.05, at least 0.1, at least 0.15, at least 0.2, at least 0.25 mg/mL, at least 0.3 mg/mL, at least 0.35 mg/mL, at least 0.4 mg/mL, at least 0.45 mg/mL, or at least 0.5 mg/mL. In some aspects, the PEGylated lipid (e.g., ALC-0159) is included in the composition at a concentration of between 0.01 and 0.05, between 0.05 and 0.1, between 0.1 and 0.15, between 0.15 and 0.2, between 0.2 and 0.25, between 0.25 and 0.3, between 0.3 and 0.35, or between 0.35 and 0.4 mg/mL. In specific aspects, the PEGylated lipid (e.g., ALC-0159) is included in the composition at a concentration of or of about 0.32 mg/mL.

In specific aspects, the PEGylated lipid (e.g., ALC-0159) is included in the composition at a concentration of or of about 0.05 to 0.15 mg/mL. In specific aspects, the PEGylated lipid (e.g., ALC-0159) is included in the composition at a concentration of or of about 0.10 to 0.15 mg/mL. In specific aspects, the PEGylated lipid (e.g., ALC-0159) is included in the composition at a concentration of or of at least, at most, exactly, between (inclusive or exclusive) any two of, or about 0.05, 0.06, 0.07, 0.08, 0.09, 0.1, 0.11, 0.12, 0.13, 0.14, or 0.15 mg/mL. In specific aspects, the PEGylated lipid (e.g., ALC-0159) is included in the composition at a concentration of or of about 0.11 mg/mL. Concentrations for lyophilized compositions are determined post-reconstitution.

In some aspects, an immunogenic composition further comprises one or more structural lipids.

The one or more structural lipids may comprise any one or more structural lipids disclosed herein. In specific aspects, the one or more structural lipids comprise a neutral lipid and a steroid or steroid analog. In specific aspects, the one or more structural lipids comprise 1,2-distearoyl-sn-glycero-3-phosphocholine (DSPC) and cholesterol. In some aspects, the one or more structural lipids (e.g., DSPC or cholesterol) are included in the composition at a concentration of at least, at most, between (inclusive or exclusive) of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/ug/mg per mL. In some aspects, the one or more structural lipids (e.g., DSPC or cholesterol) are included in the composition at a concentration of at least, at most, between (inclusive or exclusive) of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51. 0.52. 0.53, 0.54, 0.55, 0.56, 0.57, 0.58, 0.59 or 0.6 mg/mL. In some aspects, the one or more structural lipids (e.g., DSPC or cholesterol) are included in the composition at a concentration of at least 0.05, at least 0.1, at least 0.15, at least 0.2, at least 0.25, at least 0.3, at least 0.35, at least 0.4, at least 0.45, at least 0.5, at least 0.55, at least 0.6, at least 0.65, at least 0.7, at least 0.75, at least 0.8, at least 0.85, at least 0.9, at least 0.95 or at least 1 mg/mL. In some aspects, the one or more structural lipids (e.g., DSPC or cholesterol) are included in the composition at a concentration of between 0.05 and 0.1, between 0.1 and 0.15, between 0.15 and 0.2, between 0.2 and 0.25, between 0.25 and 0.3, between 0.3 and 0.35, between 0.35 and 0.4, between 0.4 and 0.45, between 0.45 and 0.5, between 0.5 and 0.55, between 0.55 and 0.6, between 0.6 and 0.65, between 0.65 and 0.7, between 0.7 and 0.75, between 0.75 and 0.8, between 0.8 and 0.85, between 0.85 and 0.9, between 0.9 and 0.95, or between 0.95 and 1 mg/mL.

In specific aspects, the one or more structural lipids include DSPC, and the DSPC is included in the composition at a concentration of or of about 0.1 to 0.25 mg/mL. In specific aspects, the one or more structural lipids include DSPC, and the DSPC is included in the composition at a concentration of or of about 0.15 to 0.25 mg/mL. In specific aspects, the one or more structural lipids include DSPC, and the DSPC is included in the composition at a concentration of or of at least, at most, exactly, between (inclusive or exclusive) any two of, or about 0.1, 0.11, 0.12, 0.13, 0.14, 0.15, 0.16, 0.17, 0.18, 0.19, 0.2, 0.21, 0.22, 0.23, 0.24, or 0.25 mg/mL. In specific aspects, the DSPC is included in the composition at a concentration of or of about 0.19 mg/mL. In specific aspects, the DSPC is included in the composition at a concentration of or of about 0.56 mg/mL.

In specific aspects, the one or more structural lipids include cholesterol, and the cholesterol is included in the composition at a concentration of or of about 0.3 to 0.45 mg/mL. In specific aspects, the one or more structural lipids include cholesterol, and the cholesterol is included in the composition at a concentration of or of about 0.3 to 0.4. In specific aspects, the one or more structural lipids include cholesterol, and the cholesterol is included in the composition at a concentration of or of about 0.35 to 0.45. In specific aspects, the one or more structural lipids include cholesterol, and the cholesterol is included in the composition at a concentration of or of at least, at most, exactly, between (inclusive or exclusive) any two of, or about 0.30, 0.31, 0.32, 0.33, 0.34, 0.35, 0.36, 0.37, 0.38, 0.39, 0.40, 0.41, 0.42, 0.43, 0.44, or 0.45 mg/mL. In specific aspects, the cholesterol is included in the composition at a concentration of and/or of about 0.37 mg/mL. Concentrations for lyophilized compositions are determined post-reconstitution.

In specific aspects, the one or more structural lipids include cholesterol, and the cholesterol is included in the composition at a concentration of or of about 1 to 1.25 mg/ml. In specific aspects, the one or more structural lipids include cholesterol, and the cholesterol is included in the composition at a concentration of or of about 1.10 to 1.15. In specific aspects, the one or more structural lipids include cholesterol, and the cholesterol is included in the composition at a concentration of or of about 1.15 to 1.20. In specific aspects, the one or more structural lipids include cholesterol, and the cholesterol is included in the composition at a concentration of or of at least, at most, exactly, between (inclusive or exclusive) any two of, or about 1, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24 or 1.25 mg/mL. In specific aspects, the cholesterol is included in the composition at a concentration of and/or of about 1.12 mg/mL. Concentrations for lyophilized compositions are determined post-reconstitution.

In some aspects, the immunogenic composition further comprises one or more buffers and stabilizing agents, and optionally, salt diluents. Thus, in some aspects, the immunogenic composition comprises a cationic lipid, a PEGylated lipid, one or more structural lipids, one or more buffers, a stabilizing agent, and optionally, a salt diluent. In some aspects, 1, 2, 3, or more of the foregoing elements are excluded from the immunogenic composition.

In some aspects, an immunogenic composition comprises one or more buffers.

The one or more buffers may comprise any one or more buffering agents disclosed herein. In specific aspects, the composition comprises a Tris buffer comprising at least a first buffer and a second buffer. In some aspects, the first buffer is tromethamine. In some aspects, the second buffer is Tris hydrochloride (HCl). In some aspects, the first buffer and second buffer of the Tris buffer (e.g., tromethamine and Tris HCl) are included in the composition at a concentration of at least, at most, between (inclusive or exclusive) of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/ug/mg per mL. Concentrations for lyophilized compositions are determined post-reconstitution.

In some aspects, the immunogenic composition is a liquid composition comprising a Tris buffer.

In some aspects, the Tris buffer comprises a first buffer. In some aspects, the first buffer is tromethamine. In some aspects, the first buffer (e.g., tromethamine) is included in the liquid composition at a concentration of at least, at most, between (inclusive or exclusive) of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the first buffer (e.g., tromethamine) is included in the liquid composition at a concentration of at least 0.01, at least 0.05, at least 0.1, at least 0.15, at least 0.2, at least 0.25, at least 0.3, at least 0.35, at least 0.4, at least 0.45, at least 0.5, at least 0.55, at least 0.6, at least 0.65, at least 0.7, at least 0.75, at least 0.8, at least 0.85, at least 0.9, at least 0.95, or at least 1 mg/mL. In some aspects, the first buffer (e.g., tromethamine) is included in the liquid composition at a concentration of between 0.05 and 0.15, between 0.15 and 0.25, between 0.25 and 0.35, between 0.35 and 0.45, between 0.45 and 0.55, between 0.55 and 0.65, between 0.65 and 0.75, between 0.75 and 0.85, or between 0.85 and 0.95. In some aspects, the first buffer (e.g., tromethamine) is included in the liquid composition at a concentration of between 0.05 and 0.1, between 0.1 and 0.15, between 0.15 and 0.2, between 0.2 and 0.25, between 0.25 and 0.3, between 0.3 and 0.35, between 0.35 and 0.4, between 0.4 and 0.45, between 0.45 and 0.5, between 0.5 and 0.55, between 0.55 and 0.6, between 0.6 and 0.65, between 0.65 and 0.7, between 0.7 and 0.75, between 0.75 and 0.8, between 0.8 and 0.85, between 0.85 and 0.9, between 0.9 and 0.95, or between 0.95 and 1 mg/mL.

In specific aspects, the first buffer (e.g., tromethamine) is included in the liquid composition at a concentration of or of about 0.1 to 0.3 mg/mL. In specific aspects, the first buffer (e.g., tromethamine) is included in the liquid composition at a concentration of or of about 0.15 to 0.25 mg/mL. In specific aspects, the first buffer (e.g., tromethamine) is included in the liquid composition at a concentration of or of at least, at most, exactly, between (inclusive or exclusive) any two of, or about 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, or 0.3 mg/mL. In specific aspects, the first buffer (e.g., tromethamine) is included in the liquid composition at a concentration of or of about 0.20 mg/mL.

In some aspects, the immunogenic composition is a liquid composition comprising a Tris buffer comprising a second buffer.

In some aspects, the second buffer comprises Tris HCl. In some aspects, the second buffer (e.g., Tris HCl) is included in the liquid composition at a concentration of at least, at most, between (inclusive or exclusive) of, or exactly 0.5, 0.55, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, or 1.5 mg/mL. In some aspects, the second buffer (e.g., Tris HCl) is included in the liquid composition at a concentration of at least 0.5, at least 0.55, at least 0.6, at least 0.65, at least 0.7, at least 0.75, at least 0.8, at least 0.85, at least 0.9, at least 0.95, at least 1, at least 1.05, at least 1.10, at least 1.15, at least 1.20, at least 1.25, at least 1.30, at least 1.35, at least 1.40, at least 1.45, or at least 1.50 mg/mL. In some aspects, the second buffer (e.g., Tris HCl) is included in the liquid composition at a concentration of between 0.5 and 0.6, between 0.6 and 0.7, between 0.7 and 0.8, between 0.8 and 0.9, between 0.9 and 1, between 1 and 1.10, between 1.10 and 1.20, between 1.20 and 1.30, between 1.30 and 1.40, or between 1.40 and 1.50 mg/mL.

In specific aspects, the second buffer (e.g., Tris HCl) is included in the liquid composition at a concentration of or of about 1.25 to 1.40 mg/mL. In specific aspects, the second buffer (e.g., Tris HCl) is included in the liquid composition at a concentration of or of about 1.30 to 1.40 mg/mL. In specific aspects, the second buffer (e.g., Tris HCl) is included in the liquid composition at a concentration of or of at least, at most, exactly, between (inclusive or exclusive) any two of, or about 1.25, 1.26, 1.27, 1.28, 1.29, 1.30, 1.31, 1.32, 1.33, 1.34, or 1.35, 1.36, 1.37, 1.38, 1.39, or 1.40 mg/mL. In one specific aspect, the second buffer (e.g., Tris HCl) is included in the liquid composition at a concentration of or of about 1.32 mg/mL.

In some aspects, the immunogenic composition is a lyophilized composition comprising a Tris buffer. In some aspects, the Tris buffer comprises a first buffer. In some aspects, the first buffer is tromethamine. In some aspects, the first buffer (e.g., tromethamine) is included in the lyophilized composition at a concentration, after reconstitution, of at least, at most, between (inclusive or exclusive) of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the first buffer (e.g., tromethamine) is included in the lyophilized composition at a concentration, after reconstitution, of at least 0.01, of at least 0.05, of at least 0.1, of at least 0.15, of at least 0.2, of at least 0.25, of at least 0.3, of at least 0.35, of at least 0.4, of at least 0.45, or of at least 0.5 mg/mL. In some aspects, the first buffer (e.g., tromethamine (Tris base)) is included in the lyophilized composition at a concentration, after reconstitution, of between 0.01 and 0.05, between 0.05 and 0.1, between 0.1 and 0.15, between 0.15 and 0.2, between 0.2 and 0.25 mg/mL, between 0.25 and 0.3 mg/mL, between 0.3 and 0.35 mg/mL, between 0.35 and 0.4 mg/mL, between 0.4 and 0.45 mg/mL, or between 0.45 and 0.5 mg/mL.

In specific aspects, the first buffer (e.g., tromethamine) is included in the lyophilized composition at a concentration, after reconstitution, of or of about 0.01 and 0.20 mg/mL. In specific aspects, the first buffer (e.g., tromethamine) is included in the lyophilized composition at a concentration, after reconstitution, of or of about 0.01 and 0.10 mg/mL. In specific aspects, the first buffer (e.g., tromethamine) is included in the lyophilized composition at a concentration, after reconstitution, of or of about 0.05 and 0.15 mg/mL. In specific aspects, the first buffer (e.g., tromethamine) is included in the lyophilized composition at a concentration, after reconstitution, of or of at least, at most, exactly, between (inclusive or exclusive) any two of, or about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19 or 0.20 mg/mL. In one specific aspect, the first buffer (e.g., tromethamine) is included in the lyophilized composition at a concentration, after reconstitution, of or of about 0.09 mg/mL. In one specific aspect, the first buffer (e.g., tromethamine) is included in the lyophilized composition at a concentration, after reconstitution, of or of about 0.09 mg/mL.

In some aspects, the immunogenic composition is a lyophilized composition comprising a Tris buffer comprising a second buffer. In some aspects, the second buffer comprises Tris HCl. In some aspects, the second buffer (e.g., Tris HCl) is included in the lyophilized composition at a concentration, after reconstitution, of at least, at most, between (inclusive or exclusive) of, or exactly 0.1, 0.15, 0.2, 0.25, 0.3, 0.35, 0.4, 0.45, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 mg/mL. In some aspects, the second buffer (e.g., Tris HCl) is included in the lyophilized composition at a concentration, after reconstitution, of at least 0.1, at least 0.2, at least 0.3, at least 0.4, at least 0.5, at least 0.6, at least 0.7, at least 0.8, at least 0.9, or at least 1 mg/mL. In some aspects, the second buffer (e.g., Tris HCl) is included in the lyophilized composition at a concentration, after reconstitution, of between 0.1 and 0.2, between 0.2 and 0.3, between 0.3 and 0.4, between 0.4 and 0.5, between 0.5 and 0.6, between 0.6 and 0.7, between 0.7 and 0.8, between 0.8 and 0.9, or between 0.9 and 1 mg/mL.

In specific aspects, the second buffer (e.g., Tris HCl) is included in the lyophilized composition at a concentration, after reconstitution, of or of about 0.5 and 0.65 mg/mL. In specific aspects, the second buffer (e.g., Tris HCl) is included in the lyophilized composition at a concentration, after reconstitution, of or of about 0.5 and 0.6 mg/mL. In specific aspects, the second buffer (e.g., Tris HCl) is included in the lyophilized composition at a concentration, after reconstitution, of or of about 0.55 and 0.65 mg/mL. In specific aspects, the second buffer (e.g., Tris HCl) is included in the lyophilized composition at a concentration, after reconstitution, of or of at least, at most, exactly, between (inclusive or exclusive) any two of, or about 0.5, 0.51, 0.52, 0.53, 0.54, 0.55, 0.56, 0.57, 0.58, 0.59, 0.6, 0.61, 0.62, 0.63, 0.64, or 0.65 mg/mL. In specific aspects, the second buffer (e.g., Tris HCl) is included in the lyophilized composition at a concentration, after reconstitution, of or of about 0.57 mg/mL.

In some aspects, an immunogenic composition comprises a stabilizing agent. The stabilizing agent may comprise any one or more stabilizing agents disclosed herein. In some aspects, the stabilizing agent also functions as a cryoprotectant. In specific aspects, the stabilizing agent comprises sucrose. In some aspects, the stabilizing agent (e.g., sucrose) is included in the composition at a concentration of at least, at most, between (inclusive or exclusive) of, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 ng/ug/mg per mL.

In some aspects, the immunogenic composition is a liquid composition, and the stabilizing agent (e.g., sucrose) is included in the liquid composition at a concentration of at least, at most, between (inclusive or exclusive) of, or exactly 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 mg/mL. In some aspects, the stabilizing agent (e.g., sucrose) is included in the liquid composition at a concentration of at least 70, at least 75, at least 80, at least 85, at least 90, at least 95, at least 100, at least 105, at least 110, at least 115, at least 120, at least 125, or at least 130 mg/mL. In some aspects, the stabilizing agent (e.g., sucrose) is included in the liquid composition at a concentration of between 70 and 80, between 80 and 90, between 90 and 100, between 100 and 110, between 110 and 120, or between 120 and 130 mg/mL.

In specific aspects, the stabilizing agent (e.g., sucrose) is included in the liquid composition at a concentration of or of about 95 to 110 mg/mL. In specific aspects, the stabilizing agent (e.g., sucrose) is included in the liquid composition at a concentration of or of about 95 to 105 mg/mL. In specific aspects, the stabilizing agent (e.g., sucrose) is included in the liquid composition at a concentration of or of about 100 to 110 mg/mL. In specific aspects, the stabilizing agent (e.g., sucrose) is included in the liquid composition at a concentration of or of at least, at most, exactly, between (inclusive or exclusive) any two of, or about 95, 96, 97, 98, 99, 100, 101, 102, 103, 104, 105, 106, 107, 108, 109, or 110 mg/mL. In specific aspects, the stabilizing agent (e.g., sucrose) is included in the liquid composition at a concentration of or of about 103 mg/mL.

In some aspects, the immunogenic composition is a lyophilized composition, and the stabilizing agent (e.g., sucrose) is or is not included in the lyophilized composition at a concentration, after reconstitution, of at least, at most, between (inclusive or exclusive) of, or exactly 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 mg/mL. In some aspects, the stabilizing agent (e.g., sucrose) is included in the lyophilized composition at a concentration, after reconstitution, of at least 20, at least 25, at least 30, at least 35, at least 40, at least 45, at least 50, at least 55, at least 60, at least 65, at least 70, at least 75, or at least 80 mg/mL. In some aspects, the stabilizing agent (e.g., sucrose) is included in the lyophilized composition at a concentration, after reconstitution, of between 20 to 30, between 30 to 40, between 40 to 50, between 50 to 60, between 60 to 70, or between 70 to 80 mg/mL.

In specific aspects, the stabilizing agent (e.g., sucrose) is included in the lyophilized composition at a concentration, after reconstitution, of or of about 35 to 50 mg/mL. In specific aspects, the stabilizing agent (e.g., sucrose) is included in the lyophilized composition at a concentration, after reconstitution, of or of about 35 to 45 mg/mL. In specific aspects, the stabilizing agent (e.g., sucrose) is included in the lyophilized composition at a concentration, after reconstitution, of or of about 40 to 50 mg/mL. In specific aspects, the stabilizing agent (e.g., sucrose) is included in the lyophilized composition at a concentration, after reconstitution, of or of at least, at most, exactly, between (inclusive or exclusive) any two of, or about 35, 36, 37, 38, 39, 40, 41, 42, 43, 44, 45, 46, 47, 48, 49, or 50 mg/mL. In specific aspects, the stabilizing agent (e.g., sucrose) is included in the lyophilized composition at a concentration, after reconstitution, of or of about 44/mL.

In some aspects, lyophilized compositions are reconstituted in a suitable carrier and/or diluent. The carrier and/or diluent may comprise any one or more carriers and/or diluents disclosed herein. In specific aspects, the carrier and/or diluent comprises a salt diluent, such as sodium chloride (NaCl) (e.g., saline, e.g., physiological or normal saline). The sodium chloride may comprise 0.9% sodium chloride for injection. In some aspects, the lyophilized compositions are or are not reconstituted in at least, at most, between (inclusive or exclusive) of, or exactly 0.1, 0.2, 0.3, 0.4, 0.5, 0.6, 0.7, 0.8, 0.9, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1 mL of saline. In some aspects, the lyophilized compositions are reconstituted in at least 0.1, at least 0.2, at least 0.3, at least 0.4, at least 0.5, at least 0.6, at least 0.7, at least 0.8, at least 0.9, or at least 1 mL of sodium chloride.

In specific aspects, the lyophilized compositions are reconstituted in or in about 0.6 to 0.75 mL of sodium chloride/saline. In specific aspects, the lyophilized compositions are reconstituted in or in about 0.65 to 0.75 mL of sodium chloride/saline. In specific aspects, the lyophilized compositions are reconstituted in or in at least, at most, exactly, between (inclusive or exclusive) any two of, or about 0.6, 0.61, 0.62, 0.63, 0.64, 0.65, 0.66, 0.67, 0.68, 0.69, 0.7, 0.71, 0.72, 0.73, 0,74, or 0.75 mL of sodium chloride/saline.

In some aspects, the salt diluent (e.g., NaCl) is included in the lyophilized composition at a concentration, after reconstitution, of at least, at most, between (inclusive or exclusive) of, or exactly 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, or 50 ng/ug/mg per mL. In some aspects, the salt diluent (e.g., NaCl) is included in the lyophilized composition at a concentration, after reconstitution, of in at least, at most, between (inclusive or exclusive) of, or exactly 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 mg/mL. In some aspects, the salt diluent (e.g., NaCl) is included in the lyophilized composition at a concentration, after reconstitution, of at least 1, at least 2, at least 3, at least 4, at least 5, at least 6, at least 7, at least 8, at least 9, at least 10, at least 11, at least 12, at least 13, at least 14, at least 15, or at least 20 mg/mL.

In specific aspects, the salt diluent (e.g., NaCl) is included in the lyophilized composition at a concentration, after reconstitution, of between or between about 5 and 15 mg/mL. In some aspects, the salt diluent (e.g., NaCl) is included in the lyophilized composition at a concentration, after reconstitution, of between or between about 5 and 10 mg/mL. In specific aspects, the salt diluent (e.g., NaCl) is included in the lyophilized composition at a concentration, after reconstitution, of or of at least, at most, exactly, between (inclusive or exclusive) any two of, or about 5, 6, 7, 8, 9, 10, 11, 12, 13, 14, or 15 mg/mL. In specific aspects, the salt diluent (e.g., NaCl) is included in the lyophilized composition at a concentration, after reconstitution, of or of about 9 mg/mL.

The pH of the immunogenic composition may be at least, at most, exactly, or between (inclusive or exclusive) of pH 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5, or any range or value derivable therein. In some aspects, the immunogenic composition is at a pH of at least 6.5, at least 7.0, at least 7.5, at least 8.0, or at least 8.5. In specific aspects, the immunogenic composition is at a pH between 6.0 and 7.5, between 6.5 and 7.5, between 7.0 and 8.0, between and 7.5 and 8.5. In specific aspects, the immunogenic composition is at a pH between 7.0 and 8.0. In specific aspects, the immunogenic composition is at least, at most, exactly, between (inclusive or exclusive) any two of, or about pH 7.0, 7.1, 7.2, 7.3, 7.4, 7.5, 7.6, 7.7, 7.8, 7.9, or 8.0. In specific aspects, the immunogenic composition is at or at about pH 7.4. In some aspects, sodium hydroxide buffer may be used for a buffer pH adjustment.

In specific aspects, an immunogenic composition comprises a RNA polynucleotide encoding a polypeptide as disclosed herein, encapsulated in LNPs with a lipid composition of a cationic lipid at a concentration of or of about 0.8 to 0.95 mg/mL, a PEGylated lipid at a concentration of or of about 0.05 to 0.15 mg/mL, a first structural lipid (e.g. DSPC) at a concentration of or of about 0.1 to 0.25 mg/mL, and a second structural lipid (e.g. cholesterol) at a concentration of or of about 0.3 to 0.45 mg/mL. In some aspects, one or more of the foregoing elements may be excluded from the immunogenic composition.

In specific aspects, the immunogenic composition is a liquid. The liquid immunogenic composition further comprises a buffer composition comprising a first buffer at a concentration of or of about 0.15 to 0.3 mg/mL, a second buffer at a concentration of or of about 1.25 to 1.4 mg/mL, and a stabilizing agent at a concentration of or of about 95 to 110 mg/mL. In specific aspects, the immunogenic composition further comprises a Tris buffer composition comprising tromethamine at a concentration of or of about 0.1 to 0.3 mg/mL, Tris HCl at a concentration of or of about 1.25 to 1.4 mg/mL, and sucrose at a concentration of or of about 95 to 110 mg/mL. In some aspects, one or more of the foregoing elements may be excluded from the immunogenic composition.

Thus, in specific aspects, a liquid immunogenic composition comprises an cationic lipid at a concentration of or of about 0.8 to 0.95 mg/mL, a PEGylated lipid at a concentration of or of about 0.05 to 0.15 mg/mL, a first structural lipid at a concentration of or of about 0.1 to 0.25 mg/mL, a second structural lipid at a concentration of or of about 0.3 to 0.45 mg/mL, and further comprises a first buffer at a concentration of or of about 0.1 to 0.3 mg/mL, a second buffer at a concentration of or of about 1.25 to 1.4 mg/mL, and a stabilizing agent at a concentration of or of about 95 to 110 mg/mL. In some aspects, one or more of the foregoing elements may be excluded from the immunogenic composition.

Thus, in specific aspects, a liquid immunogenic composition comprises ALC-0315 or ALC-0515 at a concentration of or of about 0.8 to 0.95 mg/mL, ALC-0159 at a concentration of or of about 0.05 to 0.15 mg/mL, DSPC at a concentration of or of about 0.1 to 0.25 mg/mL, cholesterol at a concentration of or of about 0.3 to 0.45 mg/mL, and further comprises a Tris buffer composition comprising tromethamine at a concentration of or of about 0.1 to 0.3 mg/mL, Tris HCl at a concentration of or of about 1.25 to 1.4 mg/mL, and sucrose at a concentration of or of about 95 to 110 mg/mL. In some aspects, one or more of the foregoing elements may be excluded from the immunogenic composition.

In specific aspects, the immunogenic composition is a lyophilized immunogenic composition, and the lyophilized immunogenic composition further comprises (after reconstitution) a first buffer at a concentration of or of about 0.01 and 0.15 mg/mL, a second buffer at a concentration of or of about 0.5 and 0.65 mg/mL, a stabilizing agent at a concentration of or of about 35 to 50 mg/mL, and a salt diluent at a concentration of between or between about 5 and 15 mg/mL. In some aspects, one or more of the foregoing elements may be excluded from the immunogenic composition.

In specific aspects, the immunogenic composition is a lyophilized immunogenic composition, and the lyophilized immunogenic composition further comprises (after reconstitution) a Tris buffer composition comprising tromethamine at a concentration of or of about 0.01 and 0.15 mg/mL, Tris HCl at a concentration of or of about 0.5 and 0.65 mg/mL, sucrose at a concentration of or of about 35 to 50 mg/mL, and sodium chloride (NaCl) at a concentration of or of about 5 to 15 mg/mL. In some aspects, one or more of the foregoing elements may be excluded from the immunogenic composition.

Thus, in specific aspects, a lyophilized immunogenic composition comprises (after reconstitution) a cationic lipid at a concentration of or of about 0.8 to 0.95 mg/mL, a PEGylated lipid at a concentration of or of about 0.05 to 0.15 mg/mL, a first structural lipid at a concentration of or of about 0.1 to 0.25 mg/mL, a second structural lipid at a concentration of or of about 0.3 to 0.45 mg/mL, and further comprises a first buffer at a concentration of or of about 0.01 and 0.15 mg/mL, a second buffer at a concentration of or of about 0.5 and 0.65 mg/mL, a stabilizing agent at a concentration of or of about 35 to 50 mg/mL, and a salt diluent at a concentration of or of about 5 to 15 mg/mL. In specific aspects, the lyophilized compositions are reconstituted in 0.6 to 0.75 mL of the salt diluent. In some aspects, one or more of the foregoing elements may be excluded from the immunogenic composition.

Thus, in some aspects, a lyophilized immunogenic composition comprises (after reconstitution) ALC-0315 or ALC-0515 at a concentration of or of about 0.8 to 0.95 mg/mL, ALC-0159 at a concentration of or of about 0.05 to 0.15 mg/mL, DSPC at a concentration of or of about 0.1 to 0.25 mg/mL, cholesterol at a concentration of or of about 0.3 to 0.45 mg/mL, and further comprises tromethamine at a concentration of or of about 0.01 and 0.15 mg/mL, Tris HCl at a concentration of or of about 0.5 and 0.65 mg/mL, sucrose at a concentration of or of about 35 to 50 mg/mL, and NaCl at a concentration of or of about 5 to 15 mg/mL. In specific aspects, the lyophilized compositions are reconstituted in 0.6 to 0.75 mL of NaCl (saline). In some aspects, one or more of the foregoing elements may be excluded from the immunogenic composition.

Concentrations in the lyophilized immunogenic composition above are determined post-reconstitution.

In some aspects, an immunogenic composition (pre-lyophilization) comprises a cationic lipid at a concentration of or of about 1.0 to 3.0 mg/mL, a PEGylated lipid at a concentration of or of about 0.10 to 0.35 mg/mL, a first structural lipid at a concentration of or of about 0.4 to 0.55 mg/mL, a second structural lipid at a concentration of or of about 0.85 to 1.0 mg/mL, and further comprises a first buffer at a concentration of or of about 0.1 and 0.3 mg/mL, a second buffer at a concentration of or of about 1.25 and 1.40 mg/mL, a stabilizing agent at a concentration of or of about 95 to 110 mg/mL. In some aspects, one or more of the foregoing elements may be excluded from the immunogenic composition.

Thus, in some aspects, a immunogenic composition (pre-lyophilization) comprises ALC-0315 or ALC-0515 at a concentration of or of about 1.0 to 3.0 mg/mL, ALC-0159 at a concentration of or of about 0.10 to 0.35 mg/mL, DSPC at a concentration of or of about 0.4 to 0.55 mg/mL, cholesterol at a concentration of or of about 0.85 to 1.0 mg/mL, and further comprises tromethamine at a concentration of or of about 0.1 and 0.3 mg/mL, Tris HCl at a concentration of or of about 1.25 and 1.40 mg/mL, sucrose at a concentration of or of about 95 to 110 mg/mL. In some aspects, one or more of the foregoing elements may be excluded from the immunogenic composition.

In specific aspects, the immunogenic composition is a liquid immunogenic composition comprising a RNA polynucleotide encoding a polypeptide as disclosed herein at a concentration of at least, at most, exactly, or between (inclusive or exclusive) of 0.01, 0.15, 0.30, 0.45, 0.60, 0.75, or 0.90 mg/mL, preferably of or of about 0.01 to 0.09 mg/mL, encapsulated in LNPs with a lipid composition of an cationic lipid at a concentration of or of about 0.8 to 0.95 mg/mL, a PEGylated lipid at a concentration of or of about 0.05 to 0.15 mg/mL, a first structural lipid at a concentration of or of about 0.1 to 0.25 mg/mL, and a second structural lipid at a concentration of or of about 0.3 to 0.45 mg/mL, and further comprising a buffer composition comprising a first buffer at a concentration of or of about 0.15 to 0.3 mg/mL, a second buffer at a concentration of or of about 1.25 to 1.4 mg/mL, and a stabilizing agent at a concentration of or of about 95 to 110 mg/mL. In some aspects, one or more of the foregoing elements may be excluded from the immunogenic composition.

In specific aspects, a liquid immunogenic composition comprises a RNA polynucleotide encoding a polypeptide as disclosed herein at a concentration of at least, at most, exactly, or between (inclusive or exclusive) of 0.01, 0.15, 0.30, 0.45, 0.60, 0.75, or 0.90 mg/mL, preferably of or of about 0.01 to 0.09 mg/mL, and more preferably of or of about 0.06 mg/mL, encapsulated in LNPs with a lipid composition of ALC-0315 or ALC-0515 at a concentration of or of about 0.8 to 0.95 mg/mL, ALC-0159 at a concentration of or of about 0.05 to 0.15 mg/mL, DSPC at a concentration of or of about 0.1 to 0.25 mg/mL, and cholesterol at a concentration of or of about 0.3 to 0.45 mg/mL, and further comprising a Tris buffer composition comprising tromethamine at a concentration of or of about 0.1 to 0.3 mg/mL, Tris HCl at a concentration of or of about 1.25 to 1.4 mg/mL, and sucrose at a concentration of or of about 95 to 110 mg/mL. In some aspects, one or more of the foregoing elements may be excluded from the immunogenic composition.

In specific aspects, the immunogenic composition is a lyophilized immunogenic composition comprising a RNA polynucleotide encoding a polypeptide as disclosed herein at a concentration of at least, at most, exactly, or between (inclusive or exclusive) of 0.01, 0.15, 0.30, 0.45, 0.60, 0.75, or 0.90 mg/mL, preferably of or of about 0.01 to 0.09 mg/mL, encapsulated in LNPs with a lipid composition of a cationic lipid at a concentration of or of about 0.8 to 0.95 mg/mL, a PEGylated lipid at a concentration of or of about 0.05 to 0.15 mg/mL, a first structural lipid at a concentration of or of about 0.1 to 0.25 mg/mL, and a second structural lipid at a concentration of or of about 0.3 to 0.45 mg/mL, and further comprising a first buffer at a concentration of or of about 0.01 and 0.15 mg/mL, a second buffer at a concentration of or of about 0.5 and 0.65 mg/mL, a stabilizing agent at a concentration of or of about 35 to 50 mg/mL, and a salt diluent at a concentration of 5 to 15 mg/mL. In some aspects, one or more of the foregoing elements may be excluded from the immunogenic composition. In specific aspects, the lyophilized compositions are reconstituted in 0.6 to 0.75 mL of the salt diluent. Concentrations in the lyophilized immunogenic composition are determined post-reconstitution.

In specific aspects, a lyophilized immunogenic composition comprises a RNA polynucleotide encoding a polypeptide as disclosed herein at a concentration of at least, at most, exactly, or between (inclusive or exclusive) of 0.01, 0.15, 0.30, 0.45, 0.60, 0.75, or 0.90 mg/mL, preferably of or of about 0.01 to 0.09 mg/mL, and more preferably of or of about 0.06 mg/mL, encapsulated in LNPs with a lipid composition of ALC-0315 or ALC-0515 at a concentration of or of about 0.8 to 0.95 mg/mL, ALC-0159 at a concentration of or of about 0.05 to 0.15 mg/mL, DSPC at a concentration of or of about 0.1 to 0.25 mg/mL, and cholesterol at a concentration of or of about 0.3 to 0.45 mg/mL, and further comprising tromethamine at a concentration of or of about 0.01 and 0.15 mg/mL, Tris HCl at a concentration of or of about 0.5 and 0.65 mg/mL, sucrose at a concentration of or of about 35 to 50 mg/mL, and NaCl at a concentration of or of about 5 to 15 mg/mL. In some aspects, one or more of the foregoing elements may be excluded from the immunogenic composition. In specific aspects, the lyophilized compositions are reconstituted in 0.6 to 0.75 mL of the NaCl diluent (saline). Concentrations in the lyophilized immunogenic composition are determined post-reconstitution.

In some aspects, an immunogenic composition (pre-lyophilization) comprises a RNA polynucleotide encoding a polypeptide as disclosed herein at a concentration of at least, at most, exactly, or between (inclusive or exclusive) of 0.01, 0.15, 0.30, 0.45, 0.60, 0.75, or 0.90 mg/mL, preferably of or of about 0.01 to 0.09 mg/mL, encapsulated in LNPs with a lipid composition of a cationic lipid at a concentration of or of about 1.0 to 3.0 mg/ml, a PEGylated lipid at a concentration of or of about 0.10 to 0.35 mg/mL, a first structural lipid at a concentration of or of about 0.4 to 0.55 mg/mL, a second structural lipid at a concentration of or of about 0.85 to 1.0 mg/mL, and further comprises a first buffer at a concentration of or of about 0.1 and 0.3 mg/mL, a second buffer at a concentration of or of about 1.25 and 1.40 mg/mL, a stabilizing agent at a concentration of or of about 95 to 110 mg/mL. In some aspects, one or more of the foregoing elements may be excluded from the composition.

Thus, in some aspects, a composition (pre-lyophilization) comprises a RNA polynucleotide encoding a polypeptide as disclosed herein at a concentration of at least, at most, exactly, or between (inclusive or exclusive) of 0.01, 0.15, 0.30, 0.45, 0.60, 0.75, or 0.90 mg/mL, preferably of or of about 0.01 to 0.09 mg/mL, and more preferably 0.15 mg/mL, encapsulated in LNPs with a lipid composition of comprises ALC-0315 or ALC-0515 at a concentration of or of about 1.0 to 3.0 mg/mL, ALC-0159 at a concentration of or of about 0.10 to 0.35 mg/mL, DSPC at a concentration of or of about 0.4 to 0.55 mg/mL, cholesterol at a concentration of or of about 0.85 to 1.0 mg/mL, and further comprises tromethamine at a concentration of or of about 0.1 and 0.3 mg/mL, Tris HCl at a concentration of or of about 1.25 and 1.40 mg/ml, sucrose at a concentration of or of about 95 to 110 mg/mL. In some aspects, one or more of the foregoing elements may be excluded from the composition.

In some aspects, the liquid immunogenic composition comprises an RNA molecule/polynucleotide encoding a polypeptide as disclosed herein at a concentration of at least, at most, exactly, or between (inclusive or exclusive) of 0.01, 0.15, 0.30, 0.45, 0.60, 0.75, or 0.90 mg/mL, preferably of or of about 0.01 to 0.09 mg/mL, encapsulated in a LNP, and further comprising or comprising about 5 to 15 mM Tris buffer and about 200 to 400 mM sucrose at a pH of or of about 7.0 to 8.0. In some aspects, one or more of the foregoing elements may be excluded from the liquid immunogenic composition.

In a preferred embodiment, the immunogenic composition comprises 300 mM sucrose. In another preferred embodiment, the immunogenic composition comprises 300 mM sucrose and 10 mM Tris. In another preferred embodiment, the immunogenic composition comprises 300 mM sucrose and 10 mM Tris and does not comprise a salt. In another preferred embodiment, the immunogenic composition comprises 300 mM sucrose and 10 mM Tris at pH 7.4. In another preferred embodiment, the immunogenic composition comprises 300 mM sucrose and 10 mM Tris at pH 7.4, and the composition does not comprise a salt.

In some aspects, the liquid immunogenic composition comprises an RNA molecule/polynucleotide encoding a polypeptide as disclosed herein at a concentration of at least, at most, exactly, or between (inclusive or exclusive) of 0.01, 0.15, 0.20, 0.30, 0.45, 0.60, 0.75, or 0.90 mg/mL, preferably about 0.2 mg/mL, and further comprising or comprising about 10 mM Tris buffer and 300 mM sucrose at a pH of or of about 7.4. In some aspects, one or more of the foregoing elements may be excluded from the liquid immunogenic composition.

In all aspects of the foregoing immunogenic compositions, the immunogenic composition may further comprise a fatty acid, derivative or salt thereof. In addition, in all aspects of the foregoing immunogenic compositions, the immunogenic composition may further comprise a cholesterol analog, e.g beta-sitosterol, wherein the ratio of cholesterol analog:cholesterol is about 6:4 or about 4:6.

### STABILITY

“Stability,” “stabilized,” and “stable” as used herein refers to the resistance of LNPs to chemical or physical changes (e.g., chemical degradation, particle size change, phase separation, aggregation, change in encapsulation, etc.) under given manufacturing, preparation, transportation, storage and/or in-use conditions, e.g., when stress is applied such as shear force, freeze/thaw stress, etc.

The “stable” formulations shall include the immunogenic compositions and combinations thereof of the disclosure. The “stable” formulations shall preferably retain at least 80%, 85%, 90%, 95%, 98%, 99%, or 99.5% of the chemical purity (e.g., chromatographic purity) of a starting, standard, or reference preparation of the LNP formulation (e.g., mRNA-loaded LNP formulation) under given manufacturing, preparation, transportation, storage and/or in-use conditions.

The “stable” formulations of the disclosure also preferably retain at least 80%, 85%, 90%, 95%, 98%, 99%, or 99.5% of the physical properties (e.g., phase homogeneity or heterogeneity, particle size, polydispersity index, RNA integrity, turbidity, encapsulation efficiency, etc.) of a starting, standard, or reference preparation of the LNP formulation (e.g., mRNA-loaded LNP formulation) under given manufacturing, preparation, transportation, storage and/or in-use conditions.

For example, the “stable” formulations of the disclosure preferably retain at least 80%, 85%, 90%, 95%, 98%, 99%, or 99.5% of the physical stability of a starting, standard, or reference preparation of the LNP formulation (e.g., mRNA-loaded LNP formulation) under given manufacturing, preparation, transportation, storage and/or in-use conditions. For example, the physical stability refers to little or no phase separation of LNP components, e.g., phase separation of a fraction of the structure lipid such as cholesterol or phase separation of a fraction of the ionizable lipid such as an ionizable amino lipid from the remainder of LNP.

For example, the “stable” formulations of the disclosure also preferably have an increase of about 20%, 10%, 5%, 1%, 0.5% or less of a starting, standard, or reference LNP mean size under given manufacturing, preparation, transportation, storage and/or in-use conditions. For example, the formulation has an increase in LNP mean size of about 20% or less (e.g., about 15%, about 10%, about 5% or less) after storage at 5° C. or lower for at least one month. For example, the formulation has an increase in LNP mean size of about 20% or less (e.g., about 15%, about 10%, about 5% or less) after 1, 2, 3, 4, 5, or more, up to 30 freeze/thaw cycles. In one embodiment, the formulation has an increase in LNP mean size of about 10% or less after 3 freeze/thaw cycles. In one embodiment, the formulation has an increase in LNP mean size of about 10% or less after 5 freeze/thaw cycles. In one embodiment, the formulation has an increase in LNP mean size of about 5% or less after 3 freeze/thaw cycles. In one embodiment, the formulation has an increase in LNP mean size of about 5% or less after 5 freeze/thaw cycles.

For example, the “stable” formulations of the disclosure also preferably have an increase of about 20%, 10%, 5%, 1%, 0.5% or less of a starting, standard, or reference LNP polydispersity index (PDI) under given manufacturing, preparation, transportation, storage and/or in-use conditions. For example, the formulation has an increase in LNP PDI of about 20% or less (e.g., about 15%, about 10%, about 5% or less) after storage at 5° C. or lower for at least one month. For example, the formulation has an increase in LNP PDI of about 20% or less (e.g., about 15%, about 10%, about 5% or less) after 1, 2, 3, 4, 5, or more, up to 30 freeze/thaw cycles. In one embodiment, the formulation has an increase in LNP PDI of about 20% or less after 3 freeze/thaw cycles. In one embodiment, the formulation has an increase in LNP PDI of about 20% or less after 5 freeze/thaw cycles. In one embodiment, the formulation has an increase in LNP PDI of about 10% or less after 3 freeze/thaw cycles. In one embodiment, the formulation has an increase in LNP PDI of about 10% or less after 5 freeze/thaw cycles.

For example, the “stable” formulation has an increase in turbidity of about 20% or less (e.g., about 15%, about 10%, about 5% or less) after storage at 5° C. or lower for at least one month, e.g., via nephelometric turbidity analysis.

For example, the “stable” formulation has an increase in turbidity of about 20% or less (e.g., about 15%, about 10%, about 5% or less) after 1, 2, 3, or more, up to 30 freeze/thaw cycles, e.g., via nephelometric turbidity analysis.

For example, the physical stability of LNPs is determined by dynamic light scattering (DLS), nanoparticle tracking analysis (NTA), turbidity analysis, flow microscopy analysis, flow cytometry, FTIR microscopy, resonant mass measurement (RMM), Raman microscopy, filtration, laser diffraction, electron microscopy, atomic force microscopy (AFM), static light scattering (SLS), multi-angle static light scattering (MALS), field flow fractionation (FFF), analytical ultracentrifugation (AUC), or any combination thereof.

For example, the “stable” formulations of the disclosure preferably retain at least 80%, 85%, 90%, 95%, 98%, 99%, or 99.5% of the encapsulation efficiency of a starting, standard, or reference preparation of the LNP formulation (e.g., mRNA-loaded LNP formulation) under given manufacturing, preparation, transportation, storage and/or in-use conditions. For example, the encapsulation efficiency is substantially the same after storage at about 5° C. or lower for at least one month. For example, the encapsulation efficiency may decrease by about 20% or less (e.g., about 15%, about 10%, about 5% or less) after storage at about 5° C. or lower for at least one month. For example, the encapsulation efficiency is substantially the same after 1, 2, 3, 4, 5, or more, up to 30 freeze/thaw cycles. In one embodiment, the encapsulation efficiency of the LNP formulation decreases by about 10% or less after 3 freeze/thaw cycles. In one embodiment, the encapsulation efficiency of the LNP formulation decreases by about 10% or less after 5 freeze/thaw cycles. In one embodiment, the encapsulation efficiency of the LNP formulation decreases by about 5% or less after 3 freeze/thaw cycles. In one embodiment, the encapsulation efficiency of the LNP formulation decreases by about 5% or less after 5 freeze/thaw cycles.

For example, the “stable” formulations of the disclosure preferably retain at least 80%, 85%, 90%, 95%, 98%, 99%, or 99.5% of the RNA integrity of a starting, standard, or reference preparation of the LNP formulation (e.g., mRNA-loaded LNP formulation) under given manufacturing, preparation, transportation, storage and/or in-use conditions. For example, the encapsulation efficiency is substantially the same after storage at about 5° C. or lower for at least one month. For example, the RNA integrity may decrease by about 20% or less (e.g., about 15%, about 10%, about 5% or less) after storage at about 5° C. or lower for at least one month. For example, the RNA integrity is substantially the same after 1, 2, 3, 4, or 5, or more, up to 30 freeze/thaw cycles. In one embodiment, the RNA integrity of the LNP formulation decreases by about 10% or less after 3 freeze/thaw cycles. In one embodiment, the RNA integrity of the LNP formulation decreases by about 10% or less after 5 freeze/thaw cycles. In one embodiment, the RNA integrity of the LNP formulation decreases by about 5% or less after 3 freeze/thaw cycles. In one embodiment, the RNA integrity of the LNP formulation decreases by about 5% or less after 5 freeze/thaw cycles.

The “stable” formulations of the disclosure may also preferably retain at least 80%, 85%, 90%, 95%, 98%, 99%, or 99.5% of the biological activity of a starting, standard, or reference preparation of the LNP formulation (e.g., mRNA-loaded LNP formulation) under given manufacturing, preparation, transportation, storage and/or in-use conditions.

For example, the immunogenicity is substantially the same after storage at about 5° C. or lower for at least one month. For example, the immunogenicity may decrease by about 20% or less (e.g., about 15%, about 10%, about 5% or less) after storage at about 5° C. or lower for at least one month. For example, the immunogenicity is substantially the same after 1, 2, 3, 4, 5 or more, up to 30 freeze/thaw cycles.

The “stable” formulations of the disclosure also preferably have an increase of about 20% 10%, 5%, 1%, 0.5% or less of a starting, standard, or reference or benchmark amount of impurities or adducts under given manufacturing, preparation, transportation, storage and/or in-use conditions.

The “stable” formulations of the disclosure also preferably have an increase of about 20% 10%, 5%, 1%, 0.5% or less of a starting, standard, or reference or benchmark amount of sub-visible particles or adducts under given manufacturing, preparation, transportation, storage and/or in-use conditions.

The purity, LNP mean size, encapsulation efficiency, biological activity, immunogenicity, amount of impurities (or adducts) can be determined using any art-recognized method. For example, the LNP mean size can be measured dynamic light scattering (DLS). For example, the concentration of a component of the formulation can be determined using routine methods such as UV-Vis spectrophotometry and high pressure liquid chromatography (HPLC). For example, amount of sub-visible particles can be determined by micro-flow imaging (MFI) and adducts by RP-HPLC.

In certain embodiments, the present formulations are stable at temperatures of about 5° C. for at least 1 month, at least 2 months, at least 3 months, at least 4 months, at least 5 months, at least 6 months, at least 7 months, at least 8 months, at least 9 months, at least 10 months, at least 11 months, at least 12 months, at least 14 months, at least 16 months, at least 18 months, at least 20 months, at least 22 months, or at least 24 months. In one embodiment, the formulation is stable for at least 6-12 months at 5° C.

In certain embodiments, the present formulations are stable at temperatures ranging from about 2 to 8° C. for at least 1 week, at least 2 weeks, at least 3 weeks, at least 4 weeks, at least 5 weeks, at least 6 weeks, at least 1 month, at least 2 months, at least 4 months, at least 6 months, at least 8 months, at least 10 months, at least 12 months, at least 14 months, at least 16 months, at least 18 months, at least 20 months, at least 22 months, or at least 24 months. In one embodiment, the formulation is stable for at least 2 months at 2 to 8° C.

In certain embodiments, the present formulations are stable at a temperature of about 5° C. for at least 1 week, at least 2 weeks, at least 3 weeks, at least 4 weeks, at least 5 weeks, at least 6 weeks, at least 7 weeks, at least 1 month, at least 2 months, at least 3 months, at least 4 months, at least 5 months, at least 6 months, at least 7 months, at least 8 months, at least 9 months, at least 10 months, at least 11 months, or at least 12 months. In one embodiment, the formulation is stable for at least 2 months at about 5° C.

In a particular embodiment, a formulation of the disclosure is stable at a temperature ranging between about −20° C. and 5° C. at a nucleic acid concentration (e.g., an mRNA concentration) of up to 2 mg/mL for at least 2 weeks, for at least 4 weeks, for at least 8 weeks, for at least 12 weeks, for at least 16 weeks, for at least 32 weeks, for at least a year, or for at least two years.

In a particular embodiment, a formulation of the disclosure is stable at a temperature ranging between about −20° C. and 5° C. at a nucleic acid concentration (e.g., an mRNA concentration) of up to 1 mg/mL for at least 2 weeks, for at least 4 weeks, for at least 8 weeks, for at least 12 weeks, or for at least 16 weeks.

### Vaccines

In some aspects, a pharmaceutical composition described herein is an immunogenic composition for inducing an immune response. For example, in some aspects, an immunogenic composition is a vaccine. In specific aspects, the immunogenic compositions comprise nucleic acids, and the immunogenic compositions are nucleic acid vaccines. In some aspects, the immunogenic compositions comprise RNA (e.g., modRNA, saRNA), and vaccines are RNA vaccines. In other aspects, the immunogenic compositions comprise DNA, and vaccines are DNA vaccines. In yet other aspects, the immunogenic compositions comprise a polypeptide, and vaccines are polypeptide vaccines. Conditions and/or diseases that may be treated with the nucleic acid and/or peptide or polypeptide compositions include, but are not limited to, those caused and/or impacted by infection, cancer, rare diseases, and other diseases or conditions caused by overproduction, underproduction, and/or improper production of protein or nucleic acids.

In some aspects, the composition is substantially free of one or more impurities or contaminants and, for instance, includes nucleic acid or polypeptide molecules that are equal to at least, at most, exactly, or between (inclusive or exclusive) of 90%, 91%, 92%, 93%, 94%, 95%, 96%, 97%, 98%, or 99% pure; at least 98% pure, or at least 99% pure.

The present disclosure includes methods for preventing, treating and/or ameliorating an infection, disease or condition in a subject, including administering to a subject an effective amount of an RNA molecule that includes at least one open reading frame encoding a polypeptide or composition described herein. As such, the disclosure contemplates vaccines for use in both active and passive immunization aspects. Immunogenic compositions, proposed to be suitable for use as a vaccine, may be prepared from RNA molecules encoding polypeptide(s). In certain aspects, immunogenic compositions are lyophilized for more ready formulation into a desired vehicle.

The preparation of vaccines that contain nucleic acid and/or peptide or polypeptide as active ingredients is generally well understood in the art, as exemplified by U.S. Pat. Nos. 4,608,251; 4,601,903; 4,599,231; 4,599,230; 4,596,792; and 4,578,770, all of which are incorporated herein by reference. Typically, such vaccines are prepared as injectables either as liquid solutions or suspensions; solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines. In specific aspects, vaccines are formulated with a combination of substances, as described in U.S. Pat. Nos. 6,793,923 and 6,733,754, which are incorporated herein by reference. In some aspects, one or more of the foregoing elements may be excluded from a vaccine.

Vaccines may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of or of about 0.5% to about 10%. In some aspects, suppositories may be formed from mixtures containing the active ingredient in the range of or of about 1% to about 2%. Oral formulations include such normally employed excipients as, for example, pharmaceutical grades of mannitol, lactose, starch, magnesium stearate, sodium saccharine, cellulose, magnesium carbonate and the like. In some aspects, 1, 2, 3, 4, 5, or more of the foregoing excipients may be excluded from an oral formulation. These compositions take the form of solutions, suspensions, tablets, pills, capsules, sustained release formulations or powders and contain or contain about 10% to about 95% of active ingredient.

Typically, vaccines are administered in a manner compatible with the dosage formulation, and in such amount as will be therapeutically effective and immunogenic. The quantity to be administered depends on the subject to be treated, including the capacity of the individual's immune system to synthesize antibodies and the degree of protection desired. Precise amounts of active ingredient required to be administered depend on the judgment of the practitioner. However, suitable dosage ranges are of the order of several hundred micrograms of active ingredient per vaccination. Suitable regimes for initial administration and booster shots are also variable, but are typified by an initial administration followed by subsequent inoculations and/or other administrations.

The manner of application may be varied widely. Any of the conventional methods for administration of a vaccine are applicable. These are believed to include oral application within a solid physiologically acceptable base or in a physiologically acceptable dispersion, parenterally, by injection and the like. The dosage of the vaccine will depend on the route of administration and will vary according to the size and health of the subject.

In certain aspects, it will be desirable to have one administration of the vaccine. In some aspects, it will be desirable to have multiple administrations of the vaccine, e.g., 2, 3, 4, 5, 6, or more administrations. The vaccinations may be at 1, 2, 3, 4, 5, 6, 7, 8, to 5, 6, 7, 8, 9, 10, 11, or 12 twelve week intervals, including all ranges there between. In some aspects, vaccinations may be at 1, 2, 3, 4, 5, 6, 7, 8, 9, 10, 11, or 12 month intervals, including all ranges there between. Periodic boosters at intervals of 1-5 years may be desirable to maintain protective levels of the antibodies.

### Administration and Dosing

The term “treating”, “treat” or “treatment” as used herein embraces both preventative, i.e., prophylactic, and palliative treatment, i.e., relieve, alleviate, or slow the progression of the patient's disease (or condition) or any tissue damage associated with the disease.

As used herein, the terms, “subject, “individual” or “patient,” used interchangeably, refer to any animal, including mammals. Mammals according to the disclosure include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting (or slowing) further development of the pathology or symptomatology or both); and
(3) ameliorating the disease; for example, ameliorating a disease, condition or disorder in an individual that is experiencing or displaying the pathology or symptomatology of the disease, condition or disorder (i.e., reversing the pathology or symptomatology or both).

Typically, a compound of the disclosure is administered in an amount effective to treat a condition as described herein. The compounds of the disclosure may be administered as compound per se, or alternatively, as a pharmaceutically acceptable salt. For administration and dosing purposes, the compound per se or pharmaceutically acceptable salt thereof will simply be referred to as the compounds of the disclosure.

The compounds of the disclosure are administered by any suitable route in the form of a pharmaceutical composition adapted to such a route, and in a dose effective for the treatment intended. The compounds of the disclosure may be administered orally, rectally, vaginally, parenterally, topically, intranasally, or by inhalation.

The compounds of the disclosure may be administered orally. Oral administration may involve swallowing, so that the compound enters the gastrointestinal tract, or buccal or sublingual administration may be employed by which the compound enters the bloodstream directly from the mouth.

In another embodiment, the compounds of the disclosure may also be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, the compounds of the disclosure may also be administered topically to the skin or mucosa, that is, dermally or transdermally. In another embodiment, the compounds of the disclosure may also be administered intranasally or by inhalation. In another embodiment, the compounds of the disclosure may be administered rectally or vaginally. In another embodiment, the compounds of the disclosure may also be administered directly to the eye or ear.

The dosage regimen for the compounds of the disclosure or compositions containing said compounds is based on a variety of factors, including the type, age, weight, sex and medical condition of the patient; the severity of the condition; the route of administration; and the activity of the particular compound employed. Thus, the dosage regimen may vary widely. In one embodiment, the total daily dose of a compound of the disclosure is typically from about 0.01 to about 100 mg/kg (i.e., mg compound of the disclosure per kg body weight) for the treatment of the indicated conditions discussed herein. In another embodiment, total daily dose of the compound of the disclosure is from about 0.1 to about 50 mg/kg, and in another embodiment, from about 0.5 to about 30 mg/kg. It is not uncommon that the administration of the compounds of the disclosure will be repeated a plurality of times in a day (typically no greater than 4 times). Multiple doses per day typically may be used to increase the total daily dose, if desired.

### Therapeutic Methods and Uses

The compounds of the disclosure may agonize or modulate the activity of TLR7 and/or TLR8 and may be useful as vaccine adjuvants.

Adjuvant formulations comprising the compounds of the disclosure may be used with an immunogen (i.e. a therapeutic agent or antigen of interest) to obtain an immunogenic composition, for example, a vaccine. In some embodiments, LNPs disclosed herein comprise an immunogen to obtain an immunogenic composition. The immunogenic composition may comprise naturally-occurring or artificially-created proteins, recombinant proteins, glycoproteins, peptides, carbohydrates, nucleic acids, haptens, whole viruses, bacteria, protozoa, or virus-like particles, or conjugates thereof as the immunogen. The immunogenic composition may be suitably used as a vaccine for chickenpox or shingles, human respiratory syncytial virus infection (RSV), Cytomegalovirus infection (CMV), Human metapneumovirus, Human parainfluenza viruses type 1 or type 3, Lyme disease, Streptococcus pneumonia, Clostridioides difficile, Escherichia coli or Klebsiella pneumoniae, influenza, HIV-1, Hepatitis A, Hepatitis B, Human Papilloma virus, Meningococcal type A meningitis, Meningococcal type B meningitis, Meningococcal type C meningitis, Tetanus, Diphtheria, Pertussis, Polio, Haemophilus influenza type B, Dengue, Hand Foot and Mouth Disease, Typhoid, Pneumococcus, Japanese encephalitis virus, Anthrax, Shingles, Malaria, Norovirus, or cancer. The immunogenic composition may be suitably used in methods for treating or preventing a disease or infection in a subject, preferably wherein the subject is a human, caused by a pathogen associated with an infectious disease wherein the pathogen is selected from Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBOV), Human herpesvirus 6 (HHV-6) and Human herpesvirus 7 (HHV-7), Human metapneumovirus (hMPV) including hMPV A and hMPV B, hMPV F protein, Human papillomavirus (HPV), Human parainfluenza viruses (HPIV), Japanese encephalitis virus, JC virus, Junin virus, Kingella kingae, Klebsiella granulomatis, Klebsiella pneumoniae, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parainfluenza virus (PIV) including PIV1, PIV2, PIV3, and PIV4, PIV1 F protein, PIV3 F protein, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella-zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

In some embodiments, adjuvant formulations comprising the compounds disclosed herein may be used with a polypeptide immunogen to obtain an immunogenic composition. In some embodiments, adjuvant formulations comprising the compounds disclosed herein may be used with more than one polypeptide immunogen to obtain an immunogenic composition, for example, 2, 3, 4, 5, or more polypeptide immunogens.

In some embodiments, adjuvant formulations comprising the compounds disclosed herein may be used with an immunogen to obtain an immunogenic composition, wherein the immunogen is a nucleic acid encoding a polypeptide. In various embodiments, adjuvant formulations comprising the compounds disclosed herein may be used with an immunogen to obtain an immunogenic composition, wherein the immunogen is DNA encoding a polypeptide. In a particular embodiment, adjuvant formulations comprising the compounds disclosed herein may be used with an immunogen to obtain an immunogenic composition, wherein the immunogen is RNA encoding a polypeptide. In some embodiments, adjuvant formulations comprising the compounds disclosed herein may be used with more than one immunogen to obtain an immunogenic composition, wherein at least one immunogen is a nucleic acid encoding a polypeptide. In other embodiments, adjuvant formulations comprising the compounds disclosed herein may be used with more than one immunogen to obtain an immunogenic composition, wherein at least one immunogen is DNA encoding a polypeptide. In a particular embodiment, adjuvant formulations comprising the compounds disclosed herein may be used with more than one immunogen to obtain an immunogenic composition, wherein at least one immunogen is RNA encoding a polypeptide.

In some embodiments, adjuvant formulations comprising the compounds disclosed herein may be used with a Streptococcus pneumoniae antigen to obtain an immunogenic composition. In other embodiments, adjuvant formulations comprising the compounds disclosed herein may be used with a Streptococcus pneumoniae polysaccharide antigen to obtain an immunogenic composition. In other embodiments, adjuvant formulations comprising the compounds disclosed herein may be used with a Streptococcus pneumoniae serotype 3 polysaccharide antigen to obtain an immunogenic composition. In some embodiments, adjuvant formulations comprising the compounds disclosed herein may be used with 1, 2, 3, 4, 5, 6, 7, 8, 9, 10, 11, 12, 13, 14, 15, 16, 17, 18, 19, 20, 21, 22, 23, 24, 25, or more Streptococcus pneumoniae polysaccharide antigens.

In some embodiments, adjuvant formulations comprising the compounds disclosed herein may be used with a conjugated Streptococcus pneumoniae antigen to obtain an immunogenic composition. In other embodiments, adjuvant formulations comprising the compounds disclosed herein may be used with a conjugated Streptococcus pneumoniae polysaccharide antigen to obtain an immunogenic composition. In other embodiments, adjuvant formulations comprising the compounds disclosed herein may be used with a conjugated Streptococcus pneumoniae serotype 3 polysaccharide antigen to obtain an immunogenic composition. In some embodiments, adjuvant formulations comprising the compounds disclosed herein may be used with 1, 2, 3, 4, 5, 6, 7, 8, 9, 10, 11, 12, 13, 14, 15, 16, 17, 18, 19, 20, 21, 22, 23, 24, 25, or more conjugated Streptococcus pneumoniae polysaccharide antigens. In some embodiments, the Streptococcus pneumoniae polysaccharide antigens are conjugated to a CRM 197 carrier. In some embodiments, the Streptococcus pneumoniae polysaccharide antigens are conjugated to a C₅a peptidase from Streptococcus (SCP) carrier. In a particular embodiment, the immunogenic composition comprises an adjuvant compound disclosed herein and a Streptococcus pneumoniae serotype 3 polysaccharide antigen conjugated to a CRM197 carrier. In another particular embodiment, the immunogenic composition comprises an adjuvant compound disclosed herein and a Streptococcus pneumoniae serotype 3 polysaccharide antigen conjugated to a SCP carrier.

In some embodiments, adjuvant formulations comprising the compounds disclosed herein may be used with an E. coli immunogen to obtain an immunogenic composition. In some embodiments, immunogenic compositions comprising the LNPs disclosed herein comprise an Escherichia coli (E. coli) immunogen. In some embodiments, immunogenic compositions comprising the LNPs disclosed herein comprise an E. coli fimbrial adhesin (FimH) immunogen. Embodiments of E. coli FimH immunogens, including polypeptides, and nucleic acids encoding the same, are provided in WO2023/111907 and WO2024/256962, incorporated by reference herein in the entirety.

In some embodiments, immunogenic compositions comprising the LNPs disclosed herein comprise a RNA molecule encoding a FimH polypeptide, variant or fragment thereof. In some embodiments, immunogenic compositions comprising the LNPs disclosed herein comprise a RNA molecule encoding a FimH polypeptide, variant or fragment thereof, comprising the mutation G15A, wherein the amino acid positions are numbers according to SEQ ID NO: 1. In some embodiments, immunogenic compositions comprising the LNPs disclosed herein comprise a RNA molecule encoding a FimH polypeptide, variant or fragment thereof, comprising the mutation G16A, wherein the amino acid positions are numbers according to SEQ ID NO: 1. In some embodiments, immunogenic compositions comprising the LNPs disclosed herein comprise a RNA molecule encoding a FimH polypeptide, variant or fragment thereof, comprising the mutation V27A, wherein the amino acid positions are numbers according to SEQ ID NO: 1. In a preferred embodiment, immunogenic compositions comprising the LNPs disclosed herein comprise a RNA molecule encoding a FimH polypeptide, variant or fragment thereof, comprising the mutations of G15A, G16A, V27A, wherein the amino acid positions are numbers according to SEQ ID NO: 1. In another preferred embodiment, immunogenic compositions comprising the LNPs disclosed herein comprise a RNA molecule encoding a FimH polypeptide, variant or fragment thereof, wherein the FimH polypeptide comprises or consists of the sequence of SEQ ID NO: 2.

In some embodiments, immunogenic compositions comprising the LNPs disclosed herein comprise a RNA molecule, wherein the RNA comprises at least one open reading frame (ORF) encoding FimH. In some embodiments, immunogenic compositions comprising the LNPs disclosed herein comprise a RNA molecule, wherein the RNA comprises at least one open reading frame (ORF) encoding FimH, a 5′ untranslated region (5′ UTR), and a 3′ untranslated region (3′ UTR). In some embodiments, immunogenic compositions comprising the LNPs disclosed herein comprise a RNA molecule, wherein the RNA comprises at least one open reading frame (ORF) encoding FimH and a polyA tail. In some embodiments, immunogenic compositions comprising the LNPs disclosed herein comprise a RNA molecule, wherein the RNA comprises at least one open reading frame (ORF) encoding FimH, a 5′ untranslated region (5′ UTR), a 3′ untranslated region (3′ UTR), and a polyA tail. In a preferred embodiment, immunogenic compositions comprising the LNPs disclosed herein comprise a RNA molecule, wherein the RNA comprises the sequence of SEQ ID NO: 3. In another preferred, immunogenic compositions comprising the LNPs disclosed herein comprise a RNA molecule, wherein the RNA consists of the sequence of SEQ ID NO: 3.

The present disclosure provides an immunogenic composition comprising an immunogen and a compound of the disclosure as described herein.

### Co-Administration

The compounds of the disclosure may be used alone, or in combination with one or more therapeutic agents. The disclosure provides any of the uses, methods or compositions as defined herein wherein the compound of the disclosure, or pharmaceutically acceptable salt thereof, is used in combination with one or more therapeutic agent discussed herein.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compounds at the same point in time but as separate dosage forms at the same or different site of administration. Examples of “in combination” include, but are not limited to, “concurrent administration,” “co-administration,” “simultaneous administration,” “sequential administration” and “administered simultaneously”.

A compound of the disclosure and the one or more therapeutic agents may be administered as a fixed or non-fixed combination of the active ingredients. The term “fixed combination” means a compound of the disclosure, or a pharmaceutically acceptable salt thereof, and the one or more therapeutic agents, are both administered to a subject simultaneously in a single composition or dosage. The term “non-fixed combination” means that a compound of the disclosure, or a pharmaceutically acceptable salt thereof, and the one or more therapeutic agents are formulated as separate compositions or dosages such that they may be administered to a subject in need thereof simultaneously or at different times with variable intervening time limits, wherein such administration provides effective levels of the two or more compounds in the body of the subject.

In one embodiment, the present disclosure provides a pharmaceutical composition comprising an immunogen disclosed herein, wherein the pharmaceutical composition is administered in combination with a pharmaceutical composition comprising an adjuvant disclosed herein or a pharmaceutically acceptable salt thereof simultaneously or at different times.

These agents and compounds of the disclosure may be combined with pharmaceutically acceptable vehicles such as saline, Ringer's solution, dextrose solution, and the like. The particular dosage regimen, i.e., dose, timing and repetition, will depend on the particular individual and that individual's medical history.

### Kits

Another aspect of the disclosure provides kits comprising the compound of the disclosure or pharmaceutical compositions comprising the compound of the disclosure. A kit may include, in addition to the compound of the disclosure or pharmaceutical composition thereof, diagnostic or therapeutic agents. A kit may also include instructions for use in a diagnostic or therapeutic method. In some embodiments, the kit includes the compound or a pharmaceutical composition thereof and a diagnostic agent. In other embodiments, the kit includes the compound or a pharmaceutical composition thereof and one or more therapeutic agents, such as an immunogen disclosed herein.

In yet another embodiment, the disclosure comprises kits that are suitable for use in performing the methods of treatment described herein. In one embodiment, the kit contains a first dosage form comprising one or more of the compounds of the disclosure in quantities sufficient to carry out the methods of the disclosure. In another embodiment, the kit comprises one or more compounds of the disclosure in quantities sufficient to carry out the methods of the disclosure and a container for the dosage.

### Synthetic Methods

Compounds of the present disclosure may be synthesized by synthetic routes that include processes analogous to those well-known in the chemical arts, particularly in light of the description contained herein. The starting materials are generally available from commercial sources or may be prepared using methods well known to those skilled in the art. Many of the compounds used herein, are related to, or may be derived from compounds in which one or more of the scientific interest or commercial need has occurred. Accordingly, such compounds may be one or more of 1) commercially available; 2) reported in the literature or 3) prepared from other commonly available substances by one skilled in the art using materials which have been reported in the literature.

For illustrative purposes, the reaction schemes depicted below provide potential routes for synthesizing the compounds of the present disclosure as well as key intermediates. For a more detailed description of the individual reaction steps, see the Examples section below. Those skilled in the art will appreciate that other synthetic routes may be used to synthesize the inventive compounds. Although specific starting materials and reagents are discussed below, other starting materials and reagents may be substituted to provide one or more of a variety of derivatives or reaction conditions. In addition, many of the compounds prepared by the methods described below may be further modified in light of this disclosure using conventional chemistry well known to those skilled in the art.

The skilled person will appreciate that the experimental conditions set forth in the schemes that follow are illustrative of suitable conditions for effecting the transformations shown, and that it may be necessary or desirable to vary the precise conditions employed for the preparation of compounds of the disclosure. It will be further appreciated that it may be necessary or desirable to carry out the transformations in a different order from that described in the schemes, or to modify one or more of the transformations, to provide the desired compound of the disclosure.

In the preparation of compounds of the disclosure it is noted that some of the preparation methods useful for the preparation of the compounds described herein may require protection of remote functionality (e.g., a primary amine, secondary amine, carboxyl, etc. in a precursor of a compound of the disclosure). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see March's Advanced Organic Chemistry: Reactions, Mechanisms, and Structure 8th Edition.

For example, if a compound contains a amine or carboxylic acid functionality, such functionality may interfere with reactions at other sites of the molecule if left unprotected. Accordingly, such functionalities may be protected by an appropriate protecting group (PG) which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines and lower alkyl or benzyl esters for carboxylic acids) which are generally not chemically reactive under the reaction conditions described and may typically be removed without chemically altering other functionality in a compound of the disclosure.

### General Experimental Details

In the non-limiting Examples and Preparations that illustrate the disclosure and that are set out in the description, and in the following Schemes, the following the abbreviations, definitions and analytical procedures may be referred to.

The compounds and intermediates described below were named using the naming convention provided with ChemDraw version 20.1.1.123. The naming convention provided with ChemDraw version 20.1.1.123 is well known by those skilled in the art and it is believed that the naming convention generally comports with the IUPAC (International Union for Pure and Applied Chemistry) recommendations on Nomenclature of Organic Chemistry and the CAS Index rules. Unless noted otherwise, all reactants were obtained commercially without further purifications or were prepared using methods known in the literature.

The following illustrate the synthesis of various compounds of the present disclosure. Additional compounds within the scope of this disclosure may be prepared using the methods illustrated in these Examples, either alone or in combination with techniques generally known in the art. All starting materials in these Preparations and Examples are either commercially available or can be prepared by methods known in the art or as described herein.

Reactions were performed in air or, when oxygen- or moisture-sensitive reagents or intermediates were employed, under an inert atmosphere (nitrogen or argon).

Unless otherwise noted, chemical reactions were performed at room temperature (about 25° C.).

In the examples, proton nuclear magnetic resonance (¹H NMR) spectra were recorded at 400 MHz, where δ is chemical shift; br is broad; CDCl₃ is deuterated chloroform; (CD₃)₂SO is deuterated dimethyl sulfoxide; d is doublet; dd is doublet of doublets; ddd is doublet of doublet of doublets; D₂O is deuterated water; dt is doublet of triplets; s is singlet; t is triplet; m is multiplet; MHz is megahertz; ppm is parts per million; q is quartet.

### Abbreviations

Å is angstrom;
AcOH is acetic acid;
MeCN is acetonitrile;
NH₄OH is ammonium hydroxide;
Boc2O is di-tert-butyl decarbonate;
9-BBN is 9-borabicyclo[3.3.1]nonane;
CHCl3 is chloroform;
° C. is degrees Celsius;
Cs2CO3 is cesium carbonate;
CuI is copper (I) iodide;
DCM is dichloromethane;
DIH is 1,3-diiodo-5,5-dimethylhydantoin;
DIPEA is N,N-diisopropylethylamine;
DMA is dimethylacetamide;
DMF is dimethylformamide;
DMP is Dess-Martin periodinane;
EtOAc is ethyl acetate;
EtOH is ethanol;
g is gram;
H2 is hydrogen;
HATU is 1-[bis(dimethylamino)methylene]-1H-1,2,3-triazolo[4,5-b]pyridinium 3-oxid hexafluorophosphate;
HCl is hydrochloric acid;
H2O is water;
IPA is isopropyl alcohol;
KOAc is potassium acetate;
K2CO3 is potassium carbonate;
KOH is potassium hydroxide;
LCMS is liquid chromatography mass spectrometry;
LiOH is lithium hydroxide monohydrate;
L is liter;
M is molar;
M+ is mass ion;
mm is millimeter;
m-CPBA is 3-chloroperoxybenzoic acid;
MeOH is methanol;
um is micromole;
mL is milliliter;
mL/min is milliliter per minute;
mmol is millimole;
mol is mole;
MTBE is methyl tert-butyl ether;
psi is pounds per square inch;
N2 is nitrogen;
nm is nanometer;
Rf is retention factor;
NaBH3CN is sodium cyanoborohydride;
NaHSO3 is sodium bisulfite;
NaHCO₃ is sodium bicarbonate;
Na2CO3 is sodium carbonate;
NaOH is sodium hydroxide;
Na2SO3 is sodium sulfite;
Na2SO4 is sodium sulfate;
Pd/C is palladium on carbon;
Pd(PPh3)4 is tetrakis(triphenylphosphine) palladium (0);
Pt/C is platinum on carbon;
TBSCl is tert-butyldimethylsilyl chloride;
TEA is triethylamine;
THF is tetrahydrofuran;
TFA is trifluoroacetic acid;
TsCl is p-toluenesulfonyl chloride;
UV is ultraviolet; and
wt % is weight percent.

### General Methods

Unless stated otherwise, the variables in Preparation P1-P8 have the same meanings as defined herein.

In some cases, compounds described herein may contain protecting groups, which may be appended or removed by additional steps in the synthetic sequence using conditions known in the art (March's Advanced Organic Chemistry: Reactions, Mechanisms, and Structure 8th Edition or Protecting Groups, 10 Georg Thieme Verlag, 1994; Wuts, Greene's Protective Groups in Organic Synthesis (Fifth edition/2014)). Compounds at every step may be purified by standard techniques, such as column chromatography, crystallization, or reverse phase SFC or HPLC.

The following substrates were synthesized according to Preparation P1-P8.

### Preparation P1

### 2-((4-Amino-2-butyl-1H-imidazo[4,5-c]quinolin-1-yl)methyl)-2-(aminomethyl)propane-1,3-diol hydrochloride (P1)

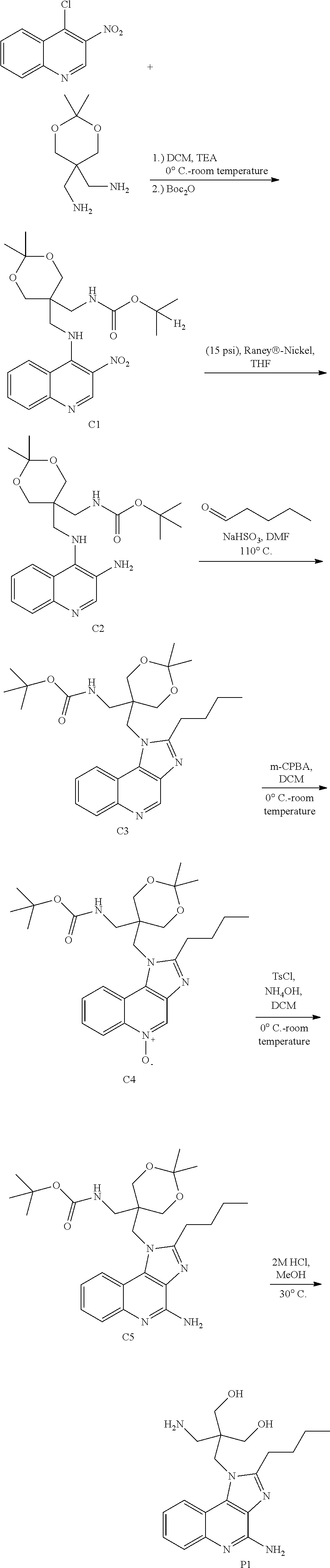

### Step 1. Preparation of tert-butyl ((2,2-dimethyl-5-(((3-nitroquinolin-4-yl)amino)methyl)-1,3-dioxan-5-yl)methyl)carbamate (C1)

To a solution of (2,2-dimethyl-1,3-dioxane-5,5-diyl)dimethanamine (CAS: 104275-10-7; 84.0 g, 482 mmol) in DCM (2.5 L) was added TEA (97.6 g, 964 mmol) and 4-chloro-3-nitroquinoline (CAS: 39061-97-7; 50.3 g, 241 mmol) at 0° C. The reaction mixture was warmed to room temperature and stirred for 3.5 hours before Boc₂O (CAS: 24424-99-5; 237 g, 1.09 mol) was added. The reaction mixture was stirred at room temperature for an additional 16 hours then was washed with the brine (1 L). The organic layer was concentrated in vacuo then MeCN (150 mL) was added, and the reaction mixture was filtered. The filter cake was washed with MeCN (100 mL) then MTBE (200 mL). The filter cake dried further to provide C1 (222 g, >99% yield) as a yellow solid. The solid was used directly in the next step without additional purification.

### Step 2. Preparation of tert-butyl ((5-(((3-aminoquinolin-4-yl)amino)methyl)-2,2-dimethyl-1,3-dioxan-5-yl)methyl)carbamate (C2)

The following reaction was conducted in 4 batches in parallel then combined for work-up. For the first batch, Raney®-Nickel (35.0 g) was added to a solution of C1 (70.0 g, 157 mmol) in THF (1 L) then was stirred under H₂ gas (15 psi) at room temperature for 16 hours.

The 3 additional batches of the same reaction were all conducted with C1 (70.0 g, 157 mmol). All 4 batches were combined then the combined reaction mixture was filtered. The filter cake was washed with DCM (300 mL×3). The combined filtrate was concentrated in vacuo to provide C2 (217 g, 48.2% yield) as a brown solid. The solid was used directly in the next step without additional purification. (LCMS) (M+H)⁺ 417.3.

### Step 3. Preparation of tert-butyl ((5-((2-butyl-1H-imidazo[4,5-c]quinolin-1-yl)methyl)-2,2-dimethyl-1,3-dioxan-5-yl)methyl)carbamate (C3)

To the reaction mixture of pentanal (44.9 g, 521 mmol) and NaHSO3 (81.3 g, 781 mmol) were added to a solution of C2 (217 g, 521 mmol) in DMF (2.5 L). The reaction mixture was stirred at 110° C. for 16 hours then diluted with H₂O (3 L) and filtered. The filter cake of the combined batches was diluted with EtOAc (1 L) then heated to 80° C. until the solid dissolved. The solution was cooled to room temperature and stirred for 2 hours which caused an off-white solid to form. The reaction mixture was filtered, and the filter cake was collected to provide C3 (74.5 g, 29.6% yield) as a white solid. The solid was used directly in the next step without additional purification. (LCMS) (M+H)⁺ 483.4. ¹H NMR (400 MHz, CDCl₃) δ 9.30 (s, 1H), 8.34 (d, 1H), 8.28 (dd, 1H), 7.67 (ddd, 1H), 7.60 (ddd, 1H), 5.07-4.90 (m, 1H), 4.78-4.68 (m, 1H), 4.65-4.48 (m, 1H), 3.90-3.65 (m, 3H), 3.39 (d, 3H), 3.10-2.87 (m, 2H), 1.97-1.87 (m, 2H), 1.54-1.43 (m, 11H), 1.31 (br s, 3H), 1.09 (br s, 3H), 1.00 (t, 3H).

### Step 4. Preparation of 1-((5-(((tert-butoxycarbonyl)amino)methyl)-2,2-dimethyl-1,3-dioxan-5-yl)methyl)-2-butyl-1H-imidazo[4,5-c]quinoline 5-oxide (C4)

Under N₂ gas at 0-10° C., m-CPBA (23.3 g, 115 mmol) was added to a solution of C3 (37.0 g, 76.7 mmol) in DCM (320 mL). The reaction mixture was stirred at room temperature for 48 hours then quenched with saturated Na₂SO₃ (80 mL). The quenched reaction mixture was washed with saturated NaHCO₃ (100 mL×4) then brine (100 mL) and dried with Na₂SO₄. The reaction mixture was filtered and concentrated in vacuo to provide C4 (50.0 g, >99% yield) as a yellow solid. The solid was used directly in the next step without additional purification. (LCMS) (M+H)⁺ 499.3.

### Step 5. Preparation of tert-butyl ((5-((4-amino-2-butyl-1H-imidazo[4,5-c]quinolin-1-yl)methyl)-2,2-dimethyl-1,3-dioxan-5-yl)methyl)carbamate (C5)

At 0° C., to a solution of C4 (50.0 g, 0.100 mol) in DCM (500 mL) was added NH₄OH solution (120 mL) then TsCl (23.9 g, 125 mmol). The reaction mixture was warmed to room temperature then stirred for 16 hours. The yellow reaction mixture was washed with saturated NaHCO₃ (100 mL×2) then brine (100 mL×2) and dried with Na₂SO₄. The reaction mixture was filtered then concentrated in vacuo to give a brown residue. The residue was purified by column chromatography (silica gel, MeOH:DCM, 1-10% MeOH over 15 minutes) then lyophilized to provide C5 (31.9 g, 69.3% yield) as a light-yellow solid. (LCMS) (M+H)⁺ 498.3. ¹H NMR (400 MHz, (CD₃)₂SO) δ 8.29 (d, 1H), 7.60 (dd, 1H), 7.40 (t, 1H), 7.23-7.13 (m, 2H), 6.52 (br s, 2H), 4.85 (d, 1H), 4.64 (d, 1H), 3.89-3.62 (m, 3H), 3.22-2.76 (m, 4H), 1.84-1.71 (m, 2H), 1.46-1.34 (m, 11H), 1.26 (br s, 4H), 1.08 (br s, 3H), 0.94 (t, 3H).

### Step 6. Preparation of 2-((4-amino-2-butyl-1H-imidazo[4,5-c]quinolin-1-yl)methyl)-2-(aminomethyl)propane-1,3-diol hydrochloride (P1)

At 0-5° C., to a solution of C₅ (31.9 g, 64.1 mmol) in MeOH (50 mL) was added 2M HCl in MeOH (600 mL). The reaction mixture was heated to 30° C. and stirred for 2 hours then was concentrated in vacuo. The residue was suspended in EtOAc/DCM/MTBE then filtered. The filter cake was collected then lyophilized to provide P1 (27.3 g, >99% yield) as a white solid. The solid was used directly in the next step without further purification. (LCMS) (M+H)⁺ 358.2. ¹H NMR (400 MHz, (CD₃)₂SO) δ 14.16-13.94 (m, 1H), 8.98 (br s, 1H), 8.67 (d, 1H), 7.98 (br s, 2H), 7.81 (d, 1H), 7.69 (t, 1H), 7.52 (t, 1H), 4.99 (d, 1H), 4.67 (d, 1H), 4.10 (br s, 2H), 3.61 (dd, 2H), 3.41 (d, 1H), 3.30 (d, 1H), 3.20-2.95 (m, 3H), 2.70-2.59 (m, 1H), 1.87-1.74 (m, 2H), 1.49-1.36 (m, 2H) 0.94 (t, 3H).

### Preparation P2

### 1-(3-Amino-2,2-bis(((tert-butyldimethylsilyl)oxy)methyl)propyl)-2-butyl-1H-imidazo[4,5-c]quinolin-4-amine (P2)

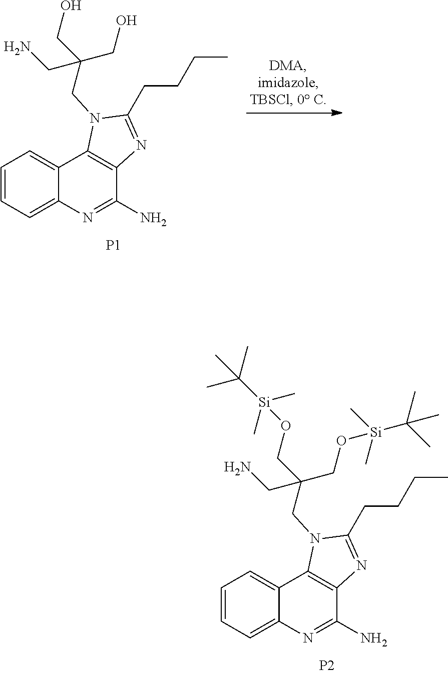 [SMILES: CCCCc1nc2c(N)nc3ccccc3c2n1CC(CN)(CO)CO ; CCCCc1nc2c(N)nc3ccccc3c2n1CC(CN)(CO[Si](C)(C)C(C)(C)C)CO[Si](C)(C)C(C)(C)C]

### Preparation of 1-(3-amino-2,2-bis(((tert-butyldimethylsilyl)oxy)methyl)propyl)-2-butyl-1H-imidazo[4,5-c]quinolin-4-amine (P2)

At 0° C. under N₂ gas, to a solution of P1 (29.3 g, 68.1 mmol) in DMA (250 mL) was added imidazole (32.4 g, 476 mmol) and TBSCl (51.3 g, 340 mmol). The reaction mixture was stirred for 2 hours then added dropwise to ice water (600 mL). The diluted reaction mixture was extracted with DCM (300 mL×3) then the combined organic layer was washed with NaHCO₃ (100 mL×4), brine (100 mL×2) and dried with Na₂SO₄. The dried organic layer was filtered and concentrated in vacuo. The yellow gum was purified by column chromatography (silica gel, ((1:10) NH₄OH: MeOH) in DCM, 0-15% ((1:10) NH₄OH in MeOH) to provide P2 (30.6 g, 76.7% yield) as a light-yellow solid. (LCMS) (M+H)⁺ 587.9. ¹H NMR (400 MHz, (CD₃)₂SO) δ 8.53 (d, 1H), 7.59 (d, 1H), 7.38 (t, 1H), 7.14 (t, 1H), 6.39 (br s, 2H), 4.94 (d, 1H), 4.52 (d, 1H), 3.76-3.63 (m, 2H), 3.54-3.45 (m, 1H), 3.41-3.36 (m, 1H), 3.13-2.70 (m, 3H), 2.34-2.22 (m, 1H), 1.84-1.57 (m, 4H), 1.40 (q, 2H), 0.92 (t, 3H), 0.84 (br s, 18H), 0.020-(−0.13) (t, 12H).

### Preparation P3

### Tert-butyl 4-(3-(3-borabicyclo[3.3.1]nonan-3-yl)propyl)piperazine-1-carboxylate (P3)

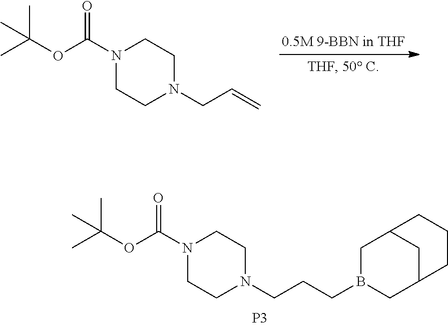 [SMILES: C=CCN1CCN(C(=O)OC(C)(C)C)CC1 ; CC(C)(C)OC(=O)N1CCN(CCCB2CC3CCCC(C2)C3)CC1]

### Preparation of tert-butyl 4-(3-(3-borabicyclo[3.3.1]nonan-3-yl)propyl)piperazine-1-carboxylate (P3)

The following reaction was conducted in 7 batches in parallel then combined for work-up. To a solution of 1,1-dimethylethyl 4-(2-propen-1-yl)-1-piperazinecarboxylate (CAS: 77278-75-2; 10.0 g, 44.0 mmol) in THF (60 mL) was added 0.5M 9-BBN in THF (88.0 mL, 44.0 mmol) to prepare the first batch. The reaction mixture of the first batch was degassed with N₂ gas then stirred at 50° C. for 30 minutes.

The additional 6 batches of the same reaction were conducted with 1,1-dimethylethyl 4-(2-propen-1-yl)-1-piperazinecarboxylate (CAS: 77278-75-2; 10.0 g, 44.0 mmol) each. The 7 batches were combined to form P3 (115 g, 96.2% yield) as a clear crude solution. The clear crude solution was used directly in the next step without further purification.

### Preparation P4

### 2-((4-Amino-2-butyl-7-(3-(piperazin-1-yl)propyl)-1H-imidazo[4,5-c]quinolin-1-yl)methyl)-2-methylpropane-1,3-diol hydrochloride (P4)

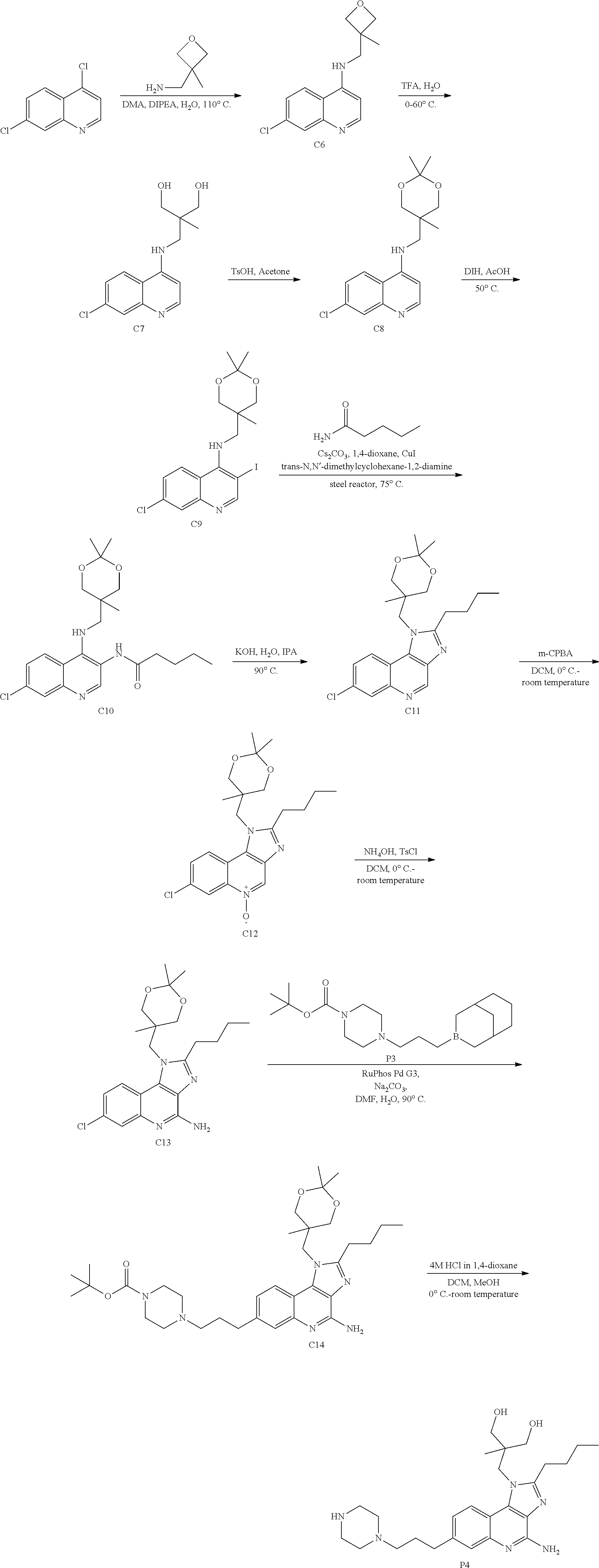

### Step 1. Preparation of 7-chloro-N-((3-methyloxetan-3-yl)methyl)quinolin-4-amine (C6)

The following reaction was conducted in 5 batches in parallel then combined for work-up. To a solution of 4,7-dichloroquinoline (CAS: 86-98-6; 50.0 g, 0.252 mol) in DMA (200 mL) was added (3-methyloxetan-3-yl) methanamine (CAS: 153209-97-3; 28.0 g, 0.277 mol), DIPEA (98.0 g, 0.758 mol) and H₂O (60 mL) to provide the first batch. The reaction mixture of the first batch was stirred at 110° C. for 72 hours then cooled to room temperature.

The additional 4 batches of the same reaction were conducted with 4,7-dichloroquinoline (CAS: 86-98-6; 50.0 g, 0.252 mol) and 1 batch of the same reaction was conducted with 4,7-dichloroquinoline (CAS: 86-98-6; 10.0 g, 0.0505 mol). The 5 batches were combined then diluted with H₂O (1.5 L) dropwise. The reaction mixture was filtered and dried in vacuo to provide C6 (200.0 g, 71.8% yield) as a white solid. The solid was used directly in the next step without further purification. (LCMS) (M+H)⁺ 264.55. ¹H NMR (400 MHz, (CD₃)₂SO) δ 8.31 (d, 1H), 8.28 (d, 1H), 7.72 (d, 1H), 7.38 (dd, 1H), 7.17 (t, 1H), 6.48 (d, 1H), 4.42 (d, 2H), 4.18 (d, 2H), 3.40 (d, 2H), 1.26 (s, 3H).

### Step 2. Preparation of 2-(((7-chloroquinolin-4-yl)amino)methyl)-2-methylpropane-1,3-diol (C7)

The following reaction was conducted in 5 batches in parallel then combined for work-up. At 0° C., to a solution of C6 (47.5 g, 0.181 mol) in H₂O (285 mL) was slowly added TFA (190 mL) to give the first batch. The reaction mixture of the first batch was heated to 60° C. and stirred for 1 hour.

The additional 3 batches of the same reaction were conducted with C6 (47.5 g, 0.181 mol) and 1 batch of the same reaction was conducted with C6 (10.0 g, 0.0381 mol). The 5 batches were combined then cooled to room temperature and concentrated in vacuo. The residue was diluted with MeOH (1.0 L) then concentrated in vacuo again before 5% aqueous NaHCO₃ (1.5 L) was slowly added. The reaction mixture was stirred at room temperature for 16 hours before filtration. The filter cake was collected then the white solid was stirred in H₂O (1.0 L) at room temperature overnight. The suspension was filtered, and the filter cake was collected then dried in vacuo to provide C7 (190.0 g, 88.9% yield) as a white solid. The solid was used directly in the next step without additional purification. (LCMS) (M+H)⁺ 281.15. ¹H NMR (400 MHz, (CD₃)₂SO) δ 8.46 (d, 1H), 8.28 (d, 1H), 8.24-8.17 (m, 1H), 7.86 (d, 1H), 7.63 (dd, 1H), 6.83 (d, 1H), 4.81 (br s, 2H), 3.44-3.29 (m, 6H), 0.87 (s, 3H).

### Step 3. Preparation of 7-chloro-N-((2,2,5-trimethyl-1,3-dioxan-5-yl)methyl)quinolin-4-amine (C8)

The following reaction was conducted in 5 batches in parallel then combined for work-up. To a solution of C7 (45.0 g, 0.160 mol) in acetone (1.0 L) was added TsOH (30.4 g, 0.176 mol) to provide the first batch. The reaction mixture of the first batch was stirred at room temperature for 16 hours.

The additional 3 batches of the same reaction were conducted with C7 (45.0 g, 0.160 mol) and 1 batch of the same reaction was conducted with C7 (10.0 g, 0.0356 mol). The 5 batches were combined then filtered. The filter cake was washed with acetone (1.2 L) then collected. The solid was diluted with DCM (4.0 L) and saturated NaHCO₃ (4.0 L). The suspension at room temperature was stirred vigorously for 2 hours then extracted with DCM (1.0 L×3), washed with brine, dried with Na₂SO₄ and concentrated in vacuo to provide C8 (179 g, 82.4% yield) as white solid. The solid was used directly in the next step without additional purification. (LCMS) (M+H)⁺ 321.10. ¹H NMR (400 MHz, (CD3) 2SO) δ 8.42-8.37 (m, 2H), 7.78 (d, 1H), 7.50-7.44 (m, 1H), 7.30 (t, 1H), 6.77 (d, 1H), 3.60 (s, 4H), 3.47 (d, 2H), 1.38 (d, 6H), 0.85 (s, 3H).

### Step 4. Preparation of 7-chloro-3-iodo-N-((2,2,5-trimethyl-1,3-dioxan-5-yl)methyl)quinolin-4-amine (C9)

The following reaction was conducted in 6 batches in parallel then combined for work-up. To a solution of C8 (42 g, 0.13 mol) in AcOH (420 mL) was added DIH (55 g, 0.14 mol) to provide the first batch. The reaction mixture of the first batch was stirred at 50° C. for 2 hours then cooled to room temperature.

The additional 3 batches of the same reaction were conducted with C8 (42 g, 0.13 mol), 1 batch of the same reaction was conducted with C8 (10.0 g, 0.031 mol) and 1 batch of the same reaction was conducted with C8 (1.0 g, 0.0031 mol). The 6 batches were combined then cooled to 0° C. before 5M NaOH (6.0 L) was added slowly until pH˜8. The combined basic reaction mixture was extracted with EtOAc (2.0 L×3). The combined organic layer was concentrated in vacuo then the residue was purified by column chromatography (silica gel, EtOAc:petroleum ether, 0:100 to 30:70) to provide C9 (150 g, 60.2% yield) as a yellow solid. (LCMS) (M+H)⁺ 447.15. ¹H NMR (400 MHz, (CD₃)₂SO) δ 8.81 (s, 1H), 8.34-8.25 (m, 1H), 7.91-7.89 (m, 1H), 7.57-7.48 (m, 1H), 5.25 (t, 1H), 3.75 (d, 2H), 3.63-3.52 (m, 4H), 1.33 (s, 3H), 1.21 (s, 3H), 0.90 (s, 3H).

### Step 5. Preparation of N-(7-chloro-4-(((2,2,5-trimethyl-1,3-dioxan-5-yl)methyl) amino)quinolin-3-yl) pentanamide (C10)

The following reaction was conducted in 8 batches in parallel then combined for work-up. To a solution of C9 (20.0 g, 44.8 mmol) in 1,4-dioxane (200 mL) was added pentanamide (CAS: 626-97-1; 7.70 g, 76.2 mmol), Cs₂CO₃ (29.2 g, 89.6 mmol), CuI (1.70 g, 8.96 mmol), and then trans-N,N′-dimethylcyclohexane-1,2-diamine (CAS: 67579-81-1; 1.30 g, 8.96 mmol) successively to form the first batch. The reaction mixture of the first batch was degassed with N₂ gas for 3 minutes then sealed and stirred at 75° C. for 16 hours in a steel reactor then cooled to room temperature.

The additional 6 batches of the same reaction were conducted with C₉ (20.0 g, 44.8 mmol) and 1 batch of the same reaction was conducted with C9 (10.0 g, 22.4 mmol). The 8 batches were combined then filtered through a celite pad. The filtrate was concentrated in vacuo then the residue was purified by column chromatography (silica gel, EtOAc:petroleum ether, 0-100% EtOAc) to provide C10 (110.0 g, 78.0% yield) as a yellow solid. (LCMS) (M+2H)⁺ 422.2.

### Step 6. Preparation of 2-butyl-7-chloro-1-((2,2,5-trimethyl-1,3-dioxan-5-yl)methyl)-1H-imidazo[4,5-c]quinoline (C11)

The following reaction was conducted in 5 batches in parallel then combined for work-up. To a solution of C10 (30.0 g, 71.4 mmol) in IPA (1100 mL) was added 3M KOH in H₂O (214 mL, 643 mmol) and stirred at 90° C. for 16 hours then cooled to room temperature to provide the first batch.

The additional 2 batches of the same reaction were conducted with C10 (30.0 g, 71.4 mmol) and 2 batches of the same reaction were conducted with C10 (10.0 g, 23.8 mmol). The 5 batches were combined then poured into H₂O (7 L). The resultant white suspension was stirred at room temperature for 1 hour then filtered to provide C11 (100.0 g, 95.0% yield) as a white solid. The solid was used directly in the next step without further purification. (LCMS) (M+H)+402.1. ¹H NMR (400 MHz, (CD₃)₂SO) δ 9.17 (s, 1H), 8.72 (d, 1H), 8.14 (d, 1H), 7.62 (dd, 1H), 4.88 (d, 2H), 3.92-3.50 (m, 4H), 3.11 (t, 2H), 1.87-1.75 (m, 2H), 1.41-1.37 (m, 8H), 0.95 (t, 3H), 0.57 (s, 3H).

### Step 7. Preparation of 2-butyl-7-chloro-1-((2,2,5-trimethyl-1,3-dioxan-5-yl)methyl)-1H-imidazo[4,5-c]quinoline 5-oxide (C12)

The following reaction was conducted in 4 batches in parallel then combined for work-up. At 0° C., to a solution of C11 (30.0 g, 74.6 mmol) in DCM (750 mL) was added m-CPBA (19.3 g, 112 mmol) to form the first batch. The reaction mixture of the first batch was warmed to room temperature slowly then stirred for 16 hours.

The additional 2 batches of the same reaction were conducted with C11 (30.0 g, 74.6 mmol) and 1 batch of the same reaction was conducted with C11 (10.0 g, 24.9 mmol). The 4 batches were combined then 5% NaHCO₃ solution (1.5 L) was added and stirred at room temperature for 2 hours. The layers were separated then the organic layer was washed with 5% NaHCO₃ (1.5 L×2), brine and dried with Na₂SO₄. The dried organic layer was filtered and concentrated in vacuo. To the brown oil was added EtOAc (1.0 L) then the solution was concentrated in vacuo to form a foam. To the foam was added MTBE (1.0 L) then the solution was concentrated in vacuo to form a brown solid. To the solid was added MTBE (0.5 L) and the reaction mixture was stirred for 16 hours then filtered. The filter cake was collected to provide C12 (80.0 g, 76.9% yield) as a brown solid. The solid was used directly in the next step without further purification. (LCMS) (M+H)⁺ 418.0. ¹H NMR (400 MHz, (CD₃)₂SO) δ 9.05 (s, 1H), 8.81-8.74 (m, 2H), 7.77 (d, 1H), 4.85 (d, 2H), 3.89-3.54 (m, 4H), 3.08-3.06 (m, 2H), 1.84-1.74 (m, 2H), 1.40-1.36 (m, 8H), 0.94 (t, 3H), 0.59 (s, 3H).

### Step 8. Preparation of 2-butyl-7-chloro-1-((2,2,5-trimethyl-1,3-dioxan-5-yl)methyl)-1H-imidazo[4,5-c]quinolin-4-amine (C13)

The following reaction was conducted in 4 batches in parallel then combined for work-up. At 0° C., to a solution of C12 (20.0 g, 48 mmol) in DCM (800 mL) was added 28% NH₄OH in H₂O (150 mL) and TsCl (11 g, 57 mmol) successively to form the first batch. The reaction mixture of the first batch was warmed to room temperature slowly then stirred for 16 hours.

The additional 3 batches of the same reaction were conducted with C12 (20.0 g, 48 mmol). The 4 batches were combined then washed with saturated NaHCO₃ (2 L×2) and brine (2 L×2). The organic layer was concentrated in vacuo then the residue was slurried in IPA (650 mL) at room temperature for 16 hours before the suspension was filtered. The filter cake was collected to provide C13 (66 g, 83% yield) as a yellow solid. The solid was used directly in the next step without further purification. (LCMS) (M+H)⁺ 417.2. ¹H NMR (400 MHz, (CD₃)₂SO) δ 8.35 (d, 1H), 7.54 (d, 1H), 7.16 (dd, 1H), 6.71 (s, 2H), 4.83-4.73 (m, 2H), 3.90-3.35 (m, 4H), 3.04 (br d, 2H), 1.75 (br s, 2H), 1.46-1.36 (m, 8H), 0.94 (t, 3H), 0.57 (s, 3H).

### Step 9. Preparation of tert-butyl 4-(3-(4-amino-2-butyl-1-((2,2,5-trimethyl-1,3-dioxan-5-yl)methyl)-1H-imidazo[4,5-c]quinolin-7-yl)propyl)piperazine-1-carboxylate (C14)

The following reaction was conducted in 7 batches in parallel then combined for work-up. To a solution of C13 (10.0 g, 24 mmol) in DMF (300 mL) was added Na2CO₃ (7.5 g, 72 mmol), RuPhos Pd G3 (4.0 g, 4.8 mmol), H₂O (40 mL) and P3 (120 mL) successively to provide the first batch. The reaction mixture of the first batch was degassed with N₂ gas then stirred at 90° C. for 16 hours under N₂ gas.

The additional 5 batches of the same reaction were conducted with C13 (10.0 g, 24 mmol) and 1 batch of the same reaction was conducted with C13 (5.0 g, 0.12 mol). The 7 batches were combined then filtered through a celite pad. The filtrate was diluted with H₂O (5 L) then extracted with EtOAc (2 L×3). The combined organic layer was washed with brine (2 L×3), dried with Na₂SO₄ and concentrated in vacuo. The residue was purified by column chromatography (silica gel, MeOH:DCM, 0:100 to 10:90) to provide a yellow oil that was slurried in MTBE (800 mL) at room temperature for 16 hours then was filtered. The filter cake was collected to provide C14 (63 g, 66% yield) as an off-white solid. (LCMS) (M+H)⁺ 609.5. ¹H NMR (400 MHz, (CD₃)₂SO) δ 8.23 (d, 1H), 7.38 (d, 1H), 7.06 (dd, 1H), 6.40 (s, 2H), 4.77 (br s, 2H), 3.92-3.51 (m, 4H), 3.41-3.34 (m, 1H), 3.29-3.26 (m, 2H), 3.12-2.89 (m, 2H), 2.68 (t, 2H), 2.36-2.35 (m, 6H), 1.84-1.67 (m, 4H), 1.44-1.36 (m, 18H), 0.94 (t, 3H), 0.58 (s, 3H).

### Step 10. Preparation of 2-((4-amino-2-butyl-7-(3-(piperazin-1-yl)propyl)-1H-imidazo[4,5-c]quinolin-1-yl)methyl)-2-methylpropane-1,3-diol hydrochloride (P4)

The following reaction was conducted in 7 batches in parallel then combined for work-up. At 0° C., to a solution of C14 (10.0 g, 16 mmol) in (1:1) MeOH:DCM (260 mL) was added 4M HCl in 1,4-dioxane (200 mL) then stirred 16 hours at room temperature to provide the first batch.

The additional 5 batches of the same reaction were conducted with C14 (10.0 g, 16 mmol) and 1 batch of the same reaction was conducted with C14 (3.0 g, 0.0049 mmol). The 7 batches were combined then concentrated in vacuo. The residue was slurried in EtOAc (500 mL) at room temperature for 2 hours then filtered. The filter cake was collected and dried in vacuo to provide P4 (57 g, >99% yield) as an off-white solid. The solid was used directly in the next step without additional purification. (LCMS) (M+H)⁺ 469.35. ¹H NMR (400 MHz, D₂O) δ 8.38 (d, 1H), 7.52 (s, 1H), 7.45 (dd, 1H), 4.76-4.67 (m, 1H), 4.56-4.42 (m, 1H), 3.79-3.32 (m, 14H), 3.06 (t, 2H), 2.94 (t, 2H), 2.27-2.16 (m, 2H), 1.92-1.82 (m, 2H), 1.56-1.45 (m, 2H), 1.01 (t, 3H), 0.63 (s, 3H).

### Preparation P5

### Tert-butyl(2-(2-(2-(2-(chlorosulfonyl)ethoxy)ethoxy)ethoxy)ethyl)carbamate (P5)

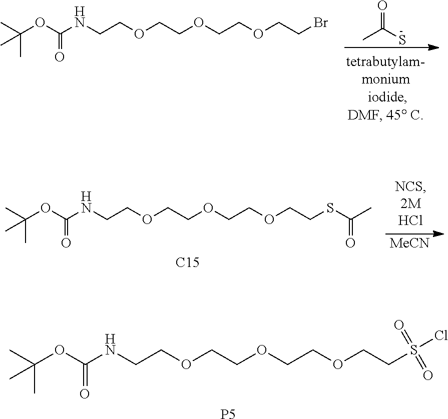

### Step 1. Preparation of S-(2,2-dimethyl-4-oxo-3,8,11,14-tetraoxa-5-azahexadecan-16-yl) ethanethioate (C15)

Under N₂ gas, to a stirred solution of tert-butyl(2-(2-(2-(2-bromoethoxy)ethoxy)ethoxy)ethyl)carbamate (CAS: 1076199-21-7; 1.00 g, 2.81 mmol) and tetrabutylammonium iodide (0.104 g, 0.281 mmol) in DMF (25 mL) was added potassium thioacetate (CAS: 10387-40-3; 0.641 g, 5.61 mmol). The reaction mixture was heated to 45° C. and stirred for 2 hours then poured into ice-water (30 mL). The diluted reaction mixture was extracted with EtOAC (30 mL×3) then the combined organic layer was washed with 1M HCl (30 mL), saturated NaHCO₃ (30 mL) and brine (30 mL). The organic layer was dried over Na₂SO₄, filtered, and concentrated in vacuo to provide C15 (1.10 g, >99% yield) as brown oil. The oil was used directly in the next step without further purification. (LCMS) (M+23H)⁺ 374.3.

### Step 2. Preparation of tert-butyl(2-(2-(2-(2-(chlorosulfonyl)ethoxy)ethoxy)ethoxy)ethyl)carbamate (P5)

At 0-5° C., to a stirred solution of C15 (0.550 g, 1.56 mmol) in MeCN (10 mL) was added aqueous 2M HCl (114 mg, 3.13 mmol) then NCS (418 mg, 3.13 mmol). The reaction mixture was warmed to room temperature and stirred for 2.5 hours. After the stir, the reaction mixture was extracted with the EtOAc (20 mL×3). The combined organic layer was washed with saturated NaHCO₃ (20 mL×3) and brine (20 mL×3). The organic layer was dried with Na₂SO₄, filtered and concentrated in vacuo to provide P5 (0.160 g, 27.2% yield) as yellow gum. The gum was used directly in the next step without further purification.

### Preparation P6

### Tert-butyl (S)-1,14-dioxo-17-palmitamido-4,7,10-trioxa-13-azaoctadecan-18-oate (P6)

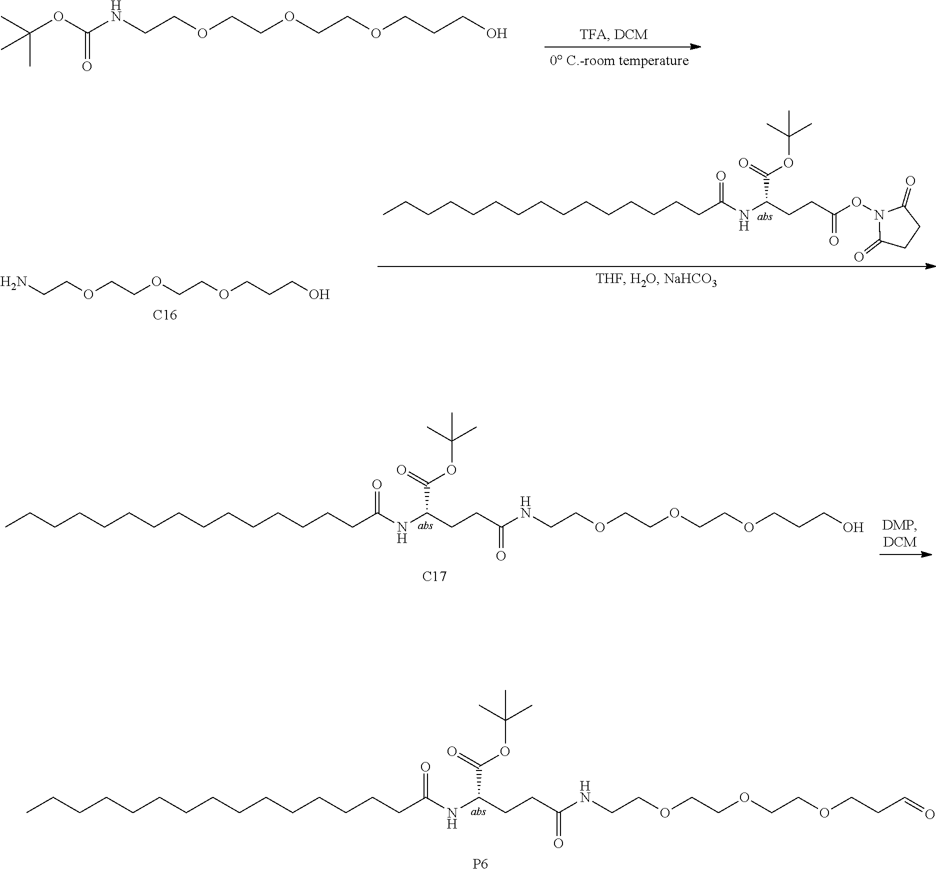

### Step 1. Preparation of 3-(2-(2-(2-aminoethoxy)ethoxy)ethoxy)propan-1-ol trifluoroacetate (C16)

At 0° C. under N₂ gas, to a stirred solution of tert-butyl(2-(2-(2-(3-hydroxypropoxy)ethoxy)ethoxy)ethyl)carbamate (CAS: 1818885-72-1; 0.200 g, 0.586 mmol) in DCM (2 mL) was added TFA (2 mL). The reaction mixture was warmed to room temperature then stirred for 1 hour before the reaction mixture was concentrated in vacuo to provide C₁₆ (325 mg, >99% yield) as a colorless oil. The oil was used directly in the next step without additional purification.

### Step 2. Preparation of tert-butyl (S)-1-hydroxy-14-oxo-17-palmitamido-4,7,10-trioxa-13-azaoctadecan-18-oate (C17)

To a stirred solution of C16 (0.700 g, 1.31 mmol) in THF (6 mL) and H₂O (6 mL) was added NaHCO₃ (468 mg, 5.57 mmol) then 1-(tert-butyl) 5-(2,5-dioxopyrrolidin-1-yl) palmitoyl-L-glutamate (CAS: 204521-63-1; 0.600 g, 1.11 mmol) successively at room temperature. The reaction mixture was stirred for 16 hours at room temperature under N₂ gas before the yellow reaction mixture was diluted with EtOAc (20 mL) and washed with saturated NaHCO₃ (20 mL).

The organic layer was dried with Na₂SO₄, filtered, and concentrated in vacuo. The yellow oil was purified by (silica gel, MeOH:DCM, 0-7% MeOH) to provide C17 (0.600 g, 85.4% yield) as a white solid. (LCMS) (M+H)⁺ 631.4. ¹H NMR (400 MHz, CDCl₃) δ 7.34 (t, 1H), 6.61 (d, 1H), 4.44-4.36 (m, 1H), 3.79 (t, 2H), 3.71 (t, 2H), 3.65-3.57 (m, 8H), 3.52 (t, 2H), 3.46-3.37 (m, 2H), 2.36-2.06 (m, 6H), 2.02-1.90 (m, 1H), 1.86-1.79 (m, 2H), 1.66-1.55 (m, 2H), 1.45 (s, 9H), 1.32-1.20 (m, 24H), 0.95-0.87 (t, 3H).

### Step 3. Preparation of tert-butyl (S)-1,14-dioxo-17-palmitamido-4,7,10-trioxa-13-azaoctadecan-18-oate (P6)

To a stirred solution of C17 (0.200 g, 0.269 mmol) in DCM (4 mL) was added DMP (229 mg, 0.539 mmol) at room temperature then was stirred for 2 hours. The yellow reaction mixture was filtered then the filtrate was washed with saturated NaHCO₃ (10 mL), dried with Na₂SO₄, filtered and concentrated in vacuo to provide P6 (0.200 g, >99% yield) as a white waxy solid. The solid was used directly in the next step without further purification. ¹H NMR (400 MHz, (CD₃)₂SO) δ 9.64 (t, 1H), 8.08-7.99 (m, 1H), 7.91-7.84 (m, 1H), 4.08-3.99 (m, 1H), 3.72 (t, 2H), 3.50-3.48 (m, 8H), 3.40-3.35 (m, 2H), 3.17 (q, 2H), 2.60 (td, 2H), 2.10 (dt, 4H), 1.90-1.81 (m, 1H), 1.78-1.66 (m, 1H), 1.52-1.42 (m, 2H), 1.38 (s, 9H), 1.23 (s, 24H), 0.85 (t, 3H).

### Preparation P7

### (S)-2-(1-(5-(Tert-butoxy)-5-oxo-4-palmitamidopentanoyl)-4-hydroxypiperidin-4-yl)acetic Acid (P7)

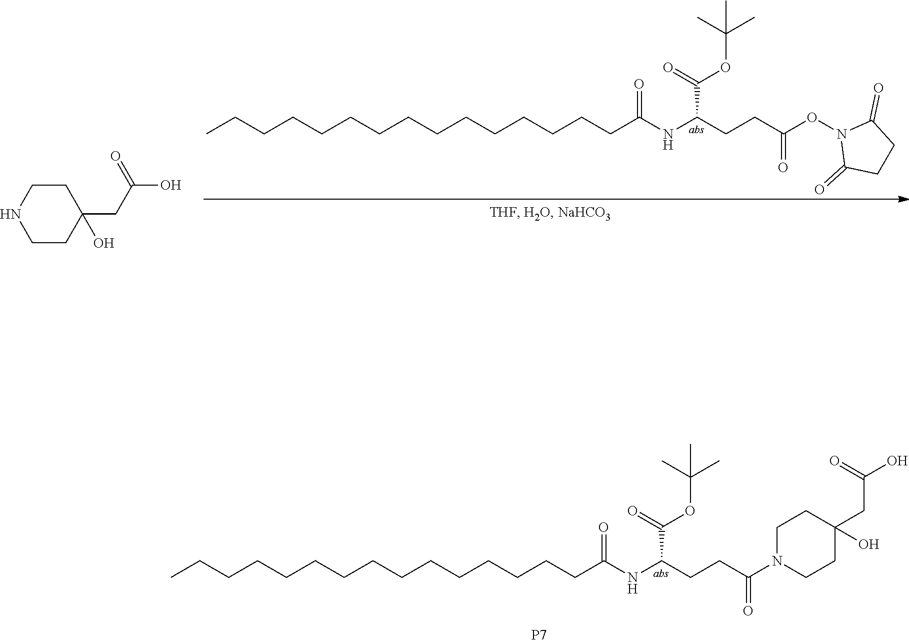 [SMILES: O=C(O)CC1(O)CCNCC1 ; CCCCCCCCCCCCCCCC(=O)N[C@@H](CCC(=O)ON1C(=O)CCC1=O)C(=O)OC(C)(C)C ; CCCCCCCCCCCCCCCC(=O)N[C@@H](CCC(=O)N1CCC(O)(CC(=O)O)CC1)C(=O)OC(C)(C)C]

### Preparation of(S)-2-(1-(5-(tert-butoxy)-5-oxo-4-palmitamidopentanoyl)-4-hydroxypiperidin-4-yl)acetic Acid (P7)

To a stirred solution of 2-(4-hydroxypiperidin-4-yl)acetic acid (CAS: 328401-29-2; 0.350 g, 0.730 mmol) in THF (2 mL) and H₂O (2 mL) was added NaHCO₃ (0.600 g, 7.14 mmol) to PH˜8 then 1-(tert-butyl) 5-(2,5-dioxopyrrolidin-1-yl) palmitoyl-L-glutamate (CAS: 204521-63-1; 0.393 g, 0.730 mmol) successively at room temperature. The reaction mixture was stirred at room temperature under N₂ gas for 2 hours then was acidified with aqueous 1M HCl to PH˜4. The acidic reaction mixture was extracted with EtOAc (20 mL×3) then the combined organic layer was dried with Na₂SO₄, filtered, and concentrated in vacuo. The yellow oil was purified by column chromatography (silica gel, MeOH:DCM, 0-7% MeOH) to provide P7 (0.350 g, 82.3% yield) as a colorless gum. (LCMS) (M+H)⁺ 583.5. ¹H NMR (400 MHz, CDCl₃) δ 6.75-6.61 (m, 1H), 4.46 (br s, 1H), 4.33 (t, 1H), 3.64-3.55 (m, 1H), 3.47-3.39 (m, 1H), 3.07 (q, 1H), 2.54-2.31 (m, 4H), 2.27-2.10 (m, 3H), 2.05-1.90 (m, 1H), 1.84-1.70 (m, 2H), 1.65-1.55 (m, 2H), 1.46 (s, 12H), 1.30-1.22 (m, 24H), 0.87 (t, 3H).

### Preparation P8

### 2-((4-Amino-2-(ethoxymethyl)-7-(3-(piperazin-1-yl)propyl)-1H-imidazo[4,5-c]quinolin-1-yl)methyl)-2-methylpropane-1,3-diol hydrochloride (P8)

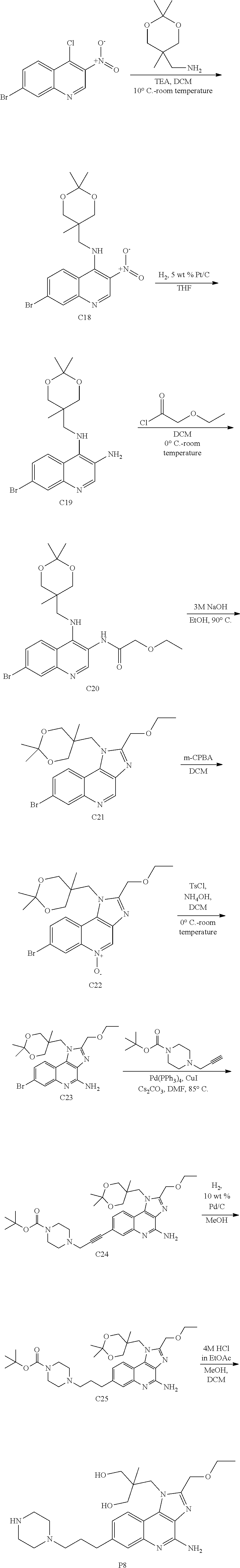

### Step 1. Preparation of 7-bromo-3-nitro-N-((2,2,5-trimethyl-1,3-dioxan-5-yl)methyl)quinolin-4-amine (C18)

At 10° C., to a yellow suspension of 7-bromo-4-chloro-3-nitroquinoline (CAS: 723280-98-6; 49.0 g, 0.170 mol) and TEA (34.5 g, 341 mmol) in DCM (820 mL) was added a solution of (2,2,5-trimethyl-1,3-dioxan-5-yl) methanamine (CAS: 493320-4; 27.1 g, 0.170 mol) in DCM (15 mL) then stirred at room temperature for 2 hours. The yellow reaction mixture changed from a yellow suspension to a clear solution. After the stir, the yellow reaction solution was diluted with DCM (300 mL) then washed with brine (200 mL), dried with Na₂SO₄, filtered and concentrated in vacuo. The yellow solid was diluted with DCM (100 mL) and stirred at room temperature for 20 minutes. The suspension was filtered, and the filter cake was collected then dried further to provide C18 (60.0 g) as a yellow solid.

The filtrate was concentrated in vacuo to provide a brown solid which was diluted with DCM (20 mL) then stirred at room temperature for 20 minutes. The yellow reaction suspension was filtered and washed with DCM (5 mL). The filter cake was collected then dried further to provide C18 (4.56 g) as a yellow solid.

The two batches of the desired intermediate were combined to provide C18 (64.6 g, 92.3% yield) as a yellow solid. The solid was used directly in the next step without additional purification. (LCMS) (M+H)⁺ 411.9. ¹H NMR (400 MHz, (CD₃)₂SO) δ 9.24-9.18 (m, 1H), 9.14 (s, 1H), 8.50 (d, 1H), 8.12 (d, 1H), 7.77 (dd, 1H), 3.93 (d, 2H), 3.65 (d, 2H), 3.55 (d, 2H), 1.35 (s, 3H), 1.22 (s, 3H), 0.91 (s, 3H).

### Step 2. Preparation of 7-bromo-N⁴-((2,2,5-trimethyl-1,3-dioxan-5-yl)methyl)quinoline-3,4-diamine (C19)

To a suspension of C18 (30.0 g, 73.1 mmol) in THF (250 mL) was added Pt/C (4.28 g, 1.10 mmol, 5 wt %). The reaction mixture was stirred under a balloon of H₂ at room temperature for 32 hours then was filtered through a pad of celite and washed with THF (600 mL). The filtrate was concentrated in vacuo then diluted with MeCN (50 mL) and stirred for 15 minutes. The reaction mixture was filtered and washed with MeCN (5 mL×2). The filter cake was collected and concentrated in vacuo to give C19 (21.0 g) as a yellow-green solid.

The filtrate was concentrated in vacuo then diluted with DCM (15 mL) and stirred at room temperature for 20 minutes. The yellow suspension was filtered and washed with DCM (5 mL). The filter cake was collected and dried to give C19 (4.61 g) as a brown solid.

The two batches of the desired intermediate were combined to provide C19 (25.6 g, 92.1% yield) as a solid. The solid was used directly in the next step without additional purification. (LCMS) (M)⁺ 380.1. ¹H NMR (400 MHz, (CD₃)₂SO) δ 8.37 (s, 1H), 8.01 (d, 1H), 7.89 (d, 1H), 7.44 (dd, 1H), 5.27 (s, 2H), 4.58 (t, 1H), 3.66 (d, 2H), 3.55 (d, 2H), 3.22 (d, 2H), 1.35 (s, 3H), 1.27 (s, 3H), 0.87 (s, 3H).

### Step 3. Preparation of N-(7-bromo-4-(((2,2,5-trimethyl-1,3-dioxan-5-yl)methyl) amino)quinolin-3-yl)-2-ethoxyacetamide (C20)

At 0° C. under N₂ gas, to a solution of C19 (12.0 g, 31.6 mmol) in DCM (240 mL) was added 2-ethoxyacetyl chloride (CAS: 14077-58-8; 3.87 g, 31.6 mmol) dropwise. The mixture was stirred from 0° C. to room temperature over 1 hour. The yellow reaction mixture was poured into saturated NaHCO₃ (300 mL) and extracted with DCM (150 mL×2). The combined organic layer was washed with brine (150 mL), dried with Na₂SO₄, filtered and concentrated in vacuo.

The brown oil was purified by column chromatography (silica gel, EtOAc, 100%) to provide C₂₀ (19.0 g, 60.7% yield) as a yellow gum. (LCMS) (M)⁺ 466.1. ¹H NMR (400 MHz, (CD₃)₂SO) δ 9.57 (s, 1H), 8.38-8.36 (m, 1H), 8.26 (d, 1H), 8.07-8.02 (m, 1H), 7.61 (dd, 1H), 5.75 (t, 1H), 4.11 (s, 2H), 3.72-3.51 (m, 8H), 1.38-1.29 (m, 3H), 1.28-1.16 (m, 6H), 0.81 (s, 3H).

### Step 4. Preparation of 7-bromo-2-(ethoxymethyl)-1-((2,2,5-trimethyl-1,3-dioxan-5-yl)methyl)-1H-imidazo[4,5-c]quinoline (C21)

The following reaction was conducted in two batches then combined for purification. Under N₂ gas, to a solution of C20 (19.2 g, 41.2 mmol) in EtOH (150 mL) was added aqueous 3M NaOH (20.6 mL) to form the first batch. The reaction mixture of the first batch was stirred at 90° C. for 2 hours.

A second batch of the same reaction was conducted with C20 (10.0 g, 21.4 mmol). The two batches were combined then concentrated in vacuo. The residue was diluted with H₂O (100 mL) and EtOAc (30 mL) then stirred at room temperature for 15 minutes before filtration. The filter cake was washed with H₂O (20 mL) then collected. The solid was diluted with (2:1) EtOAc; petroleum ether (60 mL) and stirred for 40 minutes. The yellow suspension was filtered and washed with EtOAc (10 mL×3). The filter cake was collected again then diluted with MeCN and concentrated in vacuo to provide C21 (15.8 g, 56.3% yield) as a faint yellow solid. The solid was used directly in the next step without additional purification. (LCMS) (M+H)⁺ 449.9. ¹H NMR (400 MHz, (CD₃)₂SO) δ 9.17 (s, 1H), 8.63 (d, 1H), 8.29 (d, 1H), 7.73 (dd, 1H), 5.25-4.89 (m, 3H), 4.79-4.57 (m, 1H), 3.89-3.46 (m, 6H), 1.35 (d, 6H), 1.10 (t, 3H), 0.53 (s, 3H).

### Step 5. Preparation of 4-amino-7-bromo-2-(ethoxymethyl)-1-((2,2,5-trimethyl-1,3-dioxan-5-yl)methyl)-1H-imidazo[4,5-c]quinoline 5-oxide (C22)

Under N₂ gas, to a solution of C21 (15.8 g, 35.1 mmol) in DCM (450 mL) was added m-CPBA (8.56 g, 42.2 mmol) then was stirred at room temperature for 16 hours. The reaction solution was diluted with DCM (500 mL) and washed with saturated NaHCO₃ (200 mL) then brine (200 mL). The organic layer was dried with Na₂SO₄, filtered, and concentrated in vacuo. The yellow solid was suspended in petroleum ether (60 mL) and DCM (20 mL) then stirred at room temperature for 15 minutes. The suspension was filtered then the filter cake was collected to provide C22 (14.5 g, 88.7%) as a yellow solid. (LCMS) (M)⁺ 464.2. ¹H NMR (400 MHz, (CD₃)₂SO) δ 9.11 (s, 1H), 8.95 (d, 1H), 8.73 (d, 1H), 7.93 (dd, 1H), 5.19-4.93 (m, 3H), 4.73-4.61 (m, 1H), 3.93-3.50 (m, 6H), 1.40 (d, 6H), 1.16 (t, 3H), 0.60 (s, 3H).

### Step 6. Preparation of 7-bromo-2-(ethoxymethyl)-1-((2,2,5-trimethyl-1,3-dioxan-5-yl)methyl)-1H-imidazo[4,5-c]quinolin-4-amine (C23)

At 0° C., to a solution of C22 (14.5 g, 31.2 mmol) in DCM (250 mL) was added NH₄OH (78.0 g, 623 mmol) then TsCl (7.13 g, 37.4 mmol) portion wise over 20 minutes. The reaction mixture was warmed to room temperature and stirred for 16 hours. The white suspension was diluted with DCM (400 mL), washed with saturated NaHCO₃ (300 mL) then brine (200 mL). The organic layer was dried with Na₂SO₄, filtered and concentrated in vacuo. The yellow solid was suspended in DCM (40 mL) and petroleum ether (30 mL) then stirred at room temperature for 15 minutes before filtration. The filter cake was washed with DCM (5 mL×2) then collected and dried to provide C23 (12.5 g, 86.6% yield) as a light-yellow solid. (LCMS) (M+2H)⁺ 465.0. ¹H NMR (400 MHz, (CD₃)₂SO) δ 8.31 (d, 1H), 7.71 (d, 1H), 7.39-7.24 (m, 1H), 6.87 (s, 2H), 5.18-4.80 (m, 3H), 4.68-4.56 (m, 1H), 3.91-3.48 (m, 6H), 1.41 (d, 6H), 1.14 (t, 3H), 0.58 (s, 3H).

### Step 7. Preparation of tert-butyl 4-(3-(4-amino-2-(ethoxymethyl)-1-((2,2,5-trimethyl-1,3-dioxan-5-yl)methyl)-1H-imidazo[4,5-c]quinolin-7-yl) prop-2-yn-1-yl)piperazine-1-carboxylate (C24)

Under argon gas, to a solution of C23 (6.00 g, 12.9 mmol), tert-butyl 4-(prop-2-yn-1-yl)piperazine-1-carboxylate (CAS: 199538-99-3; 3.49 g, 15.5 mmol) in DMF (240 mL) was added Pd(PPh₃)₄ (1.50 g, 1.29 mmol), CuI (0.493 g, 2.59 mmol), and Cs₂CO₃ (21.1 g, 64.7 mmol) at room temperature. The reaction mixture was heated to 85° C. and stirred for 16 hours then cooled to room temperature. The cooled reaction mixture was diluted with H₂O (700 mL) then stirred for 3 minutes. The yellow suspension was filtered then the filter cake was washed with H₂O (50 mL×2). The aqueous filtrate was discarded. The filter cake was then rinsed with DCM (˜300 mL) and the filtrate was dried with Na₂SO₄, filtered, and concentrated in vacuo. The brown gum was purified by column chromatography (silica gel, MeOH:EtOAc, 0-6% MeOH) to provide an impure C24 as a brown solid which was suspended in MeCN (20 mL) and petroleum ether (10 mL) then stirred at room temperature for 15 minutes before filtration. The filter cake was washed with MeCN (5 mL×2) then collected and dried to provide C24 (3.60 g, 45.8% yield) as a white solid. (LCMS) (M+H)⁺ 607.2. ¹H NMR (400 MHz, CDCl₃) δ 8.10 (d, 1H), 7.88-7.86 (m, 1H), 7.31 (dd, 1H), 5.51 (br s, 2H), 5.22-4.66 (m, 4H), 3.84-3.60 (m, 4H), 3.50 (t, 4H), 2.61 (t, 4H), 1.74 (br s, 4H), 1.54-1.48 (m, 6H), 1.47 (br s, 9H), 1.28-1.22 (m, 3H), 0.63 (s, 3H).

### Step 8. Preparation of tert-butyl 4-(3-(4-amino-2-(ethoxymethyl)-1-((2,2,5-trimethyl-1,3-dioxan-5-yl)methyl)-1H-imidazo[4,5-c]quinolin-7-yl)propyl)piperazine-1-carboxylate (C25)

A mixture of C24 (5.3 g, 8.7 mmol) in MeOH (270 mL) was added Pd/C (1.6 g, 1.6 mmol, 10 wt %). The reaction mixture was degassed with H₂ gas 3 times then stirred at room temperature under a balloon of H₂ for 20 hours. The suspension was filtered through a pad of celite then the filter cake was washed with MeOH (50 mL). The filtrate was concentrated in vacuo then the gray gum was purified by column chromatography (silica gel, MeOH:EtOAc, 0-4% MeOH; then MeOH:DCM, 0-6% MeOH) to provide the impure C25 brown gum. The brown gum was suspended in petroleum ether (30 mL) and EtOAc (5 mL) then stirred at room temperature for 15 minutes before filtration. The filter cake was washed with EtOAc (5 mL×2) then was collected and dried further to provide C25 (3.8 g, 71% yield) as a white solid. (LCMS) (M+H)⁺ 611.5. ¹H NMR (400 MHz, (CD₃)₂SO) δ 8.23 (d, 1H), 7.39-7.37 (m, 1H), 7.07 (dd, 1H), 6.59 (br s, 2H), 5.14-4.78 (m, 3H), 4.65-4.52 (m, 1H), 3.89-3.48 (m, 6H), 3.28-3.25 (m, 4H), 2.66 (t, 2H), 2.33-2.24 (m, 6H), 1.83-1.74 (m, 2H), 1.42-1.32 (m, 15H), 1.12 (t, 3H), 0.57 (s, 3H).

### Step 9. Preparation of 2-((4-amino-2-(ethoxymethyl)-7-(3-(piperazin-1-yl)propyl)-1H-imidazo[4,5-c]quinolin-1-yl)methyl)-2-methylpropane-1,3-diol hydrochloride (P8)

To a mixture of C25 (3.80 g, 6.22 mmol) in DCM (50 mL) and MeOH (50 mL) was added 4M HCl in EtOAc (150 mL). The reaction mixture was stirred at room temperature for 2 hours then the white suspension was filtered. The filter cake was collected then lyophilized to provide P8 (3.82 g, >99% yield) as a light-brown solid. The solid was used directly in the next step without additional purification. (LCMS) (M+H)⁺ 471.3. ¹H NMR (400 MHz, (CD₃)₂SO) δ 14.05 (s, 1H), 12.06 (br s, 1H), 10.07-9.78 (m, 2H), 9.30 (br s, 1H), 8.69 (d, 1H), 7.63 (s, 1H), 7.44 (d, 1H), 5.15-4.61 (m, 4H), 3.77-3.13 (m, 16H), 2.84 (t, 2H), 2.19-2.04 (m, 2H), 1.14 (t, 3H), 0.56 (s, 3H).

The Schemes described below are intended to provide a general description of the methodology employed in the preparation of the compounds of the present disclosure. Some of the compounds of the present disclosure contain a single chiral center. In the following Schemes, the general methods for the preparation of the compounds are shown either in racemic or enantioenriched form. It will be apparent to one skilled in the art that all of the synthetic transformations may be conducted in a precisely similar manner whether the materials are enantioenriched or racemic. Moreover, the resolution to the desired optically active material may take place at any desired point in the sequence using well known methods such as described herein and in the chemistry literature.

Compounds of the disclosure may be made according to the following Schemes 1-6, although alternative methodologies may also be utilized. One skilled in the art will appreciate that alternative reaction conditions to the ones illustrated in the schemes and examples may be utilized as deemed appropriate. Choices of solvents, additives such as acidic or basic catalysts, coupling agents, and indeed the reaction sequence may be changed as appropriate for a given target compound.

In some cases, compounds of described herein may contain protecting groups, which may be appended or removed by additional steps in the synthetic sequence using conditions known in the art (March's Advanced Organic Chemistry: Reactions, Mechanisms, and Structure 8th Edition or Protecting Groups, 10 Georg Thieme Verlag, 1994). Compounds at every step may be purified by standard techniques, such as column chromatography, crystallization, or reverse phase SFC or HPLC.

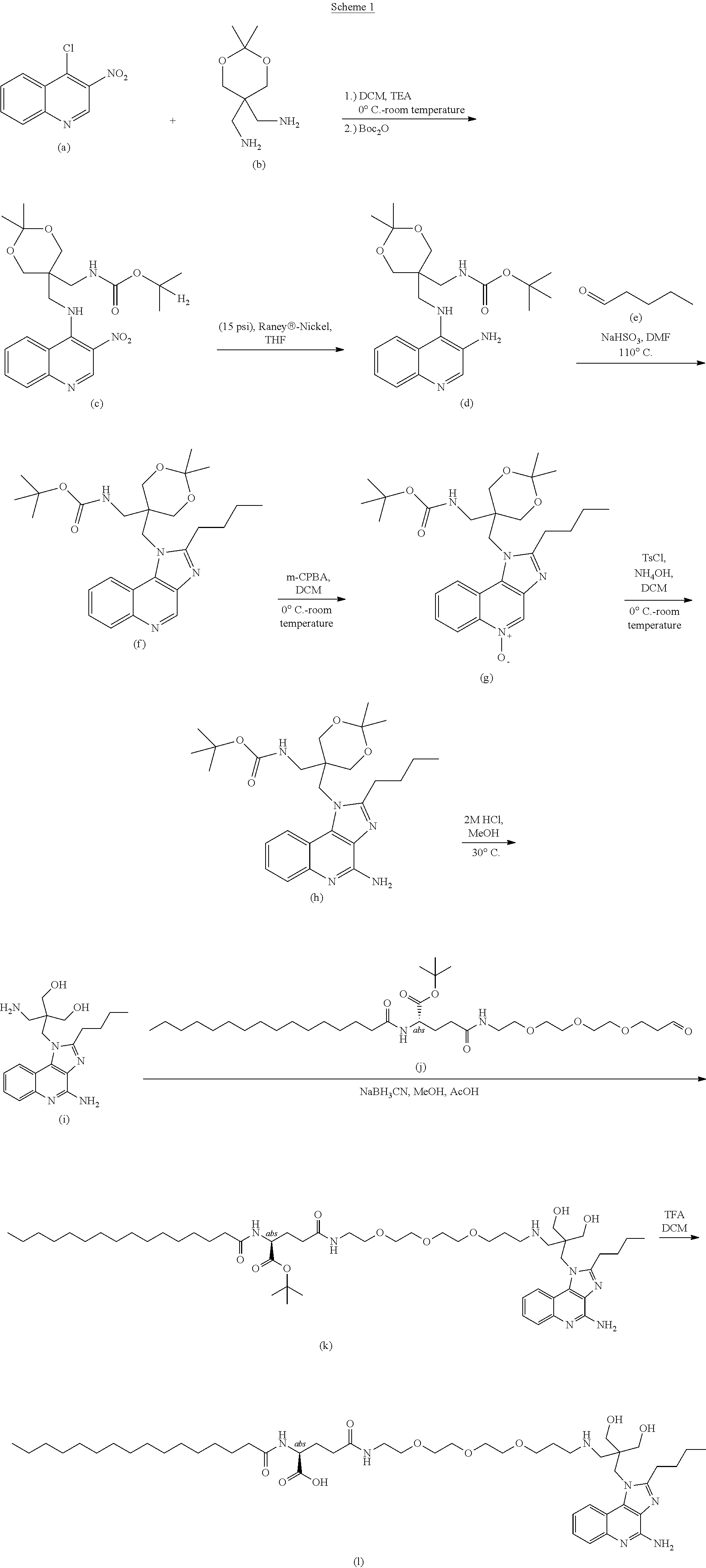

The halogen (e.g. Cl)-substituted heterocycle (a) can react with the amine (b) in a solvent such as dichloromethane (DCM) and with a base like triethylamine (TEA) from 0° C. to room temperature which then can react with di-tert-butyl decarbonate (Boc₂O) to provide the protected amine (c). The nitro-intermediate (c) can then be reduced through a hydrogenation with a catalyst such as Raney®-Nickel under hydrogen gas (H₂) in a solvent like tetrahydrofuran (THF) to afford the aniline (d). The aniline (d) can then react with pentanal (e) and cyclize under basic conditions like sodium bisulfite (NaHSO₃) in a suitable solvent such as dimethylformamide (DMF) heated from 80-110° C. then stirred from 16-24 hours to provide the tricycle (f). The tricycle (f) in a solvent such as DCM can oxidize with the treatment of 3-chloroperoxybenzoic acid (m-CPBA) with stirring from 24-48 hours at room temperature to provide the N-oxide (g). The N-oxide (g) in a solvent like DCM with a base such as ammonium hydroxide (NH₄OH) can react with p-toluenesulfonyl chloride (TsCl) from 0° C. to room temperature with stirring from 16-24 hours to afford the aniline (h). The aniline intermediate (h) can undergo deprotection under standard acidic conditions such as with hydrochloric acid (HCl) in a solvent like methanol (MeOH) and heated from 30-50° C. to form the amine (i). The amine (i) can undergo a reductive amination with the aldehyde (j) in a solvent like MeOH with a mild reducing agent such as sodium cyanoborohydride (NaBH₃CN) and with a catalytic amount of acid such as acetic acid (AcOH) to afford the protected acid intermediate (k). The protected acid intermediate (k) can undergo a deprotection with acidic conditions like trifluoroacetic acid (TFA) in a solvent like DCM from 0° C. to room temperature to form the target product (I).

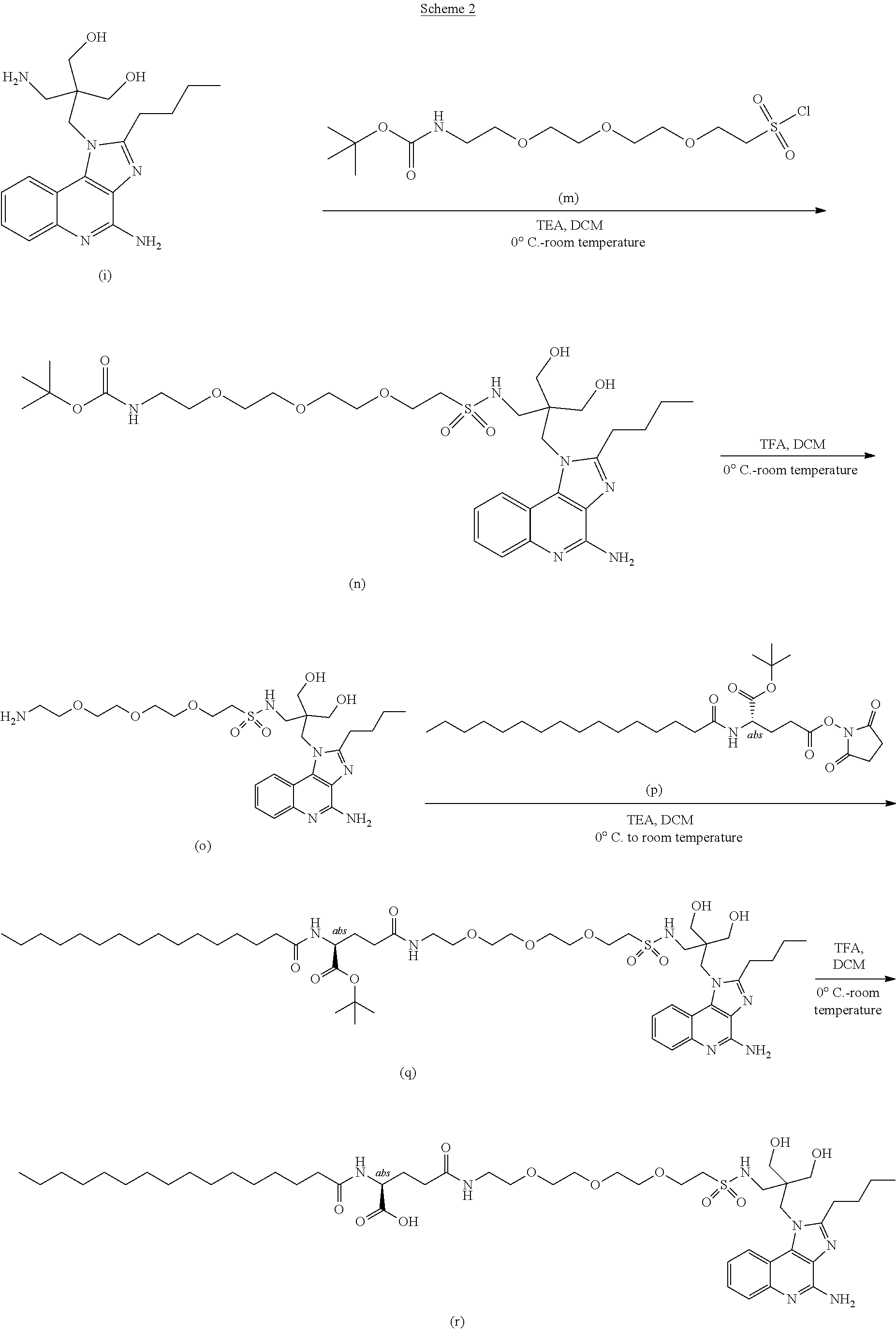

The amine (i) can react with the sulfonyl chloride (m) in a solvent like DCM and with a base like TEA from 0° C. to room temperature stirred from 16-24 hours to form the terminal protected amine intermediate (n) which can be deprotected under standard acidic conditions like with TFA in a solvent like DCM from 0° C. to room temperature to provide the terminal amine (o). The amine (o) can react with the NHS-ester (N-hydroxysuccinimide ester) (p) in a solvent like DCM and with a base like TEA from 0° C. to room temperature to afford the protected acid intermediate (q) which can be deprotected under standard acidic conditions like TFA in a solvent like DCM from 0° C. to room temperature to provide the target product (r).

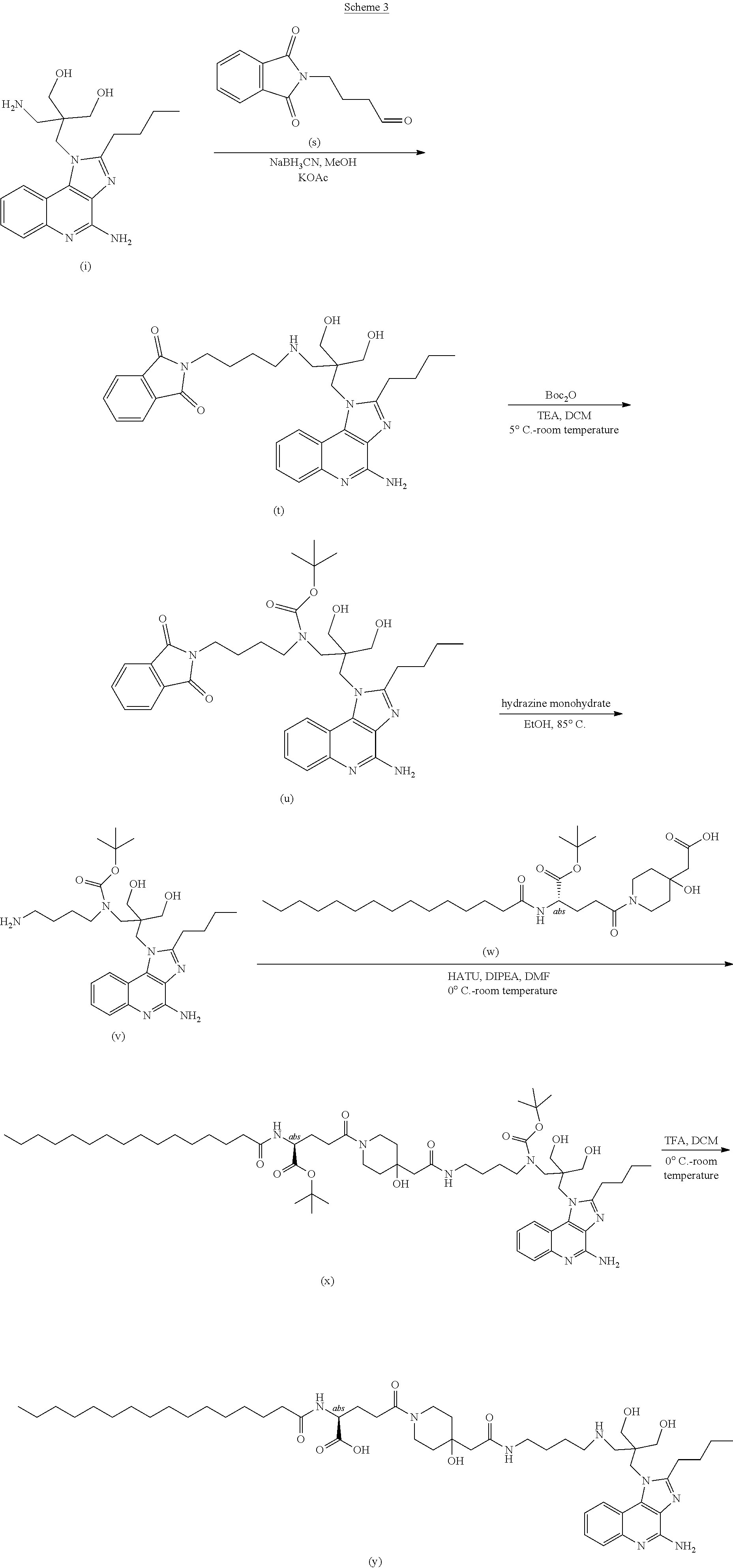

The amine (i) can undergo a reductive amination with the aldehyde(s) in a solvent like MeOH with a mild reducing agent such as sodium cyanoborohydride (NaBH₃CN) and with a weak acid like potassium acetate (KOAc) stirred from 16-24 hours to afford the protected intermediate (t). The secondary amine (t) can be protected with Boc2O with a base like TEA in a solvent like DCM from 5° C. to room temperature with stirring from 16-24 hours to provide the protected amine (u). The NHS-ester intermediate (u) can be deprotected with hydrazine monohydrate in a solvent like ethanol (EtOH) with heat from 80-100° C. with stirring from 16-24 hours to form the terminal amine (v). The terminal amine (v) can react with the acid (w) through standard amide coupling conditions like 1-[bis(dimethylamino)methylene]-1H-1,2,3-triazolo[4,5-b]pyridinium 3-oxid hexafluorophosphate (HATU) with a base like N,N-diisopropylethylamine (DIPEA) in a solvent like DMF from 0° C. to room temperature with stirring from 16-24 hours to form the amide (x). The protected acid (x) can be deprotected under standard acidic conditions like TFA in a solvent like DCM from 0° C. to room temperature to provide the target product (y).

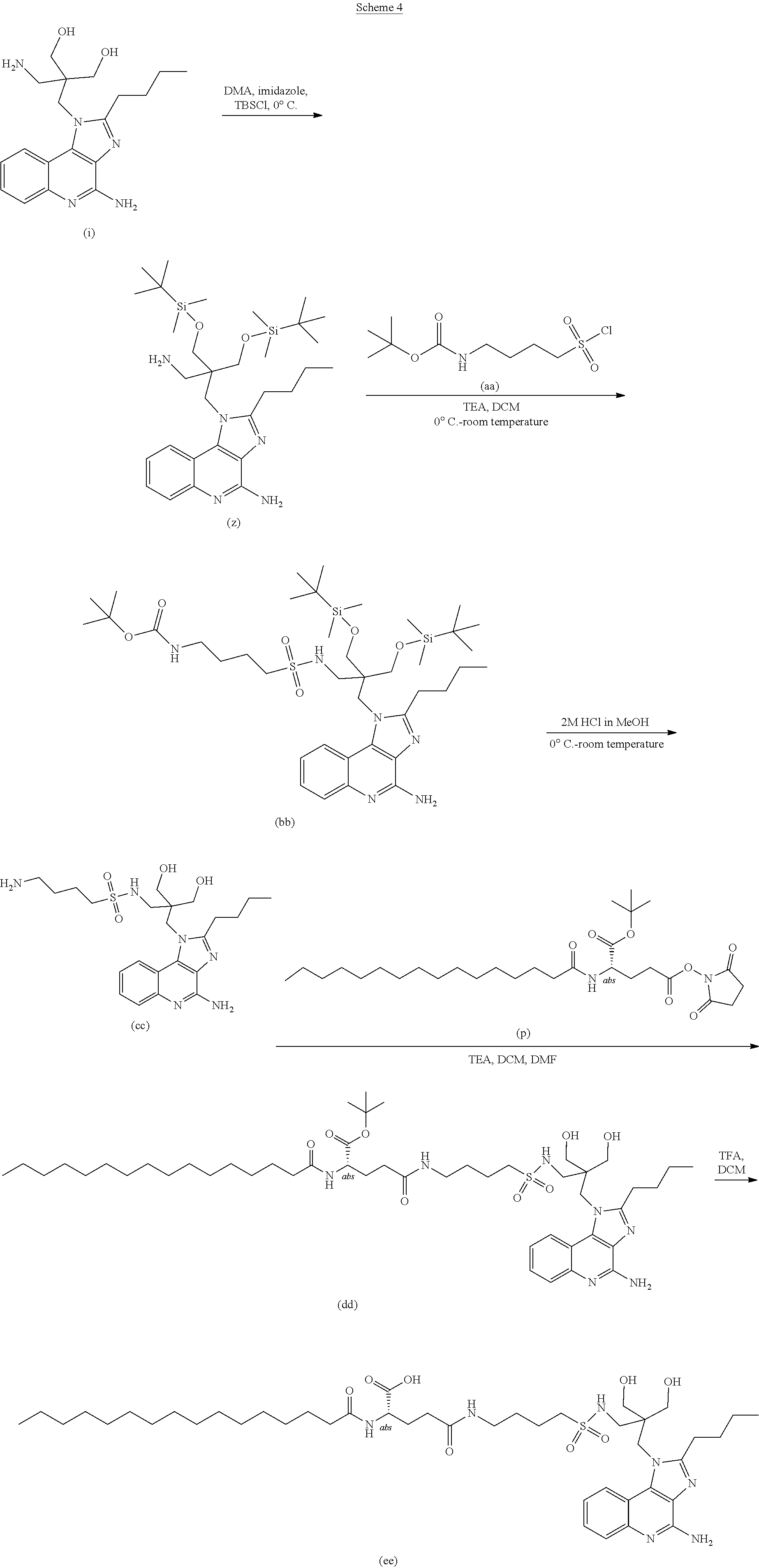

The alcohol intermediate (i) can be protected by reacting with tert-butyldimethylsilyl chloride (TBSCl) with a base like imidazole in a solvent like dimethylacetamide (DMA) from 0° C. to room temperature to provide the protected alcohol (z). The amine (z) can react with sulfonyl chloride (aa) with a base like TEA in a solvent like DCM from 0° C. to room temperature to afford the protected terminal amine (bb) which can be deprotected under acidic conditions such as with HCl in a solvent like MeOH from 0° C. to room temperature to form the amine (cc). The amine (cc) can react with the NHS-ester (p) in solvents like DCM and DMF with a base like TEA to afford the protected acid intermediate (dd) which can be deprotected under standard acidic conditions like TFA in a solvent like DCM to provide the target product (ee).

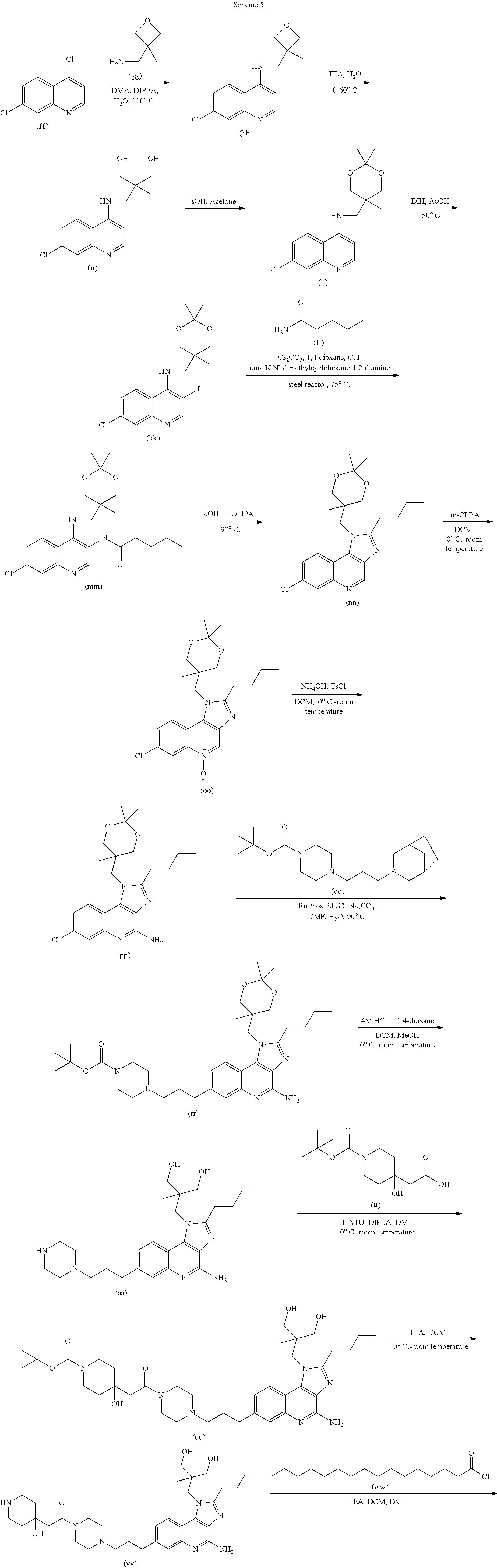

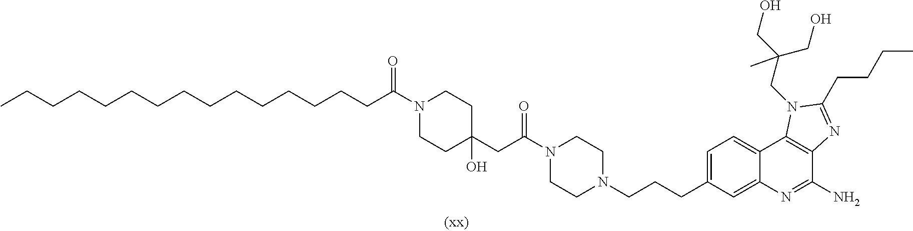 [SMILES: CCCCCCCCCCCCCCCC(=O)N1CCC(O)(CC(=O)N2CCN(CCCc3ccc4c(c3)nc(N)c3nc(CCCC)n(CC(C)(CO)CO)c34)CC2)CC1]

The halogen (e.g. Cl)-substituted heterocycle (ff) can react with the amine (gg) in solvents such as DMA and water (H₂O) with a base like DIPEA succumb to heat from 90-110° C. with stirring from 24-72 hours to provide the protected amine (hh). The protected amine (hh) can be deprotected under standard acidic conditions like TFA in a solvent like H₂O with heat from 50-80° C. to provide the alcohol (ii). The alcohol (ii) in a solvent like acetone can be protected by reacting with p-toluenesulfonic acid (TsOH) then stirring for 16-24 hours to afford the heterocycle (jj). The heterocycle (jj) can undergo an iodination by reacting with 1,3-diiodo-5,5-dimethylhydantoin (DIH) in an acid like AcOH with heat from 30-50° C. to form the iodo-substituted heterocycle (kk). The iodo-substituted heterocycle (kk) can react with the pentanamide (II) in a solvent like 1,4-dioxane, a base like cesium carbonate (Cs₂CO₃), a catalyst such as copper (I) iodide (CuI) and a ligand like trans-N,N′-dimethylcyclohexane-1,2-diamine with heat from 50-80° C. then stirring between 16-24 hours to form the amide intermediate (mm). The amide intermediate (mm) can be cyclized with a base like potassium hydroxide (KOH) in suitable solvents such as H₂O and IPA with heat from 90-110° C. and with stirring for 16-24 hours to afford the tricycle (nn). The tricycle (nn) in a solvent such as DCM can oxidize with the treatment of m-CPBA from 0° C. to room temperature with stirring from 16-24 hours to provide the N-oxide (oo). The N-oxide (oo) in a solvent like DCM with a base such as NH₄OH can react with TsCl from 0° C. to room temperature from 16-24 hours to afford the aniline (pp). The chloro-substituted heterocycle (pp) can react with the 9-BBN (9-borabicyclo[3.3.1]nonane) intermediate (qq) with a catalyst like RuPhos Pd G3 and a base like sodium carbonate (Na₂CO₃) in solvents like DMF and H₂O with heat from 80-100° C. then with stirring for 16-24 hours to afford the protected amine (rr). The protected amine (rr) can be deprotected under standard acidic conditions like HCl in 1,4-dioxane in solvents like DCM and MeOH from 0° C.—room temperature with stirring from 16-24 hours to provide the secondary amine (ss). The secondary amine (ss) can react with the acid (tt) under standard amide coupling conditions like HATU with a base like DIPEA in a solvent like DMF from 0° C.—room temperature with stirring from 16-24 hours to form the amide (uu). The protected secondary amine (uu) can be deprotected under standard acidic conditions like TFA in a solvent like DCM from 0° C.—room temperature to provide the secondary amine (vv) which can react with the acid chloride (ww) with a base like TEA in solvents like DCM and DMF to provide the target product (xx).

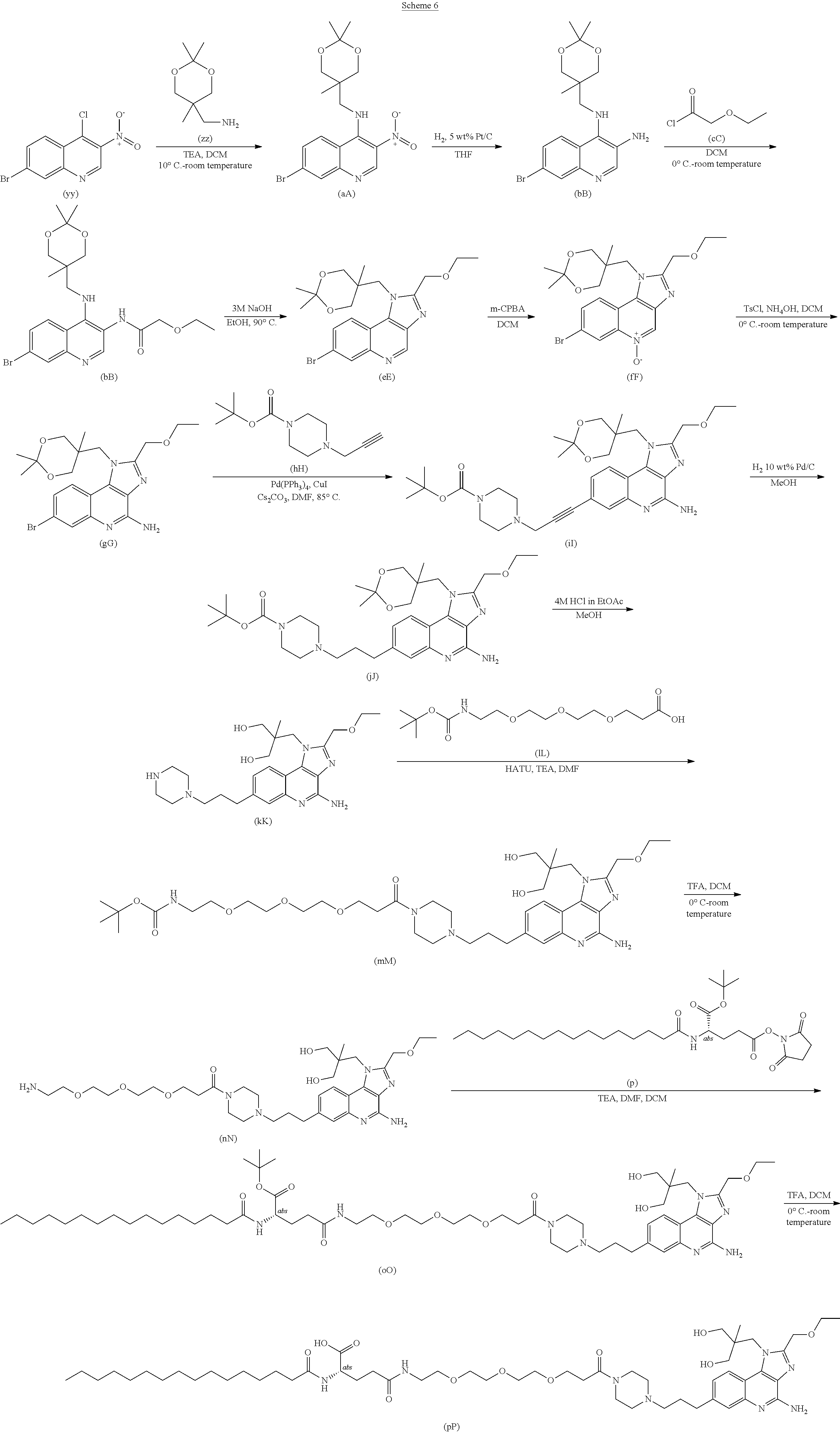

The halogen (e.g. Cl, Br)-substituted heterocycle (yy) can react with the amine (zz) in a solvent such as DCM and with a base such as TEA from 10° C. to room temperature to afford the nitro-intermediate (aA). The nitro-intermediate (aA) can then be reduced through a hydrogenation with a catalyst such as platinum on carbon (Pt/C) under H₂ gas in a solvent like THF with stirring from 24-48 hours to afford the aniline (bB). The aniline (bB) can react with acid chloride (cC) in a solvent like DCM at 0° C.—room temperature to afford the amide (dD). The amide (dD) can cyclize under basic conditions like sodium hydroxide (NaOH) in a suitable solvent such as EtOH heated from 80-110° C. to provide the tricycle (eE). The tricycle (eE) in a solvent such as DCM can oxidize with the treatment of m-CPBA then with stirring from 16-24 hours to provide the N-oxide (fF). The N-oxide (fF) in a solvent like DCM with a base such as NH₄OH can react with TsCl from 0° C. to room temperature with stirring from 16-24 hours to afford the aniline (gG). The Br-substituted tricycle (gG) can react with the acetylene (hH) under standard cross-coupling conditions like with a catalyst such as tetrakis (triphenylphosphine) palladium (0) (Pd (PPh3) 4) and CuI with a base like Cs₂CO₃ in a solvent like DMF under argon gas while heating from 80-100° C. with stirring from 16-24 hours to afford the acetylene intermediate (il). The acetylene intermediate (il) can be reduced through standard hydrogenation conditions like with a catalyst such as palladium on carbon (Pd/C) under H₂ gas in a solvent like MeOH with stirring from 16-24 hours to afford the protected amine (jJ) which can undergo deprotection under acidic conditions such as with HCl in EtOAc with additional solvents like DCM and MeOH to form the secondary amine (kK). The secondary amine (kK) can react with the acid (lL) under standard amide coupling conditions like HATU with a base like TEA in a solvent like DMF while stirring from 16-24 hours to form the protected amine (mM) which can then be deprotected under standard acidic conditions like TFA in a solvent like DCM from 0° C.—room temperature to provide the amine (nN). The amine (nN) can react with the NHS-ester (p) in a solvents like DCM and DMF with a base like TEA and stirring from 16-24 hours to afford the protected acid intermediate (oO) which can be deprotected under standard acidic conditions like TFA in a solvent like DCM from 0° C. to room temperature to provide the target product (pP).

### EXAMPLES

In order that this disclosure may be better understood, the following examples are set forth. These examples are for purposes of illustration only and are not to be construed as limiting the scope of the disclosure in any manner.

Unless noted otherwise (below or in the schemes or preparations above), all reactants were obtained commercially without further purifications or were prepared using methods known in the literature.

### Example 1

### N⁵-(4-(N-(3-(4-Amino-2-butyl-1H-imidazo[4,5-c]quinolin-1-yl)-2,2-bis(hydroxymethyl)propyl)sulfamoyl)butyl)-N²-palmitoyl-L-glutamine (1)

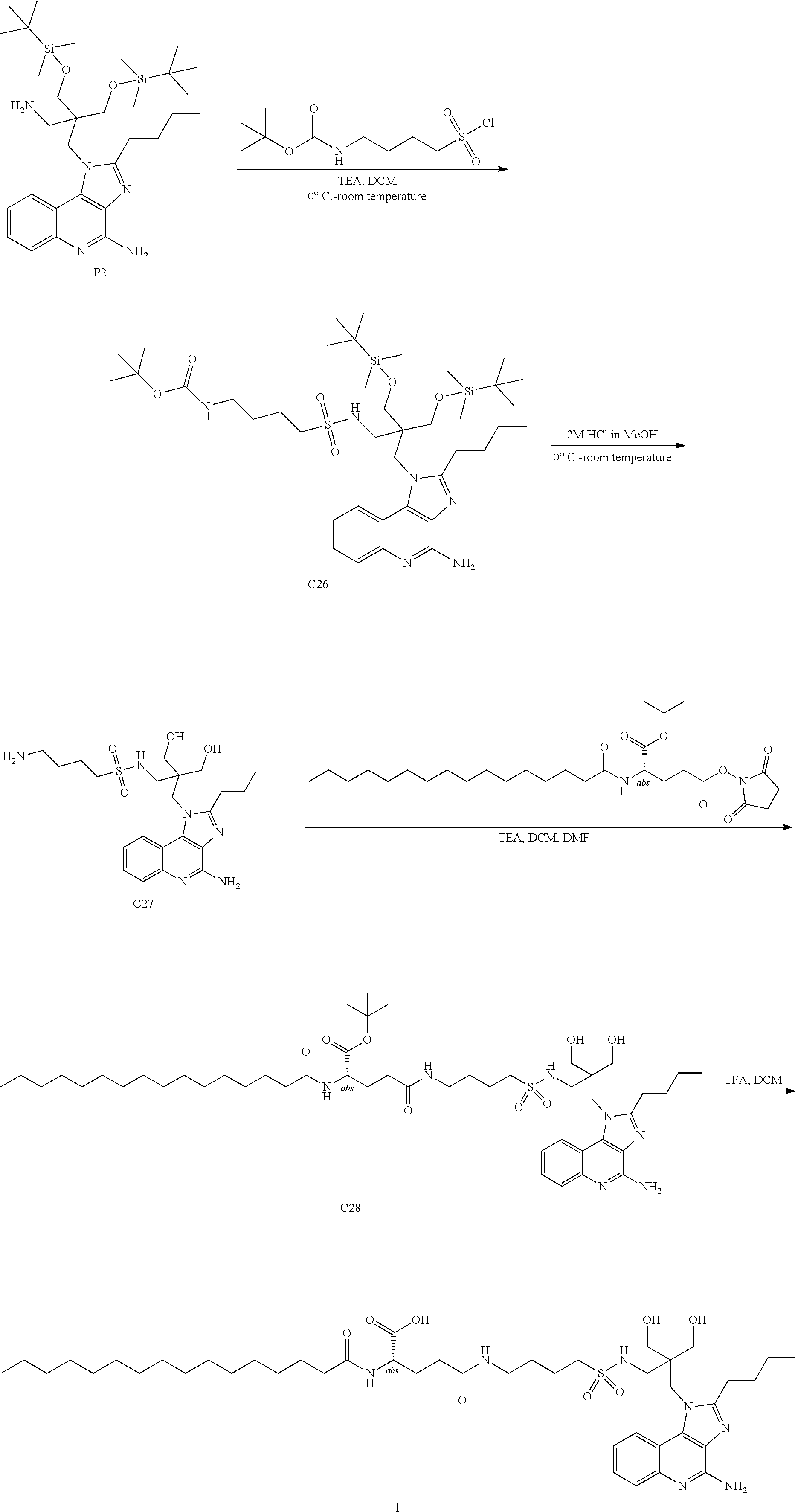

### Step 1. Preparation of tert-butyl (4-(N-(3-(4-amino-2-butyl-1H-imidazo[4,5-c]quinolin-1-yl)-2,2-bis(((tert-butyldimethylsilyl)oxy)methyl)propyl)sulfamoyl)butyl)carbamate (C26)

At 0° C. under N₂ gas, to a stirred solution of P2 (14 g, 24 mmol) in DCM (140 mL) was added TEA (9.5 g, 94 mmol) then tert-butyl (4-(chlorosulfonyl)butyl)carbamate (CAS: 2167808-49-1; 7.0 g, 26 mmol) successively. The reaction mixture was warmed to room temperature then stirred for 2 hours before H₂O (100 mL) was added. The diluted reaction mixture was extracted with DCM (200 mL×2) then the combined organic layer was washed with brine (50 mL×2), dried over Na₂SO₄ and concentrated in vacuo. The yellow gum was purified by column chromatography (silica gel, MeOH:DCM, 0-15% MeOH) to provide an impure batch of C26 (16 g) as a yellow solid. The impure batch of C26 (16 g) was repurified by column chromatography (silica gel, MeOH:EtOAc, 0-15% MeOH) to provide C26 (13 g, 67% yield) as a light-yellow solid. (LCMS) (M)⁺ 821.5.

### Step 2. Preparation of 4-amino-N-(3-(4-amino-2-butyl-1H-imidazo[4,5-c]quinolin-1-yl)-2,2-bis(hydroxymethyl)propyl) butane-1-sulfonamide hydrochloride (C27)

At 0° C., to a stirred solution of C26 (36.7 g, 44.7 mmol) in MeOH (50 mL) was added 2M HCl in MeOH (500 mL). The reaction mixture was warmed to room temperature and stirred for 3 hours then concentrated in vacuo to give a yellow gum. The yellow gum was lyophilized to provide C27 (50.4 g, >99% yield) as a light-yellow solid. The solid was used directly in the next step without any additional purification. (LCMS) (M+H)⁺ 493.2. ¹H NMR (400 MHz, (CD₃)₂SO) δ 14.11 (br s, 1H), 8.86-8.53 (m, 2H), 8.07 (br s, 2H), 7.81 (d, 1H), 7.70 (t, 1H), 7.49 (t, 1H), 7.43 (t, 1H), 4.97-4.87 (m, 1H), 4.66-4.53 (m, 1H), 3.52-3.32 (m, 3H), 3.21-2.89 (m, 9H), 2.81-2.71 (m, 2H), 1.87-1.61 (m, 6H), 1.49-1.37 (m, 2H), 0.94 (t, 3H).

### Step 3. Preparation of tert-butyl N⁵-(4-(N-(3-(4-amino-2-butyl-1H-imidazo[4,5-c]quinolin-1-yl)-2,2-bis(hydroxymethyl)propyl)sulfamoyl)butyl)-N²-palmitoyl-L-glutaminate (C28)

Under N2 gas, to a stirred solution of C27 (2.52 g, 4.46 mmol) in DCM (30 mL) and DMF (10 mL) was added TEA (1.35 g, 13.4 mmol) and 1-tert-butyl 5-(N-succinimidyl)N-palmitoyl-L-glutamate (CAS: 204521-63-1; 2.40 g, 4.45 mmol). The reaction mixture was stirred at room temperature for 2 hours before H₂O (50 mL) was added and then extracted with DCM (50 mL×2). The combined organic layer was washed with brine (50 mL), dried with Na₂SO₄, filtered, and concentrated in vacuo. The light-yellow gum was purified by column chromatography (silica gel, MeOH:DCM, 0-20% MeOH) to provide C₂₈ (3.50 g, 85.7% yield) as a light-yellow solid. (LCMS) (M)⁺ 916.8. ¹H NMR (400 MHz, (CD3) 2SO) δ 8.64 (d, 1H), 8.07 (d, 1H), 7.85 (t, 1H), 7.66 (d, 1H), 7.33-7.15 (m, 3H), 7.24 (br s, 1H), 7.09 (t, 1H), 4.99-4.84 (m, 2H), 4.64-4.47 (m, 2H), 4.14-3.98 (m, 1H), 3.57-3.48 (m, 1H), 3.18-3.07 (m, 3H), 3.03-2.90 (m, 6H), 2.17-2.06 (dt, 4H), 1.94-1.84 (m, 1H), 1.82-1.69 (m, 3H), 1.63-1.52 (m, 2H), 1.51-1.40 (m, 6H), 1.39 (br s, 9H), 1.22 (br s, 26H), 0.94 (t, 3H), 0.84 (t, 3H).

### Step 4. Preparation of N⁵-(4-(N-(3-(4-Amino-2-butyl-1H-imidazo[4,5-c]quinolin-1-yl)-2,2-bis(hydroxymethyl)propyl)sulfamoyl)butyl)-N²_palmitoyl-L-glutamine (1)

To a stirred solution of C28 (3.50 g, 3.82 mmol) in DCM (15 mL) was added TFA (15 mL) dropwise at room temperature then stirred for 2 hours. The reaction mixture was dissolved in (1:2) H₂O:THF (30 mL) then LiOH (962 mg, 22.9 mmol) was added to adjust the pH˜8. The reaction was stirred for 1 hour at room temperature then purified by reverse phase HPLC (Welch Xtimate C₁₈ 150 mm×25 mm×5 μm, water (0.05% NH₄HCO₃)/MeCN, 35 to 75% over 11 minutes, 100% MeCN hold time for 3 minutes, flow rate was 60 (mL/min)) and lyophilized to provide 1 (1.75 g, 53.3% yield) as a white solid. LCMS m/z (M)⁺=860.4. ¹H NMR (400 MHz, (CD₃)₂SO) δ 8.60 (d, 1H), 7.95-7.84 (m, 1H), 7.82-7.74 (m, 1H), 7.65 (d, 1H), 7.45 (t, 2H), 7.26 (t, 2H), 7.07-6.97 (m, 1H), 4.95-4.84 (m, 1H), 4.60-4.48 (m, 1H), 4.16-4.07 (m, 1H), 3.60-3.30 (m, 4H), 3.22-3.08 (m, 2H), 3.03-2.66 (m, 8H), 2.16-2.06 (m, 4H), 2.01-1.90 (m, 1H), 1.83-1.70 (m, 3H), 1.54-1.33 (m, 8H), 1.21 (br s, 24H), 0.94 (t, 3H), 0.84 (t, 3H).

### Example 2

### (S)-1-(N-(3-(4-Amino-2-butyl-1H-imidazo[4,5-c]quinolin-1-yl)-2,2-bis(hydroxymethyl)propyl)sulfamoyl)-13-oxo-16-palmitamido-3,6,9-trioxa-12-azaheptadecan-17-oic Acid (2)

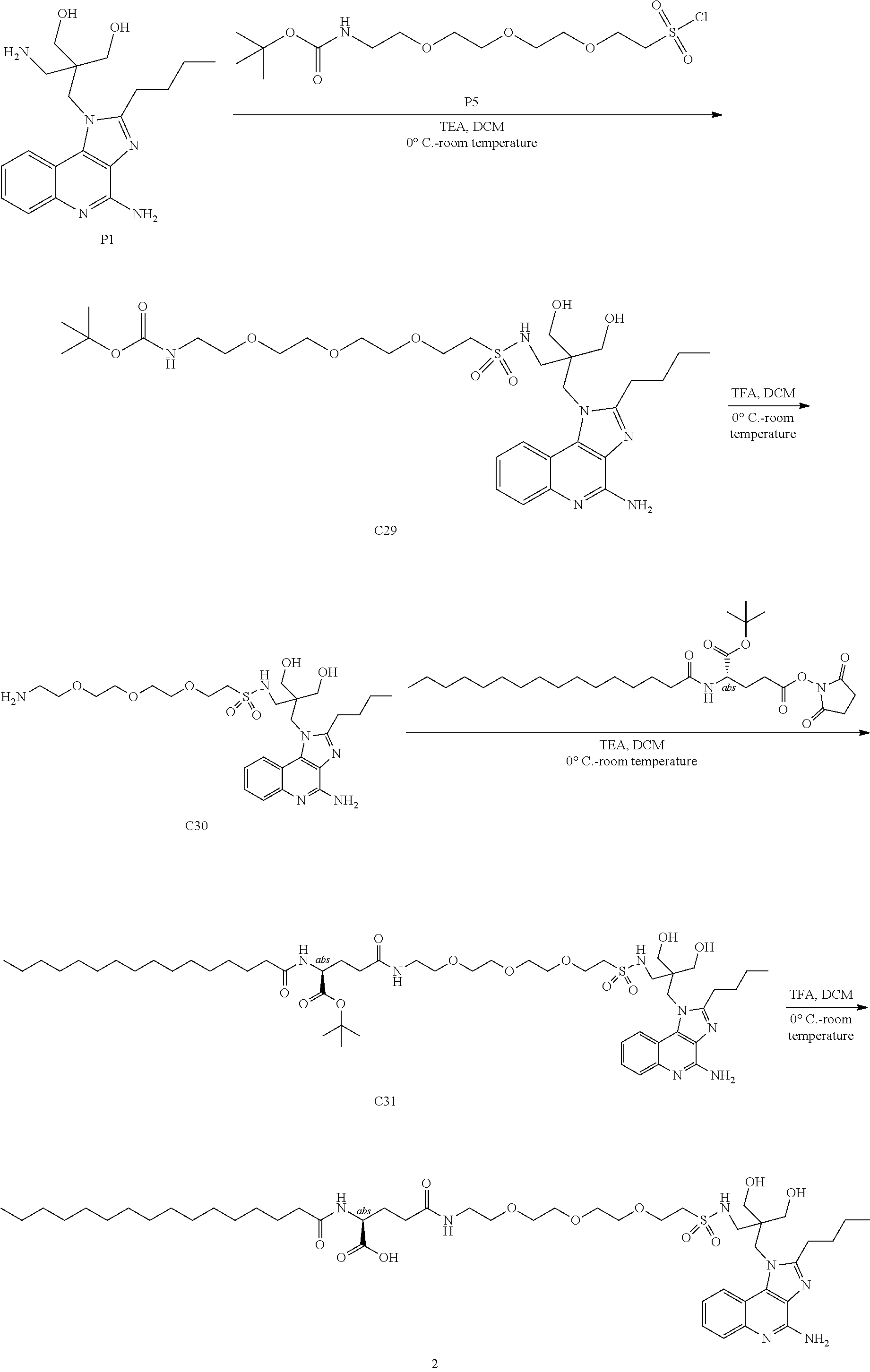

### Step 1. Preparation of tert-butyl(2-(2-(2-(2-(N-(3-(4-amino-2-butyl-1H-imidazo[4,5-c]quinolin-1-yl)-2,2-bis(hydroxymethyl)propyl)sulfamoyl)ethoxy)ethoxy)ethoxy)ethyl)carbamate (C29)

The reaction was conducted in two batches then combined for purification. At 0-5° C. under N₂ gas, to a stirred solution of P1 (91.6 mg, 0.213 mmol) in DCM (10 mL) was added TEA (64.6 mg, 0.639 mmol) and P5 (0.160 g, 0.426 mmol) to form the first batch. The reaction mixture of the first batch was warmed to room temperature then stirred for 16 hours.

A second batch of the same reaction was conducted with P1 (22.9 mg, 0.0532 mmol). The 2 batches were combined then an additional portion of TEA (64.6 mg, 0.639 mmol) and P5 (0.120 g, 0.319 mmol) were added. The combined reaction mixture was stirred at room temperature for 16 hours was extracted with EtOAc (20 mL×3). The combined organic layer was washed with brine (20 mL×3), dried with Na₂SO₄, filtered, and concentrated in vacuo to provide C29 (95.0 mg, 51.2% yield) as a yellow gum. The gum was used directly in the next step without further purification. LCMS m/z (M+H)⁺=697.4.

### Step 2. Preparation of N-(3-(4-amino-2-butyl-1H-imidazo[4,5-c]quinolin-1-yl)-2,2-bis(hydroxymethyl)propyl)-2-(2-(2-(2-aminoethoxy)ethoxy)ethoxy)ethane-1-sulfonamide trifluoroacetate (C30)

At 0-5° C. under N₂ gas, to a stirred solution of C₂₉ (95.0 mg, 0.14 mmol) in DCM (2 mL) was added TFA (1 mL) dropwise. The reaction mixture was stirred at room temperature for 1 hour then concentrated in vacuo and lyophilized to provide C30 (0.100 g, >99% yield) as a yellow gum. The gum was used directly in the next step without further purification. LCMS m/z (M+H)⁺=597.4.

### Step 3. Preparation of tert-butyl (S)-1-(N-(3-(4-amino-2-butyl-1H-imidazo[4,5-c]quinolin-1-yl)-2,2-bis(hydroxymethyl)propyl)sulfamoyl)-13-oxo-16-palmitamido-3,6,9-trioxa-12-azaheptadecan-17-oate (C31)

At 0-5° C. under N₂ gas, to a stirred solution of C30 (0.100 g, 0.168 mmol) and 1-(tert-butyl) 5-(2,5-dioxopyrrolidin-1-yl) palmitoyl-L-glutamate (CAS: 204521-63-1; 90.3 mg, 0.168 mmol) in DCM (5 mL) was added TEA (50.9 mg, 0.503 mmol). The reaction mixture was stirred at room temperature for 2 hours then was extracted with the DCM (15 mL×3). The combined organic layer was washed with brine (20 mL×3), dried with Na₂SO₄, filtered, and concentrated in vacuo to provide C31 (0.180 g, >99% yield) as yellow gum. The gum was used directly in the next step without further purification.

### Step 4. Preparation of(S)-1-(N-(3-(4-amino-2-butyl-1H-imidazo[4,5-c]quinolin-1-yl)-2,2-bis(hydroxymethyl)propyl)sulfamoyl)-13-oxo-16-palmitamido-3,6,9-trioxa-12-azaheptadecan-17-oic Acid (2)

At 0-5° C. under N₂ gas, to a stirred solution of C₃₁ (0.180 g, 0.176 mmol) in DCM (5 mL) was added TFA (2 mL) dropwise. The reaction was warmed to room temperature and stirred for 3 hours then concentrated in vacuo. The yellow gum was purified by reverse phase HPLC (C18 150 mm×30 mm×5 μm, water (0.05% formic acid)/MeCN, 41 to 81% over 9 minutes, 100% MeCN hold time for 2 minutes, flow rate was 30 (mL/min)) and lyophilized to provide 2 (14.3 mg, 8.45% yield) as a white solid. LCMS m/z (M+H)⁺=965.7. ¹H NMR (400 MHz, (CD3) 2SO) δ 8.62 (d, 1H), 8.16 (s, 1H), 7.95-7.94 (m, 1H), 7.65 (d, 1H), 7.47 (t, 1H), 7.28 (t, 1H), 4.95-4.85 (m, 1H), 4.60-4.50 (m, 1H), 4.15-4.07 (m, 1H), 3.49 (s, 14H), 3.38 (t, 4H), 3.22-3.09 (m, 4H), 3.02-2.88 (m, 2H), 2.15-2.06 (m, 4H), 1.99-1.89 (m, 1H), 1.82-1.70 (m, 3H), 1.49-1.37 (m, 4H), 1.21 (d, 26H), 0.93 (t, 3H), 0.83 (t, 3H).

### Example 3

### (S)-1-(4-Amino-2-butyl-1H-imidazo[4,5-c]quinolin-1-yl)-2,2-bis(hydroxymethyl)-18-oxo-21-palmitamido-8,11,14-trioxa-4,17-diazadocosan-22-oic Acid (3)

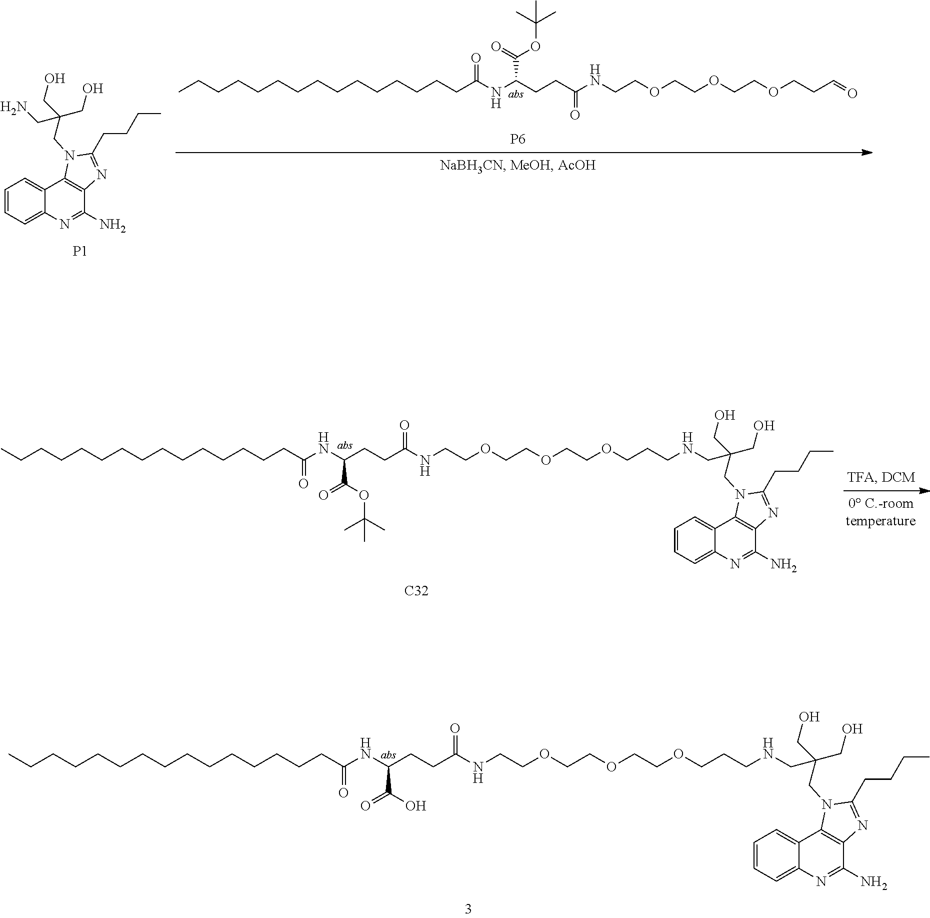

### Step 1. Preparation of tert-butyl (S)-1-(4-amino-2-butyl-1H-imidazo[4,5-c]quinolin-1-yl)-2,2-bis(hydroxymethyl)-18-oxo-21-palmitamido-8,11,14-trioxa-4,17-diazadocosan-22-oate (C32)

To a stirred solution of P6 (93.8 mg, 0.119 mmol), 4 Å molecular sieves (CAS: 70955-01-0; 60.0 mg) and P1 (64.2 mg, 0.119 mmol) in MeOH (2.0 mL) was added NaBH3CN (37.5 mg, 0.596 mmol) which caused bubbles to form. The reaction mixture was stirred at room temperature for 2 hours before AcOH (0.1 mL) was added dropwise. After the addition, the reaction mixture was stirred for an additional 2 hours before another portion of P6 (0.107 g, 0.170 mmol) was added. The suspension was stirred for 15 minutes at room temperature before another portion of NaBH3CN (30.0 mg, 0.477 mmol) was added. The reaction mixture was stirred for 2 hours at room temperature then poured into H₂O (15.0 mL) and NaHCO₃ (10.0 mL) was added. The suspension was extracted with DCM (15.0 mL×3). The combined organic layer was dried over Na₂SO₄, filtered, and concentrated in vacuo. The yellow gum was purified by prep-TLC ((10:1:0.1) DCM:MeOH:NH₄OH, Rf˜0.3, UV 254 nm) to provide C₃₂ (50.0 mg, 43.3% yield) as a colorless solid. LCMS m/z (M)⁺=970.7.

### Step 2. Preparation of(S)-1-(4-amino-2-butyl-1H-imidazo[4,5-c]quinolin-1-yl)-2,2-bis(hydroxymethyl)-18-oxo-21-palmitamido-8,11,14-trioxa-4,17-diazadocosan-22-oic Acid (3)

At 0-5° C. under N₂ gas, to a stirred solution of C₃₂ (50.0 mg, 0.0515 mmol) in DCM (3 mL) was added TFA (1 mL) dropwise. The reaction mixture was warmed to room temperature and stirred for 2 hours then concentrated in vacuo. The yellow gum was purified by reverse phase HPLC (Boston Prime C₁₈ 150 mm×30 mm×5 μm, water (0.05% NH₄OH)/MeCN, 60 to 80% over 11 minutes, 100% MeCN hold time for 2 minutes, flow rate was 35 (mL/min)) to provide 3 (14.5 mg, 30.7% yield) as a white solid. LCMS m/z (M)⁺=914.6. ¹H NMR (400 MHz, CDCl₃) δ 8.42-8.34 (m, 1H), 7.79-7.72 (m, 1H), 7.68-7.61 (m, 1H), 7.51-7.43 (m, 1H), 7.38 (t, 1H), 6.79-6.70 (m, 1H), 4.97-4.84 (m, 1H), 4.56-4.41 (m, 1H), 4.34-4.24 (m, 1H), 3.99-3.89 (m, 1H), 3.80-3.67 (m, 1H), 3.52 (s, 13H), 3.44-3.29 (m, 3H), 3.20-3.00 (m, 3H), 2.89-2.69 (m, 3H), 2.39-2.11 (m, 6H), 2.03-1.92 (m, 1H), 1.85-1.76 (m, 3H), 1.67-1.59 (m, 2H), 1.50-1.40 (m, 2H), 1.32-1.22 (m, 26H), 0.96 (t, 3H), 0.87 (t, 3H).

### Example 4

### (S)-5-(4-(2-((4-((3-(4-Amino-2-butyl-1H-imidazo[4,5-c]quinolin-1-yl)-2,2-bis(hydroxymethyl)propyl)amino)butyl)amino)-2-oxoethyl)-4-hydroxypiperidin-1-yl)-5-oxo-2-palmitamidopentanoic Acid (4)

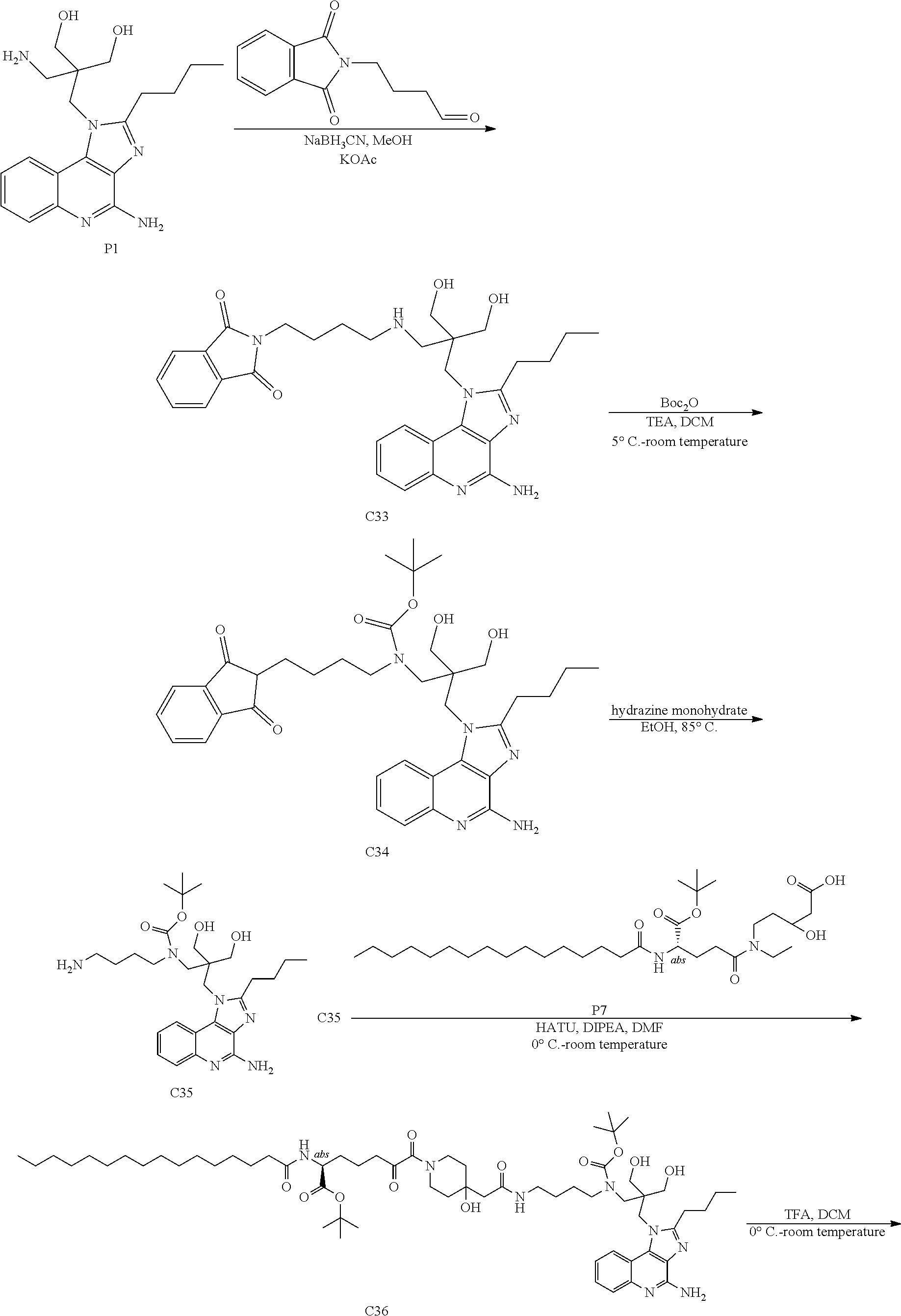

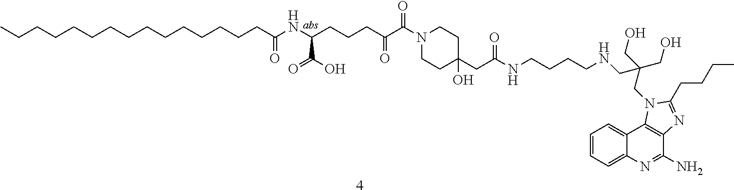 [SMILES: CCCCCCCCCCCCCCCC(=O)N[C@@H](CCCC(=O)C(=O)N1CCC(O)(CC(=O)NCCCCNCC(CO)(CO)Cn2c(CCCC)nc3c(N)nc4ccccc4c32)CC1)C(=O)O ; "4"]

### Step 1. Preparation of 2-(4-((3-(4-amino-2-butyl-1H-imidazo[4,5-c]quinolin-1-yl)-2,2-bis(hydroxymethyl)propyl)amino)butyl)isoindoline-1,3-dione (C33)

A solution of P1 (0.200 g, 0.465 mmol), 4-(1,3-dioxoisoindolin-2-yl) butanal (CAS: 3598-60-5; 0.106 g, 0.486 mmol) and KOAc (95.4 mg, 0.972 mmol) in MeOH (8 mL) was stirred for 30 minutes at room temperature. To the reaction mixture was added NaBH3CN (41.7 mg, 0.663 mmol). The suspension was stirred at room temperature for 18 hours before the reaction mixture was quenched with ice (3 mL). The quenched reaction mixture was adjusted to pH=9 with the aqueous Na₂CO₃ then extracted with (4:1) CHCl₃:IPA (10 mL×5). The combined organic layer was dried over Na₂SO₄ and concentrated in vacuo to provide C₃₃ (260 mg, >99% yield) as yellow gum. The gum was used directly in the next step without additional purification. LCMS m/z (M+H)⁺=559.4.

### Step 2. Preparation of tert-butyl (3-(4-amino-2-butyl-1H-imidazo[4,5-c]quinolin-1-yl)-2,2-bis(hydroxymethyl)propyl)(4-(1,3-dioxoisoindolin-2-yl)butyl)carbamate (C34)

At 5-10° C., to a solution of C₃₃ (0.260 g, 0.465 mmol) and TEA (0.141 g, 1.40 mmol) in DCM (8 mL) was added Boc2O (0.152 g, 0.698 mmol). The reaction mixture was warmed to room temperature and stirred for 18 hours then poured into H₂O (10 mL). The quenched reaction mixture was extracted with DCM (10 mL×3). The combined organic layer was dried with Na2SO4 and concentrated in vacuo. The brown gum was purified by column chromatography (silica gel, MeOH:DCM, 0-10% MeOH) to provide C34 (166 mg, 54.1% yield) as yellow gum. LCMS m/z (M+H)⁺=659.4. ¹H NMR (400 MHz, (CD₃)₂SO) δ 8.41-8.31 (m, 1H), 7.92-7.83 (m, 4H), 7.52 (d, 1H), 7.32-7.24 (m, 1H), 7.18-7.10 (m, 1H), 6.52 (br s, 2H), 5.24 (s, 1H), 4.91-4.69 (m, 2H), 4.65-4.51 (m, 1H), 3.57-3.47 (m, 2H), 3.46-3.36 (m, 4H), 3.25-3.05 (m, 4H), 3.06-2.78 (m, 2H), 1.85-1.65 (m, 2H), 1.51-1.34 (m, 6H), 1.28 (br s, 9H), 0.94 (t, 3H).

### Step 3. Preparation of tert-butyl (3-(4-amino-2-butyl-1H-imidazo[4,5-c]quinolin-1-yl)-2,2-bis(hydroxymethyl)propyl)(4-aminobutyl)carbamate (C35)

Under N₂ gas, to a stirred solution of C34 (0.166 g, 0.252 mmol) in EtOH (8 mL) was added hydrazine monohydrate (0.260 g, 5.19 mmol) at room temperature. The reaction mixture was heated to 85° C. and stirred for 16 hours before filtration. The filter cake was washed with EtOH (15 mL) then the filtrate was concentrated in vacuo. The residue was suspended in DCM (20 mL) and filtered again. The filtrate was dried with Na₂SO₄ then concentrated in vacuo and lyophilized to provide C35 (131 mg, 98.3% yield) as white solid. The solid was used directly in the next step without further purification. LCMS m/z (M+H)⁺=529.5.

### Step 4. Preparation of tert-butyl (S)-5-(4-(2-((4-((3-(4-amino-2-butyl-1H-imidazo[4,5-c]quinolin-1-yl)-2,2-bis(hydroxymethyl)propyl) (tert-butoxycarbonyl)amino)butyl)amino)-2-oxoethyl)-4-hydroxypiperidin-1-yl)-5-oxo-2-palmitamidopentanoate (C36)

At 0° C. under N₂ gas, to a stirred solution of C35 (0.131 g, 0.248 mmol) and P7 (0.144 g, 0.248 mmol) in DMF (4 mL) was added HATU (98.9 mg, 0.260 mmol) then DIPEA (0.192 g, 1.49 mmol) dropwise. The reaction mixture was stirred for 16 hours at room temperature then poured into H₂O (10 mL). The diluted reaction mixture was extracted with EtOAc (10 mL×3) then the combined organic layer was dried with Na₂SO₄ and concentrated in vacuo. The yellow gum was purified by column chromatography (silica gel, ((1:10) NH₄OH: MeOH) in DCM, 0-10% ((1:10) NH₄OH: MeOH)) to provide C36 (205 mg, 74.7% yield) as white solid.

### Step 5. Preparation of(S)-5-(4-(2-((4-((3-(4-amino-2-butyl-1H-imidazo[4,5-c]quinolin-1-yl)-2,2-bis(hydroxymethyl)propyl)amino)butyl)amino)-2-oxoethyl)-4-hydroxypiperidin-1-yl)-5-oxo-2-palmitamidopentanoic Acid (4)

At 0-5° C. under N₂ gas, to a stirred solution of C36 (0.205 g, 0.185 mmol) in DCM (4 mL) was added TFA (5 mL) dropwise. The reaction mixture was stirred at room temperature for 4 hours then concentrated in vacuo. The yellow gum was purified by reverse phase HPLC (Boston Prime C₁₈ 150 mm×30 mm×5 μm, water (0.05% NH₄OH+NH₄HCO₃)/MeCN, 26 to 56% over 8 minutes, 100% MeCN hold time for 3 minutes, flow rate was 30 (mL/min)) then lyophilized to provide 4 (38.5 mg, 14.9% yield) as a light-yellow glass. LCMS m/z (M+H)⁺=938.6. ¹H NMR (400 MHz, (CD₃)₂SO) δ 8.70-8.61 (m, 1H), 7.97-7.88 (m, 1H), 7.84-7.74 (m, 1H), 7.63 (d, 1H), 7.42 (t, 1H), 7.23 (d, 1H), 7.15-6.89 (m, 2H), 5.02-4.86 (m, 2H), 4.59-4.47 (m, 1H), 4.17-4.08 (m, 1H), 4.03-3.95 (m, 1H), 3.64-3.23 (m, 14H), 3.14-2.81 (m, 7H), 2.28-2.22 (m, 1H), 2.18 (s, 1H), 2.13-2.06 (m, 2H), 1.93-1.86 (m, 1H), 1.81-1.73 (m, 3H), 1.50-1.31 (m, 11H), 1.22 (s, 24H), 0.94 (t, 3H), 0.84 (t, 3H).

### Example 5

### 1-(4-(2-(4-(3-(4-Amino-2-butyl-1-(3-hydroxy-2-(hydroxymethyl)-2-methylpropyl)-1H-imidazo[4,5-c]quinolin-7-yl)propyl) piperazin-1-yl)-2-oxoethyl)-4-hydroxypiperidin-1-yl)hexadecan-1-one (5)

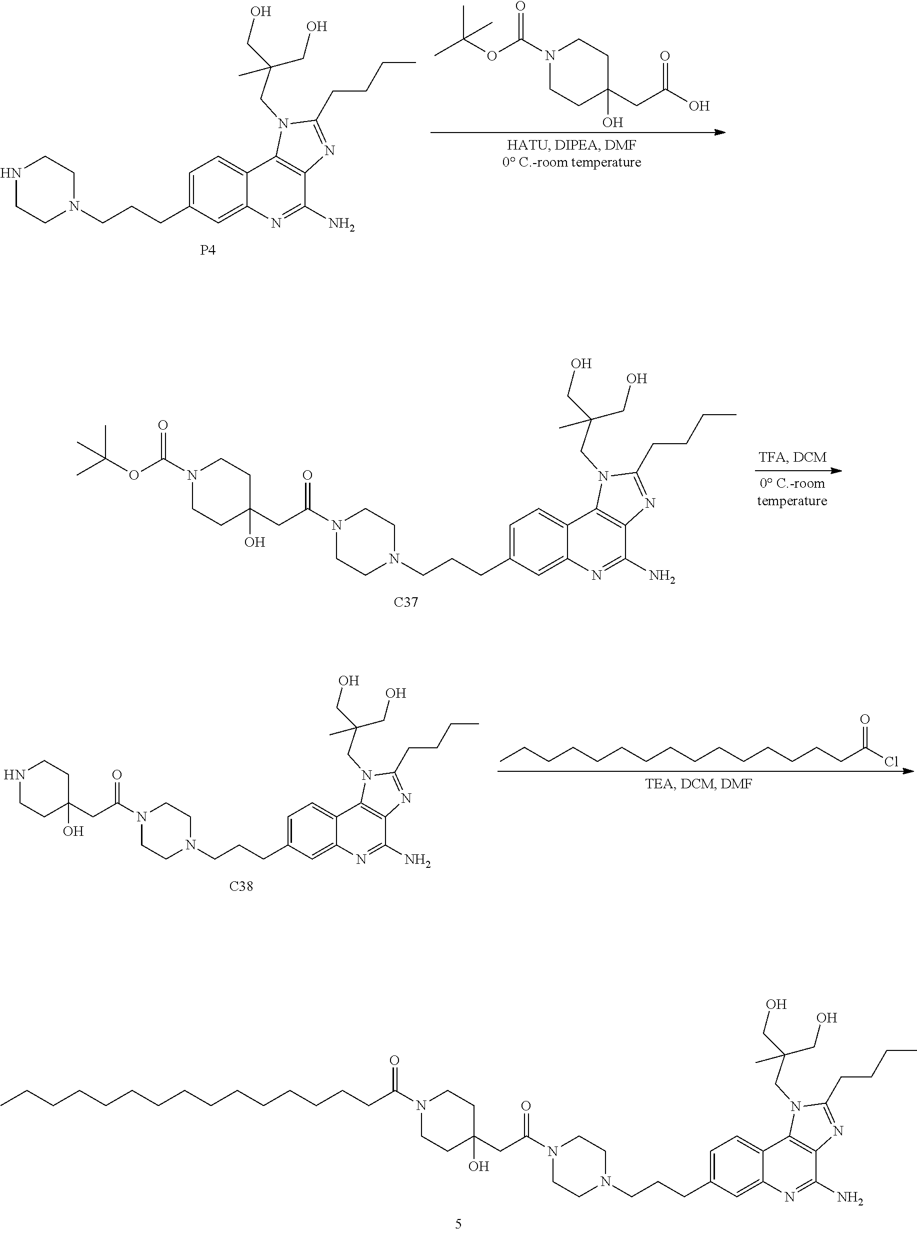

### Step 1. Preparation of tert-butyl 4-(2-(4-(3-(4-amino-2-butyl-1-(3-hydroxy-2-(hydroxymethyl)-2-methylpropyl)-1H-imidazo[4,5-c]quinolin-7-yl)propyl) piperazin-1-yl)-2-oxoethyl)-4-hydroxypiperidine-1-carboxylate (C37)

At 0° C. under N₂ gas, to a stirred solution of 2-(1-(tert-butoxycarbonyl)-4-hydroxypiperidin-4-yl)acetic acid (CAS: 502482-52-2; 37.7 mg, 0.145 mmol) in DMF (3 mL) was added HATU (57.9 mg, 0.152 mmol) then DIPEA (89.4 mg, 0.692 mmol) successively. The reaction mixture was stirred for 10 minutes then P4 (80.0 mg, 0.138 mmol) was added. The resulting reaction mixture was stirred at room temperature for 16 hours. The yellow suspension was concentrated in vacuo then filtered through a pad of silica gel. The silica pad was rinsed with EtOAc (˜100 mL) then (1:10) MeOH:DCM. The filtrate was combined then discarded. The silica pad was then rinsed with (10:100:1) MeOH:DCM:NH₄OH (˜200 mL) then the filtrate was concentrated in vacuo to provide C37 (142 mg, >99% yield) as yellow gum. The gum was used directly in the next step without further purification. LCMS m/z (M+H)⁺=710.5. ¹H NMR (400 MHz, CDCl₃) δ 8.52 (dd, 1H), 8.34 (d, 1H), 8.19 (d, 1H), 7.79 (s, 1H), 7.24-7.17 (m, 2H), 5.24-5.07 (m, 1H), 4.91-4.57 (m, 1H), 3.81 (br s, 2H), 3.73-3.56 (m, 7H), 3.51-3.45 (m, 2H), 3.26-3.14 (m, 1H), 3.13-3.03 (m, 4H), 2.72 (t, 2H), 2.47-2.30 (m, 8H), 1.87-1.66 (m, 6H), 1.52 (t, 4H), 1.45 (s, 9H), 0.96 (t, 3H), 0.64 (s, 3H).

### Step 2. Preparation of 1-(4-(3-(4-amino-2-butyl-1-(3-hydroxy-2-(hydroxymethyl)-2-methylpropyl)-1H-imidazo[4,5-c]quinolin-7-yl)propyl) piperazin-1-yl)-2-(4-hydroxypiperidin-4-yl)ethan-1-one trifluoroacetate (C38)

At 0° C. under N₂ gas, to a stirred solution of C37 (98.0 mg, 0.138 mmol) in DCM (1.5 mL) was added TFA (0.5 mL). The reaction mixture was stirred at room temperature for 1 hour then concentrated in vacuo and lyophilized to provide C38 (0.170 g, >99% yield) as a yellow gum. The gum was used directly in the next step without further purification. LCMS m/z (M+H)⁺=610.8.

### Step 3. Preparation of 1-(4-(2-(4-(3-(4-amino-2-butyl-1-(3-hydroxy-2-(hydroxymethyl)-2-methylpropyl)-1H-imidazo[4,5-c]quinolin-7-yl)propyl) piperazin-1-yl)-2-oxoethyl)-4-hydroxypiperidin-1-yl)hexadecan-1-one (5)

At room temperature under N₂ gas, to a stirred solution of C38 (0.133 g, 0.140 mmol) in DMF (1 mL) was added a solution of TEA (113 mg, 1.12 mmol) in DCM (1 mL) dropwise then a solution of palmitoyl chloride (CAS: 112-67-4; 40.3 mg, 0.147 mmol) in DCM (2 mL). The reaction mixture was stirred at room temperature for 1.5 hours then concentrated in vacuo. The light-yellow solid was purified by reverse phase HPLC (Boston Prime C₁₈ 150 mm×30 mm×5 μm, water (0.05% NH₄OH)/(1:1) MeCN: THF, 50 to 80% over 8 minutes, 100% MeCN hold time for 3 minutes, flow rate was 30 (mL/min)) then lyophilized to provide 5 (17.6 mg, 14.9% yield) as a white solid. LCMS m/z (M)⁺=848.6. ¹H NMR (400 MHz, CDCl₃) δ 8.25 (d, 1H), 7.64-7.62 (m, 1H), 7.16 (dd, 1H), 6.16 (br s, 2H), 5.30-5.16 (m, 1H), 4.93-4.77 (m, 1H), 4.71-4.56 (m, 1H), 4.38 (d, 1H), 3.82-3.43 (m, 9H), 3.09-2.99 (m, 3H), 2.78 (t, 2H), 2.48-2.27 (m, 10H), 1.96-1.72 (m, 9H), 1.65-1.56 (m, 2H), 1.52-1.35 (m, 4H), 1.32-1.21 (m, 24H), 0.98 (t, 3H), 0.88 (t, 3H), 0.64 (s, 3H).

### Example 6

### (S)-1-(4-(3-(4-Amino-2-(ethoxymethyl)-1-(3-hydroxy-2-(hydroxymethyl)-2-methylpropyl)-1H-imidazo[4,5-c]quinolin-7-yl)propyl) piperazin-1-yl)-1, 14-dioxo-17-palmitamido-4,7,10-trioxa-13-azaoctadecan-18-oic Acid (6)

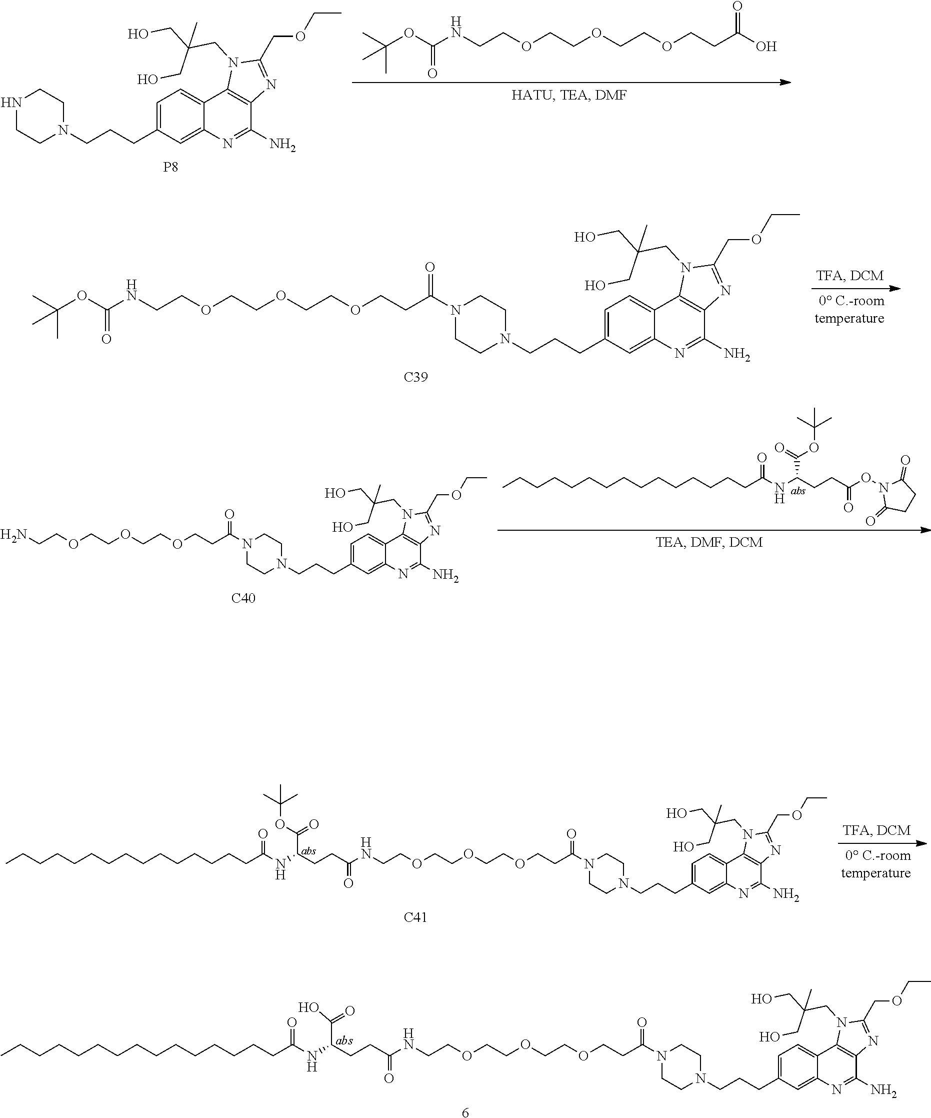

### Step 1. Preparation of tert-butyl(2-(2-(2-(3-(4-(3-(4-amino-2-(ethoxymethyl)-1-(3-hydroxy-2-(hydroxymethyl)-2-methylpropyl)-1H-imidazo[4,5-c]quinolin-7-yl)propyl) piperazin-1-yl)-3-oxopropoxy)ethoxy)ethoxy)ethyl)carbamate (C39)

Under N₂ gas, to a stirred solution of P8 (0.200 g, 0.345 mmol) and 2,2-dimethyl-4-oxo-3,8,11,14-tetraoxa-5-azaheptadecan-17-oic acid (CAS: 1347750-75-7; 111 mg, 0.345 mmol) in DMF (6 mL) was added HATU (144 mg, 0.379 mmol) then DIPEA (334 mg, 2.59 mmol) successively. The reaction mixture was stirred at room temperature for 16 hours then concentrated in vacuo. The yellow gum was purified by column chromatography (silica gel, (10% NH₄OH in MeOH): DCM, 0-15% of (10% NH₄OH in MeOH)) and lyophilized to provide C39 (0.200 g, 75.0% yield) as a yellow solid. LCMS m/z (M+H)⁺=774.4. ¹H NMR (400 MHz, (CD₃)₂SO) δ 8.70-8.67 (m, 1H), 8.51-8.44 (m, 1H), 7.49-7.42 (m, 1H), 7.20-7.07 (m, 2H), 6.77-6.70 (m, 1H), 5.09-4.95 (m, 2H), 4.87-4.57 (m, 4H), 3.62 (t, 2H), 3.56-3.43 (m, 16H), 3.39-3.35 (m, 4H), 3.01-3.08 (m, 2H), 2.75-2.66 (m, 2H), 2.59-2.53 (m, 2H), 2.43-2.30 (m, 6H), 1.87-1.79 (m, 2H), 1.36 (s, 9H), 1.13 (t, 3H), 0.56 (s, 3H).

### Step 2. Preparation of 1-(4-(3-(4-amino-2-(ethoxymethyl)-1-(3-hydroxy-2-(hydroxymethyl)-2-methylpropyl)-1H-imidazo[4,5-c]quinolin-7-yl)propyl) piperazin-1-yl)-3-(2-(2-(2-aminoethoxy)ethoxy)ethoxy)propan-1-one (C40)

At 0-5° C. under N₂ gas, to a stirred solution of C39 (0.200 g, 0.258 mmol) in DCM (3 mL) was added TFA (3 mL) dropwise. The reaction mixture was stirred at room temperature for 1 hour then concentrated in vacuo and lyophilized to provide C40 (0.320 g, >99% yield) as a yellow solid. The solid was used directly in the next step without further purification. LCMS m/z (M+H)⁺=674.4.

### Step 3. Preparation of tert-butyl (S)-1-(4-(3-(4-amino-2-(ethoxymethyl)-1-(3-hydroxy-2-(hydroxymethyl)-2-methylpropyl)-1H-imidazo[4,5-c]quinolin-7-yl)propyl) piperazin-1-yl)-1,14-dioxo-17-palmitamido-4,7,10-trioxa-13-azaoctadecan-18-oate trifluoroacetate (C41)

Under N₂ gas, to a stirred solution of C40 (0.320 g, 0.355 mmol) and 1-(tert-butyl) 5-(2,5-dioxopyrrolidin-1-yl) palmitoyl-L-glutamate (CAS: 204521-63-1; 0.127 g, 0.237 mmol) in DCM (4 mL) was added a solution of TEA (0.168 g, 1.66 mmol) in DCM (2 mL) dropwise. The reaction mixture was stirred at room temperature for 10 minutes before DMF (1 mL) was added. The reaction mixture was stirred for an additional 16 hours then concentrated in vacuo. The yellow gum was purified through a pad of silica gel then washed with EtOAc (50 mL). The filtrate was discarded. The pad of silica gel was then washed with (10:1:0.1) DCM:MeOH:NH₄OH (80 mL; 8.0 mL: 0.8 mL). The filtrate was concentrated in vacuo and lyophilized to provide C₄₁ (0.400 g, >99% yield) as a yellow gum. The gum was used directly in the next step without additional purification. LCMS m/z (M)⁺=1097.8.

### Step 4. Preparation of(S)-1-(4-(3-(4-amino-2-(ethoxymethyl)-1-(3-hydroxy-2-(hydroxymethyl)-2-methylpropyl)-1H-imidazo[4,5-c]quinolin-7-yl)propyl) piperazin-1-yl)-1, 14-dioxo-17-palmitamido-4,7,10-trioxa-13-azaoctadecan-18-oic Acid (6)

At 0-5° C. under N₂ gas, to a stirred solution of C41 (0.400 g, 0.364 mmol) in DCM (6 mL) was added TFA (6 mL) dropwise. The reaction mixture was stirred at room temperature for 1 hour then concentrated in vacuo and lyophilized. The yellow gum was dissolved in (1:1) THF:H₂O (10 mL) then K₂CO₃ (0.126 g, 0.911 mmol) was added to pH=9. The suspension was stirred at room temperature for 2 hours then concentrated in vacuo. The yellow gum was purified by reverse phase HPLC (Boston Prime C₁₈ 150 mm×30 mm×5 μm, water (0.05% NH₄OH)/MeCN, 37 to 58% over 10 minutes, 100% MeCN hold time for 2 minutes, flow rate was 25 (mL/min)) then lyophilized to provide 6 (0.100 g, 26.3% yield) as a white solid. LCMS m/z (M+H)⁺=1042.1. ¹H NMR (400 MHz, (CD3) 2SO) δ 8.47 (d, 1H), 7.90-7.82 (m, 2H), 7.65 (br s, 2H), 7.48-4.67 (m, 1H), 7.18-7.13 (m, 1H), 5.10-4.99 (m, 2H), 4.85-4.74 (m, 1H), 4.69-4.56 (m, 1H), 4.16-4.08 (m, 1H), 3.63-3.58 (m, 4H), 3.49-3.46 (m, 17H), 3.20-3.13 (m, 4H), 2.72 (t, 2H), 2.58-2.53 (m, 4H), 2.40-2.27 (m, 6H), 2.16-2.06 (m, 4H), 2.00-1.90 (m, 1H), 1.86-1.70 (m, 2H), 1.51-1.43 (m, 2H), 1.21 (s, 24H), 1.12 (t, 3H), 0.84 (t, 3H), 0.55 (s, 3H).

The compounds in Table 1 were prepared using general methods or according/analogous to the methods of Schemes 1-6 and Examples 1-6, including modification, as appropriate.

TABLE 1
COMPOUNDS OF EXAMPLES 1-6

| Com- | |
|---|---|
| pound | |
| # | |
| and | |
| Meth- | |
| od | |
| Ex- | |
| ample | Structure/Compound Name |
| 1 | 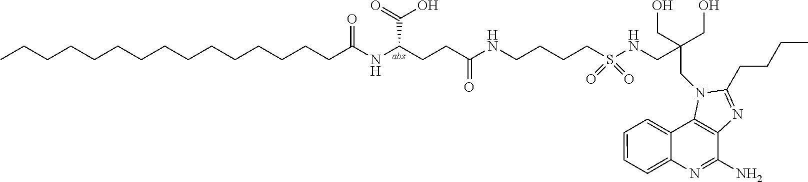 [SMILES: CCCCCCCCCCCCCCCC(=O)N[C@@H](CCC(=O)NCCCCS(=O)(=O)NCC(CO)(CO)Cn1c(CCCC)nc2c(N)nc3ccccc3c21)C(=O)O] |
| | N⁵-(4-(N-(3-(4-Amino-2-butyl-1H-imidazo[4,5-c]quinolin-1-yl)-2,2- |
| | bis(hydroxymethyl)propyl)sulfamoyl)butyl)-N²-palmitoyl-L-glutamine |
| 2 | 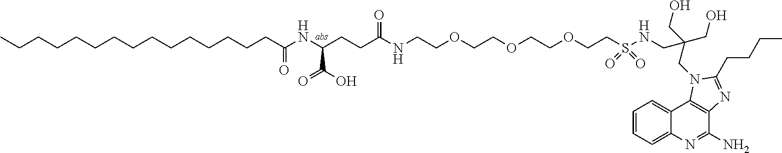 [SMILES: CCCCCCCCCCCCCCCC(=O)N[C@@H](CCC(=O)NCCOCCOCCOCCS(=O)(=O)NCC(CO)(CO)Cn1c(CCCC)nc2c(N)nc3ccccc3c21)C(=O)O] |
| | (S)-1-(N-(3-(4-Amino-2-butyl-1H-imidazo[4,5-c]quinolin-1-yl)-2,2- |
| | bis(hydroxymethyl)propyl)sulfamoyl)-13-oxo-16-palmitamido-3,6,9-trioxa-12- |
| | azaheptadecan-17-oic acid |
| 3 | 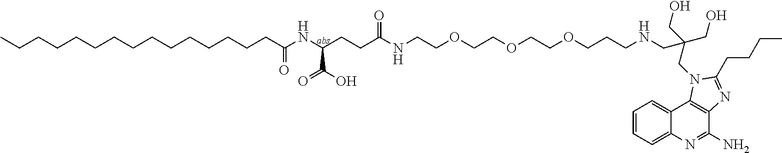 [SMILES: CCCCCCCCCCCCCCCC(=O)N[C@@H](CCC(=O)NCCOCCOCCOCCCNCC(CO)(CO)Cn1c(CCCC)nc2c(N)nc3ccccc3c21)C(=O)O] |
| | (S)-1-(4-Amino-2-butyl-1H-imidazo[4,5-c]quinolin-1-yl)-2,2-bis(hydroxymethyl)-18-oxo-21- |
| | palmitamido-8,11,14-trioxa-4,17-diazadocosan-22-oic acid |
| 4 | 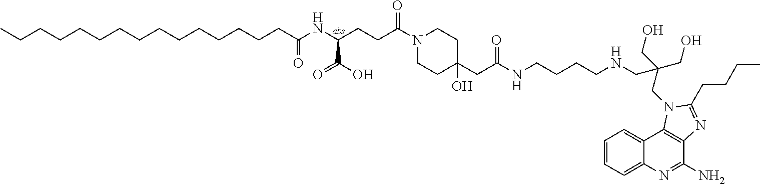 [SMILES: CCCCCCCCCCCCCCCC(=O)N[C@@H](CCC(=O)N1CCC(O)(CC(=O)NCCCCNCC(CO)(CO)Cn2c(CCCC)nc3c(N)nc4ccccc4c32)CC1)C(=O)O] |
| | (S)-5-(4-(2-((4-((3-(4-Amino-2-butyl-1H-imidazo[4,5-c]quinolin-1-yl)-2,2- |
| | bis(hydroxymethyl)propyl)amino)butyl)amino)-2-oxoethyl)-4-hydroxypiperidin-1-yl)-5-oxo- |
| | 2-palmitamidopentanoic acid |
| 5 | 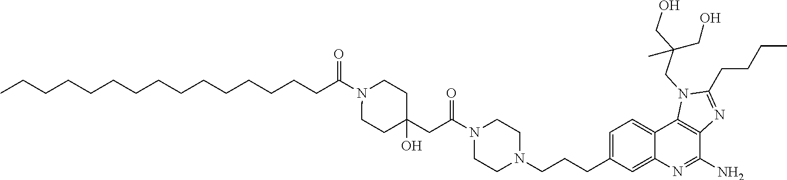 [SMILES: CCCCCCCCCCCCCCCC(=O)N1CCC(O)(CC(=O)N2CCN(CCCc3ccc4c(c3)nc(N)c3nc(CCCC)n(CC(C)(CO)CO)c34)CC2)CC1] |
| | 1-(4-(2-(4-(3-(4-Amino-2-butyl-1-(3-hydroxy-2-(hydroxymethyl)-2-methylpropyl)-1H- |
| | imidazo[4,5-c]quinolin-7-yl)propyl)piperazin-1-yl)-2-oxoethyl)-4-hydroxypiperidin-1- |
| | yl)hexadecan-1-one |
| 6 | 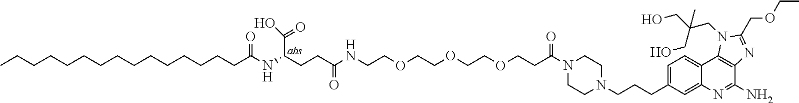 [SMILES: CCCCCCCCCCCCCCCC(=O)N[C@@H](CCC(=O)NCCOCCOCCOCCC(=O)N1CCN(CCCc2ccc3c(c2)nc(N)c2nc(COCC)n(CC(C)(CO)CO)c23)CC1)C(=O)O] |
| | (S)-1-(4-(3-(4-Amino-2-(ethoxymethyl)-1-(3-hydroxy-2-(hydroxymethyl)-2-methylpropyl)- |
| | 1H-imidazo[4,5-c]quinolin-7-yl)propyl)piperazin-1-yl)-1,14-dioxo-17-palmitamido-4,7,10- |
| | trioxa-13-azaoctadecan-18-oic acid |

### Biological Testing

### Example 7

To determine the ability of test compounds to activate the human toll like receptor 7 (hTLR7) or human toll like receptor 8 (hTLR8), cell-based reporter systems were utilized. HEK293 cells stably overexpressing either hTLR7 or hTLR8 along with a reporter gene containing an optimized secreted embryonic alkaline phosphatase gene (SEAP), under the control of the IFN-b minimal promoter fused to five NF-κB and AP-1-binding sites, were obtained from Invivogen (HEK-Blue™ hTLR7, cat #Hkb-htlr7; HEK-Blue™ hTLR8, cat #Hkb-10 htlr8). Stimulation of hTLR7 or hTLR8 in these cells activates NF-κB and AP-1 and induces the production of SEAP which can be quantified using an alkaline phosphatase detection reagent.

Cells were maintained in DMEM growth media containing heat inactivated (10%), Glutamax (2 mM), Penicillin/Streptomycin, Blasticidin (10 μg/ml), Zeocin (100 μg/ml) and Normocin (100 μg/ml) according to the manufacturer suggestion. On day one of the assay, compounds were prepared using 11-point half-log serial dilutions from a 10 mM DMSO stock solution and 50 nL was spotted into 384-well plates (PerkinElmer, cat #6007480). Positive and negative controls were also spotted within the assay plate and were used to determine percent effect during the analysis process. After resuspension in DMEM assay media containing FBS heat inactivated (10%), Glutamax (2 mM) and Penicillin/Streptomycin, 10,000 cells/20 μl/well were added to previously prepared compound plates. Plates were incubated overnight (16-20 hrs) at 37° C. in a 5% CO₂ environment. Prewetted Microclime lids (Labcyte, LLS-0310) were used to prevent evaporation. On day two of the assay, QUANTI-Blue™ detection reagent was prepared by reconstituting QUANTI-Blue™ powder (InvivoGen, Rep-qb1) with 100 ml of sterile water and allowed to equilibrate to 37° C. for 15 minutes. 20 μl of QUANTI-Blue™ detection reagent was added to each well and plates were incubated at room temperature for 180 min. At the end of the incubation, plates were read on an Envision (Perkin Elmer) plate reader capturing absorbance at 650 nm.

Using Positive (tool compound) and Negative (DMSO) controls, the percent (%) effect was calculated for each sample using the following equation:

$$\% effect = 100 - 100 * ( ( Sample - Positive ) / ( Negative - Positive ) )$$

The % effect at each concentration of compound was calculated utilizing the ABase software suite (IBDS) and was relative to the amount of SEAP produced in the positive and negative control wells contained within each assay plate. The concentrations and % effect values for test compounds were fit using a 4-parameter logistic model in ABase and the concentration of compound that produced 50% response (EC₅₀) was calculated.

TABLE 2

| | hTLR7 HEK Blue | hTLR8 HEK Blue |
|---|---|---|
| Compound/ Example Number and Name | EC50 (nM) | EC50 (nM) |
| 1 | 221 | 316 |
| N⁵-(4-(N-(3-(4-Amino-2-butyl-1H-imidazo[4,5- | | |
| c]quinolin-1-yl)-2,2- | | |
| bis(hydroxymethyl)propyl)sulfamoyl)butyl)-N²- | | |
| palmitoyl-L-glutamine | | |
| 2 | 766 | 2066 |
| (S)-1-(N-(3-(4-Amino-2-butyl-1H-imidazo[4,5- | | |
| c]quinolin-1-yl)-2,2- | | |
| bis(hydroxymethyl)propyl)sulfamoyl)-13-oxo- | | |
| 16-palmitamido-3,6,9-trioxa-12- | | |
| azaheptadecan-17-oic acid | | |
| 3 | 1213 | 2471 |
| (S)-1-(4-Amino-2-butyl-1H-imidazo[4,5- | | |
| c]quinolin-1-yl)-2,2-bis(hydroxymethyl)-18- | | |
| oxo-21-palmitamido-8,11,14-trioxa-4,17- | | |
| diazadocosan-22-oic acid | | |
| 4 | 1390 | 2766 |
| (S)-5-(4-(2-((4-((3-(4-Amino-2-butyl-1H- | | |
| imidazo[4,5-c]quinolin-1-yl)-2,2- | | |
| bis(hydroxymethyl)propyl)amino)butyl)amino)- | | |
| 2-oxoethyl)-4-hydroxypiperidin-1-yl)-5-oxo-2- | | |
| palmitamidopentanoic acid | | |
| 5 | 2938 | 3333 |
| 1-(4-(2-(4-(3-(4-Amino-2-butyl-1-(3-hydroxy-2- | | |
| (hydroxymethyl)-2-methylpropyl)-1H- | | |
| imidazo[4,5-c]quinolin-7-yl)propyl)piperazin-1- | | |
| yl)-2-oxoethyl)-4-hydroxypiperidin-1- | | |
| yl)hexadecan-1-one | | |
| 6 | 1026 | 3449 |
| (S)-1-(4-(3-(4-Amino-2-(ethoxymethyl)-1-(3- | | |
| hydroxy-2-(hydroxymethyl)-2-methylpropyl)- | | |
| 1H-imidazo[4,5-c]quinolin-7- | | |
| yl)propyl)piperazin-1-yl)-1,14-dioxo-17- | | |
| palmitamido-4,7,10-trioxa-13-azaoctadecan- | | |
| 18-oic acid | | |

### Lipid Nanoparticle Formulation

### Example 8

### Preparation of Lipid Nanoparticle Formulation

Lipid nanoparticles (LNPs) containing a lipidated TLR 7/8 modulating compound were prepared for use as an adjuvant. Specifically, N⁵-(4-(N-(3-(4-Amino-2-butyl-1H-imidazo[4,5-c]quinolin-1-yl)-2,2-bis(hydroxymethyl)propyl)sulfamoyl)butyl)-N²-palmitoyl-L-glutamine (Compound 1 shown in Table 1 above) was incorporated into the LNPs. The manufacturing process included the formulating of aqueous and organic phases, mixing in a microfluidic device, dialysis, compounding, and sterile filtration.

### Solution Preparation

The first manufacturing step involved formulating the aqueous and organic phases. The aqueous formulation buffer contained 40 mM sodium citrate, pH 4.0±0.5. The aqueous formulation buffer was filtered through a 0.22 μm filter prior to use.

The organic phase contained the following: 10.32 mg/mL of Compound 1, 17.21 mg/mL of ALC-0315, 7.45 mg/ml of Cholesterol, 3.74 mg/ml of DSPC, and 2.13 mg/ml of ACL-0159 (mPEG-DTA). The lipids were dissolved in ethanol q.s. to the desired volume.

### Microfluidic T-Mixing

The LNPs were formed by mixing the aqueous formulation buffer with the organic phase through a microfluidic T-mixer (IDEX® P-727) at ambient room temperature. Specifically, 16.5 mL of aqueous formulation buffer at a volumetric flowrate of 45 mL/min was mixed with 5.5 mL of organic phase at a volumetric flowrate of 15 mL/min. The first 2 mL of effluent was discarded as the ‘leading edge’ to ensure that LNPs were not collected prior to the system reaching steady state. The resulting LNP formulation was a white homogeneous suspension.

### Dialysis

After microfluidic mixing, the LNP formulation was processed via dialysis to remove the ethanol and exchange the buffer to a 10 mM Tris, pH 7.4 buffer. Specifically, 20 mL of the liposome formulation (containing 2.58 mg/ml of Compound 1) was transferred into a 10 kDa, 30 mL volume dialysis cassette. The volume of the LNP formulation transferred into the dialysis cassette was recorded for the purpose of calculating the concentration of Compound 1 after dialysis. The dialysis cassette was floated in a beaker containing 100 times volume (2000 mL) of 10 mM Tris, pH 7.4 buffer stirred at 100 rpm for 2 hours. Then, the buffer was replaced by a fresh 10 mM Tris, pH 7.4 buffer for another 22 hour dialysis.

### Compounding

After dialysis, the Compound 1 concentration was calculated based on the volume change of the LNP formulation and was adjusted to 1.147 mg/mL. The LNP formulation was then spiked with 10 mM Tris, 1.2 M sucrose, pH 7.4 buffer at a volume ratio of 3:1 (liposome:buffer). Specifically, the LNP formulation volume after dialysis was 45 mL and the Compound 1 concentration was 1.147 mg/mL. The 45 mL liposome formulation volume was spiked with 15 mL of 10 mM Tris, 1.2 M sucrose, pH 7.4 buffer to obtain an LNP formulation containing 0.86 mg/ml of Compound 1.

### Sterile Filtration

Following the spiking with sucrose, the LNP formulation underwent sterilizing filtration and was stored at 2-8° C. after filtration. Specifically, the LNP formulation was collected into a 30 mL sterile luer lock syringe. A Sartorius® 0.22 μm polyethersulfone (PES) membrane was connected to the syringe. The LNP formulation was filtered through the membrane into a sterile bottle. The process was repeated until a total of 60 mL of LNP formulation was filtered.

Preparation of Liposomal Adjuvant Formulation with QS-21

In order to prepare LNPs with QS-21, after sterile filtration (described above) the LNP formulation was aseptically compounded with QS-21. Specifically, the liposome formulation was diluted in 10 mM Tris, 300 mM sucrose, pH 7.4 buffer to achieve a Compound 1 concentration of 0.1376 mg/mL. The diluted LNP formulation was dispensed into 5 appropriate compounding vessels and allowed to equilibrate to ambient room temperature. The LNP formulation was gently mixed to form a slight vortex. 5 stock solutions of QS-21 were prepared at 0.018-0.8 mg/ml of QS-21 in 10 mM Tris, 300 mM sucrose, pH 7.4 buffer. Compounding was performed to achieve concentration targets of 0.0688 mg/mL of Compound 1 and 0.009-0.4 mg/ml of QS-21, respectively. The stock solutions were respectively added to the LNP formulations using a pipette at 1:1 volume ratio with subsequent gentle mixing for 2 hours at room temperature. The LNPs with QS-21 were white homogeneous suspensions. After compounding, the LNPs with QS-21 were stored at 2-8° C.

### Size and PDI Measurement

The size and PDI of the LNPs with and without QS-21 was measured by Malvern dynamic light scattering (DLS). The results are shown in Table 3, below. The component molar ratio in all the formulations was as follows: ALC-0315: DSPC:Cholesterol:ALC-0159=0.475:0.1:0.407:0.018.

For the LNP formulation with a QS-21 concentration of 0.1 mg/mL and a Compound 1 concentration of 0.0688 mg/mL, the size of the LNPs increased from 92 nm to 125 nm and the PDI increased from 0.206 to 0.404 over the course of 3 months.

The size and PDI of the LNP formulations without QS-21 was stable. The size and PDI of the LNP formulations with a QS-21 concentration below 0.1 mg/ml and a Compound 1 concentration of 0.0688 mg/mL was also stable.

The size and PDI was not monitored over the 3 month time frame for the liposomes with a QS-21 concentration of 0.4 mg/mL due to the large LNP size and PDI at TO, as well as the observed hemolytic activity, shown in Table 4.

TABLE 3
Size and PDI of LNPs over 3 months

| | Compound | QS-21 | Size (nm) | | | PDI | | |
|---|---|---|---|---|---|---|---|---|
| # | 1 (mg/mL) | (mg/mL) | T0 | 1M | 3M | T0 | 1M | 3M |
| 1 | 0.86 | 0 | 64 | 68 | 74 | 0.077 | 0.103 | 0.106 |
| 2 | 0.0688 | 0.009 | 69 | 71 | 75 | 0.112 | 0.116 | 0.126 |
| 3 | 0.0688 | 0.023 | 70 | 71 | 75 | 0.117 | 0.095 | 0.112 |
| 4 | 0.0688 | 0.045 | 73 | 71 | 74 | 0.113 | 0.122 | 0.123 |
| 5 | 0.0688 | 0.100 | 92 | 113 | 125 | 0.206 | 0.412 | 0.404 |
| 6 | 0.0688 | 0.400 | 240 | N/A | N/A | 0.653 | N/A | N/A |

### Hemolytic Assay

The cytotoxicity of LNPs with various concentrations of QS-21 was tested in a hemolytic assay. The formulations tested corresponded to samples 2-6 as shown in Table 3 above. As shown in Table 4, below, hemolytic activity was only observed when the QS-21 concentration was 0.4 mg/mL.

TABLE 4
Hemolytic Activity of LNPs with QS-21

| # | Compound 1 (mg/mL) | QS-21 (mg/mL) | Hemolytic activity |
|---|---|---|---|
| 2 | 0.0688 | 0.009 | No |
| 3 | 0.0688 | 0.023 | No |
| 4 | 0.0688 | 0.045 | No |
| 5 | 0.0688 | 0.100 | No |
| 6 | 0.0688 | 0.400 | Yes |

### Example 9

Preparation of LNPs with E. coli modRNA

LNPs containing Compound 1 and a modRNA molecule encoding the E. coli FimH protein of SEQ ID NO: 2 (modRNA LNPs) were prepared. Two preparation methods were used. In the first preparation method, oleic acid was added in the organic phase, as described below. In the second preparation method, oleic acid was spiked in after dialysis of the LNP formulation.

### Preparation Method 1 (Formulations 1-3)

### Solution Preparation

The first manufacturing step involved formulating the aqueous and organic phases. The aqueous formulation buffer contained 40 mM sodium citrate, pH 4.0±0.5. The aqueous formulation buffer was filtered through a 0.22 μm filter prior to use.

The components of the organic phase of the 3 different formulations are described below. In all 3 formulations, the lipids were dissolved in ethanol q.s. to the desired volume.
Formulation 1: 0.21 mg/ml of Compound 1, 17.21 mg/mL of ALC-0315, 2.99 mg/ml of Cholesterol, 4.81 mg/ml of β-Sitosterol, 3.74 mg/ml of DSPC, 2.17 mg/ml of ACL-0159 (mPEG-DTA), and 8.88 mg/ml of oleic acid
Formulation 2: 1.03 mg/ml of Compound 1, 17.21 mg/mL of ALC-0315, 2.99 mg/ml of Cholesterol, 4.81 mg/ml of β-Sitosterol, 3.74 mg/ml of DSPC, 2.17 mg/ml of ACL-0159 (mPEG-DTA), and 8.88 mg/ml of oleic acid.
Formulation 3: 4.13 mg/ml of Compound 1, 17.21 mg/mL of ALC-0315, 2.99 mg/ml of Cholesterol, 4.81 mg/ml of β-Sitosterol, 3.74 mg/ml of DSPC, 2.17 mg/ml of ACL-0159 (mPEG-DTA), and 8.88 mg/ml of oleic acid

### Microfluidic T-Mixing

The adjuvanted LNP formulation was formed by mixing the aqueous formulation buffer with the organic phase through a microfluidic T-mixer (IDEX® U-429) at ambient room temperature. Specifically, 3.6 mL of aqueous formulation buffer at a volumetric flowrate of 45 mL/min was mixed with 1.2 mL of organic phase at a volumetric flowrate of 15 mL/min. The first 0.5 mL of effluent was discarded as the ‘leading edge’ to ensure that liposome is not collected prior to the system reaching steady state. The resulting liposome was a white homogeneous suspension.

### Dialysis

After microfluidic mixing, the adjuvanted LNP formulation was processed via dialysis to remove ethanol and exchange the buffer to a 10 mM Tris, pH 7.4 buffer. Specifically, 3 mL of the adjuvanted LNP formulation was transferred into a 10 kDa, 3 mL volume dialysis cassette. The volume of the adjuvanted LNP formulation transferred into the dialysis cassette was recorded for the purpose of calculating the RNA concentration after dialysis. The dialysis cassette was floated in a beaker containing 100 times volume (300 mL) of 10 mM Tris, pH 7.4 buffer and stirred at 100 rpm for 2 hours. Then, the buffer was replaced by a fresh 10 mM Tris, pH 7.4 buffer for another 22 hours dialysis.

### Compounding

After dialysis, the RNA concentration was calculated based on the volume change of the adjuvanted LNP formulation and was adjusted to 0.133 mg/mL using 10 mM Tris, pH 7.4 buffer. The adjusted adjuvanted LNP formulation was spiked with 10 mM Tris, 1.2 M sucrose, pH 7.4 buffer at a volume ratio of 3:1 (adjuvanted LNP formulation: buffer). Specifically, the adjuvanted LNP formulation after dialysis was 4.66 mL and the RNA concentration was 0.174 mg/mL. 4.66 mL of adjuvanted LNP formulation was diluted with 3.04 mL of 10 mM Tris, pH 7.4 buffer and then spiked with 2.57 mL of 10 mM Tris, 1.2 M sucrose, pH 7.4 buffer to form the adjuvanted LNP formulation containing 0.1 mg/ml of RNA.

### Sterile Filtration

Following the spiking with sucrose, the adjuvanted LNP formulation underwent sterilizing filtration and was stored at 2-8° C. after filtration. Specifically, the adjuvanted LNP formulation was collected into a 30 mL sterile luer lock syringe. A Sartorius® 0.22 μm polyethersulfone (PES) membrane was connected to the syringe. The adjuvanted LNP formulation was filtered through the membrane into a sterile bottle. The process was repeated until a total of 10.27 ml of adjuvanted LNP formulation was filtered.

### Preparation Method 2 (Formulations 4-6)

### Solution Preparation

The first manufacturing step involved formulating the aqueous and organic phases. The aqueous formulation buffer contained 40 mM sodium citrate, pH 4.0±0.5. The aqueous formulation buffer was filtered through a 0.22 μm filter prior to use.

The components of the organic phase of the 3 different formulations are described below. In all 3 formulations, the lipids were dissolved in ethanol q.s. to the desired volume.
Formulation 1: 0.21 mg/ml of Compound 1, 17.21 mg/ml of ALC-0315, 2.98 mg/ml of Cholesterol, 4.79 mg/ml of β-Sitosterol, 3.74 mg/ml of DSPC, and 2.13 mg/ml of ACL-0159 (mPEG-DTA)
Formulation 2: 1.03 mg/ml of Compound 1, 17.21 mg/ml of ALC-0315, 2.98 mg/ml of Cholesterol, 4.79 mg/ml of β-Sitosterol, 3.74 mg/ml of DSPC, and 2.13 mg/mL of ACL-0159 (mPEG-DTA)
Formulation 3: 4.13 mg/ml of Compound 1, 17.21 mg/mL of ALC-0315, 2.98 mg/ml of Cholesterol, 4.79 mg/ml of β-Sitosterol, 3.74 mg/ml of DSPC, and 2.13 mg/ml of ACL-0159 (mPEG-DTA)

### Microfluidic T-Mixing

The adjuvanted LNP formulation was formed by mixing the aqueous formulation buffer with the organic phase through a microfluidic T-mixer (IDEX® U-429) at ambient room temperature. Specifically, 9 mL of aqueous formulation buffer at a volumetric flowrate of 45 mL/min was mixed with 3 mL of organic phase at a volumetric flowrate of 15 mL/min. The first 1 mL of the effluent was discarded as the ‘leading edge’ to ensure that liposome was not collected prior to the system reaching steady state. The resulting liposome was a white homogeneous suspension.

### Dialysis

After microfluidic mixing, the adjuvanted LNP formulation was processed via dialysis to remove ethanol and exchange the buffer to a 10 mM Tris, pH 7.4 buffer. Specifically, 9.5 mL of the adjuvanted LNP formulation was transferred into a 10 kDa, 15 mL volume dialysis cassette. The volume of the adjuvanted LNP formulation transferred into the dialysis cassette was recorded for the purpose of calculating the RNA concentration after dialysis. The dialysis cassette was floated in a beaker containing 100 times volume (500 mL) of 10 mM Tris, pH 7.4 buffer and stirred at 100 rpm for 2 hours. Then, the buffer was replaced by a fresh 10 mM Tris, pH 7.4 buffer for another 22 hours dialysis.

### Compounding

After dialysis, 10 mg/mL of sodium oleate in 10 mM Tris, pH 7.4 buffer was added to the adjuvanted LNP formulation at a weight ratio of 1:8 (RNA to sodium oleate). The RNA concentration was calculated based on the volume change of the adjuvanted LNP formulation during dialysis and the volume of sodium oleate added, then adjusted to 0.133 mg/mL using 10 mM Tris, pH 7.4 buffer. The adjusted adjuvanted LNP formulation was then spiked with 10 mM Tris, 1.2 M sucrose, pH 7.4 buffer at volume ratio of 3:1 (adjuvanted LNP: buffer). Specifically, the adjuvanted LNP formulation after dialysis was 19 mL and the RNA concentration was 0.15 mg/mL. 2.28 mL of 10 mg/ml of sodium oleate in 10 mM Tris, pH 7.4 buffer was added to the adjuvanted LNP formulation. Then, the adjuvanted LNP formulation was diluted with 0.1 mL of 10 mM Tris, pH 7.4 buffer and then spiked with 7.13 mL of 10 mM Tris, 1.2 M sucrose, pH 7.4 buffer to get the adjuvanted LNP formulation containing 0.1 mg/mL of RNA.

### Sterile Filtration

Following the spiking with sucrose, the adjuvanted LNP formulation underwent sterilizing filtration and was stored at 2-8° C. after filtration. Specifically, the adjuvanted LNP formulation was collected into a 30 mL sterile luer lock syringe. A Sartorius® 0.22 μm polyethersulfone (PES) membrane was connected to the syringe. The adjuvanted LNP formulation was filtered through the membrane into a sterile bottle. The process was repeated until a total of 28.51 mL of adjuvanted LNP formulation was filtered.

### Size and Pdi Measurement

In vitro assessments of the modRNA LNPs were completed. The assessments of the physical properties of the LNPs are shown in Table 5, below, and include determinations of size, polydispersity index (PDI), and percentage of encapsulation efficiency (% EE). Particle size and PDI were measured using Malvern dynamic light scattering (DLS). Percent encapsulation efficiency for the FimH modRNA was determined using RiboGreen® (Invitrogen®).

TABLE 5
Physical Properties of modRNA LNPs

| | | | | | | Measured | | | |
|---|---|---|---|---|---|---|---|---|---|
| | Target | Measured RNA | | | Target | Compound 1 | % EE | | |
| | RNA | concentration (mg/mL) | | % EE | Compound 1 | concentration | Compound | Size | |
| # | (mg/mL) | Free RNA | Total RNA | RNA | (mg/mL) | (mg/mL) | 1 | (nm) | PDI |
| 1 | 0.1 | 0.006 | 0.056 | 90 | 0.017 | 0.002 | 12 | 78 | 0.07 |
| 2 | 0.1 | 0.007 | 0.061 | 89 | 0.086 | 0.007 | 8 | 79 | 0.06 |
| 3 | 0.1 | 0.01 | 0.065 | 84 | 0.344 | 0.024 | 7 | 80 | 0.09 |
| 4 | 0.1 | 0.040 | 0.112 | 65 | 0.017 | 0.02 | 118 | 124 | 0.07 |
| 5 | 0.1 | 0.073 | 0.102 | 29 | 0.086 | 0.08 | 93 | 137 | 0.12 |
| 6 | 0.1 | 0.109 | 0.120 | 10 | 0.344 | 0.31 | 90 | 100 | 0.10 |

From these results, it was determined that in formulations 1-3, adding oleic acid in the organic phase resulted in a low encapsulation efficiency of Compound 1, ranging from 7% to 12%. In contrast, in formulation 4-6, spiking sodium oleate after the dialysis step did not affect the encapsulation efficiency of Compound 1. In formulations 4-6, the Compound 1 encapsulation efficiencies were above 90%.

In formulations 1-3, the encapsulation efficiency of RNA was above 80%. In formulations 4-6, the encapsulation efficiency of RNA decreased with an increasing concentration of Compound 1, from 65% encapsulation efficiency to 10% encapsulation efficiency.

### In-Vitro Expression (IVE)

The in-vitro expression (IVE) of the FimH protein was tested in human foreskin fibroblast (HFF-1) (ATCC®) and THP-1 human leukemia monocytic cells (ATCC®) and is shown in Table 6, below. Cells were seeded into 12-well plates. The following day, cells were transfected with multiple dilutions of LNP samples and incubated overnight at 37° C. The next day, cells were harvested using TrypLE™ Express enzyme (Gibco™). The harvested cells were incubated with a Live-Dead staining Dye, washed once with wash buffer, and then fixed and permeabilized. Cells were incubated with the primary and secondary antibodies diluted in wash buffer. Finally, the cells were resuspended in wash buffer, and expression was measured using a BD FACSLyric™ flow cytometry system. The data was analyzed using FLOWJO™ or OMIQ™ software.

The values obtained for percentage of cells with FimH protein expression and EC50 are shown in Table 6. Control LNPs without a TLR 7/8 modulating molecule, and LNPs with Compound 1 (Formulations 4-6 shown in Table 5, above) were tested.

TABLE 6
IVE of LNPs with E. coli FimH modRNA

| | | | | IVE (HFF-1 cells) | | IVE (THP-1 cells) | |
|---|---|---|---|---|---|---|---|
| | | | | % Protein | | % Protein | |
| Formulation | | Concentration | | Expression | | Expression | |
| Description | | RNA | Compound | (1.37 | EC50 | (12.5 | EC50 |
| and Number | | (mg/mL) | 1 (mg/mL) | ng/well) | (ng/well) | ng/well) | (ng/well) |
| LNPs, no TLR | | 0.1 | 0 | 72 | 0.6 | 79 | 4.6 |
| 7/8 (Control) | | | | | | | |
| LNPs with | 4 | 0.1 | 0.017 | 65 | 1.9 | 63 | 7.4 |
| TLR 7/8 | 5 | 0.1 | 0.086 | 55 | 1.2 | 62 | 8.8 |
| | 6 | 0.1 | 0.344 | 50 | 1.3 | 57 | 8.8 |

In both HFF-1 and THP-1 cells, when the concentration of Compound 1 increased, the in-vitro expression of FimH decreased. These results indicated that the addition of higher concentrations of Compound 1 could result in lower FimH expression.

### In-Vitro Cytokine Responses

In-vitro cytokine responses for TNF-α, IL-6, and IFN-β were evaluated in a mouse RAW 264.7 macrophage cell line (ATCC®). In-vitro cytokine response for IL-8 was evaluated in a THP-1 cell line (ATCC®). Cells were seeded into 24-well plates. The following day, cells were transfected with multiple dilutions of LNP samples and incubated overnight at 37° C. The next day, cell culture supernatants were collected and subjected to ELISA. To evaluate TNF-α, IL-6, IFN-β, and IL-8 responses upon LNP transfection in cells, mouse ELISA kits from Abcam™ were used according to the manufacturer's protocols. The signal was immediately detected using the SpectraMax® plate reader at 450 nm, and the data was analyzed using GRAPHPAD PRISM®.

In-vitro cytokine responses for TNF-α, IL-6, IFN-β, and IL-8 are summarized in Table 7, below. Cytokine responses including TNF-α, IL-6 and INF-β are stimulated in all the groups with Compound 1 compared to the LNP control group. The IL-8 cytokine response was increased over the LNP control group for formulations 5 and 6.

TABLE 7
In-vitro Cytokine Response of LNPs with E. coli modRNA

| | | Concentration | | Cytokine Response (pg/mL) | | | |
|---|---|---|---|---|---|---|---|
| Formulation | | RNA | Compound | (RAW) | (RAW) | (RAW) | (THP-1) |
| Description | | (mg/mL) | 1 (mg/mL) | TNF-α | IL-6 | INF-β | IL-8 |
| LNPs Control, no | | 0.1 | 0 | 9 | 0 | 27 | 121 |
| TLR 7/8 | | | | | | | |
| LNPs | 4 | 0.1 | 0.017 | 2229 | 81 | 130 | 101 |
| with | 5 | 0.1 | 0.086 | 2134 | 194 | 200 | 149 |
| TLR | 6 | 0.1 | 0.344 | 1560 | 242 | 254 | 309 |
| 7/8 | | | | | | | |

1. A compound of Formula (I): 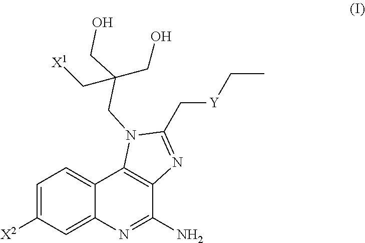
or a pharmaceutically acceptable salt thereof, wherein:
Y is —O— or —CH₂—; and
one of X¹ and X² is H, and the other of X¹ and X² has a formula selected from the group consisting of formula (a), formula (b), and formula (c): 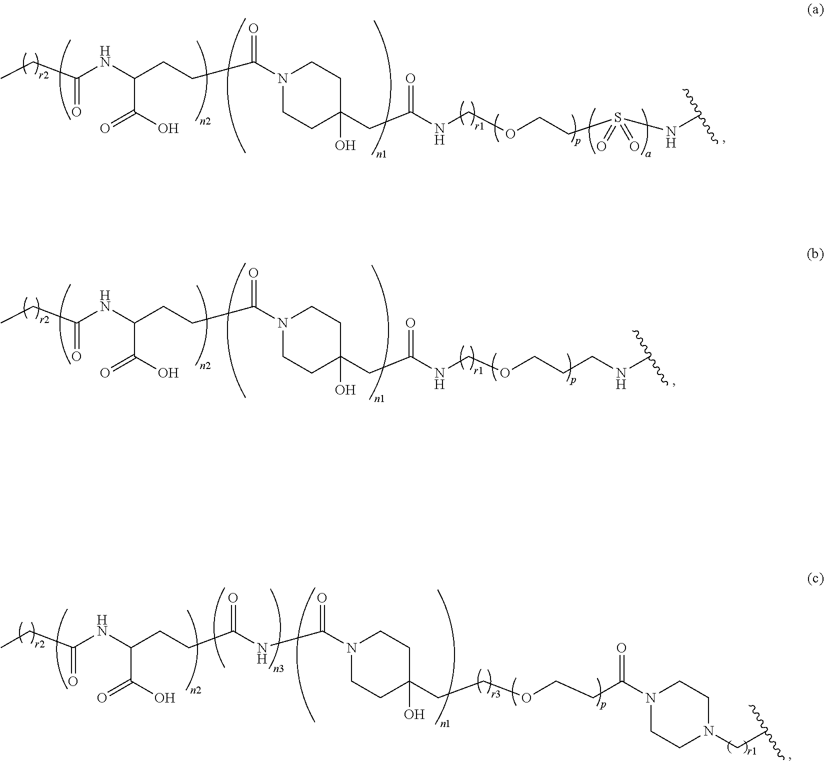
wherein:
a is 0 or 1;
r1 is an integer from 2 to 6;
r2 is an integer from 10 to 20;
r3 is an integer from 0 to 6;
n1 is 0 or 1 and n2 is 0 or 1, wherein at least one of n1 and n2 is 1;
n3 is 0 or 1; and
p is an integer from 0 to 6.

2-52. (canceled)

53. A lipid nanoparticle (LNP) formulation, comprising a plurality of LNPs, wherein the LNPs comprise a compound of Formula (I): 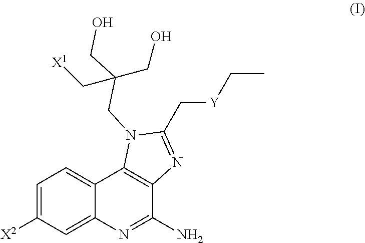
or a pharmaceutically acceptable salt thereof, wherein:
Y is —O— or —CH₂—; and
one of X¹ and X² is H, and the other of X¹ and X² has a formula selected from the group consisting of formula (a), formula (b), and formula (c): 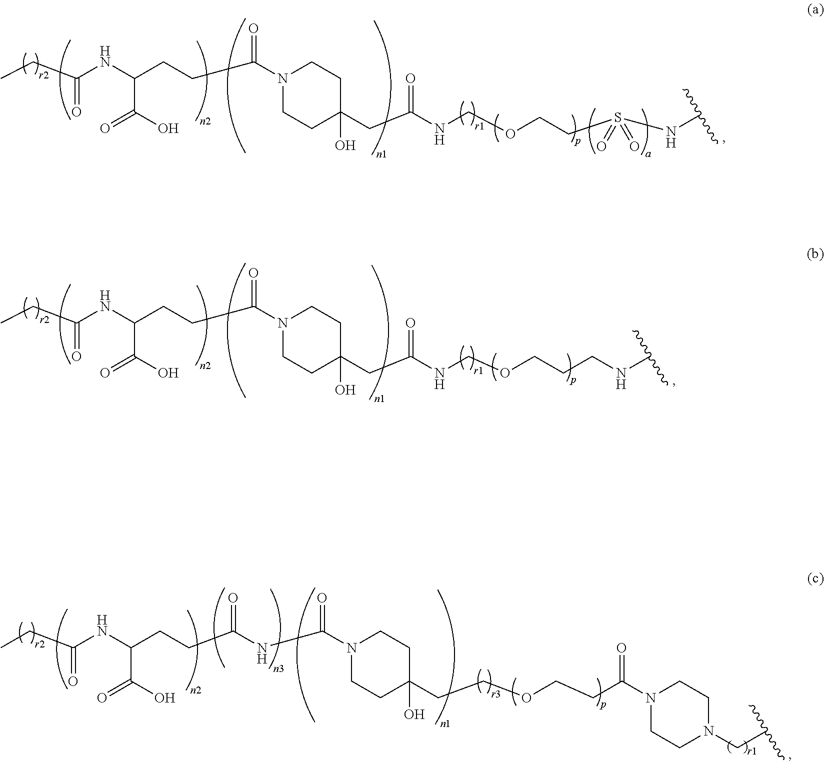
wherein:
a is 0 or 1;
r1 is an integer from 2 to 6;
r2 is an integer from 10 to 20;
r3 is an integer from 0 to 6;
n1 is 0 or 1 and n2 is 0 or 1, wherein at least one of n1 and n2 is 1;
n3 is 0 or 1; and
p is an integer from 0 to 6.

54. The LNP formulation of claim 53, wherein X¹ has formula a.

55. The LNP formulation of claim 54, wherein a is 1, n1 is 0, and n2 is 1, r1 is 4, and p is 0.

56. (canceled)

57. (canceled)

58. The LNP formulation of claim 54, wherein a is 1, n1 is 0, and n2 is 1, r1 is 2, and p is 3.

59. (canceled)

60. The LNP formulation of claim 53, wherein X¹ has formula b.

61. The LNP formulation of claim 60, wherein n1 is 0, n2 is 1, r1 is 2, and p is 3.

62. (canceled)

63. The LNP formulation of claim 60, wherein n1 is 1, n2 is 1, r1 is 3, and p is 0.

64. (canceled)

65. The LNP formulation of claim 53, wherein X² has formula c.

66. The LNP formulation of claim 65, wherein n1 is 1, n2 is 0, n3 is 0, r1 is 3, r3 is 0, and p is 0.

67. (canceled)

68. The LNP formulation of claim 65, wherein n1 is 0, n2 is 1, n3 is 1, r1 is 3, r3 is 2, and p is 3.

69. (canceled)

70. The LNP formulation of claim 53, wherein r2 is 13, 14, or 15.

71. (canceled)

72. A lipid nanoparticle (LNP) formulation, comprising a plurality of LNPs, wherein the LNPs comprise a compound, which is N⁵-(4-(N-(3-(4-Amino-2-butyl-1H-imidazo[4,5-c]quinolin-1-yl)-2,2-bis(hydroxymethyl)propyl)sulfamoyl)butyl)-N²-palmitoyl-L-glutamine, or a pharmaceutically acceptable salt thereof; or
a lipid nanoparticle (LNP) formulation, comprising a plurality of LNPs, wherein the LNPs comprise a compound, which is (S)-1-(N-(3-(4-Amino-2-butyl-1H-imidazo[4,5-c]quinolin-1-yl)-2,2-bis(hydroxymethyl)propyl)sulfamoyl)-13-oxo-16-palmitamido-3,6,9-trioxa-12-azaheptadecan-17-oic acid, or a pharmaceutically acceptable salt thereof; or
a lipid nanoparticle (LNP) formulation, comprising a plurality of LNPs, wherein the LNPs comprise a compound, which is (S)-1-(4-Amino-2-butyl-1H-imidazo[4,5-c]quinolin-1-yl)-2,2-bis(hydroxymethyl)-18-oxo-21-palmitamido-8,11,14-trioxa-4,17-diazadocosan-22-oic acid, or a pharmaceutically acceptable salt thereof; or
a lipid nanoparticle (LNP) formulation, comprising a plurality of LNPs, wherein the LNPs comprise a compound, which is (S)-5-(4-(2-((4-((3-(4-Amino-2-butyl-1H-imidazo[4,5-c]quinolin-1-yl)-2,2-bis(hydroxymethyl)propyl)amino)butyl)amino)-2-oxoethyl)-4-hydroxypiperidin-1-yl)-5-oxo-2-palmitamidopentanoic acid, or a pharmaceutically acceptable salt thereof; or
a lipid nanoparticle (LNP) formulation, comprising a plurality of LNPs, wherein the LNPs comprise a compound, which is 1-(4-(2-(4-(3-(4-Amino-2-butyl-1-(3-hydroxy-2-(hydroxymethyl)-2-methylpropyl)-1H-imidazo[4,5-c]quinolin-7-yl)propyl) piperazin-1-yl)-2-oxoethyl)-4-hydroxypiperidin-1-yl)hexadecan-1-one, or a pharmaceutically acceptable salt thereof; or
a lipid nanoparticle (LNP) formulation, comprising a plurality of LNPs, wherein the LNPs comprise a compound, which is (S)-1-(4-(3-(4-Amino-2-(ethoxymethyl)-1-(3-hydroxy-2-(hydroxymethyl)-2-methylpropyl)-1H-imidazo[4,5-c]quinolin-7-yl)propyl) piperazin-1-yl)-1,14-dioxo-17-palmitamido-4,7,10-trioxa-13-azaoctadecan-18-oic acid, or a pharmaceutically acceptable salt thereof.

73-77. (canceled)

78. The LNP formulation of claim 53, wherein the LNPs further comprise:
a) an ionizable cationic lipid;
b) cholesterol, a cholesterol analog, or cholesterol and a cholesterol analog;
c) a neutral lipid; and
d) a polymer-conjugated lipid.

79. The LNP formulation of claim 78, wherein the LNPs further comprise RNA.

80. (canceled)

81. The LNP formulation of claim 79, wherein the RNA comprises a 5′ cap, a 5′ UTR, a 3′ UTR, and a poly-A tail.

82-87. (canceled)

88. The LNP formulation of claim 78, wherein the LNPs further comprise a saponin selected from the group consisting of QS-7 QS-18, and QS-21.

89-91. (canceled)

92. The LNP formulation of claim 78, wherein the ionizable cationic lipid is ALC-0315 (4-hydroxybutyl)azanediyl)bis(hexane-6,1-diyl)bis(2-hexyldecanoate) (ALC-0315) having the structure: 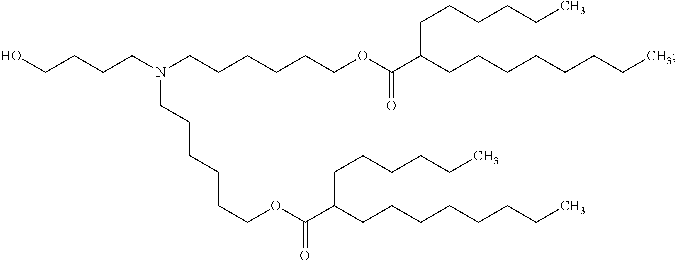 [SMILES: CCCCCCCCC(CCCCCC)C(=O)OCCCCCCN(CCCCO)CCCCCCOC(=O)C(CCCCCC)CCCCCCCC]
or
2-hexyldecyl 6-[(2-{[4-(heptylcarbonylamino)butyl]-N-methylamino}ethyl) [5-(2-hexyldecyloxycarbonyl)pentyl]amino]hexanoate (ALC-0515) having the structure:

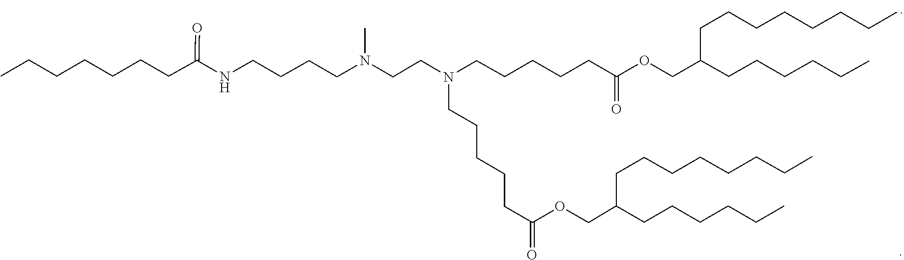 [SMILES: CCCCCCCCC(CCCCCC)COC(=O)CCCCCN(CCCCCC(=O)OCC(CCCCCC)CCCCCCCC)CCN(C)CCCCNC(=O)CCCCCCC]

93-95. (canceled)

96. The LNP formulation of claim 78, wherein the LNPs comprise cholesterol and a cholesterol analog, and wherein the cholesterol analog is β-sitosterol.

97-100. (canceled)

101. The LNP formulation of claim 78, wherein the neutral lipid is 1,2-distearoyl-sn-glycero-3-phosphocholine (DSPC).

102-103. (canceled)

104. The LNP formulation of claim 78, wherein the polymer-conjugated lipid is ALC-0159 (2-[(polyethylene glycol)-2000]-N,N-ditetradecylacetamide) having the structure:

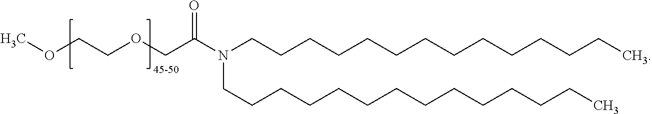

105-111. (canceled)

112. The LNP formulation of claim 78, wherein the LNPs have a mean diameter size between about 60 nm and about 140 nm, and a PDI between about 0.05 and about 0.2.

113-116. (canceled)

117. The LNP formulation of claim 78, wherein the ionizable cationic lipid comprises between about 40 mol % and about 50 mol % of the total lipid, the cholesterol analog, or the cholesterol and the cholesterol analog comprise between about 35 mol % and about 45 mol % of the total lipid, the neutral lipid comprises between about 5 mol % and about 15 mol % of the total lipid, and the polymer-conjugated lipid comprises between about 0.5 mol % and about 10 mol % of the total lipid.

118-127. (canceled)

128. An immunogenic composition comprising the LNP formulation of claim 78, wherein the LNPs comprise RNA, and wherein the RNA comprises at least one open reading frame (ORF) encoding an immunogen of interest.

129-152. (canceled)

153. A method of inducing an immune response in a subject against the immunogen of interest, comprising administering to the subject the immunogenic composition of claim 128; or
a method for immunizing a subject against a disease or disorder caused by or associated with the immunogen of interest, comprising administering to the subject the immunogenic composition of claim 128; or
a method for preventing a disease or disorder caused by or associated with the immunogen of interest in a subject, comprising administering to the subject the immunogenic composition of claim 128; or
a method for treating a disease or disorder caused by or associated with the immunogen of interest in a subject, comprising administering to the subject the immunogenic composition of claim 128; or
a method for increasing an immune response to the immunogen of interest in a subject, comprising administering to the subject the immunogenic composition of claim 128.

154-176. (canceled)

177. A method of making the LNP formulation of claim 78, comprising the steps of:
(i) dissolving the compound, ionizable cationic lipid, cholesterol and/or cholesterol analog, neutral lipid, and polymer-conjugated lipid in an organic solvent to form an organic phase;
(ii) dissolving the RNA in water or buffer to form an aqueous phase; and
(iii) mixing the organic phase and the aqueous phase to form the LNP formulation.

178-187. (canceled)
